(19) United States
(12) Patent Application Publication
(10) Pub. No.: US 2008/0280955 A1
(43) Pub. Date: Nov. 13, 2008

(54) Methods and compositions for screening and treatment of disorders of blood glucose regulation

(75) Inventor: Mark A. McCamish, Cupertino, CA (US)

Correspondence Address: PERLEGEN SCIENCES, INC.;LEGAL DEPARTMENT, 2021 STIERLIN COURT, MOUNTAIN VIEW, CA, 94043, US

(73) Assignee: Perlegen Sciences, Inc., Mountain View, CA (US)

(21) Appl. No.: 11/541,495

(22) Filed: Sep. 29, 2006

Related U.S. Application Data
(60) Provisional application No. 60/722,357, filed on Sep. 30, 2005.
(60) Provisional application No. 60/722,636, filed on Sep. 30, 2005.

(51) Int. Cl. A61K 31/4436; C12Q 1/68; C07H 21/04; C07K 2/00

(52) U.S. Cl. 514/342; 435/6; 536/24.5; 530/300

(57) ABSTRACT

In one aspect, the invention provides a method of screening and, optionally, treatment of an individual suffering from an insulin resistance disorder by screening an individual in need of treatment for an insulin resistance disorder for one or more genetic variations indicating a predisposition to a response to an insulin sensitizer; and, optionally, administering or not administering an insulin sensitizer to the individual based on the results of the screening. The insulin sensitizer for which the individual is screened and the insulin sensitizer that is administered or not administered may be the same or different. In another aspect, the invention provides methods comprising identifying one or more genetic variations, e.g., one or more single nucleotide polymorphisms, that at least partly differentiate between a subset of a plurality of individuals who experience a response when administered an insulin sensitizer, and a subset of said plurality of individuals who do not experience a response when administered the insulin sensitizer. The invention also provides nucleic acids, polypeptides, antibodies, kits, and business methods associated with these screening and association methods.

### CROSS-REFERENCES TO RELATED APPLICATIONS

The present application is a nonprovisional of and claims the benefit of U.S. Provisional Application No. 60/722,357 and U.S. Provisional Application No. 60/722,636, both filed Sep. 30, 2005, and both of which are incorporated herein by reference in their entireties for all purposes.

### BACKGROUND OF THE INVENTION

Recent advances in genetic analysis, e.g., genotyping, indicate that variations in both the natural history of various diseases, as well as responses to treatments, including drug treatment, are linked to genetic variations among individuals. As an example, disorders of blood glucose regulation, including insulin resistance disorders, are a serious and growing health concern worldwide. These disorders, which include Type I and Type II diabetes, Syndrome X, and obesity are treatable by a variety of lifestyle changes, as well as by drugs. Of the latter, one of the most effective categories is the peroxisome proliferator-activated receptor (PPAR) modulators, especially PPAR-gamma agonists or partial agonists. However, an obstacle to the optimal use of drugs to treat disorders of blood glucose regulations is the incidence of adverse effects, which are in some cases serious enough to cause use of the drug to be discontinued. For example, troglitazone, which was marketed in the U.S. starting in April, 1997 for the treatment of Type II diabetes, was voluntarily withdrawn in March 2000 due to incidents of idiosyncratic liver damage. Although these incidents occurred with a frequency of only about 1 in 100,000 patients, their severity was such that the drug was withdrawn. More common adverse effects such as edema, weight gain, and adverse effects on lipid profiles, while not as severe as idiosyncratic liver damage, can nonetheless limit the therapeutic potential of insulin resistance modulators.

Generally, distinct subgroups of patients respond to a given drug with a given adverse effect. Experience with adverse effect profiles for other drugs indicates that the phenotype of proclivity for a given adverse effect is a reflection of genotype. Great progress has been made in relating genotype to phenotype in regards to disease etiology and response to treatment. See, e.g., U.S. patent application Ser. No. 11/043,689, filed Jan. 24, 2005, entitled “Associations Using Genotypes and Phenotypes;” U.S. Provisional Application Ser. No. 60/280,530, filed Mar. 30, 2001, entitled “Identifying Human SNP Haplotypes, Informative SNPs and Uses Thereof”; U.S. Provisional Application Ser. No. 60/313,264 filed Aug. 17, 2001, entitled “Identifying Human SNP Haplotypes, Informative SNPs and Uses Thereof”; U.S. Provisional Application Ser. No. 60/327,006, filed Oct. 5, 2001, entitled “Identifying Human SNP Haplotypes, Informative SNPs and Uses Thereof”; U.S. Provisional Application Ser. No. 60/332,550, filed Nov. 26, 2002, entitled “Methods for Genomic Analysis”; U.S. application Ser. No. 10/106,097, filed Mar. 26, 2002, entitled “Methods for Genomic Analysis”; U.S. Pat. No. 6,897,025, issued May 24, 2005, entitled “Genetic Analysis Systems and Methods”; and U.S. application Ser. No. 10/284,444, filed Oct. 31, 2002, entitled “Human Genomic Polymorphisms”, U.S. Provisional Patent Application No. 60/648,957, filed Jan. 31, 2005, entitled “Compositions and Methods For Treating, Preventing, and Diagnosing Alzheimer's Disease,” U.S. application Ser. No. 10/956,224, filed Sep. 30, 2004, entitled “Methods for genetic analysis,” U.S. Provisional Patent Application No. 60/653,672, filed Feb. 16, 2005, entitled “Parkinson's Disease-Related Disease Compositions and Methods,” U.S. application Ser. No. 10/768,788, filed Jan. 30, 2004, entitled “Apparatus and Methods for Analyzing and Characterizing Nucleic Acid Sequences,” U.S. application Ser. No. 10/351,973, filed Jan. 27, 2003, entitled “Apparatus and Methods for Determining Individual Genotypes,” U.S. application Ser. No. 10/970,761, filed Nov. 20, 2004, entitled “Improved Analysis Methods and Apparatus for Individual Genotyping,” U.S. application Ser. No. 10/786,475, filed Feb. 24, 2004, entitled “Analysis Methods for Individual Genotyping,” and U.S. Provisional Patent Applications Nos. 60/643,006, 60/635,281 filed Jan. 11, 2005, and Dec. 9, 2004, respectively, both entitled “Markers for Metabolic Syndrome Obesity and Insulin Resistance,” the disclosures all of which are specifically incorporated herein by reference. The present invention builds upon this previous work.

### SUMMARY OF THE INVENTION

The present application discloses methods for screening an individual suffering from a disorder of blood glucose regulation, e.g., an insulin resistance disorder, that include screening the individual for a genetic variation indicating a predisposition to a response to an insulin sensitizer. In some embodiments, the individual is also screened for a phenotype indicating a predisposition to a response to an insulin sensitizer. In some embodiments the invention further includes converting the results of said screening into data that is capable of transmission, and, in some cases, transmitting said data to a location different from the location at which the data was created. The methods can also further include administering or not administering an insulin sensitizer to the individual based on the results of the screening of the individual; in some cases, administering or not administering the insulin sensitizer is done as part of a drug trial of the insulin sensitizer. The insulin sensitizer for which the individual is screened may be the same as or different from the insulin sensitizer that is administered or not administered to the individual. If the method includes administering an insulin sensitizer, they may also further include modulating the administration of the insulin sensitizer based on the results of the screening of the individual. Modulation of administration can include, e.g., administering another therapeutic agent in addition to the insulin sensitizer, or adjusting the dosage of the insulin sensitizer, route of administration of the insulin sensitizer, frequency of administration of the insulin sensitizer, type of carrier of the insulin sensitizer, duration of treatment with the insulin sensitizer, enantiomeric form of the insulin sensitizer, crystal form of the insulin sensitizer, or a combination thereof, compared to if the screening had not been performed. If the method includes not administering an insulin sensitizer, it may further include administering treatment for the disorder of blood glucose regulation based on the results of the screening of the individual. In certain embodiments, the disorder of blood glucose regulation is diabetes (e.g., Type II diabetes), obesity, or Syndrome X. The genetic variation can be a single nucleotide polymorphism (SNP). In some embodiments, the response to the insulin sensitizer is a therapeutic response, and in some embodiments, the response to the insulin sensitizer is an adverse effect, such as peripheral edema, dependent edema, generalized edema, pitting edema, weight increase, anemia, hypoglycemia, headache, increase in micturation frequency, diarrhea, increased appetite, transient ischemic attack, elevated liver enzymes, and combinations thereof.

In some embodiments, the insulin sensitizer for which the individual is screened and/or that is administered or not administered to the individual is a thiazolidinedione PPAR modulator, such as rosiglitazone, pioglitazone, troglitazone, netoglitazone and 5-BTZD. In some embodiments, the insulin sensitizer for which the individual is screened is a thiazolidinedione PPAR modulator, such as rosiglitazone, pioglitazone, troglitazone, netoglitazone and 5-BTZD, and the insulin sensitizer that is administered or not administered to the individual is netoglitazone. In some embodiments, the insulin sensitizer for which the individual is screened and the insulin sensitizer that is administered or not administered to the individual is netoglitazone. If an insulin sensitizer of the methods is netoglitazone, in certain embodiments the netoglitazone is in an E crystal form. In some embodiments of the methods of the invention, the disorder of blood glucose regulation is Type II diabetes, the genetic variation is a SNP, the insulin sensitizer that is both screened and administered or not administered is netoglitazone, and the response is a therapeutic response. In some embodiments, the disorder of blood glucose regulation is Type II diabetes, the genetic variation is a SNP, the insulin sensitizer that is screened and administered or not administered is netoglitazone, and the response is an adverse effect. In some embodiments of the methods, the screening includes genotyping at least about 1, 10, 100, 1000, 10,000, 100,00, 500,000, 1,000,000, 2,000,000, or substantially all of the genetic variations of the individual.

The invention also provides an isolated nucleic acid that specifically hybridizes to a region of a genomic sequence extending downstream and upstream of an insulin sensitizer response nucleic acid, for use in diagnostics, prognostics, prevention, treatment, or study of an disorder of blood glucose regulation. In some embodiments, the region extends from 10 kb upstream to 10 kb downstream of an insulin sensitizer response nucleic acid, or from 5 kb upstream to 5 kb downstream of an insulin sensitizer response nucleic acid, or from 1 kb upstream to 1 kb downstream of an insulin sensitizer response nucleic acid. The insulin sensitizer can be a PPAR modulator, e.g., a thiazolidinedione PPAR modulator, such as netoglitazone, rosiglitazone, pioglitazone, troglitazone, isaglitazone, 5-BTZD, and R 119702. The disorder of blood glucose regulation may be Type II diabetes.

The invention further provides methods for predicting a presence or absence of a predisposition toward response to an insulin sensitizer in an individual by contacting a sample obtained from the individual with a nucleic acid that specifically hybridizes to a region of genomic sequence extending upstream and downstream of an insulin sensitizer response nucleic acid; and detecting the presence or absence of a hybridization complex, where the presence or absence of a hybridization complex is predictive of the presence or absence of said predisposition toward response to said insulin sensitizer in said individual. In some embodiments, the region extends from 10 kb upstream to 10 kb downstream of an insulin sensitizer response nucleic acid, or from 5 kb upstream to 5 kb downstream of an insulin sensitizer response nucleic acid, or from 1 kb upstream to 1 kb downstream of an insulin sensitizer response nucleic acid. In some embodiments, these methods further include administering or not administering an effective amount of an insulin sensitizer to said patient, based on the presence or absence of the hybridization complex, where the insulin sensitizer that is administered or not administered may be the same as or different from the insulin sensitizer for which the individual was screened.

The invention still further provides an isolated polypeptide partially or fully encoded by a nucleic acid that specifically hybridizes to a region of a genomic sequence upstream and downstream of an insulin sensitizer response nucleic acid, for use in screening, diagnostics, prognostics, prevention, treatment, or study of response to an insulin sensitizer. In some embodiments, the region extends from 10 kb upstream to 10 kb downstream of an insulin sensitizer response nucleic acid, or from 5 kb upstream to 5 kb downstream of an insulin sensitizer response nucleic acid, or from 1 kb upstream to 1 kb downstream of an insulin sensitizer response nucleic acid. The invention also provides an antibody, or an antigen-binding fragment thereof, which selectively binds to a polypeptide as described for use in diagnostics, prognostics, prevention, treatment, or study of response to an insulin sensitizer.

Also provided by the invention are kits for use in diagnostics, prognostics, prevention, treatment, or study of response to an insulin sensitizer that include a nucleic acid that specifically hybridizes to a region of a genomic sequence upstream and downstream of an insulin sensitizer response nucleic acid or an antibody an antibody, or an antigen-binding fragment thereof, which selectively binds to a polypeptide partially or fully encoded by a nucleic acid that specifically hybridizes to a region of a genomic sequence extending upstream and downstream of an insulin sensitizer response nucleic acid. In some embodiments, the region extends from 10 kb upstream to 10 kb downstream of an insulin sensitizer response nucleic acid, or from 5 kb upstream to 5 kb downstream of an insulin sensitizer response nucleic acid, or from 1 kb upstream to 1 kb downstream of an insulin sensitizer response nucleic acid.

The invention yet further provides a method for predicting a response to an insulin sensitizer comprising comparing a level of expression or activity of a polypeptide partially or fully encoded by a nucleic acid that specifically hybridizes to a region of a genomic sequence extending upstream and downstream of an insulin sensitizer response nucleic acid. In some embodiments, the region extends from 10 kb upstream to 10 kb downstream of an insulin sensitizer response nucleic acid, or from 5 kb upstream to 5 kb downstream of an insulin sensitizer response nucleic acid, or from 1 kb upstream to 1 kb downstream of an insulin sensitizer response nucleic acid.

The invention yet further provides business methods that include using one or more genetic variations of the human genome in association studies with susceptibility to response to an insulin sensitizer; and using associations found in the association study step to collaboratively or independently market products related to the insulin sensitizer.

### INCORPORATION BY REFERENCE

All publications and patent applications mentioned in this specification are herein incorporated by reference in their entireties to the same extent as if each individual publication or patent application was specifically and individually indicated to be incorporated by reference. For example, publications and patent applications of particular relevance to the present invention include: U.S. application Ser. No. 10/956,224, filed Sep. 30, 2004, entitled “Methods for Genetic Analysis” and U.S. application Ser. No. 11/043,689, filed Jan. 24, 2005, entitled “Associations Using Genotypes and Phenotypes.”

### BRIEF DESCRIPTION OF THE DRAWINGS

Certain novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings:

FIG. 1 is a high-level block diagram of a system that can be used for data entry, processing, display, storage, and transfer in the methods and compositions of the invention.

FIG. 2 is a flow chart showing steps in the use of an association study for one drug to modulate the clinical trial for a second drug.

### DETAILED DESCRIPTION OF THE INVENTION

### I. Introduction

The invention provides methods, compositions, and kits. Methods of the invention include methods of screening and treatment of an individual including screening an individual for one or more genetic variations indicating a predisposition to a response to first drug and administering or not administering a second drug to the individual based on the results of the screening. In some embodiments the individual suffers from a disorder. In some embodiments the first drug and the second drug are the same; in other embodiments they are different, e.g., different members of a class of drugs. Such screening can be used, for example, to identify individuals who may benefit (or not benefit) from treatment with a drug, individuals who may be enrolled (or excluded) from a clinical trial, and/or individuals who may suffer (or not suffer) an adverse reaction from a drug. In some embodiments one or more phenotypes may also be included in the screening step. Compositions and kits for use in the methods are also provided by the invention.

In some embodiments, methods of the invention include methods of screening and treatment of an individual suffering from a disorder of blood glucose regulation, e.g., an insulin resistance disorder, that include screening an individual in need of treatment for a disorder of blood glucose regulation, e.g., an insulin resistance disorder, for a genetic variation indicating a predisposition to a response to an insulin sensitizer; and administering or not administering an insulin sensitizer to the individual based on the results of the screening. The insulin sensitizer for which the individual is screened may be the same as or different from the insulin sensitizer that is administered or not administered to the individual. Such screening can be used, for example, to identify individuals who may benefit (or not benefit) from treatment with an insulin sensitizer, individuals who may be enrolled (or excluded) from a clinical trial, and/or individuals who may suffer (or not suffer) an adverse reaction to an insulin sensitizer. In some embodiments one or more phenotypes may also be included in the screening step.

In one aspect, the methods of the invention include identifying one or more genetic variations that at least partly differentiate between a subset of a plurality of individuals who experience a particular response when administered a drug, e.g., an insulin sensitizer, and a subset of the plurality of individuals who do not experience the particular response when administered the drug, e.g., insulin sensitizer. In some embodiments the methods further include identifying one or more phenotypes that at least partly differentiate between the subset who experience a particular response when administered a drug, e.g., insulin sensitizer, and the subset who do not experience the particular response when administered the drug, e.g., insulin sensitizer. The methods may further include predicting, based upon said one or more of the genetic variations and/or phenotypes identified, whether a given individual is or is not predisposed to a particular response to a drug, e.g., an insulin sensitizer, where the drug for which the prediction is made may be the same as or may be different from the drug for which one or more genetic variations were identified. In embodiments in which the drugs are different, the methods include predicting, based upon said one or more of the genetic variations and/or phenotypes identified for the first drug, e.g., insulin sensitizer, whether a given individual is or is not predisposed to a particular response to a second drug in the same class of drugs, e.g., in the class of insulin sensitizers.

In some embodiments, if the individual is administered a drug, e.g., an insulin sensitizer, on the basis of screening for one or more genetic variations indicating a response to a drug, the methods further include modulating the administration of the drug, e.g., insulin sensitizer, based on the results of the screening. As in other embodiments, the drug for which the modulation is performed may be the same as or different from the drug for which genetic variations indicate a response. Modulating administration may include but is not limited to adjusting the dosage of the drug, e.g., insulin sensitizer, frequency of administration of the drug, duration of treatment with the drug, form of the drug, or a combination thereof. In some embodiments, if the individual is not administered the drug, e.g., insulin sensitizer, the methods further include administering another treatment for the disorder, e.g., for the disorder of blood glucose regulation. In some embodiments, the methods include methods of screening and treatment of an individual suffering from an disorder of blood glucose regulation by screening the individual for a genetic variation indicating a predisposition to a particular response to a first insulin sensitizer; and administering, modulating the administration, or not administering a second insulin sensitizer to the individual based on the results of the screening. In some of these embodiments the second insulin sensitizer is the subject of research, e.g., a clinical trial.

In one aspect, compositions of the invention include an isolated nucleic acid that specifically hybridizes to a region of a genomic sequence extending upstream and downstream of a nucleic acid associated with a response to a drug, e.g., an insulin sensitizer response nucleic acid, for use in diagnostics, prognostics, prevention, treatment, or study of a disorder, e.g., a disorder of blood glucose regulation. In one aspect, compositions of the invention include an isolated nucleic acid that specifically hybridizes to a region of a genomic sequence that is in linkage disequilibrium with a nucleic acid associated with a response to a drug, e.g., an insulin sensitizer response nucleic acid, for use in diagnostics, prognostics, prevention, treatment, or study of a disorder, e.g., a disorder of blood glucose regulation. Treatment of the disorder can include treatment with another drug in the same class of drugs as the drug for which a response is associated with the nucleic acid. In some embodiments, the region extends from 10 kb upstream to 10 kb downstream of the nucleic acid, or from 5 kb upstream to 5 kb downstream of the nucleic acid, or from 1 kb upstream to 1 kb downstream of the nucleic acid.

In another aspect, compositions of the invention include an isolated polypeptide encoded at least in part by an isolated nucleic acid that specifically hybridizes to a region of a genomic sequence extending downstream and upstream of a nucleic acid associated with a response to a drug, e.g., an insulin sensitizer response nucleic acid for use in diagnostics, prognostics, prevention, treatment, or study of a disorder, e.g., a disorder of blood glucose regulation. Treatment of the disorder can include treatment with another drug in the same class of drugs as the drug for which a response is associated with the nucleic acid. In some embodiments, the region extends from 10 kb upstream to 10 kb downstream of the nucleic acid, or from 5 kb upstream to 5 kb downstream of the nucleic acid, or from 1 kb upstream to 1 kb downstream of the nucleic acid.

In yet another aspect, the invention provides an antibody or an antigen-binding fragment thereof, which selectively binds to an isolated polypeptide encoded at least in part by an isolated nucleic acid that specifically hybridizes to a region of a genomic sequence extending downstream and upstream of a nucleic acid associated with a response to a drug, e.g., an insulin sensitizer response nucleic acid for use in diagnostics, prognostics, prevention, treatment, or study of a disorder, e.g., a disorder of blood glucose regulation. Treatment of the disorder can include treatment with another drug in the same class of drugs as the drug for which a response is associated with the nucleic acid. In some embodiments, the region extends from 10 kb upstream to 10 kb downstream of the nucleic acid, or from 5 kb upstream to 5 kb downstream of the nucleic acid, or from 1 kb upstream to 1 kb downstream of the nucleic acid.

The invention further provides kits that include one or more of the nucleic acids, polypeptides, and/or antibodies of the invention.

In addition the invention provides methods for assaying the presence of a nucleic acid, e.g., an insulin sensitizer response nucleic acid in a sample for use in diagnostics, prognostics, prevention, treatment, or study of a disorder, e.g., a disorder of blood glucose regulation, by contacting the sample with a nucleic acid of the invention under stringent hybridization conditions; and detecting a presence of a hybridization complex. In some embodiments, methods include assaying the expression level of an RNA encoded by a nucleic acid, e.g., an insulin sensitizer response nucleic acid in a sample for use in diagnostics, prognostics, prevention, treatment, or study of a disorder, e.g., a disorder of blood glucose regulation, by contacting the sample with a nucleic acid of the invention under stringent hybridization conditions; and detecting a presence of a hybridization complex. In some embodiments, methods include predicting a presence or absence of a predisposition toward response to a drug, e.g., an insulin sensitizer in an individual by detecting the presence or absence of the hybridization complex. The drug may be the same as or different from the drug for which the nucleic acid is associated to a response. The invention further provides methods for assaying the presence or amount of a polypeptide of the invention for use in diagnostics, prognostics, prevention, treatment, or study of a disorder, e.g., a disorder of blood glucose regulation, by contacting a sample with an antibody of the invention under conditions appropriate for binding; and assessing the sample for the presence or amount of binding of the antibody to the polypeptide. The invention yet further provides methods for predicting a response to a drug, e.g., an insulin sensitizer by comparing the level of expression or activity of a polypeptide of the invention in a test sample from a patient with the level of expression or activity of the same polypeptide in a control sample, where a difference in the level of expression or activity between the test sample and control sample is predictive of a response to a drug, e.g., an insulin sensitizer.

The invention also provides business methods that include using the genetic variations found in the human genome in association studies with responsiveness to an insulin sensitizer; using associations from the association step in a discovery process; and collaboratively or independently, marketing products from the discovery process. The invention further provides business methods that include using the genetic variations found in the human genome in association studies with responsiveness to a first drug in a class of drugs; using associations from the association step in a discovery process involving at least a second drug in the class of drugs; and collaboratively or independently, marketing products from the discovery process.

### II. Disorders of Blood Glucose Regulation

The invention relates to methods and compositions for the diagnosis, prognosis, prevention, treatment, or study of disorder of blood glucose regulation, including insulin resistance disorders.

“Disorders of blood glucose regulation,” as used herein, encompasses disorders that are associated with an inability to regulate blood glucose within optimal limits (often, though not always, due to an insulin resistance disorder) as well as related phenotypes (e.g. complications that result from insulin resistance). Disorders of blood glucose regulation include, but are not limited to, diabetes (both Type I and Type II), Syndrome X, and associated symptoms or complications including such conditions as impaired glucose tolerance (IGT), impaired fasting glucose (IFG), obesity, nephropathy, neuropathy, retinopathy, atherosclerosis, polycystic ovary syndrome, hypertension, ischemia, stroke, heart disease, irritable bowel disorder, inflammation, and cataracts. Examples of a prediabetic state include IGT and IFG.

Insulin, which is produced by the pancreas in response to changes in blood glucose levels, regulates tissue uptake of glucose. As used herein, “insulin resistance disorders” includes disorders in which there is a physiological state wherein normal tissue responsiveness to insulin is impaired. In most cases, to compensate for insulin resistance, the pancreas will produce more insulin. As used herein, “insulin resistance disorders” are considered a subgroup of disorders of blood glucose regulation, whether or not they yet manifest as inability to regulate blood glucose within optimal limits.

Diabetes is a major category of disorders of blood glucose regulation. It is a chronic disorder affecting carbohydrate, fat and protein metabolism in animals. Type I diabetes mellitus, which comprises approximately 10% of all diabetes cases, was previously referred to as insulin-dependent diabetes mellitus (“IDDM”) or juvenile-onset diabetes. This disease is characterized by a progressive loss of insulin secretory function by beta cells of the pancreas. This characteristic is also shared by non-idiopathic, or “secondary”, diabetes having its origins in pancreatic disease. Type I diabetes mellitus is associated with the following clinical signs or symptoms: persistently elevated plasma glucose concentration or hyperglycemia; polyuria; polydipsia and/or hyperphagia; chronic microvascular complications such as retinopathy, nephropathy and neuropathy; and macrovascular complications such as hyperlipidemia and hypertension which can lead to blindness, end-stage renal disease, limb amputation and myocardial infarction.

Type II diabetes mellitus (non-insulin-dependent diabetes mellitus or NIDDM) is a metabolic disorder involving the dysregulation of glucose metabolism and impaired insulin sensitivity. Type II diabetes mellitus usually develops in adulthood and is associated with the body's inability to utilize or make sufficient insulin. In addition to the insulin resistance observed in the target tissues, patients suffering from Type II diabetes mellitus have a relative insulin deficiency—that is, patients have lower than predicted insulin levels for a given plasma glucose concentration. Type II diabetes mellitus is characterized by the following clinical signs or symptoms: persistently elevated plasma glucose concentration or hyperglycemia; polyuria; polydipsia and/or hyperphagia; chronic microvascular complications such as retinopathy, nephropathy and neuropathy; and macrovascular complications such as hyperlipidemia and hypertension which can lead to blindness, end-stage renal disease, limb amputation and myocardial infarction.

Syndrome X, also termed Insulin Resistance Syndrome (IRS), Metabolic Syndrome, or Metabolic Syndrome X, presents symptoms or risk factors for the development of Type II diabetes mellitus and cardiovascular disease, including impaired glucose tolerance (IGT), impaired fasting glucose (IFG), hyperinsulinemia, insulin resistance, dyslipidemia (e.g., high triglycerides, low HDL), hypertension and obesity.

Therapy for Type I diabetes patients has consistently focused on administration of exogenous insulin, which may be derived from various sources (e.g., human, bovine, porcine insulin). The use of heterologous species material gives rise to formation of anti-insulin antibodies which have activity-limiting effects and result in progressive requirements for larger doses in order to achieve desired hypoglycemic effects.

Typical treatment of Type II diabetes mellitus focuses on maintaining the blood glucose level as near to normal as possible with lifestyle modification relating to diet and exercise, and when necessary, the treatment with antidiabetic agents, insulin or a combination thereof.

Although insulin resistance is not always treated in all Syndrome X patients, those who exhibit a prediabetic state (e.g., IGT, IFG), where fasting glucose levels may be higher than normal but are not at the diabetes diagnostic criterion, are treated in some countries (e.g., Germany) with metformin to prevent diabetes. The anti-diabetic agents may be combined with pharmacological agents for the treatment of the concomitant co-morbidities (e.g., antihypertensives for hypertension, hypolipidemic agents for lipidemia).

### III. Drugs of Use in the Invention

The methods and compositions of the invention include the study and use of drugs, e.g., insulin sensitizers, and include performing association studies for determining genotypic and/or phenotypic traits associated with responsiveness to drugs, e.g., insulin sensitizers, screening individuals for predisposition to response to drugs, e.g., insulin sensitizers, e.g., adverse response, and/or administering or not administering drugs, e.g., insulin sensitizers to the individual based on such screening. This section describes certain drugs of use in embodiments of the invention. Further useful drugs for the invention are described in section IVC, Association studies and methods for classes of drugs.

### A. A. Insulin Sensitizers

One class of drugs included in certain embodiments of the invention is an insulin sensitizer. The term “insulin sensitizer,” or “insulin sensitizing agent,” as used herein, refers to any agent capable of enhancing either secretion of or, more typically, tissue sensitivity to, insulin. Non-exclusive examples of insulin sensitizers include metformin, sulfonylureas, alpha glucosidase inhibitors and PPAR modulators, including thiazolidinediones. Further examples of insulin sensitizers are described below.

The thiazolidinediones are examples of PPAR modulators, which are one class of insulin sensitizers for use in the present invention. The term “PPAR modulator,” as used herein, refers to peroxisome proliferator-activated receptor agonists, partial agonists, and antagonists. The modulator may, selectively or preferentially, affect PPAR alpha, PPAR gamma, or both receptors. Typically, the modulator increases insulin sensitivity. According to one aspect, the modulator is a PPAR gamma agonist. One PPAR gamma agonist used in embodiments of the invention is 5-[{6-(2-fluorobenzyl)oxy-2-naphthyl}methyl]-2,4-thiazolidinedione; (MCC-555 or “netoglitazone”).

### B. B. Insulin Sensitizers—PPAR Modulators

One class of insulin sensitizers of the invention is PPAR modulators, and in particular PPAR-gamma modulators, e.g., PPAR-gamma agonists. PPAR modulators include the PPAR-alpha, PPAR-delta (also called PPAR-beta), and PPAR-gamma agonists. Especially useful are the thiazolidinediones (TZDs), which were developed in the 70's and 80's by screening newly synthesized compounds for their ability to lower blood glucose in diabetic rodents. Three molecules from this class, troglitazone, rosiglitazone, and pioglitazone, were ultimately approved for the treatment of patients with Type II diabetes. Although these compounds were developed without an understanding of their molecular mechanism of action, by the early 90's evidence began to accumulate linking the thiazolidinediones to the nuclear receptor PPAR-gamma. It was ultimately demonstrated that these molecules were high affinity ligands of PPAR-gamma and that they increased transcriptional activity of the receptor. Without wishing to be bound by theory, multiple lines of evidence now indicate that the antidiabetic activities of the thiazolidinediones are mediated by their direct interaction with the receptor and the subsequent modulation of PPAR-gamma target gene expression.

Thiazolidinediones of use in the methods of the invention include: (1) rosiglitazone; (2) pioglitazone; (3) troglitazone; (4) netoglitazone (also known as MCC-555 or isaglitazone or neoglitazone); and (5) 5-BTZD.

Accordingly, in some embodiments, the invention provides a method of screening of an individual suffering from a disorder of blood glucose regulation that includes screening the individual for a genetic variation indicating a predisposition to a response to an insulin sensitizer, where the insulin sensitizer for which the individual is screened is a thiazolidinedione PPAR modulator such as rosiglitazone, pioglitazone, troglitazone, netoglitazone, or 5-BTZD; in some embodiments, the invention provides a method of screening of an individual suffering from a disorder of blood glucose regulation that includes screening the individual for a genetic variation indicating a predisposition to a response to an insulin sensitizer, the insulin sensitizer that is administered or not administered to the individual is selected from the group consisting of rosiglitazone, pioglitazone, troglitazone, netoglitazone, or 5-BTZD. Non-thiazolidinedione PPAR modulators that may also be included in certain methods of the invention include muraglitazar and farglitazar, described below.

Other PPAR modulators of use in the invention include modulators that have recently been the subject of clinical trials: (1) Muraglitazar (PPAR gamma and alpha agonist, Bristol-Myers/Merck); (2) Galida tesaglitazar (PPAR gamma and alpha agonist, AstraZeneca); (3) 677954 (PPAR gamma, alpha, and delta agonist, GlaxoSmithKline); (4) MBX-102 (PPAR gamma partial agonist/antagonist, Metabolex); (5) T131 (PPAR gamma selective modulator, Tularik/Amgen); (6) LY818 (PPAR gamma and alpha partial agonist, Eli Lilly/Ligand); (7) LY929 (PPAR gamma and alpha agonist, Eli Lilly/Ligand); and (8) PLX204 (PPAR gamma, alpha, and delta agonist, Plexxikon). See, e.g., BioCentury, Jun. 14, 2004. Further PPAR modulators include LY 519818, L-783483, L-165461, and L-165041.

Additionally, the non-thiazolidinediones that act as insulin-sensitizing agents include, but are not limited to: (1) JT-501 (JTT 501, PNU-1827, PNU-716-MET-0096, or PNU 182716: 4-(4-(2-(5-methyl-2-phenyl-oxazol-4-yl)ethoxy)benzyl)isoxazolidine-3,5-dione; (2) KRP-297 (5-(2,4-dioxothiazolidin-5-ylmethyl)-2-methoxy-N-(4-(tri-fluoromethyl)benzyl)benzamide or 5-((2,4-dioxo-5-thiazolidinyl)methyl)-2-methoxy-N-((4-(trifluoromethyl)phenyl)methyl)benzamide); and (3) Farglitazar (L-tyrosine, N-(2-benzoylphenyl)-o-(2-(5-methyl-2-phenyl-4-oxazolyl)ethyl) or N-(2-benzoylphenyl)-O-(2-(5-methyl-2-phenyl-4-oxazolyl)ethyl)-L-tyrosine, or (S)-2-(2-benzoylphenylamino)-3-(4-12-(5-methyl-2-phenyl-2-oxazo-4-yl)ethoxyphenyl)propionic acid, or GW2570 or GI-262570).

Other agents have also been shown to have PPAR modulator activity such as PPAR-gamma, SPPAR-gamma, and/or PPAR-alpha/delta agonist activity. Examples are: (1) AD 5075 (5-(4-(2-hydroxy-2-(5-methyl-2-phenyloxazol-4-yl)ethoxy)benzyl)-thiazolidine-2,4-dione); (2) R 119702 (or Cl 1037 or CS 011); (3) CLX-0940 (peroxisome proliferator-activated receptor alpha agonist/peroxisome proliferator-activated receptor gamma agonist); (4) LR-90 (2,5,5-tris(4-chlorophenyl)-1,3-dioxane-2-carboxylic acid, PPAR alpha/gamma agonist); (5) CLX-0921 (PPAR gamma agonist); (6) CGP-52608 (PPAR agonist); (7) GW-409890 (PPAR agonist); (8) GW-7845 (2((S)-1-carboxy-2-(4-(2-(5-methyl-2-phenyl-oxazol-4-yl)-ethoxy)-phenyl)-ethyamino)-benzoic acid methyl ester, PPAR agonist); (9) L-764406 (2-benzenesulphonylmethyl-3-chloroquinoxaline, PPAR agonist); (10) LG-101280 (PPAR agonist); (11) LM-4156 (PPAR agonist); (12) Risarestat (CT-112, (±)-5-(3-ethoxy-4-(pentyloxy)phenyl-2,4-thiazolidinedione aldose reductase inhibitor); (13) YM 440 (PPAR agonist); (14) AR-H049020 (PPAR agonist); (15) GW 0072 ((±)-(2S,5S)-4-(4-(5-((dibenzy carbomoyl)methyl)-2-heptlyl-4-oxothiazolidin-3-yl butyl)benzoic acid); (16) GW 409544 (GW-544 or GW-409544); (17) NN 2344 (DRF 2593); (18) NN 622 (DRF 2725); (19) AR-H039242 (AZ-242); (20) GW 9820 (fibrate); (21) GW 1929 (N-(2-benzoylphenyl)-O-(2-(methyl-2-pyridinylamino)ethyl)-L-tyrosine, known as GW 2331, PPAR agonist); (22) SB 219994 ((S)-4-(2-(2-benzoxazolylmethylamino)ethoxy)-alpha-(2,2,2-trifluoroethoxy)benzen epropanoic acid or 3-(4-(2-(N-(2-benzoxazolyl)-N-methylamino)ethoxy)phenyl)-2 (S)-(2,2,2-trifluoroethoxy)propionic acid or benzenepropanoic acid, 4-(2-(2-benzoxazolylmethylamino)ethoxy)-alpha-(2,2,2-trifluoroethox-γ)-, (alpha S)-, PPAR alpha/gamma agonist); (23) L-796449 (PPAR alpha/gamma agonist); (24) Fenofibrate (propanoic acid, 2-[4-(4-chlorobenzoyl)phenoxy]-2-methyl-, 1-methylethyl ester, known as TRICOR, LIPCOR, LIPANTIL, LIPIDIL MICRO PPAR alpha agonist); (25) GW-9578 (PPAR alpha agonist); (26) GW-2433 (PPAR alpha/gamma agonist); (27) GW-0207 (PPAR gamma agonist); (28) LG-100641 (PPAR gamma agonist); (29) LY-300512 (PPAR gamma agonist); (30) NID525-209 (NID-525); (31) VDO-52 (VDO-52); (32) LG 100754 (peroxisome proliferator-activated receptor agonist); (33) LY-510929 (peroxisome proliferator-activated receptor agonist); (34) bexarotene (4-(1-(3,5,5,8,8-pentamethyl-5,6,7,8-tetrahydro-2-naphthalenyl)ethenyl)benzoic acid, known as TARGRETIN, TARGRETYN, TARGREXIN; also known as LGD 1069, LG 100069, LG 1069, LDG 1069, LG 69, RO 264455); and (35) GW-1536 (PPAR alpha/gamma agonist).

Other thiazolidinedione and non-thiazolidinedione insulin sensitizers of use in the invention are described in, e.g., Leff and Reed (2002) Curr. Med. Chem.—Imun., Endoc.,& Metab. Agents 2:33-47; Reginato et al. (1998) J. Biol. Chem., 278 32679-32654; Way et al. (2001) J. Biol. Chem. 276 25651-25653; Shiraki et al. (2005) JBC Papers in Press, published on Feb. 4, 2005, as Manuscript M500901200, and U.S. Pat. Nos. 4,703,052; 6,008,237; 5,594,016; 6,838,442; 6,329,423; 5,965,589; 6,677,363; 4,572,912; 4,287,200; 4,340,605; 4,438,141; 4,444,779; 4,572,912; 4,687,777; 4,725,610; 5,232,925; 5,002,953; 5,194,443; 5,260,445; 6,300,363; 6,034,110; and 6,541,493; U.S. Patent Application Publications 2002/0042441; 2004/0198774 and 2003/0045553; EP Patent Nos. 0139421 and 0332332; and PCT Publication Nos. WO 95/35314; WO 00/31055; WO 01/3640, all of which are incorporated by reference herein in their entirety.

### C. Netoglitazone

One thiazolidinedione PPAR modulator for use in the methods of the invention is netoglitazone (5-[{6-(2-fluorobenzyl)oxy-2-naphthyl}methyl]-2,4-thiazolidinedione; MCC-555). Structures and methods of preparation of netoglitazone and various forms of netoglitazone of use in the invention are described in, e.g., U.S. Pat. Nos. 5,594,016; 6,541,493; 6,541,493; 6,838,442; U.S. Patent Application No. 2004/0198774 and 2003045553; PCT Publication Nos. WO 00/31055; WO 01/36401; WO 03/018010, and WO 00/73252; Japanese Patent Unexamined Publication (KOKAI) Nos. (Hei) 6-247945/1994 and (Hei) 10-139768/1998; Japanese Patents 2001172179 and 2003040877; and Reginato et al. (1998) J. Biol. Chem. 273: 32679-32684; all of which are incorporated by reference herein in their entirety.

It has been reported that netoglitazone is more efficacious than pioglitazone and troglitazone in lowering plasma glucose, insulin, and triglyceride levels and that it is about three-fold more potent than rosiglitazone. The activity of netoglitazone appears to be context-specific, as in some cell types it behaves as a full agonist of PPAR-gamma and as a partial agonist or antagonist in others. In addition, it appears to modulate PPAR-alpha and delta as well. See, e.g., U.S. Patent Application Publication No. 2004/0198774.

### D. Forms of Drugs

Some compounds useful in the invention, including the TZD PPAR modulators such as netoglitazone, may have one or more asymmetric carbon atoms in their structure. It is intended that the present invention include within its scope the stereochemically pure isomeric forms of the compounds as well as their racemates. Stereochemically pure isomeric forms may be obtained by the application of art known principles. Diastereoisomers may be separated by physical separation methods such as fractional crystallization and chromatographic techniques, and enantiomers may be separated from each other by the selective crystallization of the diastereomeric salts with optically active acids or bases or by chiral chromatography. Pure stereoisomers may also be prepared synthetically from appropriate stereochemically pure starting materials, or by using stereospecific reactions.

Some compounds useful in the invention may have various individual isomers, such as trans and cis, and various alpha and beta attachments (below and above the plane of the drawing). In addition, where the processes for the preparation of the compounds according to the invention give rise to mixture of stereoisomers, these isomers may be separated by conventional techniques such as preparative chromatography. The compounds may be prepared as a single stereoisomer or in racemic form as a mixture of some possible stereoisomers. The non-racemic forms may be obtained by either synthesis or resolution. The compounds may, for example, be resolved into their components enantiomers by standard techniques, such as the formation of diastereomeric pairs by salt formation. The compounds may also be resolved by covalent linkage to a chiral auxiliary, followed by chromatographic separation and/or crystallographic separation, and removal of the chiral auxiliary. Alternatively, the compounds may be resolved using chiral chromatography. Unless otherwise noted, the scope of the present invention is intended to cover all such isomers or stereoisomers per se, as well as mixtures of cis and trans isomers, mixtures of diastereomers and racemic mixtures of enantiomers (optical isomers) as well.

In addition, compounds of the invention may be prepared in various polymorphic forms. For example, insulin sensitizers of use in the invention can occur in polymorphic forms, and any or all of the polymorphic forms of these insulin sensitizers are contemplated for use in the invention. Polymorphism in drugs may alter the stability, solubility and dissolution rate of the drug and result in different therapeutic efficacy of the different polymorphic forms of a given drug. The term polymorphism is intended to include different physical forms, crystal forms, and crystalline/liquid crystalline/non-crystalline (amorphous) forms. Polymorphism of compounds of therapeutic use has is significant, as evidenced by the observations that many antibiotics, antibacterials, tranquilizers etc., exhibit polymorphism and some/one of the polymorphic forms of a given drug may exhibit superior bioavailability and consequently show much higher activity compared to other polymorphs. For example, Sertraline, Frentizole, Ranitidine, Sulfathiazole, and Indomethacine are some of the pharmaceuticals that exhibit polymorphism.

Some embodiments of the invention include the use of netoglitazone in one of its polymorphic forms. Netoglitazone can be prepared in various polymorphic forms. Any polymorphic forms of netoglitazone known in the art may be used in the methods of the invention, either separately or in combination. Thus, the methods of the invention include association studies using any or all of the polymorphic forms of netoglitazone, as well as screening and treatment using any or all of the polymorphic forms of netoglitazone, compositions and kits based on these forms, and the like.

Polymorphic forms of netoglitazone include the A, B, C, D, E and amorphous crystal forms described in PCT Published Application No. WO 01/36401 and in U.S. Pat. No. 6,541,493; for example, the E form is described in PCT Published Application No. WO 01/36401.

Some of the compounds described herein may exist with different points of attachment of hydrogen coupled with double bond shifts, referred to as tautomers. An example is a carbonyl (e.g. a ketone) and its enol form, often known as keto-enol tautomers. The individual tautomers as well as mixtures thereof are encompassed within the invention.

Prodrugs are compounds that are converted to the claimed compounds as they are being administered to a patient or after they have been administered to a patient. The prodrugs are compounds of this invention, and the active metabolites of the prodrugs are also compounds of the invention.

### E. Responses to Drugs

Responses to drugs, e.g., insulin sensitizers, that are observed or predicted in the methods of the invention include therapeutic responses and responses that are not therapeutic (e.g., side effects, such as adverse effects.). Included in “response” and “responsiveness” as those terms are used herein is no response or no detectable response. Hence, in some cases, a genetic variation or phenotype associated with a response to a drug, e.g., insulin sensitizer, is associated with an effect (response) when that drug, e.g., insulin sensitizer is administered, whereas in other cases a genetic variation or phenotype associated with a response to a drug, e.g., insulin sensitizer is associated with a lack of a detectable effect (response) when that drug, e.g., insulin sensitizer is administered.

Therapeutic responses include any response to a drug that results in an improvement or amelioration of the condition for which the drug is administered and/or complications due to the condition for which the drug is administered. For example, if the drug is an insulin sensitizer, “therapeutic response” includes any response that results in an improvement or amelioration of sensitivity to insulin and/or complications due to lack of insulin sensitivity. One therapeutic response is increased sensitivity to insulin, which may be evidenced by a decrease in blood glucose levels, either with or without the administration of exogenous insulin. Therapeutic responses to a drug, e.g., insulin sensitizer, may be further classified by degree of response; any suitable gradation of degree of response may be used, including relatively broad gradations (e.g., strong responder, moderate responder, and weak responder) and relatively more narrow gradations (e.g., ranking responses as a percentile of maximum observed response and dividing responses into, e.g., deciles, quartiles, and the like).

Responses to a drug that are not therapeutic effects can include adverse effects. For example, some PPAR modulators are known to exhibit non-therapeutic effects, including adverse effects. Although certain PPAR modulators have been shown to be safe and effective in FDA testing, all show varying degrees of adverse effects, some of which are serious enough to halt clinical trials or to stop the use of the approved drug. For example, troglitazone, which was marketed in the U.S. starting in April, 1997 for the treatment of Type II diabetes, was voluntarily withdrawn in March 2000 due to incidents of idiosyncratic liver damage. Although these incidents occurred with a frequency of only about 1 in 100,000 patients, their severity was such that the drug was withdrawn. More common adverse effects such as edema, weight gain, and adverse effects on lipid profiles, while not as severe as idiosyncratic liver damage, can nonetheless limit the therapeutic potential of insulin resistance modulators.

In association and diagnostic methods of the invention, “adverse effects,” of a drug, e.g., of a PPAR modulator, include an adverse effect on the user of the drug. Exemplary adverse effects of insulin sensitizers include peripheral edema, dependent edema, generalized edema, pitting edema, weight increase or decrease, anemia, hypoglycemia, headache, increase in micturation frequency, diarrhea, increased or decreased appetite, transient ischemic attack, elevated liver enzymes, and combinations thereof. Non-therapeutic effects, e.g., adverse effects, may be measured by any means known in the art or apparent to the skilled artisan. As with therapeutic responses, nontherapeutic responses, e.g., adverse effects, to a drug, e.g., insulin sensitizer, may be further classified by degree of response and any suitable gradation of degree of response may be used.

### F. Additional Agents for Use in the Invention

Methods of the invention include screening an individual for a genetic variation and/or phenotypic variation that indicates responsiveness to a drug, e.g., an insulin sensitizer prior to determining whether or not to administer a drug, e.g., an insulin sensitizer to an individual.

Such screening can be used, for example, to identify individuals who may benefit (or not benefit) from treatment with the drug, e.g., insulin sensitizer, individuals who may be enrolled (or excluded) from a clinical trial, and/or individuals who may suffer (or not suffer) an adverse response to the drug, e.g., insulin sensitizer. In some embodiments, the results of the screening determine whether drug, e.g., insulin sensitizer is administered or is not administered to a particular individual. In some embodiments in which the drug, e.g., insulin sensitizer is administered, the administration of the drug is modulated based on the results of the screening of the individual. Such modulation may involve adjustment of the administration of the drug. For example, modulating administration may include: adjusting the dosage of the drug, route of administration of the drug, duration of treatment with the drug, or frequency of administration of the drug; changing the type of carrier of the drug, enantiomeric form of the drug, crystal form of the drug, tautomeric form of the drug; administering a fragment, analog, and/or variant of the drug; or a combination thereof.

Modulation of the administration of the drug, e.g., insulin sensitizer can also include administration of one or more other therapeutic agents that are not the drug for which the individual is screened, in addition to administration of the drug for which the individual is screened. For non-insulin sensitizers, another drug in the class of drugs may be administered. Drug classes are described in detail below. In the case of insulin sensitizers, the other therapeutic agent may be, e.g., another insulin sensitizer, or an agent that is not an insulin sensitizer, as described herein. Thus, in some cases, the methods of the invention involve the administration of one or more agents that are not the agent, e.g., insulin sensitizer for which the individual is screened. In some embodiments, the methods of the invention involve the administration of one or more agents that are not the insulin sensitizer for which the individual is screened in combination with the insulin sensitizer for which the individual is screened.

In some embodiments in which the insulin sensitizer for which the individual is screened is not administered, another therapeutic agent can be administered to the individual in place of the insulin sensitizer for which the individual is screened. For example, an individual may be screened for responsiveness to netoglitazone, and if the results of the screening indicate that the individual is predisposed to adverse effects of netoglitazone, another PPAR modulator, such as rosiglitazone, pioglitazone, or troglitazone, may be administered instead. Alternatively, another insulin sensitizer that is not a TZD PPAR modulator may be administered to the individual based on the results of the screening.

Other therapeutic agents include the insulin sensitizers described above, or non-insulin sensitizing agents. See, e.g., Leff and Reed (2002) Curr. Med. Chem.—Imun., Endoc.& Metab. Agents 2:33-47; Reginato et al. (1998) J. Biol. Chem., 278 32679-32654; Way et al. (2001) J. Biol. Chem. 276 25651-25653; Shiraki et al. (2005) JBC Papers in Press, published on Feb. 4, 2005, as Manuscript M500901200, and U.S. Pat. Nos. 4,703,052; 6,008,237; 5,594,016; and 6,541,493; U.S. Patent Application Publication 2004/0198774 and 2003/0045553; and PCT Publication Nos. WO 00/31055; WO 01/3640, all of which are incorporated by reference herein in their entirety.

Other agents useful in the methods of the invention include, but are not limited to:

1. Biguanides, which decrease liver glucose production and increases the uptake of glucose. Examples include metformin such as: (1) 1,1-dimethylbiguanide (e.g., Metformin-DepoMed, Metformin-Biovail Corporation, or METFORMIN GR (metformin gastric retention polymer)); and (2) metformin hydrochloride (N,N-dimethylimidodicarbonimidic diamide monohydrochloride, also known as LA 6023, BMS 207 150, GLUCOPHAGE, or GLUCOPHAGE XR.

2. Alpha-glucosidase inhibitors, which inhibit alpha-glucosidase, and thereby delay the digestion of carbohydrates. The undigested carbohydrates are subsequently broken down in the gut, reducing the post-prandial glucose peak. Examples include, but are not limited to: (1) acarbose (D-glucose, O-4,6-dideoxy-4-(((1S-(1alpha,4alpha,5beta,6alpha))-4,5,6-trihydroxy-3-(hydroxymethyl)-2-cyc-lohexen-1-yl)amino)-alpha-D-glucopyranosyl-(1-4)-O-alpha-D-glucopyranosyl-(1-4)-, also known as AG-5421, Bay-g-542, BAY-g-542, GLUCOBAY, PRECOSE, GLUCOR, PRANDASE, GLUMIDA, or ASCAROSE); (2) Miglitol (3,4,5-piperidinetriol, 1-(2-hydroxyethyl)-2-(hydroxymethyl)-, (2R(2alpha, 3beta, 4alpha, 5beta))- or (2R,3R,4R,5S)-1-(2-hydroxyethyl)-2-(hydroxymethyl-3,4,5-piperidinetriol, also known as BAY 1099, BAY M 1099, BAY-m-1099, BAYGLITOL, DIASTABOL, GLYSET, MIGLIBAY, MITOLBAY, PLUMAROL); (3) CKD-711 (0-4-deoxy-4-((2,3-epoxy-3-hydroxymethyl-4,5,6-trihydro-xycyclohexane-1-yl)amino)-alpha-b-glucopyranosyl-(1-4)-alpha-D-glucopyran-osyl-(1-4)-D-glucopyranose); (4) emiglitate (4-(2-((2R,3R,4R,5S)-3,4,5-trihydroxy-2-(hydroxymethyl)-1-piperidinyl)ethoxy)benzoic acid ethyl ester, also known as BAY o 1248 or MKC 542); (5) MOR 14 (3,4,5-piperidinetriol, 2-(hydroxymethyl)-1-methyl-, (2R-(2alpha, 3beta, 4alpha, 5beta))-, also known as N-methyideoxynojirimycin or N-methylmoranoline); and (6) Voglibose (3,4-dideoxy-4-((2-hydroxy-1-(hydroxymethyl)ethyl)amino)-2-C-(hydroxymethyl)-D-epi-inositol or D-epi-lnositol,3,4-dideoxy-4-((2-hydroxy-1-(hydroxymethyl)ethyl)amino)-2-C-(hydroxymethyl)-, also known as A 71100, AO 128, BASEN, GLUSTAT, VOGLISTAT.

3. Insulins include regular or short-acting, intermediate-acting, and long-acting insulins, injectable, non-injectable or inhaled insulin, transderamal insulin, tissue selective insulin, glucophosphokinin (D-chiroinositol), insulin analogues such as insulin molecules with minor differences in the natural amino acid sequence and small molecule mimics of insulin (insulin mimetics), and endosome modulators. Examples include, but are not limited to: (1) Biota; (2) LP 100; (3) (SP-5-21)-oxobis(1-pyrrolidinecarbodithioato-S, S′) vanadium, (4) insulin aspart (human insulin (28B-L-aspartic acid) or B28-Asp-insulin, also known as insulin X14, INA-X14, NOVORAPID, NOVOMIX, or NOVOLOG); (5) insulin detemir (Human 29B-(N6-(1-oxotetradecyl)-L-lysine)-(1A-21A), (1B-29B)-Insulin or NN 304); (6) insulin lispro (“28B-L-lysine-29B-L-proline human insulin, or Lys (B28), Pro (B29) human insulin analog, also known as lys-pro insulin, LY 275585, HUMALOG, HUMALOG MIX 75/25, or HUMALOG MIX 50/50); (7) insulin glargine (human (A21-glycine, B31-arginine, B32-arginine) insulin HOE 901, also known as LANTUS, OPTISULIN); (8) Insulin Zinc Suspension, extended (Ultralente), also known as HUMULIN U or ULTRALENTE; (9) Insulin Zinc suspension (Lente), a 70% crystalline and 30% amorphous insulin suspension, also known as LENTE ILETIN II, HUMULIN L, or NOVOLIN L; (10) HUMULIN 50/50 (50% isophane insulin and 50% insulin injection); (11) HUMULIN 70/30 (70% isophane insulin NPH and 30% insulin injection), also known as NOVOLIN 70/30, NOVOLIN 70/30 PenFill, NOVOLIN 70/30 Prefilled; (12) insulin isophane suspension such as NPH ILETIN II, NOVOLIN N, NOVOLIN N PenFill, NOVOLIN N Prefilled, HUMULIN N; (13) regular insulin injection such as ILETIN II Regular, NOVOLIN R, VELOSULIN BR, NOVOLIN R PenFill, NOVOLIN R Prefilled, HUMULIN R, or Regular U-500 (Concentrated); (14) ARIAD; (15) LY 197535; (16) L-783281; and (17) TE-17411.

4. Insulin secretion modulators such as (1) glucagon-like peptide-1 (GLP-1) and its mimetics; (2) glucose-insulinotropic peptide (GIP) and its mimetics; (3) exendin and its mimetics; (4) dipeptyl protease (DPP or DPPIV) inhibitors such as (4a) DPP-728 or LAF 237 (2-pyrrolidinecarbonitrile,1-(((2-((5-cyano-1-2-pyridinyl)amino)ethyl)amino)acetyl), known as NVP-DPP-728, DPP-728A, LAF-237); (4b) P 3298 or P32/98 (di-(3N-((2S,3S)-2-amino-3-methyl-pentanoyl-)-1,3-thiazolidine) fumarate); (4c) TSL 225 (tryptophyl-1,2,3,4-tetrahydroisoquinoline-3-carboxyli-c acid); (4d) Valine pyrrolidide (valpyr); (4e) 1-aminoalkylisoquinolinone-4-carboxylates and analogues thereof; (4f) SDZ 272-070 (1-(L-Valyl) pyrrolidine); (4g) TMC-2A, TMC-2B, or TMC-2C; (4h) Dipeptide nitriles (2-cyanopyrrolodides); (41) CD26 inhibitors; and (4j) SDZ 274-444; (5) glucagon antagonists such as AY-279955; and (6) amylin agonists which include, but are not limited to, pramlintide (AC-137, Symlin, tripro-amylin or pramlintide acetate).

5. Insulin secretagogues, which increase insulin production by stimulating pancreatic beta cells, such as: (1) asmitiglinide ((2 (S)-cis)-octahydro-gamma-oxo-alpha-(phenylmet-hyl)-2H-isoindole-2-butanoic acid, calcium salt, also known as mituglimide calcium hydrate, KAD 1229, or S 21403); (2) Ro 34563; (3) nateglinide (trans-N-((4-(1-methylethyl)cyclohexyl) carbonyl)-D-phenylalanine, also known as A 4166, AY 4166, YM 026, FOX 988, DJN 608, SDZ DJN608, STARLIX, STARSIS, FASTIC, TRAZEC); (4) JTT 608 (trans-4-methyl-gamma-oxocyclohexanebutanoic acid); (5) sulfonylureas such as: (5a) chlorpropamide(1-[(p-chlorophenyl)sulfonyl]-3-propylurea, also known as DIABINESE); (5b) tolazamide(TOLINASE or TOLANASE); (5c) tolbutamide(ORINASE or RASTINON); (5d) glyburide (1-[[p-[2-(5-chloro-o-anisamido)ethyl]phenyl]sulfon-yl]-3-cyclohexylurea, also known as Glibenclamide, DIABETA, MICRONASE, GLYNASE PresTab, or DAONIL); (5e) glipizide (1-cyclohexyl-3-[[p-[2-(5-ethylpyrazinecarboxamido)e-thyl]phenyl]sulfonyl]urea, also known as GLUCOTROL, GLUCOTROL XL, MINODIAB, or GLIBENESE); (5f) glimepiride (1H-pyrrole-1-carboxamide, 3-ethyl-2,5-dihydro-4-m-ethyl-N-[2-[4-[[[[(4-methylcyclohexyl)amino]carbonyl]amino]sulfonyl]phenyl-]ethyl]-2-oxo-, trans-, also known as Hoe-490 or AMARYL); (5g) acetohexamide (DYMELOR); (5h) gliclazide (DIAMICRON); (5i) glipentide (STATICUM); (5j) gliquidone (GLURENORM); and (5k) glisolamide (DIABENOR); (6) K⁺ channel blockers including, but not limited to, meglitinides such as (6a) Repaglinide ((S)-2-ethoxy-4-(2-((3-methyl-1-(2-(1-piperidinyl)phenyl)butyl)amino)-2-oxoethyl) benzoic acid, also known as AGEE 623, AGEE 623 ZW, NN 623, PRANDIN, or NovoNorm); (6b) imidazolines; and (6c) α-2 adrenoceptor antagonists; (7) pituitary adenylate cyclase activating polypeptide (PAcAP); (8) vasoactive intestinal peptide (VIP); (9) amino acid analogs; and (10) glucokinase activators.

6. Growth Factors such as: (1) insulin-like growth factors (IGF-1, IGF-2); (2) small molecule neurotrophins; (3) somatostatin; (4) growth hormone-releasing peptide (GHRP); (5) growth hormone-releasing factor (GHRF); and (6) human growth hormone fragments.

7. Immunomodulators such as: (1) vaccines; (2) T-cell inhibitors; (3) monoclonal antibodies; (4) interleukin-1 (IL-1) antagonists; and (5) BDNF.

8. Glucose resorption inhibitors such as those described in U.S. Patent Application No. 2003/0045553.

9. Other antidiabetic agents: (1)ᵣHu-Glucagon; (2) DHEA analogs; (3) carnitine palmitoyl transferase (CPT) inhibitors; (4) islet neurogenesis; (5) pancreatic p amyloid inhibitors; and (6) UCP (uncoupling protein)-2 and UCP-3 modulators.

Additional agents of use in the invention include any agents known in the art for treatment of disorder of blood glucose regulations and/or their complications. Such agents include, but are not limited to, cholesterol lowering agents such as (i) HMG-CoA reductase inhibitors (lovastatin, simvastatin and pravastatin, fluvastatin, atorvastatin, rivastatin, pitavastatin, and other statins), (ii) sequestrants (cholestyramine, colestipol and a dialkylaminoalkyl derivatives of a cross-linked dextran), (iii) nicotinyl alcohol, nicotinic acid or a salt thereof, (iv) PPAR.alpha. agonists such as fenofibric acid derivatives (gemfibrozil, clofibrate, fenofibrate and bezafibrate), (v) inhibitors of cholesterol absorption for example beta-sitosterol and (acyl CoA:cholesterol acyltransferase) inhibitors for example melinamide and (vi) probucol; PPARdelta agonists such as those disclosed in WO97/97/28149; antiobesity compounds such as fenfluramine, dexfenfluramine, phentiramine, sulbitramine, orlistat, neuropeptide Y5 inhibitors, and, β₃ adrenergic receptor agonist; and ileal bile acid transporter inhibitors.

### IV. Association Methods

In one aspect the invention provides methods of identifying one or more genetic variations that at least partly differentiate between a subset of a plurality of individuals who experience a response, or are likely to experience a response, when administered a drug, e.g., an insulin sensitizer, and a subset of said plurality of individuals who do not experience the response, or who are not likely to experience the response, when administered the insulin sensitizer. In some embodiments, the methods of the invention also include identifying one or more phenotypes that at least partly differentiate between the subset who experience or are likely to experience a response when administered an insulin sensitizer, and the subset who do not experience or are not likely to experience the response when administered the insulin sensitizer. The methods may also include predicting, based on one or more identified genotypes and/or phenotypes, whether a particular individual is predisposed to a response to an insulin sensitizers

A “response” to drug, e.g., an insulin sensitizer can be any response described herein, such as a therapeutic response or a non-therapeutic response (e.g., side effect, such as an adverse effect, as described herein). In some embodiments, the genetic variations are SNPs, in some of these embodiments, the SNPs include at least one informative SNP, as described herein. In some embodiments the drug is an insulin sensitizer such as a thiazolidinedione PPAR modulator, for example, netoglitazone.

Although the association methods described herein are suitable for use with any drug, for convenience in some details are described in terms of insulin sensitizers. It is understood that this is for convenience only, and that any drug may be studied by these methods. The methods utilize techniques of genomics and, in particular, pharmacogenomics.

As used herein, the terms “differentiate at least in part” and “at least partly differentiate” mean a clinically useful result that can be used to differentiate cases from controls and is at least about 50% sensitive, or at least about 60% sensitive, or at least about 70% sensitive, or at least about 80% sensitive, or at least about 90% sensitive, or at least about 95% sensitive, or at least about 99% sensitive; or a clinically useful result that can be used to differentiate cases from controls and is at least about 50% specific, or at least about 60% specific, or at least about 70% specific, or at least about 80% specific, or at least about 90% specific, or at least about 95% specific, or at least about 99% specific.

The DNA that makes up human chromosomes provides the instructions that direct the production of all proteins in the body. These proteins carry out vital functions of life. Variations in DNA are directly related to almost all human diseases, including infectious diseases, cancers, inherited disorders, and autoimmune disorders. Variations in DNA contributing to a phenotypic change, such as a disease or a disorder, may result from a single variation that disrupts the complex interactions of several genes or from any number of mutations within a single gene. For example, Type I and II diabetes have been linked to multiple genes, each with its own pattern of mutations. In contrast, cystic fibrosis can be caused by any one of over 300 different mutations in a single gene. Phenotypic changes may also result from variations in non-coding regions of the genome. For example, a single nucleotide variation in a regulatory region can upregulate or downregulate gene expression or alter gene activity.

Technological developments in the field of human genomics have enabled the development of pharmacogenomics, the use of human DNA sequence variability in the development and prescription of drugs. Pharmacogenomics is based on the correlation or association between a given genotype and a resulting phenotype. Since the first association study over half-a-century ago linking adverse drug response with amino acid variations in two drug-metabolizing enzymes (plasma cholinesterase and glucose-6-phosphate dehydrogenase), other correlation studies have linked sequence polymorphisms in drug metabolism enzymes, drug targets and drug transporters with compromised levels of drug efficacy or safety. Pharmacogenomics information is especially useful in clinical settings where association information is used to prevent drug toxicities. For example, patients may be screened for genetic differences in the thiopurine methyltransferase gene that cause decreased metabolism of 6-mercaptopurine or azathiopurine.

Processes that may be used in specific embodiments of the methods herein are described in more detail in the following patent applications, all of which are specifically incorporated herein by reference: U.S. patent application Ser. No. 11/043,689, filed Jan. 24, 2005, entitled “Associations Using Genotypes and Phenotypes;” U.S. Provisional Application Ser. No. 60/280,530, filed Mar. 30, 2001, entitled “Identifying Human SNP Haplotypes, Informative SNPs and Uses Thereof”; U.S. Provisional Application Ser. No. 60/313,264 filed Aug. 17, 2001, entitled “Identifying Human SNP Haplotypes, Informative SNPs and Uses Thereof”; U.S. Provisional Application Ser. No. 60/327,006, filed Oct. 5, 2001, entitled “Identifying Human SNP Haplotypes, Informative SNPs and Uses Thereof”; U.S. Provisional Application Ser. No. 60/332,550, filed Nov. 26, 2002, entitled “Methods for Genomic Analysis”; U.S. application Ser. No. 10/106,097, filed Mar. 26, 2002, entitled “Methods for Genomic Analysis”; U.S. Pat. No. 6,897,025, issued May 24, 2005, entitled “Genetic Analysis Systems and Methods”; and U.S. application Ser. No. 10/284,444, filed Oct. 31, 2002, entitled “Human Genomic Polymorphisms”, U.S. Provisional Patent Application No. 60/648,957, filed Jan. 31, 2005, entitled “Compositions and Methods For Treating, Preventing, and Diagnosing Alzheimer's Disease,” U.S. application Ser. No. 10/956,224, filed Sep. 30, 2004, entitled “Methods for genetic analysis,” U.S. Provisional Patent Application No. 60/653,672, filed Feb. 16, 2005, entitled “Parkinson's Disease-Related Disease Compositions and Methods,” U.S. application Ser. No. 10/768,788, filed Jan. 30, 2004, entitled “Apparatus and Methods for Analyzing and Characterizing Nucleic Acid Sequences,” U.S. application Ser. No. 10/351,973, filed Jan. 27, 2003, entitled “Apparatus and Methods for Determining Individual Genotypes,” U.S. application Ser. No. 10/970,761, filed Nov. 20, 2004, entitled “Improved Analysis Methods and Apparatus for Individual Genotyping,” U.S. application Ser. No. 10/786,475, filed Feb. 24, 2004, entitled “Analysis Methods for Individual Genotyping,” and U.S. Provisional Patent Applications No. 60/643,006, 60/635,281 filed Jan. 11, 2005, and Dec. 9, 2004, respectively, both entitled “Markers for Metabolic Syndrome Obesity and Insulin Resistance,” the disclosures all of which are specifically incorporated herein by reference.

Sequencing the human genome has revealed that there is a high degree of homology in genetic information between individuals. In particular, any two humans share approximately 99.9% the same DNA sequence and have up to about 20,000 to about 30,000 or so genes similarly situated in one of twenty-three chromosomes. However, genomic variations between any two individuals still exist. For example, approximately 0.1%, or one out of every 1,000 DNA letters, is variable in a population of humans.

Genetic variations between individuals can occur in many forms. Examples of genetic variations include, but are not limited to, deletions or insertions of one or more nucleic acids, variations in the number of repetitive DNA elements, and changes in a single nitrogenous base position, also known as “single nucleotide polymorphisms” or “SNPs”. It is noted that any of the genetic variations herein can appear in DNA as well as RNA.

SNPs that may be used in the methods and compositions of the invention include those described in Hinds et al. (2005) Science 307:1072-1079 and available at genome.perlegen.com; research.calit2.net/hep/wgha/; hapmap.org; sciencemag.org/cgi/content/full/307/5712/1072/DC1; dbSNP; and genewindow.nci.nih.gov.

It is estimated that there are 3-4 million common SNPs. Typically, SNPs are biallelic, which means that they occur in two forms, a major allele and a minor allele, with the major allele being more frequently observed than the minor allele. Typically, the major allele occurs in more than 50% of the population; while the minor allele occurs in less than 50% of the population. Common SNPs are those SNPs that have a minor allele frequency of at least about 10%, meaning that within a given population the minor allele is present at the SNP locus at least about 10% of the time. Furthermore, common SNPs do not occur independently but are inherited together from generation to generation in genetic disequilibrium with other SNPs, forming patterns across genomic DNA and RNA. Groups of SNPs that are in linkage disequilibrium with one another define genomic regions that are referred to herein as haplotype blocks.

The term “haplotype block” as used herein refers to a region of a chromosome that contains one or more polymorphic sites (e.g., 1-10) that tend to be inherited together (i.e., are in linkage disequilibrium) (see Patil, et al., Science, 294:1719-1723 (2001); US 20030186244)) or that are together associated with a phenotypic trait of interest. In other words, combinations of polymorphic forms at the polymorphic sites within a block cosegregate in a population (e.g., with a phenotypic trait of interest) more frequently than combinations of polymorphic sites that occur in different haplotype blocks. In some embodiments, adjacent haplotype blocks do not overlap one another (i.e., are “nonoverlapping”). In some embodiments, a haplotype block may also be a linkage disequilibrium bin (see Hinds, et al., Science, 307:1072-1079 (2005)). A haplotype block is further characterized by one or more haplotype patterns. A haplotype pattern is the set of SNP alleles on a single nucleic acid strand within a single haplotype block (e.g., on a single chromosome of a single individual). SNP alleles, haplotype patterns, and allelic variations that do not have a frequency of at least about 10% in a given population can be described as rare. Therefore, SNPs with a minor allele frequency of less than about 10% may be referred to herein as “rare SNPs”, and haplotype patterns and allelic variations that have a frequency of less than 10% in the population may be referred to herein “rare haplotype patterns” and “rare allelic variations,” respectively.

Table 1 below illustrates nucleotide bases in six positions in a DNA molecule from three individuals. The nucleotide base positions can be in genomic DNA or RNA (U replaces T in RNA).

TABLE 1

Nucl. Position:

| | 1 | 2 | 3 | 4 | 5 | 6 |
|---|---|---|---|---|---|---|
| Individual 1: | T | A | G | T | C | G |
| Individual 2: | T | A | A | T | C | C |
| Individual 3: | T | A | G | T | C | G |

At nucleotide positions 1-2 and 4-5, all three individuals have the same nucleotide bases. At nucleotide positions 3 and 6, individual 2 has SNP alleles represented by underlined nucleotide bases A and C, respectively, as compared with individuals 1 and 3 who have SNP alleles G and G at the same nucleotide positions.

If both major and minor alleles of SNPs found at positions 3 and 6 have an allele frequency of at least about 10% in the population (e.g., major and minor SNP alleles occur at a ratio of 90% and 10%, or 70% and 30%, but not 95% and 5%, respectively), then such SNPs are referred to as common SNPs. Furthermore, if the two SNP alleles (e.g., A and C) at positions 3 and 6 consistently appear together (i.e., are in linkage disequilibrium with one another), then they are part of a haplotype pattern. A haplotype pattern refers to genotyped SNP alleles that appear together more frequently than expected by chance. The SNP locations of the SNP alleles in a haplotype pattern form a haplotype block. Haplotype blocks can include known as well as currently unknown SNPs. A SNP whose genotype is predictive of a genotype of one or more other SNPs in a haplotype block is often referred to as an “informative SNP”. For purposes of conducting association studies to predict a phenotype-of-interest, it may be sufficient to scan only one, only two, or only a few informative SNPs from one or more haplotype blocks.

In some embodiments, the present invention contemplates scanning an initial set of nucleotide bases from a plurality of individuals to identify one or more genetic variations (e.g., common SNPs). Such a scanning step can occur prior to, contemporaneous with, or after receiving data on the set of phenotypes for such individuals that are selected for an association study, i.e., responsiveness to an insulin sensitizer, where the response may be a therapeutic response or a non-therapeutic response, e.g., a side effect such as an adverse effect. This initial set of bases can come from the same and/or different individuals as those selected for the association study.

In some cases a surrogate marker is used as an indication of a response to a drug, e.g., insulin sensitizer. Such markers are well-known in the art and are typically chosen for their ease of measurement, and often because they indicate a therapeutic or non-therapeutic effect before such an effect becomes apparent from clinical observation. An example is the use of blood lipid levels or ratios as markers for potential adverse (or therapeutic) cardiovascular effects. Any suitable surrogate marker may be used in the methods of the invention. One useful marker is levels of expression of proteins coded for by genes involved in the disorder being treated; increases and/or decreases in protein expression levels and ratios of expression of certain proteins can be useful and powerful indicators of therapeutic or non-therapeutic effects. Methods for determining protein expression levels are well-known in the art.

Methods for identifying genetic variations are known in the art. For example, the identity of SNPs and SNP haplotype blocks across one representative chromosome (e.g., Chromosome 21) are disclosed in U.S. Provisional Serial No., 60/323,059, filed Oct. 31, 2002, entitled “Human Genomic Polymorphisms” assigned to the assignee of the present invention; and U.S. application Ser. No. 10/284,444, filed Oct. 31, 2002, entitled “Human Genomic Polymorphisms”, incorporated herein by reference for all purposes. See also Patil, N. et al., “Blocks of Limited Haplotype Diversity Revealed by High-Resolution Scanning of Human Chromosome 21” Science 294, 1719-1723 (2001), disclosing SNPs and haplotype structure of Chromosome 21.

In some embodiments, whole genome analysis is performed to identify genetic variations across the entire genome (DNA and/or RNA). Methods for whole genome analysis can be used both to identify known and/or new variations. Such methods are described in U.S. Provisional Application No. 60/327,006, filed Oct. 5, 2001, entitled “Identifying Human SNP Haplotypes, Informative SNPs and Uses Thereof,” and U.S. Pat. No. 6,969,589, both of which are assigned to the assignee of the present invention, all of which are incorporated herein by reference for all purposes. Additional descriptions of genome analysis, i.e., methods of sequencing, are suggested in U.S. Pat. Nos. 6,767,706; 6,818,395; 6,833,242; 6,344,325; and 6,221,654, all of which are incorporated herein by reference for all purposes.

Briefly, in order to scan full genomes, full sets of chromosomes may be separated from samples from individuals (e.g., more than 10, more than 20, more than 30, more than 40, or more than 50 individuals). This results in multiple unique genomes. In some embodiments, haploid genomes (or genomes derived from a single set of chromosomes) are used.

In some embodiments, RNA (e.g. mRNA) may be scanned to identify genetic variations. In order to scan RNA, RNA is first isolated from a cell, group of cells, or individuals. Methods for isolating RNA are known in the art. RNA can be isolated from more than 10, more than 20, more than 30, more than 40, or more than 50 individuals. Differences in expression patterns and/or genetic variations in RNA can be identified using any means known in the art or disclosed herein. See e.g. U.S. application Ser. Nos. 10/438,184 and 10/845,316, and PCT/US/04/010699, which are incorporated herein by reference for all purposes.

In some embodiments, all or a significant portion of an individual's genetic material (e.g., DNA, RNA, mRNA, cDNA, other nucleotide bases or derivative thereof) is scanned or sequenced using, e.g., conventional DNA sequencers or chip-based technologies to identify a set of SNPs and their corresponding alleles. In some embodiments, whole-wafer technology from Affymetrix, Inc. of Santa Clara, Calif. is used to read each individual's genome and/or RNA at single-base resolution.

A scanning step or diagnostic tool (whether to identify new genetic variations or to genotype an individual) can involve scanning at least about 1 base, at least about 10 bases, at least about 100 bases, at least about 1000 bases, at least about 10,000 bases, at least about 20,000 bases, at least about 50,000 bases, at least about 100,000 bases, at least about 200,000 bases, at least about 500,000 bases, at least about 1,000,000 bases, at least about 2,000,000 bases, at least about 5,000,000 bases, at least about 10,000,000 bases, at least about 20,000,000 bases, at least about 50,000,000 bases, at least about 100,000,000 bases, at least about 200,000,000 bases, at least about 500,000,000 bases, at least about 1,000,000,000 bases, at least about 2,000,000,000 bases, or at least about 3,000,000,000 bases, or substantially all of an individual's genetic material.

In some embodiments, a scanning step or diagnostic tool that identifies or genotypes genetic variations scans less than 100,000,000 bases, less than 50,000,000 bases, less than 10,000,000 bases, less than 5,000,000 bases, less than about 3,000,000,000 bases, less than about 2,000,000,000 bases, less than about 1,000,000,000 bases, less than about 500,000,000 bases, less than about 200,000,000 bases, less than about 100,000,000 bases, less than about 50,000,000 bases, less than about 20,000,000 bases, less than about 10,000,000 bases, loess than about 5,000,000 bases, less than about 2,000,000 bases, less than about 1,000,000 bases, less than about 500,000 bases, less than about 200,000 bases, less than about 100,000 bases, less than about 50,000 bases, less than about 20,000 bases, less than about 10,000 bases, less than about 5,000 bases, less than about 2,000 bases, less than about t 1,000 bases, less than about 500 bases, less than about 200 bases, less than about 100 bases, less than about 50 bases, less than about 20 bases, or less than about 10 bases.

Scanning nucleotide bases in a first set of individuals (e.g., at least about 10 individuals, at least about 20 individuals, at least about 30 individuals, at least about 40 individuals, or at least about 50 individuals) allows for identification of new genetic variations and/or genetic variations between individuals. Genetic variation data generated from each individual e.g. is compared with genetic variation data generated from other individuals in the first set of individuals in order to discover 1, 2, 3, 4, 5, 6, 7, 8, 9, 10 or more or 10, 20, 30, 40, 50, 60, 70, 80, 90, 100 or more or 1,000, 5,0000, 100,000, 500,000, 1,000,000 or more, or substantially all or all genetic variations among the first group of individuals.

The variations identified in the first set of individuals can be used in subsequent association studies in which such variations are analyzed to determine if they are associated with a phenotype-of-interest. These variations include, e.g., SNPs, common SNPs, informative SNPs, rare SNPs, deletions, insertions, frameshift mutations, etc. Such genetic variations can be detected in, for example, genomic DNA, RNA, mRNA, or derivatives thereof. In some embodiments, genetic variations scanned and/or identified are informative SNPs. Identification of informative SNPs can reduce the cost and increase the efficiency of association studies because the genotype of a single informative SNP can predict the genotype of one or more other SNP locations. Further descriptions of informative SNP and use thereof are further discussed in: U.S. Provisional Application Ser. No. 60/280,530, filed Mar. 30, 2001, entitled “Identifying Human SNP Haplotypes, Informative SNPs and Uses Thereof”; U.S. Provisional Application Ser. No. 60/313,264 filed Aug. 17, 2001, entitled “Identifying Human SNP Haplotypes, Informative SNPs and Uses Thereof”; U.S. Provisional Application Ser. No. 60/327,006, filed Oct. 5, 2001, entitled “Identifying Human SNP Haplotypes, Informative SNPs and Uses Thereof”; U.S. Provisional Application Ser. No. 60/332,550, filed Nov. 26, 2002, entitled “Methods for Genomic Analysis”; U.S. Pat. No. 6,969,589; and U.S. application Ser. No. 10/284,444, filed Oct. 31, 2004, entitled “Human Genomic Polymorphisms,” all of which are incorporated herein by reference for all purposes.

For example, in conducting whole genome association studies, instead of scanning and reading all 3 billion bases from each genome or about 3 to 4 million common SNPs, it is possible to scan or read simply about 200,000 to 500,000 informative (or “tag”) SNPs, which may provide a majority of the information as would scanning the entire genome (Hinds, et al., Science, 307:1072-1079 (2005)). Thus, while in some embodiments the present invention contemplates scanning whole genomes for association studies, in other embodiments only specific chromosomes, genomic regions, SNPs, common SNPs, or informative SNPs are scanned (e.g., genotyped) and/or used to conduct association studies. Specific chromosomes, genomic regions, SNPs, common SNPs, or informative SNPs may be selected for association studies based on prior knowledge that such regions are or may be related to a particular phenotype-of-interest (e.g., disease state or lack thereof). For example, they may have been previously identified in other association studies (e.g., linkage disease mapping studies) or based on homology with genes of known function.

Thus, in some embodiments the methods of the invention utilize known SNPs, available from databases herein or from any suitable source known in the art. In these embodiments, it is not necessary to scan the entire genome. In some embodiments, known sites of genetic variation, e.g., SNPs, are scanned. These embodiments can involve genotyping less than about 15,000; 200,000; 500,000; 1,000,000; 2,000,000, or substantially all genetic variations of an individual. In some embodiments, known sites of genetic variation, e.g., SNPs, are scanned. These embodiments can involve genotyping at least about 10,000; 100,000; 500,000; 1,000,000; 2,000,000; or substantially all genetic variations of an individual. In some embodiments, about 1 to about 10, about 10 to about 100, about 100 to about 1000, about 1000 to about 10,000, about 10,000 to about 100,000, about 10,000 to about 1,000,000, about 10,000 to about 2,000,000, or about 100,000 to about 2,000,000, or about 500,000 to about 2,000,000, or about 1,000,000 to about 2,000,000, or about 10,000 to about 1,000,000, or about 10,000 to about 500,000, or about 10,000 to about 100,000, or about 100,000 to about 2,000,000, or about 100,000 to about 1,000,000, or about 100,000 to about 500,000 genetic variations in an individual are genotyped.

The present invention contemplates association studies using genetic variations and phenotypes of individuals from both case and control groups. Case group individuals are those who express a phenotype-of-interest, e.g., responsiveness to drug such as an insulin sensitizer, where the responsiveness may be therapeutic or non-therapeutic response. Control group individuals are those who do not express the phenotype-of-interest, i.e., who do not exhibit responsiveness to an insulin sensitizer. In some embodiments, a case group includes at least 2, 5, 10, 20, 50, 100, 200, 500, or 1000 individuals and a control group includes at least 2, 5, 10, 20, 50, 100, 200, 500, or 1000 individuals. Methods for performing genotype association studies using case and control groups are described, e.g., in U.S. Ser. No. 10/351,973, filed Jan. 27, 2003, entitled “Apparatus and Methods for Determining Individual Genotypes”; in U.S. Ser. No. 10/786,475, filed Feb. 24, 2004, entitled “Improvements to Analysis Methods for Individual Genotyping”; U.S. Ser. No. 10/970,761, filed Oct. 20, 2004, entitled “Analysis Methods and Apparatus for Individual Genotyping”, U.S. application Ser. No. 10/427,696, filed Apr. 30, 2003, entitled Method for Identifying Matched Groups,” U.S. application Ser. No. 10/768,788, filed Jan. 30, 2004, entitled Apparatus and Methods for Analyzing and Characterizing Nucleic Acid Sequences,” and in U.S. application Ser. No. 10/956,224, filed Sep. 30, 2004, entitled “Methods for Genetic Analysis”, all of which are incorporated herein by reference for all purposes.

To increase efficiency of collecting genotyping data, cases and/or controls can be pooled prior to scanning as is described in U.S. application Ser. No. 10/447,685, filed May 28, 2003, entitled “Liver Related Disease Compositions and Methods”, U.S. application Ser. No. 10/427,696; filed Apr. 30, 2003; entitled “Methods for Identifying Matched Groups”; and 10/768,788; filed Jan. 30, 2004; entitled “Apparatus and Methods for Analyzing and Characterizing Nucleic Acid Sequences” which are incorporated herein by reference. For example, samples obtained from all or some case individuals may be pooled together prior to scanning and/or all or samples from all or some control individuals may be pooled together separately (pool of cases and pool of controls) prior to scanning. In another example, data on genetic variations and/or phenotypes from some or all case individuals and/or some or all control individuals may be pooled together. Furthermore, in any of the embodiments herein, genetic variation data collected can be stored in a computer readable medium for further analysis.

In any of the embodiments herein, a scanning step (for either identifying or genotyping variations) may be supplemented and/or substituted by receiving data on the genetic variations from database(s). Such databases can provide, for example, a list of identified genetic variations (e.g., SNPs or haplotypes) or genotyping data on particular individuals. Examples of publicly available databases that identify genetic variations include, but are not limited to, genome.perlegen.com; research.calit2.net/hep/wgha/; hapmap.org; sciencemag.org/cgi/content/ful/307/5712/1072/DC1; genewindow.nci.nih.gov. NCBI's dbSNP (ncbi.nlm.nih.gov/SNP/index.html); MIT's human SNP database (broad.mit.edu/snp/human/); University of Geneva's human Chromosome 21 SNP database (csnp.unige.ch/). and the University of Tokyo's SNP database (snp.ims.u-tokyo.ac.jp/). Other databases known in the art may be used in conjunction with the methods herein.

In some embodiments, described herein, the present invention contemplates the use of one or more genetic variations between individuals (e.g., SNP alleles, and haplotype patterns) in association studies to predict if an individual has or does not have responsiveness to an insulin sensitizer. In other embodiments, the present invention contemplates using phenotypic variations in addition to genotypic variations in association studies. Association studies using only genetic variations are described in U.S. application Ser. No. 10/447,685, filed May 28, 2003, entitled “Liver Related Disease Compositions and Methods”, U.S. Provisional Patent Application No. 60/648,957, filed Jan. 31, 2005, entitled “Compositions and Methods For Treating, Preventing, and Diagnosing Alzheimer's Disease,” and U.S. Provisional Patent Application No. 60/653,672, filed Feb. 16, 2005, entitled “Parkinson's Disease-Related Disease Compositions and Methods”, which are incorporated herein by reference.

Association studies using genetic variations and phenotypic variations are described in U.S. patent application Ser. No. 11/043,689, filed Jan. 24, 2005, entitled “Associations Using Genotypes and Phenotypes;” which is incorporated herein by reference. Like genotyping data, data on a set of phenotypes of the individuals is received for both case individuals and control individuals. The data on a set of phenotypes can include data on at least about 1, 2, 3, 4, 5, 6, 7, 8, 9, or 10 different phenotypes, or on at least about 10, 25, 30, 35, 40, 45 or 50 different phenotypes of the individuals in the association study. The data on the set of phenotypes can be collected prior to, subsequent to, or simultaneous with the collection/gathering of genotyping data. Phenotype data collected can (like the genotyping data) also be stored in a computer readable medium for further use.

### A. Genotyping Data

An association study may be performed to identify genetic loci associated with responsiveness to a drug, e.g., to an insulin-sensitizing drug. A two-stage approach can be used in which, first, pooled sample sets are used to identify genetic regions that may be associated with the trait of interest. While actual allele frequency differences between the case and control sample pools cannot be measured in these sample sets, allele frequency differences may be estimated, and used to select a subset of SNPs for further evaluation. This subset, which may have contained false positives in addition to true positives, is then genotyped in the individual case and control samples, and the exact allele frequency differences between the populations are calculated. In some embodiments, the population that is individually genotyped is the same as the population subjected to pooled genotyping; in other embodiments, the population that is individually genotyped is different from the population subjected to pooled genotyping; in still other embodiments, individual genotyping may be performed both on the population that was subjected to pooled genotyping as well as at least one additional population. In some embodiments, the pooled populations for cases and for controls can be matched with regard to population structure in order to reduce variation not associated with the phenotype(s) of interest. Such an analysis may also be performed after pooling to verify that the populations are matched. Methods of matching populations may be found in, e.g. Hinds, D. A. et al. Matching strategies for genetic association studies in structured populations. Am J Hum Genet 74, 317-25 (2004), U.S. patent application Ser. No. 10/427,696, filed Apr. 30, 2003, entitled “Method for Identifying Matched Groups,” and Bacanu, S. A., et al. The power of genomic control. Am J Hum Genet 66, 1933-44 (2000), all of which are incorporated by reference herein in their entirety.

In some embodiments, a third stage may be performed in which SNPs showing significant association with susceptibility (or resistance) to adverse effects of insulin-sensitizing drugs in the original sample set are then analyzed in a one or more additional sample sets, in order to verify or validate their association. These studies are referred to as validation studies.

The human genome is scanned to identify or genotype genetic variants using microarray technology platforms such as described in U.S. Pat. No. 6,586,750, entitled “High Performance Substrate Scanning”, U.S. Pat. No. 6,969,589, assigned to the same assignee as the present application; U.S. Ser. No. 10/284,444, entitled “Chromosome 21 SNPs, SNP Groups and SNP Patterns,” filed on Oct. 31, 2002, assigned to the same assignee as the present application; and U.S. Pat. No. 6,897,025, entitled “Genetic Analysis Systems and Methods,” issued on May 24, 2005, assigned to the same assignee as the present application, all of which are incorporated herein by reference. The microarrays are manufactured using a process adapted from semiconductor manufacturing to achieve cost effectiveness and high quality.

Variants identified are grouped into haplotype blocks using methods disclosed in U.S. Pat. No. 6,969,589, incorporated herein by reference. Representative variants and haplotype blocks from an entire human chromosome (chromosome 21) are disclosed in, for example, U.S. application Ser. No. 10/284,444, filed Oct. 31, 2002, entitled “Human Genomic Polymorphisms”; and Patil, N. et al, “Blocks of Limited Haplotype Diversity Revealed by High-Resolution Scanning of Human Chromosome 21” Science 294, 1719-1723 (2001) and the associated supplemental materials, incorporated herein by reference.

Case samples are obtained from individuals who have demonstrated responsiveness when given the drug, e.g., insulin sensitizer. Individuals are evaluated clinically. A “response” is as described herein, and can be a therapeutic response or a non-therapeutic response. Non-therapeutic responses include adverse effects, including, but not limited to (in the case of insulin sensitizers) peripheral edema, dependent edema, generalized edema, weight increase, anemia, hypoglycemia, headache, increase in micturation frequency, diarrhea, increased appetite, transient ischemic attack, elevated liver enzymes, and combinations thereof. Control samples are obtained from individuals who have not demonstrated the same responsiveness as the case individuals when given the drug, e.g., insulin sensitizer; controls and cases have different responses, e.g., one may have low efficacy and the other high efficacy, etc. Criteria for inclusion in the case and control groups are determined prior to the commencement of the study.

Pooled genotyping Arrays are designed to assay for all or a subset of the total SNPs of the genome. Any subset may be used that produces meaningful data; for example, numerous publicly available databases describe subsets of SNPs in the human genome and are available, e.g., as described elsewhere herein.

Oligonucleotide arrays are designed such that each SNP is interrogated by a set of probes. For example, in some embodiments an array may be designed so that each SNP is interrogated by, e.g., forty distinct 25 bp probes. These forty features consist of four sets of ten features, corresponding to the forward and reverse strands of the two SNP alleles (reference and alternate). Each set of ten features consists of two sets of five features, with offsets of −2, −1, 0, +1, and +2 bases between the center of the 25 bp probe and the SNP position. For each offset, one perfect-match feature and one mismatch feature (complement of the perfect match at the interrogation position only) are tiled at the central position of the probe. Thus, for each allele there is a total of ten perfect-match probes and ten mismatch probes. The oligonucleotide features necessary to query the SNPs are arrayed on one or more distinct array designs. See: U.S. application Ser. No. 10/970,761, filed Oct. 20, 2004, entitled “Improved Analysis Methods and Apparatus for Individual Genotyping” for further description.

Samples can be analyzed for quality, e.g., as follows: (1) concentration and volume are measured to make sure that they match the expected values, and are adequate for the study; (2) gel electrophoresis is performed on a subset of samples to examine DNA integrity; and (3) PCR assays are performed to establish the ability of the DNA samples, or a subset thereof, to be amplified.

After passing QC the samples are diluted to an appropriate concentration, and re-quantified. The samples can be divided into pools in any suitable manner. In any given pooled analysis, the samples may be pooled in such a manner as to provide the desired information regarding genotype and/or variations due to sample handling. In some embodiments, cases and controls may each be divided into individual pools, and each individual pool can be analyzed. The number of pools may be one, two, three, four, five, six, seven, eight, nine, ten, or more than ten. For example, the samples may be divided into a total of, e.g., eight pools, four containing case samples and four containing control samples. Thus, each pool contains, e.g., 100 samples, randomly selected from either the cases or controls, with each sample present in just one pool. Equimolar amounts of each sample are transferred into one of the eight pools. Each pool may then be re-quantified and diluted to a standard concentration for use as a PCR template. Alternatively, in some embodiments, replicate pools, each of which consists of the same subset of the cases or controls may be analyzed. The number of pools may be one, two, three, four, five, six, seven, eight, nine, ten, or more than ten. For example, the samples may be divided into a total of, e.g., eight pools, four containing case samples and four containing control samples. Thus, each pool contains, e.g., 100 samples, randomly selected from either the cases or controls, but in this embodiment, each sample is present in each of the four case pools or in each of the four control pools. Equimolar amounts of each sample are transferred into one of the eight pools. Each pool may then be re-quantified and diluted to a standard concentration for use as a PCR template. The latter embodiment allows the study of variations introduced by the procedures used in analysis. In other embodiments, aliquots of all the case samples may be present in more than one “case pool,” and aliquots of all the control samples may be present in more than one “control pool.”

The pools are independently amplified using, e.g., multiplexed PCR with a single primer pair for each SNP. The amplified products are pooled, labeled and hybridized to the chip or chips that individually or together query the set of SNPs selected for genotyping. The hybridized chips are washed and stained. The hybridization of labeled sample is detected. See, e.g., U.S. Ser. No. 11/344,975, filed Jan. 31, 2006, entitled “Genetic Basis of Alzheimer's Disease and Diagnosis and Treatment Thereof,” U.S. Ser. No. 11/299,298, filed Dec. 9, 2005, entitled “Markers for Metabolic Syndrome Obesity and Insulin Resistance,” and U.S. Ser. No. [unassigned], docket no. 300/1081-10, filed Sep. 27, 2006, entitled “Genetic Basis of Rheumatoid Arthritis and Diagnosis and Treatment Thereof.”

After removing SNP measurements that fail quality control (see below), the estimated allele frequency difference between case and control pools, termed delta p-hat, is automatically derived for each SNP from intensity ratios for hybridization to the allele-specific 25-mer features. The fluorescence intensities of the reference and alternate perfect-match features on the arrays correlate with the concentration of the corresponding SNP allele in the DNA sample. Estimates of allele frequency, p-hat, are computed from ratios of trimmed means of intensities of the perfect-match features, after subtracting a measure of background computed from trimmed means of intensities of mismatch features. The case pool p-hats and control pool p-hats are separately averaged, and the delta p-hat is calculated. Finally, the standard error of the estimate, based on the within pool variance of the measurements, t-statistic p-value, and empirical p-values (which were obtained as rank of T_TEST_P_VALUE on each chip design divided by the total number of passing SNP measurements for each chip design) for the delta p-hat are calculated for each of the SNPs that passed the QC filters. See, e.g., U.S. Ser. No. 10/768,788, filed Jan. 30, 2004, entitled “Apparatus and Methods for Analyzing and Characterizing Nucleic Acid Sequences.”

The following quality control filters can be applied to the data to assess the reliability of the fluorescence intensities of the features for each SNP in an array scan. Applying these filters, which are based on findings from numerous previous association studies, increases the quality of the passing SNPs, thereby reducing false-positive associations. SNP measurements are removed from consideration if they had any of the following: (1) conformance of <0.9; (2) saturated probes; and (3) signal-to-background ratio of <1.5. U.S. application Ser. No. 10/970,761, filed Oct. 20, 2004, entitled “Improved Analysis Methods and Apparatus for Individual Genotyping” has further descriptions of the implementation of these quality measures.

The conformance of alleles is defined as the fraction of feature pairs for which the perfect-match feature is brighter than the corresponding mismatch feature. A conformance of <0.9 can indicate the absence of target DNA. Both saturated probes and low signal-to-background ratios can lead to unreliable p-hat measurements.

In some embodiments, SNPs are selected for further evaluation (individual genotyping) if they pass other criteria, such as threshold for p-values for the delta p-hat measurements, standard error (SE) of delta p-hat measurements, and other QC filters. In addition, individual genotyping may also include SNPs not amplified in the pooled genotyping phase; SNPs to control for population stratification (see, e.g., U.S. patent application Ser. No. 10/427,696, filed Apr. 30, 2003, entitled “Method for identifying matched groups”); and/or SNPs from candidate regions.

### Individual Genotyping

### SNPs Genotyped in Individual Samples

Selected SNPs are individually genotyped in each of the case and control samples. These can include SNPs selected on the basis of the pooled genotyping results, SNPs from the candidate regions, and SNPs to control for population stratification. The case and control samples that are individually genotyped may be the same as or different from those that were subjected to pooled genotyping.

### High-Density Oligonucleotide Arrays

A new array can be designed to individually genotype the selected SNPs, such that all selected SNPs can be assayed using a single chip for each individual DNA sample.

### Individual Genotyping of Case and Control Samples

The SNPs from the case and control samples are amplified. The amplified samples from each individual are pooled and hybridized to oligonucleotide arrays, thereby querying each SNP for only one individual in each pooled sample. In addition, the samples may be pooled prior to being amplified. The hybridized chips are washed and stained, and the resulting fluorescence detected as for the pooled genotyping.

Methods for individual genotyping are detailed, e.g., in U.S. Ser. No. 10/351,973, filed Jan. 27, 2003, entitled “Apparatus and Methods for Determining Individual Genotypes,” U.S. Ser. No. 10/786,475, filed Feb. 24, 2004, entitled “Analysis Methods for Individual Genotyping,” U.S. Ser. No. 10/970,761, filed Oct. 20, 2004, entitled “Analysis Methods and Apparatus for Individual Genotyping,” and U.S. Ser. No. 11/173,809, filed Jul. 1, 2005, entitled “Algorithm for Estimating Accuracy of Genotype Assignment.” In certain embodiments, individual genotypes for each SNP are determined by clustering the intensity measurements of all samples, in the two-dimensional space defined by background-adjusted trimmed mean intensities of the perfect-match features for the reference and alternate alleles. See U.S. application Ser. No. 10/970,761, filed Oct. 20, 2004, entitled “Improved Analysis Methods and Apparatus for Individual Genotyping”; Hinds, D. A. et al. Matching strategies for genetic association studies in structured populations. Am J Hum Genet 74, 317-25 (2004); Hinds, D. A. et al. Application of pooled genotyping to scan candidate regions for association with HDL cholesterol levels. Human Genomics 1, 421-34 (2004); and Hinds, D. A. et al. Whole genome patterns of common DNA variation in human populations. Science 307, 1072-1079 (2005). A K—means algorithm can be used to assign the measurements to clusters representing the three distinct diploid genotypes that are possible: homozygous-reference, heterozygous, and homozygous-alternate. The K-means and background optimization steps are iterated until cluster membership and background estimates converge. To determine the appropriate number of genotype clusters, the analysis can be repeated for 1, 2, and 3 clusters, and selecting the most likely solution, considering likelihoods of the data and the cluster parameters.

### Quality Control

Quality control filters can be applied to the data to assess the reliability of the fluorescence intensities of the features for each SNP in an array scan. Applying these filters, which are based on findings from numerous previous association studies, increases the quality of the passing SNPs. SNPs that pass the individual genotyping quality filters are analyzed further. Such filters can be based on, e.g., combinations of call rate and Hardy-Weinberg equilibrium p-values. The specific combination chosen depends on the degree of increase in quality of the passing SNPs desired. For example, one filter requires a call-rate of 0.8, meaning that the SNP has an unambiguous genotype call in at least 80% of the samples; and a Hardy-Weinberg equilibrium p-value of >0.0001. SNP call rates may be computed after discarding genotypes that obtained <0.2 score with an individual genotyping error probability metric. In some embodiments, the metric uses machine learning algorithm to approximate a probability of a genotype being discordant with outside platforms from 15 QC and SNP-property based inputs. See, e.g., U.S. application Ser. No. 10/970,761, filed Oct. 20, 2004, entitled “Analysis Methods and Apparatus for Individual Genotyping,” and U.S. application Ser. No. 11/173,809, filed Jul. 1, 2005, entitled “Algorithm for Estimating Accuracy of Genotype Assignment,” the disclosures of which are incorporated by reference in their entireties.

### Individual Genotyping Results

### Assessing the False-Positive and False-Discovery Rates

The number of SNPs with significant trend test p-values that would be expected to be found purely by chance can be calculated, assuming no enrichment of large allele frequency differences in the pooling phase of the study. The expected value may be compared with the actual number, and if the number of observed SNPs below each of the different p-value cutoffs is greater than the expected number, then the pooled genotyping did indeed enrich the SNP set for SNPs with large allele frequency differences. False discovery rates may also be calculated as the ratio of the expected number of false positives to the number of observed SNPs with significant trend test p-values below a certain cutoff.

Thus, using high density array technology, SNPs associated with susceptibility to one or more adverse effects of a drug or class of drugs are identified by measuring SNP allele frequency differences between cases and controls. In some embodiments, all or substantially all of the SNPs thus identified may be used in other studies or in the clinical setting to predict a response or non-response to an insulin sensitizer. In some embodiments, a subset of the SNPs thus identified may be used in other studies or in the clinical setting to predict a response or non-response to an insulin sensitizer

B. Association Studies with Both Genotype and Phenotype Data

Both genotype and phenotype data may be used in an association study in methods of the invention. Association studies using genetic variations as well as phenotypic variations are described in U.S. patent application Ser. No. 11/043,689, filed Jan. 24, 2005, entitled “Associations Using Genotypes and Phenotypes” which is incorporated herein by reference.

Data on genetic variations from a plurality of individuals with and without a phenotype-of-interest, i.e., responsiveness to an insulin sensitizer, is received, as described above. It will be appreciated that the phenotype of interest, e.g., response to an insulin sensitizer, is not the same as the phenotypes described in this section; these phenotypes are used to determine the presence or absence between one or more of them and the phenotype of interest.

In the methods of the invention, the phenotype-of-interest is a response to an insulin sensitizer that would include or exclude an individual from a drug trial or a drug therapy. See U.S. Provisional No. 60/566,302, filed Apr. 28, 2004, entitled “Methods for Genetic Analysis”; U.S. Provisional No. 60/590,534, filed Jul. 22, 2004, entitled “Methods for Genetic Analysis,” U.S. Ser. No. 10/956,224, filed Sep. 30, 2004, entitled “Methods for Genetic Analysis,” and U.S. Ser. No. 11/510,261, filed Aug. 25, 2006, entitled “Methods for Genetic Analysis,” all of which are incorporated herein by reference for all purposes.

Data on a group of phenotypes of the plurality of individuals can also be received. The group of phenotypes includes the phenotype-of-interest. Data on the group of phenotypes can be received prior to, after, and/or concurrent with the receipt the data of the genetic variations. In some embodiments, data on the group of phenotypes is generated by a practitioner of the present invention by, for example, observation (e.g., gross phenotypic trait), biochemical testing (e.g. blood or urine analysis), or other diagnostic test (e.g., X-ray, MRI, CAT scan, CT scan, Doppler shift, etc.).

Examples of phenotype data that may be received/collected include, but are not limited to, data about the individuals': ability to roll the tongue, ability to taste PTC, acute inflammation, adaptive immunity, addiction(s), adipose tissue, adrenal gland, age, aggression, amino acid level, amyloidosis, anogenital distance, antigen presenting cells, auditory system, autonomic nervous system, avoidance learning, axial defects or lack thereof, B cell deficiency, B cells, B lymphocytes (e.g. antigen presentation), basophils, bladder size/shape, blinking, blood chemistry, blood circulation, blood glucose level, blood physiology, blood pressure, body mass index, body weight, bone density, bone marrow formation/structure, bone strength, bone/skeletal physiology, breast size/shape, bursae, cancellous bone, cardiac arrest, cardiac muscle contractility, cardiac output, cardiac stoke volume, cardiomyopathy, cardiovascular system/disease, carpal bone, catalepsy, cell abnormalities, cell death, cell differentiation, cell morphology, cell number, cell-mediated immunity, central nervous system, central nervous system physiology, chemotactic factors, chondrodystrophy, chromosomal instability, chronic inflammation, circadian rhythm, circulatory system, cleft chin, clonal anergy, clonal deletion, T and B cell deficiencies, conditioned emotional response, congenital skeletal deformities, contextual conditioning, cortical bone thickness, craniofacial bones, craniofacial defects, crypts of Lieberkuhn, cued conditioning, cytokines, delayed bone ossification, dendritic cells (e.g. antigen presentation), Di George syndrome, digestive function, digestive system, digit dysmorphology, dimples, discrimination learning, drinking behavior, drug abuse, drug response, ear size/shape including ear lobe attachment, eating behavior, ejaculation function, embryogenesis, embryonic death, embryonic growth/weight/body size, emotional affect, enzyme/coenzyme level, eosinophils, epilepsy, epiphysis, esophagus, excretion physiology, extremities, eye blink conditioning, eye color/shape, eye physiology, eyebrows shape, eyelash length, face shape, facial cleft, femur, fertility/fecundity, fibula, finger length/shape, fluid regulation, fontanels, foregut, fragile skeleton, freckles, gall bladder, gametogenesis, gastrointestinal hemorrhage, germ cells (e.g., morphology, depletion), gland dysmorphology, gland function, glucagon level, glucose homeostasis, glucose tolerance, glycogen catabolism, granulocytes, granulocytes (e.g., bactericidal activity, chemotaxis), grip strength, grooming behavior, hair color, hair follicle structure/orientation, hair growth, hair on mid joints, hair texture, handedness, harderian glands, head, hearing function, heart, heart rate, heartbeat (e.g. rate, irregularity), height, hemarthrosis, hemolymphoid system, hepatic system, hitchhiker's thumb, homeostasis, humerus, humoral immune response, hypoplastic axial skeleton, hypothalamus, immune cell, immune system (e.g., hypersensitivity), immune system response/function, immune tolerance, immunodeficiency, inability to urinate, increased sensitivity to gamma-irradiation, inflammatory mediators, inflammatory response, innate immunity, inner ear, innervation, insulin level, insulin resistance, intestinal bleeding, intestine, ion homeostasis, jaw, kidney hemorrhage, kidney stones, kidney/renal system, kyphoscoliosis, kyphosis, lacrimal glands, larynx, learning/memory, leukocyte, ligaments, limb dysmorphology, limb grasping, lipid chemistry, lipid homeostasis, lips size/shape, liver (e.g. development/function), liver/hepatic system, locomotor activity, lordosis, lung, lung development, lymph organ development, macrophages (e.g. antigen presentation), mammary glands, maternal/paternal behavior, mating patterns, meiosis, mental acuity, mental stability, mental state, metabolism of xenobiotics, metaphysis, middle ear, middle ear bone, morbidity and mortality, motor coordination/balance, motor learning, mouth, movement, muscle, muscle contractility, muscle degeneration, muscle development, muscle physiology, muscle regeneration, muscle spasms, muscle twitching, musculature, myelination, myogenesis, nervous system, neurocranium, neuroendocrine glands, neutrophils, NK cells, nociception, nose, nutrients/absorption, object recognition memory, ocular reflex, odor preference, olfactory system, oogenesis, operant or “target response”, orbit, osteogenesis, osteogenesis/developmental, osteomyelitis, osteoporosis, outer ear, oxygen consumption, palate, pancreas, paralysis, parathyroid glands, pelvis girdle, penile erection function, perinatal death, peripheral nervous system, phalanxes, pharynx, photosensitivity, piloerection, pinna reflex, pituitary gland, PNS glia, postnatal death, postnatal growth/weight/body size, posture, premature death, preneoplasia, propensity to cross the right arm over the left of vice versa, propensity to cross the right thumb over the left thumb when clasping hands or vise versa, pulmonary circulation, pupillary reflex, radius, reflexes, reproductive condition, reproductive system, resistance to fatty liver development, resistance to hyperlipidemia, respiration (e.g., rate, shallowness), respiratory distress or failure, respiratory mucosa, respiratory muscle, respiratory system, response to infection, response to injury, response to new environment (transfer arousal), ribs, salivary glands, scoliosis, sebaceous glands, secondary bone resorption, seizures, self tolerance, senility, sensory capabilities, sensory system physiology/response, sex, sex glands, shoulder, skin, skin color, skin texture/condition, skull, skull abnormalities, sleep pattern, social intelligence, somatic nervous system, spatial learning, sperm count, sperm motility, spermatogenesis, startle reflex, sternum defect, stomach, suture closure, sweat glands, T cell deficiency, T cells (e.g., count), tarsus, taste response, teeth, temperature regulation, temporal memory, tendons, thyroid glands, tibia, touch/nociception, trachea, tremors, trunk curl, tumor incidence, tumorigenesis, ulna, urinary system, urination pattern, urine chemistry, urogenital condition, urogenital system, vasculature, vasoactive mediators, vertebrae, vesicoureteral reflux, vibrissae, vibrissae reflex, viscerocranium, visual system, weakness, widows peak or lack thereof, etc. See, e.g.,: U.S. patent application Ser. No. 11/043,689, filed Jan. 24, 2005, entitled “Associations Using Genotypes and Phenotypes,” the disclosure of which is incorporated herein in its entirety.

Additional examples of phenotype data that may be received/collected about individuals can include phenotype data about previous medical conditions or medical history (e.g., whether an individual has had surgery, experienced a particular illness, given vaginal or nonvaginal childbirth, been diagnosed with mental illness, has allergies, etc.).

In some embodiments, phenotype data may also be received/collected on the individuals' family history. For example, data can be collected on relatives suffering from or affected by baldness, cancer, diabetes, hypertension, mental illness, mental retardation, attention deficit, infertility, erectile dysfunction, cardiovascular disease, allergies, drug addiction, etc.

Data on one or more phenotypes is received for individuals with a phenotype-of-interest and without the phenotype-of-interest (i.e., responsiveness to an insulin sensitizer). In some embodiments, a larger set of possible phenotypes is used in the association study to provide the greatest probability of identifying the phenotype-of-interest in an individual who may or may not be in case or control groups. For example, data on more than 2, more than 3, more than 5, more than 7, more than 10, more than 15, more than 20, more than 25, more than 30, more than 35, more than 40, more than 45, more than 50, more than 60, more than 70, more than 80, more than 90, or more than 100 phenotypes may be used in an association study.

Data on the group of phenotypes may be received in a binary system (e.g., 0's and 1's) or a greater-fold system (e.g., three-fold, four-fold, etc., such as 0's, 1's 2's, etc.) on a phenotype-by-phenotype basis. An example of phenotypic data that may be received in a binary system includes the presence (or absence) of a disease. If an individual has a particular phenotype (e.g., disease) from a group of phenotypes, that phenotype may be designated as “1”. Conversely, if an individual does not have a particular phenotype from a group of phenotypes, that phenotype may be designated as “0”.

Similarly, data on the group of phenotypes may also be received in a greater-fold system, such as a three-fold, four-fold system, or a greater-fold system (e.g., more than 10-fold, more than 20-fold, or more than 40-fold). In greater-fold systems each of the multiple forms of a phenotype may be designated with a different number. For example, if an individual expresses a first form (e.g., blue eyes) of a phenotype (e.g., eye color) of a group of phenotypes, that phenotype may be designated as “1”, a second form (e.g., green eyes) of the phenotype of a group of phenotypes may be designated as “2”, a third form (e.g., brown eyes) of the phenotype of a group of phenotypes may be designated as “3”, etc.

Data on the plurality of phenotypes about an individual can also include data about a degree to which such phenotypes or plurality of phenotypes is present (or absent) in the individual. For example, the degree of skin pigmentation can be expressed as a gradient from 1 to 10 wherein “1” represents the lightest skin color and “10” represents the darkest skin color. Determination of the degree of skin pigmentation can be made by an observer (e.g., clinician) or can be made based on a plurality of other determinants using various mathematical-statistical methods including, but not limited to, multiple comparison (Bonferroni), variance analysis, regression and correlation analysis, and multivariant discriminant analysis (see U.S. Pat. No. 4,791,998, which is incorporated herein by reference for all purposes).

The genetic variations and the data on the group of phenotypes are used collectively in association studies with one (or more) phenotypes-of-interest. Alternatively, or in addition, the correlation may be conducted through pooling samples to reduce overall costs or by genotyping individual samples, as described for genotyping studies.

One or more phenotypes from the group of phenotypes are identified that can differentiate at least in part among individuals having and not having responsiveness to an insulin sensitizer. This can be achieved by identifying phenotypes from the group of phenotypes with significant frequency differences between cases and controls. In certain embodiments, steps the identification of phenotypes and genotypes that can differentiate at least in part among individuals having and not having responsiveness to an insulin sensitizer occur simultaneously.

In some embodiments, it is predicted whether an individual (that can be from neither the case nor the control groups) has or does not have responsiveness to an insulin sensitizer. This step optional. Further, a treatment, such as a drug treatment is administered (or not administered) to a patient, or a patient is enrolled in a clinical trial, based on the results of the predictive step.

Table 2 below illustrates hypothetical data received from six individuals. The data includes information on four genetic variations (common SNPs) and four phenotypes. For SNPs, the following letter symbols are used: (A) adenine (T) thymine (C) cytosine, and (G) guanine to indicate SNP alleles.

TABLE 2
Association Study Using Common SNPs (CSs) and Phenotypes (Phs)

| | Phenotype-of- | | | | | | | | |
|---|---|---|---|---|---|---|---|---|---|
| | interest | | | | | | | | |
| | (responsiveness | | | | | | | | |
| | to an insulin | | | | | | | | |
| Individual | sensitizer) | SNP 1 | SNP 2 | SNP 3 | SNP 4 | Phenotype 1 | Phenotype 2 | Phenotype 3 | Phenotype 4 |
| 1 | 1 | A | C | G | T | 1 | 0 | 2 | 7 |
| 2 | 1 | A | T | G | T | 1 | 0 | 1 | 8 |
| 3 | 0 | T | C | C | A | 0 | 1 | 0 | 1 |
| 4 | 0 | T | A | C | A | 0 | 1 | 2 | 2 |
| 5 | 1 | A | T | G | T | 1 | 0 | 2 | 9 |
| 6 | 0 | T | T | C | A | 0 | 1 | 0 | 1 |

As illustrated by Table 2, individuals 1, 2, and 5 have responsiveness to an insulin sensitizer (symbolized by a “1”) are cases, while individuals 3, 4, and 6 do not have responsiveness to an insulin sensitizer (symbolized by a “0”) are controls. The presence of “A” allele at SNP1, a “G” allele at SNP3, and/or a “T” allele at SNP4 are associated with an individual having responsiveness to an insulin sensitizer (“1”); while the presence of an “T” allele at SNP 1, “C” allele at SNP3, and/or an “A” allele at SNP4 is associated with an individual not having responsiveness to an insulin sensitizer (“0”).

Similarly, a phenotype score of “1” for phenotype 1, a phenotype score of “0” for phenotype 2, and/or a phenotype score of “7 or higher” for phenotype 4 is associated with an individual having responsiveness to an insulin sensitizer (“1”); while a phenotype score of “0” for phenotype 1, a phenotype score of “1” score for phenotype 2, and/or a phenotype score of “2 or less” is associated with an individual not having responsiveness to an insulin sensitizer (“0”).

Combining these data into a single association study, one can predict that an individual with an “A” allele at SNP1, “G” allele at SNP3, and/or “T” at SNP4, having a phenotype score of “1” for phenotype 1, phenotype score “0” for phenotypes 2, and/or phenotype score of “7 or higher” for phenotype 4, will have responsiveness to an insulin sensitizer (“1”). Conversely, an individual with a “T” allele at SNP1, a “C” allele SNP3, and/or an “A” allele at SNP4, having a phenotype score of “0” for phenotype 1, phenotype score of “1” for phenotype 2, and/or phenotype score of “2 or less” for phenotype 4 will not have responsiveness to an insulin sensitizer (“0”).

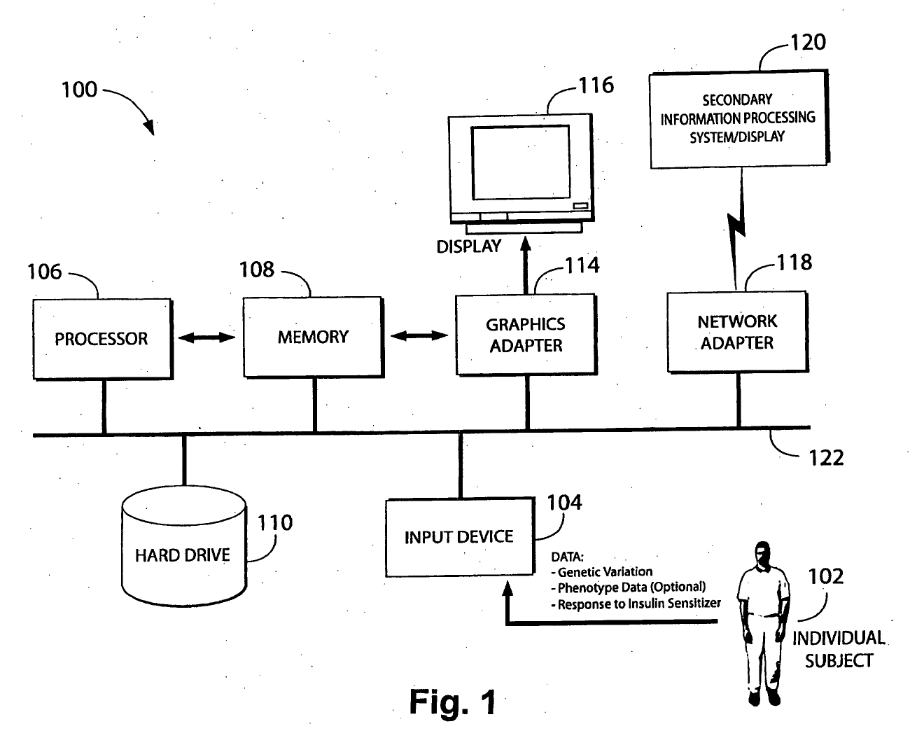

Data analysis and use FIG. 1 is a high-level block diagram of a computer system 100 for storing and processing data from one or more individuals 102. The data can include genetic data (e.g., genetic variations) and, optionally, phenotype data. For screening uses, one or more genetic variations and/or phenotypes may be input. For association studies, the data may further include data regarding response to one or more insulin sensitizers. The data are entered into the system via an input device 104. Illustrated are at least one processor 106 coupled to a bus 122. Also coupled to the bus 122 are a memory 108, a storage device 110, an input device 104, a graphics adapter 114, and a network adapter 118. A display 116 may be coupled to the graphics adapter 114. A secondary information processing and/or display system 120 is illustrated, which may be, e.g., a computer or other device that has access to a network, e.g., the Internet. Not all components described must be present for the use of methods and compositions of the invention.

The at least one processor 104 may be any general-purpose microprocessor. The storage device 110 may be any device capable of holding data, like a hard drive, compact disk read-only memory (CD-ROM), DVD, or a solid-state memory device. The memory 108 holds instructions and data used by the processor 106. The input device 104 may be, e.g., a mouse, track ball, light pen, touch-sensitive display, or other type of pointing device, that is used in combination with a keyboard to input data into the computer system. Data may also be input directly from devices or instruments that are used to assay genetic, phenotypic, and/or insulin sensitizer response data from the individual 102. The graphics adapter 114 displays images and other information on the display 116. The network adapter 118 couples the computer system 100 to a local or wide area network.

In some embodiments, data from one or more individuals are input into the computer system in one location, and further analyzed, displayed, or otherwise manipulated at a remote location. For example, some or all of the data of an association study may be acquired in one country and transferred to another country for analysis. Another example is that data from an individual to be screened may be acquired at a laboratory, e.g., a reference laboratory such as a Clinical Laboratory Improvement Amendments (CLIA) laboratory, and transferred to another location, e.g., a doctor's office, where it is further processed and/or displayed. The data may be moved to the remote location by any means, e.g., as data on a CD-ROM, or via a network, e.g., the Internet.

In a related embodiment, SNPs associated with the efficacy of a drug, e.g., an insulin sensitizer drug may be used to improve the efficacy of the drug by stratifying patient populations to exclude probable nonresponders from treatment. In one example, ˜32% of patients exposed to an insulin sensitizer drug are classified as responders. An association study is performed with a case group of responders and a control group of nonresponders, and 25 SNPs are found to be associated with the responder phenotype. Based on the scores calculated for the cases and controls it is found that 81% of responders and 40% of nonresponders have a score of >19. Therefore, using 19 as a threshold value to stratify a patient population prior to administering the insulin sensitizer drug improves the overall efficacy of the drug from ˜32% to ˜50%. In doing so, the number of nonresponders exposed to the insulin sensitizer drug is decreased substantially, and those excluded may then be treated with alternative therapies sooner. A change in efficacy of this magnitude could help to get a new drug approved, or could encourage wider use of an already approved drug.

### C. Association Studies and Methods for Classes of Drugs.

The invention also includes methods and compositions for screening individuals for one or more genetic variations (e.g., SNPs) and/or one or more phenotypic variations that predicts responsiveness to a first drug, and using this association to determine whether or not to modulate the treatment of an individual with a second drug, where the first and second drugs belong to the same class of drugs. In some embodiments, the class of drugs is insulin sensitizers. In some embodiments, the first and second drugs are the same drug. In some of these embodiments, the second drug is netoglitazone and the first drug is an insulin sensitizer for which clinical or other data is available as well as either association studies with genotypes, or materials with which to perform such studies. Drugs for which clinical data is available that are insulin sensitizers include troglitazone, rosiglitazone, pioglitazone, muraglitazar, galida tesaglitazar, 677954, MBX-102, T131, LY818, LY929, and PLX204. Using these methods and compositions, the use of the second drug may be refined and targeted, so that, e.g., clinical trials for the second drug are more likely to be performed on patient populations likely to benefit from the drug and/or unlikely to suffer adverse effects. The methods and compositions of the invention thus allow much more targeted and precise testing and clinical use of drugs in a class of drugs.

A “class of drugs,” as used herein, includes a group of two or more drugs that are placed in the group through some common characteristic or characteristics. A “characteristic” is any trait that may be repeatably associated with a drug or a composition containing the drug, such as, but not limited to, structure, mechanisms, stereochemistry, crystal form, formulation, dosage, dosage route, dosage frequency and/or duration, effects in an animal model, empirically-found traits, use in combination with other therapies or drugs, combinations thereof, and the like. The characteristic(s) may be associated with significant predictability of an effect of the drugs, i.e., drugs placed in the same class because they share a common characteristic have a greater than random chance of having the same or similar therapeutic or non-therapeutic effect. A drug may be in more than one class, depending on the characteristic(s) used as criteria for the classes.

Rather than focusing on a single drug, the methods provide information on the likelihood that an individual will be a responder or non-responder for a series of related drugs, and, if a responder, whether the response will be a therapeutic or a non-therapeutic effect, e.g. an adverse effect. Thus, in some embodiments, an association study is performed to identify one or more genetic variations, e.g., SNPs and/or phenotypes, associated with one or more responses to a first drug in a class of drugs, and those variations are used to modulate the administration of a second drug in the class of drugs. The administration of the second drug may be in a research setting (e.g., a clinical trial), in a clinical setting (e.g., use of the drug in treatment of a disease), or in any other setting in which it is useful to predict the effect of the second drug (e.g., in commercialization of the second drug). “Modulation” of administration includes not administering the second drug, administering the second drug in a manner similar to the first drug (e.g., dosage, dosing schedule, duration of treatment, and the like), or altering the administration of the second drug in comparison to administration of the first drug. Modulation of administration is discussed in more detail elsewhere herein.

Screening of an individual for genetic variations associated with a response to a first drug in a class of drugs is useful, for example, to identify individuals who may be enrolled in (or excluded from) a clinical trial of a second drug in the class of drugs, and/or individuals who may suffer (or not suffer) an adverse reaction from a second drug in the class of drugs. For example, in some embodiments, results of an association study with a first drug in a class of drugs are used to screen individuals in patient populations for clinical trials of second drug in the class, in order to exclude individuals predicted to have adverse response to the second drug, and/or to include individuals predicted to have a desired response or degree of response to the second drug, and/or to otherwise modulate the administration of the second drug.

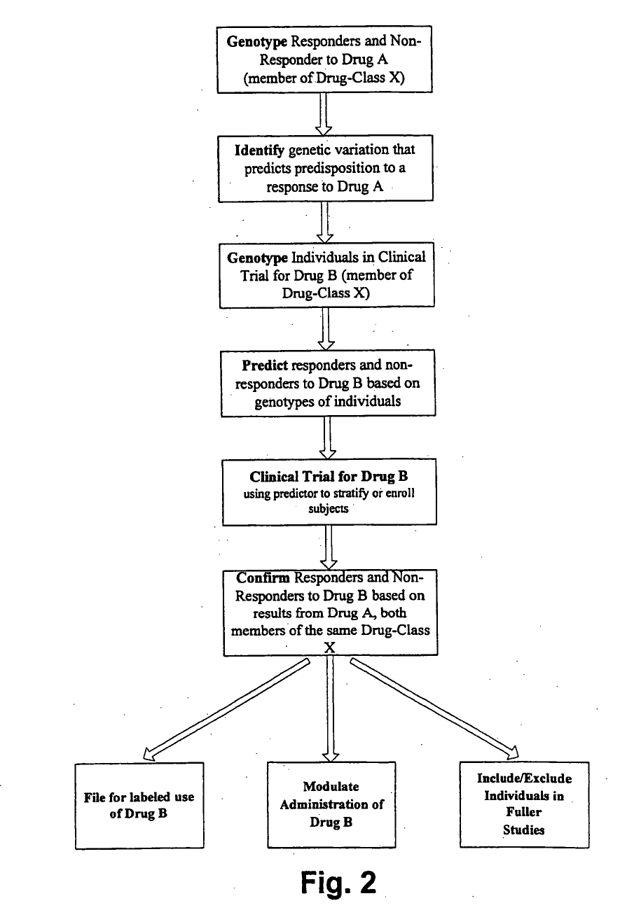

An exemplary embodiment is illustrated in FIG. 2. During, e.g., a clinical trial or routine clinical use of drug A that is member of class of drugs (in this example, drugs that act through mechanism X are placed in the same class), samples may be taken from individuals that are genotyped at sets or subsets of polymorphic loci, e.g., SNPs, and association studies may be performed to determine the relationship between responses of individuals to drug A and genotypes of the set or subsets of SNPS, as described herein. Responses may include therapeutic responses, including degrees of responsiveness (e.g., minimal responders vs. average responders vs. “superresponders”). Responses may also include non-therapeutic responses, e.g. side effects such as adverse effects. When another drug B, which also acts or is thought to act through mechanism X, is subjected to clinical trials, the results of the association studies for drug A may be used to modulate, e.g. the design of the clinical study, the enrollment in the clinical study, stratification of individuals enrolled in the study, and/or prediction of or analysis of results of the clinical trial of drug B. Of course, association studies may also identify phenotypic traits that are associated with the drug response, e.g., as described in U.S. Ser. No. 11/043,689, filed Jan. 24, 2005, entitled “Associations Using Genotypes and Phenotypes.”

In some embodiments, the results of the association studies for drug A are used to include or exclude individuals from the clinical trials for drug B. Typically, individuals are subject to exclusion if their genotype and/or phenotype indicates susceptibility to adverse effects or lack of responsiveness, or a combination thereof. Individuals excluded from clinical trials for drug B may be treated by alternative drugs or methods; in some cases the alternative drug and/or treatment may also be based at least in part on the results of the association studies of drug A (e.g., the individual may be treated with a drug of a different class than drug A and drug B). In some embodiments in which the drug in clinical trials is not administered to certain individuals, those individuals can be placed in a different clinical trial of another therapeutic agent acting through a different mechanism than the drug for which the individual was screened.

In some embodiments, the results of the association studies for drug A are used to modulate a clinical trial for drug B by altering the administration of drug B. For example, various aspects of treatment with drug B may be modulated based on association studies of drug A. For example, modulating administration may include: adjusting the dosage of the drug, route of administration of the drug, duration of treatment with the drug, or frequency of administration of the drug; changing the type of carrier of the drug, enantiomeric form of the drug, crystal form of the drug, tautomeric form of the drug; administering a fragment, analog, and/or variant of the drug; or a combination thereof. Thus, for example, the dose size and/or frequency may be adjusted for drug B based on predictions from association studies of drug A as to an individual's degree of therapeutic and/or non-therapeutic responsiveness to drug B. For example, those predicted to be mild responders but who are also predicted to have few or no adverse effects could be given a larger relative dose than those predicted to be normal or superresponders. Another example is in, e.g., cancer chemotherapy trials, where the dosage of the chemotherapeutic agent is often adjusted based on the individual's therapeutic response coupled with the individual's adverse effects. Thus, an individual who is predicted from genotype data, based on association data from a chemotherapeutic drug in a given class, to be a mild responder with large adverse effects to another drug in the class for which association studies are not available, may receive a lower dose, or no dose, of the drug, compared to an individual predicted to be a superresponder with few adverse effects, who would receive a high dose of the drug.

The association studies with drug A are used to predict the response of an individual to drug B, e.g., in a clinical trial or in the course of clinical treatment. For any given genotype (e.g., at one or more SNPs) and/or phenotype, a predictive probability of a response to drug A may be established from association studies of drug A. Decisions regarding modulation of the administration of drug B based on the genotype and/or phenotype of an individual can be made based on a pre-determined level of predictability for a response (desired, undesired, or a combination thereof) in that individual to drug A. In some embodiments, the degree of probability that a response or combination of responses to drug A will occur that is used as a cutoff for decision as to the use of drug B (e.g., inclusion or exclusion in a clinical trial) is greater than about 99.9, 99.5, 99, 98, 97, 96, 95, 94, 93, 92, 91, 90, 85, 80, 75, 70, 65, 60, 55, 50, 45, 40, 35, 30, 25, 20, 15, 10, 5, 4, 3, 2, 1, 0.5, or 0.1%. In some embodiments, the degree of probability that a response or combination of responses to drug A will occur that is used as a cutoff for decision as to the use of drug B (e.g., inclusion or exclusion in a clinical trial) is less than about 99.9, 99.5, 99, 98, 97, 96, 95, 94, 93, 92, 91, 90, 85, 80, 75, 70, 65, 60, 55, 50, 45, 40, 35, 30, 25, 20, 15, 10, 5, 4, 3, 2, 1, 0.5, or 0.1%. The use of more than one genotype and/or phenotype can increase the predictiveness for drug A used as a cutoff for drug B. For example, the use of genotype at more than one genetic variation, e.g., SNP, can increase the degree of predictiveness as to a given response to a drug. In some embodiments about 2, 3, 4, 5, 6, 7, 8, 9, 10, 15, 20, 25, 30, 35, 40, 45, 50, 60, 70, 80, 90, 100, 150, 200, 500, 1000, 10,000, 100,000, 1,000,000, or more than about 1,000,000 genetic variations, e.g., SNPs, are used to reach the desired level of probability for outcome with drug A used as a cutoff for deciding whether or not to modulate use of drug B.

In some embodiments, the response to the drug is an adverse effect, and a probability cutoff is used to limit the patient population administered a second drug in the same class. It will be appreciated that the probability cutoff can be lower or higher depending on the severity of the adverse effect, in combination with the type of patient population to be treated. For example, in a cancer chemotherapeutic trial in which a fatal adverse effect may occur, but where the patient population is composed of terminal patients, a higher probability cutoff would be tolerated than in a study of an antiinflammatory drug to reduce pain and inflammation with the same fatal adverse effect, but studied in a patient population composed of individuals suffering from mild to moderate inflammation. Thus, in the former case, a probability for the fatal adverse effect based on genotype association studies of a known drug in the class may be set at less than about 50, 45, 40, 35, 30, 25, 20, 15, 10, or 5%. In the latter case (e.g., for COX-2 inhibitors and other drugs where the adverse effects were of sufficient severity that the drug was withdrawn from the market) a low probability, e.g., less than about 10, 5, 1, 0.5, 0.1, 0.05, 0.01, 0.005, or 0.001% may be used. Thus, in some embodiments, the probability that a fatal or life-threatening adverse effect would occur for a given genotype for drug A is used in deciding whether to administer a drug B in the same class as A to an individual (e.g., in a clinical trial), based on the genotype of an individual.

In other embodiments it may be desired merely to increase the probability that a sufficient number of responders to a new drug be included in a clinical trial to provide significant results. This can be the case where a drug A in a class of drugs was found to cause a therapeutic response in only a certain percentage of people, and the percentage was not high enough to justify its use and/or approval. If drug B in the same class of drugs is to be tested, it is desired to increase the number of responders to a sufficient level to indicate the efficacy of drug B for use. In such cases, the cutoff probability for modulation of administration of drug B to an individual (e.g., inclusion or exclusion in a clinical trial) may be greater than about 50, 60, 70, 80, 90, 95, 98, 99%. An example of a drug that would benefit from such a prediction is the use of altered peptide ligands (APLs) in the treatment of multiple sclerosis. It appears that APLs are only effective in small subgroups of patients. The methods and compositions of the invention allow researchers and clinicians to increase the degree of certainty in designing clinical trials for emerging APLs, as well as in selecting patients for administration of an APL, thus increasing the value and usefulness of these drugs. Without such increased certainty, researchers are less likely to persist in investigation, and clinicians are less likely to prescribe the drugs, given the inability to predict their effectiveness.

It will be appreciated that various combinations of responses, as well as desired results, can result in a probability cutoff for modulation of administration of drug B that may be any desired probability for response to drug A. This can be done in combination with inclusion or exclusion in the clinical trial of drug B based on association studies using drug A. Predictions to be tested may include non-therapeutic effects, such as adverse effects, or lack thereof (e.g., if those thought to be susceptible to adverse effects are excluded from the trial). Predictions to be tested may also include degree of response, e.g., mild response, normal response, or superresponse.

In addition to predicting responses for new drugs in a class, drugs that were withdrawn from the market or from clinical trials for adverse effects (e.g., troglitazone, various COX-2 inhibitors) or because average efficacy was poor (e.g., vilazodone and eptrapirone for depression) may be “rehabilitated” when genetic profiles and association with efficacy and/or adverse effects are known for one or more other drugs in the same class. Thus, for example, troglitazone can be retested when genetic profiles for other PPAR agonists and their associated adverse effects are known. A further example is the retesting of vilazodone and eptrapirone when efficacy associations with genetic profiles for other serotonin agonists become available. Patient populations can be pre-screened and those with genetic profiles associated with a pre-determined probability of adverse effects can be excluded the trial, and/or those with genetic profiles associated with a predetermined probability of increased efficacy may be selected for the trial. In this way, drugs that would otherwise not be available to patients due to their failure in clinical trials or their withdrawal from the market, but that are effective (possibly highly effective) in a subgroup of patients, may become available and benefit select patient populations.

The invention also provides a drug that is approved for use by a regulatory agency, where the drug is a member of a class of drugs, and where the drug is tested for approval by the regulatory agency in a method that comprises screening an individual for one or more genetic variations and/or phenotypes associated with response to another drug in the class of drugs, and modulating or not modulating the administration of the drug based on the results of said testing. In some embodiments, the decision to modulate or not modulate the administration of the drug to be tested, and/or the modulation which is chosen, is based on a predetermined probability of response to the drug that has already been tested, based on the one or more genetic variations in the individual. In some embodiments the drug to be tested is a drug that has not previously been tested for approval by the regulatory agency. In some embodiments, the drug to be tested is a drug that was previously tested but not approved for use, or approved for use but withdrawn from the market. In some embodiments, the regulatory agency is the Food and Drug Administration (FDA). In some embodiments, the testing is a Phase I, Phase II, Phase III, or Phase IV clinical trial. In some embodiments, the drug is an insulin sensitizer, e.g., netoglitazone. In some embodiments, the drug is a drug that was withdrawn from use; in some embodiments the drug is selected from the group consisting of azarabine, troglitazone, fenfluramine, dexfenfluramine, terfenadine, mibefradil, astemizole, cisapride, alosetron, grepafloxacin, bromfenac, rapacuronium bromide, valdecoxib, rofecoxib, thalidomide, diethylstilbersterol, ticrynafen, methaquinone, trazolam, cerivastatin, fluoroxamine maleate, natalizumab, and hydromorphone HCl extended release. Further drugs withdrawn from use in the U.S. include adenosine phosphate, azaribine, benoxaprofen, bithionol, parenteral butamben, oral gel drug products containing carbetapentane citrate, chlorhexidine gluconate for use on skin, chlormadinone acetate, chloroform, diamthazole dihydrochloride, dibromsalan, dihydrostreptomycin sulfate, dipyrone, encainide hydrochloride, flosequinan, mepazine hydrochloride or mepazine acetate, metabromsalan, parenteral methamphetamine hydrochloride, methapyrilene, methopholine, nitrofurazone, nomifensine maleate, oxyphenisatin, oxyphenisatin acetate, phenacetin, phenformin hydrochloride, pipamazine, potassium arsenite, povidone, reserpine (more than 1 mg in oral dosage), sparteine sulfate, sulfadimethoxine, sulfathiazole, suprofen, temafloxacin hydrochloride, 3,3′,4′,5-tetrachlorosalicylanilide, tetracycline for pediatric use at greater than 25 mg/ml, tribromsalan, trichloroethane, and zomepirac sodium. Drugs withdrawn from use in European Union include valdecoxib, parecoxib, sildenalfil, rosiglitazone, apomorphine hydrocloride, desloratadine, dofetilede, votumamab, olanzapine, fomivirsen, imiquimod, ganiciclovir, rotavirus vaccine, combined diptheria, tetanus, and acellular pertussis vaccine, dodecafluoropentane, and levacetylmethadol. The methods may also be applied to drugs that have not been withdrawn or failed clinical trials, but that have warnings concerning their use. The methods may also be applied to drugs for which no withdrawal or warning is presently in effect.

The methods may also be used in the treatment of individuals. In some embodiments, an individual suffering from a disorder may be screened for a genetic variation and/or phenotype that indicates responsiveness to a class of drugs used to treat that disorder. In some embodiments, a study to determine the association between one or more genetic variations and responsiveness to a first drug in a class of drugs may be used to determine a treatment of an individual with a second drug in the class of drugs, or may be used to determine that the individual should be treated with a drug in a different class of drugs. Typically, the second drug will be a drug for which association studies have not been performed, or have not been performed to the same extent as for the first drug. In some embodiments, the disorder is a disorder of blood glucose regulation, e.g., an insulin resistance disorder, as described herein. In some embodiments, the class of drugs is PPAR modulator insulin sensitizers.

The invention further encompasses a database containing data regarding one or more drugs, and associations between one or more genetic variations and/or phenotypic variations, one or more responses of individuals to the drug or drugs, and one or more characteristics of the drugs. The database may contain data for at least about 1, 2, 3, 4, 5, 6, 7, 8, 9, 10, 11, 11, 12, 13, 14, 15, 20, 25, 30, 35, 40, 45, 50, 60, 70, 80, 90, 100, 150, 200, 500, 1000, or more than 1000 drugs. The drugs may be in a single class of drugs, or in more than one class of drugs. In some embodiments, all or substantially all drugs in a class for which data are available are included the database. Alternatively, the database may contain information concerning drugs in at least about 2, 3, 4, 5, 6, 7, 8, 9, 10, or more than 10 classes of drugs. In some embodiments, the database contains association data for all or substantially all drugs for which data are available. The database can be updatable, and updates can be available for those using the database, e.g., through the internet. Such a database allows empirical associations to be made between drugs in different mechanistic or structural classes which nonetheless have similar genetic variation profiles for their therapeutic effects. In some embodiments one or more of the genetic variations are SNPs.

In some embodiments the database is recorded in a tangible medium, such as a computer data storage medium, e.g., hard drive, compact disc, or the like. In some embodiments the invention encompasses the transmission of data contained in a database as described herein from one location to another by, e.g., electronic means such as transmission via the Internet.

It will be appreciated that as data is accumulated for members of a class of drugs or classes of drugs, the predictive power of associations will become greater, and that further refinements in the classification of the drugs can become apparent.

As one example, a subgroup of drugs within a particular class of drugs may exhibit the same or similar association profiles between response to the drugs and one or more genetic variations, e.g., SNPs and/or phenotypes, and these association profiles may differ from other members of the class. In some embodiments a new classification may be formed for these drugs based on a characteristic common to this subgroup but absent in some, most or all of the other members of the class. This characteristic may then be used in future association studies for drugs in this class to further subclassify them into the new subgroup in order to increase the predictive power of genotypes and/or phenotypes of individuals for responsiveness to new drugs in this subgroup. In some embodiments, the characteristic common to the drugs in this new subgroup but absent in some, most, or all of the other members of the class may also be investigated in drugs in other classes of drugs to determine if it correlates with greater degrees of predictability of a particular response in these other drugs. In some embodiments, the new common characteristic will be used to model a new mechanism common to the drugs with this characteristic that explains some or all of the drug responses associated with this characteristic. In some embodiments the new common characteristic will be used to design new drugs that cause the response associated with the characteristic in individuals with the genotype and/or phenotype associated with this response. For example, it may be found that drugs with characteristic X are found to have a response of longer half-life in individuals with genotype A. New drugs may be designed with characteristic X in the expectation that they will have a longer half-life in individuals with genotype A.

As another example, in some embodiments a drug that has not been tested for association between genotype and/or phenotype and response to the drug is assigned a “closest relative” in the database of drugs, based on one or, typically, more than one characteristic of the untested drug. The closest relative is the drug that most closely matches the untested drug in the characteristic(s). The characteristic(s) may be any characteristic(s) associated with the new drug and the closest relative. The closest relative is used to provide predictive association data for responses of individuals to the new drug based on the genotypes and/or phenotypes of the individuals. The actual responses of individuals to the new drug and their genotypes and/or phenotypes can then be monitored and used to refine the criteria for picking the closest relative. For example, data for the new drug can be compared with predicted responses for some or all of the other drugs in the database that share one or more characteristics with the new drug, and with predicted responses for its closest relative, to determine whether the chosen closest relative was, indeed, the drug with the highest predictive power for the new drug. If not, the algorithm for choosing a closest relative is revised to reflect the difference, e.g., the criteria what constitutes a “match” between characteristics of an untested drug and drugs in the database can be changed based on the new data. In this and similar ways, as data accumulates in the database, characteristics used to group drugs into classes can be refined and altered in order to increase the predictive power of the classifications, to model new mechanisms of drug action, to design new drugs, and the like.

The invention further provides software to determine whether a drug has a characteristic or characteristics; to determine a drug's closest relative and/or class or classes given the characteristic or characteristics; to predict the probability that a given response will occur for a drug (e.g., an untested drug's closest relative, or a member of the class of drugs in which the untested drug has been placed) in the database given the presence of a genotype and/or phenotype; to suggest best modulations for administration of an untested drug based on a genotype of an individual; algorithms for refining the database and for refining other algorithms in the software based on new data for new drugs, genotypes, phenotypes, or responses; and the like.

In one aspect, the invention provides kits. The kits contain a testing component for testing one or more genetic variations in an individual in order to genotype the individual for the one or more variations, contained in packaging. In some embodiments, the testing component comprises one or more nucleic acids, as described herein. In some embodiments, the genetic variations are SNPs. In some embodiments, the testing component is a microarray, e.g., a SNP chip. In some embodiments, the kits further include a database or access to a database for one or more drugs and the associations between genetic variations and/or phenotypic variations that may be tested with the testing component or that may be otherwise observed, and responses to the one or more drugs. The kits may further include software or access to software to determine whether a drug has a characteristic or characteristics; to determine a drug's closest relative and/or class or classes given the characteristic or characteristics; to predict the probability that a given response will occur for a drug (e.g., an untested drug's closest relative, or a member of the class of drugs in which the untested drug has been placed) in the database given the presence of a genotype and/or phenotype; to suggest best modulations for administration of an untested drug based on a genotype of an individual; algorithms for refining the database and for refining other algorithms in the software based on new data for new drugs, genotypes, phenotypes, or responses; and the like. The kit may further contain instructions for use of the components of the kit, as well as other components useful in sampling for and using the means for genetic testing, components useful in sample preparation such as components for amplifying nucleic acids (e.g., PCR components), gloves, eye protection, cleaning substances, buffers, primers, enzymes, labels, and the like.

In one aspect, the methods of the invention include business methods. In one embodiment, the invention provides a method comprising using the results of an association study that predicts the association between one or more genetic variations (and/or phenotypes) and responsiveness to a first drug to market a second drug, where the first and the second drugs are members of the same class of drugs. For example, new drug A may belong to the same class of drugs as old drug B, for which association data are available. New drug A may be marketed for use with those individuals exhibiting genotypes and/or phenotypes that have been found for old drug B to predict a high degree of efficacy, less severe or low rate of adverse effects, or other desirable effects. Such marketing may be directed to health care professionals and/or to patients.

In some embodiments the invention further provides an isolated nucleic acid that specifically hybridizes to a genomic sequence within a region containing a nucleic acid associated with a response to a first drug in a class of drugs, for use in diagnostics, prognostics, prevention, treatment, or study of a response to a second drug in the class of drugs. In some embodiments the region extends from about 10 kb upstream to about 10 kb downstream of the nucleic acid. In some embodiments the region extends from about 5 kb upstream to about 5 kb downstream of the nucleic acid. In some embodiments the region extends from about 2 kb upstream to about 2 kb downstream of the nucleic acid. In some embodiments the region extends from about 1 kb upstream to about 1 kb downstream of the nucleic acid. In some embodiments the invention provides an isolated nucleic acid that specifically hybridizes to the nucleic acid sequence itself. In some embodiments, a set of nucleic acids is provided, where the nucleic acids of the set are related (e.g., are complementary) to some or all of the genetic variations associated with a response to a first drug in a class of drugs, for use with a second drug in said class of drugs, in diagnostics, prognostics, prevention, treatment, or study of a response to the second drug. In some embodiments, the class of drugs is insulin sensitizers, e.g., PPAR modulator insulin sensitizers such as thiazolidinedione PPAR modulators. In some embodiments, the nucleic acid is immobilized on a solid support. In some embodiments, a set of nucleic acids immobilized on a solid support is provided, where the nucleic acids of the set are related (e.g., are complementary) to some or all of the genetic variations, e.g., SNPs, associated with a response to a drug in a class of drugs.

The identification of genetic variations in the individual may be done by any suitable means, as described herein. See e.g., U.S. Pat. No. 6,897,025; and U.S. patent application Ser. Nos. 10/448,773 entitled “Methods for Genomic Analysis, filed May 29, 2003; 10/042,819, entitled “Genetic Analysis Systems and Methods,” filed Aug. 21, 2003; 10/786,475 entitled “Analysis Methods for Individual Genotyping”, filed Oct. 21, 2004; and 10/845,316 entitled “Allele-Specific Expression Patterns,” filed Jan. 6, 2003. Suitable means also include gel-based genotyping, nanofluidics, hybridization to nucleic acid probe arrays, and single base addition sequencing (see, e.g., U.S. Pat. No. 6,911,345).

### 1. Classes of Drugs

Drugs may be classed into mechanistic classes, structural classes, classes based on pharmacological effect, and other classes of drugs that are based on the chemical or biological nature of the drugs, or that are empirically based.

Mechanistic classifications are based on the mechanism of action of drugs, e.g., receptor targets or other targets of the drugs. For example, drugs that primarily act on the autonomic nervous system may be classed as cholinoreceptor-activating drugs, or cholinesterase-inhibiting drugs, or cholinoceptor-blocking drugs, or adrenoceptor-activating drugs, or adrenoceptor-blocking drugs.

However, as is known in the art, often drugs do not have a known target or a precisely defined mechanism, and may be classed according to similarities in other aspects the drugs, such as similarities of the chemical structure that are thought to be important to the action of the drugs. Such similarities include structural components, optical isomerism, crystal structure, and the like.

Drugs may also be classed based on their major pharmacological action, e.g., lipid-lowering drugs, antidepressants, anxiolytics, and the like. The second drug may be placed in the same class as the first drug by in vitro and/or in vivo studies; in some embodiments, action through the same or similar mechanism may be predicted from structural analysis.

In some embodiments, drugs are classified based on their effects in one or more in vitro, cellular, tissue, organ, or animal models. Such effects may be molecular, supramolecular, cellular, tissue, organ, or whole-organism effects, or combinations thereof. In some embodiments, drugs are classified based on their effects in one or more animal models together with associations between genotypes and response in the animal models. For example, drug A may cause response M in a mammal, e.g. a rat, mouse, or primate, of genotype X (e.g., genotype at one or more SNPs), and may cause response N in a primate of genotype Y. If drug B is found to cause response M in a mammal of genotype X and response N in a mammal of genotype Y, then drug B is considered to be in the same class as drug A. It will be appreciated that such classification may be greatly refined based on the number of genetic variations included in the genotype, the number of responses measured, and the like. The animal model allows a much wider range of drugs to be tested, as well as more invasive parameters to be measured as indications of response, and can allow a much more extensive database to be established in a relatively short time, compared to human testing.

In other embodiments, expression profiles for a drug in a model system may be used to classify the drug. For example, all, most, or some of the known drugs of a class of drugs that has an effect in humans (e.g., statins that lower the risk of heart disease) may be tested in an animal model. Animals administered the drug may show consistent profiles of gene expression in response to the drug (e.g., increases in expression of a gene or set of genes related to antiinflammatory activity). Other drugs of other classes may be tested in animal models. The expression profiles associated with the drugs in a particular class may be correlated. A new drug may be assigned to a drug class based on its expression profile in one or more animal models. The associations of one or more drugs in that class between one or more genetic variations and a response to the drug(s) may be used to modulate the use of the new drug, for example, in research (e.g., clinical trials) and/or in the clinical setting.

In some embodiments, a new drug in a class of drugs is first tested in a model, e.g. an animal model, in which other drugs in the class of drugs have been tested, and in which a genotype for the animal is used to predict responses to the new drug. The results of the animal studies can be used to refine predictions for the association between genetic variations and response to a new drug in humans. Animal models may be developed or existing animal models may be used. The animal model can be for a particular physiological, biochemical, or metabolic state, e.g., a disease or pathological state. Healthy or superhealthy states may also be modeled (e.g., decelerated aging).

Drugs may be further put into classes, or into subclasses of the same class, by classifications based on their mode administration (e.g., intravascular, intramuscular, subcutaneous, ocular, inhalation, oral, sublingual, suppository, skin, via pump, and the like), formulation type (e.g., rapid acting, sustained release, enterically coated, etc.), mode of uptake and delivery to site of action, metabolism (e.g., drugs metabolized through Phase I reactions such as oxidation via hepatic microsomal P450 system and subclasses thereof, through oxidation via nonmicrosomal mechanisms and subclasses thereof, through reduction, through hydrolysis and subclasses thereof; drugs metabolized through Phase II reactions such as glucoronidation, acetylation, mercapturic acid formation, sulfate conjugation, N-, O-, and S-methylation, trans-sulfuration; and combinations thereof), metabolic products and/or byproducts and their structure and/or function, pharmacokinetics, pharmacodynamics, elimination, and the like.

It will be appreciated that these classifications are exemplary only, and that any means of classifying drugs that allows a non-random predictability of the effects of drugs in the class may be used. Further systems of drug classification and specific drugs within each class may be found in the art. See, e.g., Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, New York, 1990; Katzung, ed., Basic and Clinical Pharmacology, Ninth Edition, McGraw Hill, 20037ybg; Goodman and Gilman, eds., The Pharmacological Basis of Therapeutics, Tenth Edition, McGraw Hill, 2001; Remingtons Pharmaceutical Sciences, 20th Ed., Lippincott Williams & Wilkins., 2000; Martindale, The Extra Pharmacopoeia, Thirty-Second Edition (The Pharmaceutical Press, London, 1999); all of which are incorporated by reference herein in their entirety.

Any suitable class of drugs for which genotyping and association studies are possible for at least one member of the class may be the subject of the described methods and compositions. Classes include the insulin sensitizers as described herein, e.g., PPAR modulators. Thus, in some embodiments, the invention provides a method for predicting an individual's responsiveness to an insulin sensitizer, e.g., a PPAR modulator based on the individual's genotype (and/or phenotype) and the results of association studies between genotype (and/or phenotype) and responsiveness to another insulin sensitizer, e.g., PPAR modulator. In some embodiments, the prediction of an individual's responsiveness to an insulin sensitizer, e.g., PPAR modulator is used to include or exclude the individual in a clinical trial. In some embodiments, the prediction of an individual's responsiveness to an insulin sensitizer, e.g., PPAR modulator is used to modulate the individual's administration of another insulin sensitizer, e.g., PPAR modulator. In some embodiments such modulation occurs in a clinical trial. In some embodiments, the prediction of an individual's responsiveness to an insulin sensitizer, e.g., PPAR modulator is used to determine that the individual should be treated with a drug other than an insulin sensitizer, or in some embodiments a PPAR modulator.

a. Mechanistic Classes of Drugs

One non-exclusive exemplary class of drugs for which genotyping (and/or phenotyping) and association studies with one member may be used to predict effects of another member include, mechanistic classes of drugs used in the treatment of diabetes (including PPAR modulators). This class of drugs also illustrates how drugs can also be subclassed by, e.g., mode of administration. For example, insulin and insulin analogs may be formulated for administration by injection, nasal spray, transdermal, oral or inhalation routes. Each type of formulation can have unique profiles of responses and associated genetic variations. An example of classifications of such drugs by mechanism, together with representative members of the mechanistic classes, is given in Table 3.

TABLE 3
Classes of Drugs for Treatment of Diabetes

| Class | Mechanism of Action | Examples |
|---|---|---|
| Peroxisome | Target PPAR-gamma or PPAR-gamma and -alpha (see below). | Rosiglitazone, Pioglitazone, |
| Proliferator- | PPAR are nuclear receptors that help regulate glucose and lipid | Balaglitazone, see also |
| Activated Receptor | metabolism. Activation of PPAR-gamma improves insulin | others described herein |
| (PPAR) Agonists | sensitivity and thus improves glycemic control. | |
| Dual-Action | Act on both PPAR-gamma and PPAR-alpha. PPAR-alpha | TAK-559, Muraglitazar, |
| Peroxisome | activation has effects on cellular uptake of fatty acids and their | Tesaglitazar, Netoglitazone, |
| Proliferator- | oxidation, and on lipoprotein metabolism. May also act to reduce | see also others described |
| Activated Receptor | inflammatory response in vascular endothelial cells. | herein |
| Agonists | | |
| Biguanidines | Complete mechanism is not known. Reduces gluconeogenesis in | Metformin, Metformin GR |
| | the liver by inhibiting glucose-6-phosphatase. | |
| Sulfonylureas | Induce insulin secretion by binding to cellular receptors that | Glimepride, |
| | cause membrane depolarization and insulin exocytosis. | Glyburide/glibenclamide, |
| | | Glipizide, Gliclazide. |
| | | Tobutamide |
| Insulin and Insulin | Supplements endogenous insulin. Insulin analogs have a variety | Insulin lispro, Insulin aspart, |
| Analogs (Injectable, | of amino acid changes and have altered onset of action and | Insulin glargine, Exubera, |
| Inhaled, Oral, | duration of action, as well as other properties, compared to native | AERx Insulin Diabetes |
| Transdermal, | insulin. Inhaled insulin is absorbed through the alveoli. Spray | Management System, HIM- |
| Intranasal) | oral insulin is absorbed by the buccal mucosa and intranasal | 2, Oaralin, Insulin detemir, |
| | through the nasal mucosa. Transdermal insulin is absorbed | Insulin glulisine |
| | through the skin. | |
| Meglitinides | Are thought to bind to a nonsulfonylurea beta cell receptor and | Repaglinide, Nateglinide, |
| | act to cause insulin secretion by mechanism similar to | Mitiglinide |
| | sulfonylureas | |
| Alpha-Glucosidase | Inhibit carbohydrate digestion. Act at brush border of intestinal | Acarbose, Miglitol, |
| Inhibitors | epithelium. | Voglibose |
| Glucagon-Like | Diabetic patients may lack native GLP-1, and anlalogs act as | Exenatide, Exenatide LAR, |
| Peptide(GLP)-1 | substitutes. GLP-1 is an intestinal peptide hormone that induces | Liraglutide, ZP 10, |
| Analogs | glucose-dependent insulin secretion, controls gastric emptying, | BN51077, |
| | inhibits appetite, and modulates secretion of glucagon and | |
| | somatostatin. | |
| Dipeptidyl Peptidase | Inhibit DPP-IV, a ubiquitous enzyme that cleaves and inactivates | LAF-237, p-32/98, MK- |
| (DPP)-IV Inhibitors | GLP-1, thus inhibition of DPP-IV increases GLP-1 activity | 431, P3298, NVP LAF 237, |
| Pancreatic Lipase | Inhibits lipases, thus inhibiting uptake of dietary fat. This causes | Orlistat |
| Inhibitors | weight loss, improves insulin sensitivity and lowers | |
| | hyperglycemia. | |
| Amylin Analogs | Act to augment amylin, which acts with insulin by slowing | Pramlintide |
| | glucose absorption from the gut and slows after-meal glucose | |
| | release from liver. | |
| Dopamine D2 | Thought to act to alleviate abnormal daily variations in central | Bromocriptine |
| receptor agonists | neuroendocrine activity that can contribute to metabolic and | |
| | immune system disordered. | |
| Immunosuppressants | Suppress autoimmune response thought to be implicated in | Daclizumab, NBI 6024, |
| | Type I and possibly Type II diabetes. Example: Humanized | TRX-TolerRx, OKT3- |
| | monoclonal antibody that recognizes and inhibits the alpha | gamma-1-ala-ala |
| | subunit of IL-2 receptors; humanized Mab that binds to T cell | |
| | CD3 receptor to block function of T-effector cells that attack the | |
| | body and cause autoimmune disease | |
| Insulin-like growth | Recombinant protein complex of insulin-like growth factor-1 and | Somatomedin-1 binding |
| factor-1 agonists | binding protein-3; regulates the delivery of somatomedin to target | protein 3 |
| | tissues. Reduces insulitis severity and beta cell destruction | |
| Insulin sensitizers | Insulin sensitizers, generally orally active | S15261, Dexlipotam, CLX |
| | | 0901, R 483, TAK 654 |
| Growth hormone | Mimic the action of native GHRF | TH9507, SOM 230 |
| releasing factor | | |
| agonists | | |
| Glucagon antagonists | Inhibit glucagon action, stimulating insulin production and | Liraglutide, NN 2501 |
| | secretion, resulting in lower postprandial glucose levels | |
| Diabetes type 1 | Prevents destruction of pancreatic beta cells that occurs in type 1 | Q-Vax, Damyd vaccine |
| vaccine | diabetes | |
| Sodium-glucose co- | Selectively inhibits the sodium glucose co-transporter, which | T 1095 |
| transporter inhibitor | mediates renal reabsorption and intestinal absorption of glucose | |
| | to maintain appropriate blood glucose levels. | |
| Glycogen | Inhibit glycogen phosphorylase, thus slowing release of glucose | Ingliforib |
| phosphorylase | | |
| inhibitors | | |
| Undefined | Drugs that act in ways beneficial to those with Type I or Type II | FK 614, INGAP Peptide, R |
| mechanisms | Diabetes Mellitus, e.g., by reducing blood glucose and | 1439 |
| | triglyceride levels, whose mechanisms have not been elucidated. | |
| Antisense | Bind to RNA and cause its destruction, thereby decreasing | ISIS 113715 |
| oligonucleotides | protein production from corresponding gene. | |
| Insulinotropin | Stimulate insulin release | CJC 1131 |
| agonists | | |
| Gluconeogenesis | Inhibit gluconeogenesis, thus modulating blood glucose levels | CS 917 |
| inhibitors | | |
| Hydroxysteroid | Inhibit hydroxysteroid dehydrogenase, which are responsible for | BVT 3498 |
| dehydrogenase | excess glucocorticoid production and hence, visceral obesity | |
| inhibitors | | |
| Beta 3 adrenoceptor | Agonist for beta 3 adrenoceptor, decreases blood glucose and | YM 178, Solabegron, |
| agonist | suppresses weight gain | N5984, |
| Nitric oxide | Decreases effects of NO | NOX 700 |
| antagonist | | |
| Carnitine | Inhibits carnitine palmitoyltransferase | ST 1326 |
| palmitoyltransferase | | |
| inhibitor | | |

In other embodiments, mechanistic classes of drugs used in the treatment of abnormal cholesterol and/or triglyceride levels in the blood are used in conjunction with a method or composition of the invention. Broad mechanistic classes include the statins, fibrates, cholesterol absorption inhibitors, nicotinic acid derivatives, bile acid sequestrants, cholesteryl ester transfer protein inhibitors, reverse lipid transport pathway activators, antioxidants/vascular protectants, acyl-CoA cholesterol acyltransferase inhibitors, peroxisome proliferator activated receptor agonists, microsomal triglyceride protein inhibitors, squalene synthase inhibitors, lipoprotein lipase activators, lipoprotein (a) antagonists, and bile acid reabsorption inhibitors. An example of classification of such drugs by mechanism, together with representative members of the mechanistic classes, is given in Table 4.

TABLE 4
Classes of Drugs for Treatment of Abnormal Cholesterol and/or Triglyceride Levels in the Blood

| Class | Mechanism of Action | Examples |
|---|---|---|
| Statins | Competitive inhibitors of HMG-CoA reductase | Atorvastatin, Simvastatin, |
| | | Pravastatin, Fluvastatin, |
| | | Rosuvastatin, Lovastatin, |
| | | Pitavastatin, Cerivastatin |
| | | (withdrawn), |
| Fibrates | PPARα activators | Fenofibrate, Bezafibrate, |
| | | Gemfibrozil, clofibrate, |
| | | ciprofibrate |
| Cholesterol | May inhibit NCP1L1 in gut | Ezetimibe |
| Absorption Inhibitors | | |
| Nicotinic Acid | Inhibits cholesterol and triglyceride synthesis, exact mechanism | Niacin |
| Derivatives | unknown | |
| Bile Acid | Interrupt the enterohepatic circulation of bile acids | Colesevelam, |
| Sequestrants | | Cholestyramine, |
| | | Colestimide, Colestipol |
| Cholesteryl Ester | Inhibit cholesteryl ester transfer protein, a plasma protein that | JTT-705, CETi-1, |
| Transfer Protein | mediates the exchange of cholesteryl esters from antiatherogenic | Torcetrapib |
| Inhibitors | HDL to proatherogenic apoliprotein B-containing lipoproteins | |
| Reverse Lipid | Stimulate reverse lipid transport, a four-step process form | ETC-216, ETC-588, ETC- |
| Transport Pathway | removing excess cholesterol and other lipids from the walls of | 642, ETC-1001, ESP-1552, |
| Activators | arteries and other tissues | ESP-24232 |
| Antioxidants/Vascular | Inhibit vascular inflammation and reduce cholesterol levels; | AGI-1067, Probucol |
| Protectants | block oxidant signals that switch on vascular cellular adhesion | (withdrawn) |
| | molecule (VCAM)-1 | |
| Acyl-CoA | Inhibit ACAT, which catalyzes cholesterol esterification, | Eflucimibe, Pactimibe, |
| Cholesterol | regulates intracellular free cholesterol, and promotes cholesterol | Avasimibe (withdrawn), |
| Acyltransferase | absorption and assemble of VLDL | SMP-797 |
| (ACAT) Inhibitors | | |
| Peroxisome | Activate PPARs, e.g., PPARα, γ, and possibly δ, which have a | Tesaglitazar, GW-50516, |
| Proliferator Activated | variety of gene regulatory functions | GW-590735, LY-929, LY- |
| Receptor Agonists | | 518674, LY-465608, LY- |
| | | 818 |
| Microsomal | Inhibit MTTP, which catalyze the transport of triglycerides, | Implitapide, CP-346086 |
| Triglyceride Transfer | cholesteryl ester, and phosphatidylcholine between membranes; | |
| Protein (MTTP) | required for the synthesis of ApoB. | |
| Inhibitors | | |
| Squalene Synthase | Interfere with cholesterol synthesis by halting the action of liver | TAK-475, ER-119884 |
| Inhibitors | enzymes; may also slow or stop the proliferation of several cell | |
| | types that contribute to atherosclerotic plaque formation | |
| Lipoprotein Lipase | Directly activate lipoprotein lipase, which promotes the | Ibrolipim (NO-1886) |
| Activators | breakdown of the fat portion of lipoproteins | |
| Liproprotein(a) | Not yet established | Gembacene |
| Antagonists | | |
| Bile Acid | Inhibit intestinal epithelial uptake of bile acids. | AZD-7806, BARI-1453, S- |
| Reabsorption | | 8921 |
| Inhibitors | | |

In other embodiments, mechanistic classes of drugs used in the treatment of depression are used in conjunction with a method or composition of the invention. Current or emerging antidepressant drugs act by a variety of mechanisms, e.g., selective serotonin reuptake inhibitors (SSRIs), serotonergic/noradrenergic agents, serotonin/noradrenergic/dopaminergic agents, tricyclic antidepressants, monoamine oxidase inhibitors (MAOIs), noradrenergic/dopaminergic agents, serotonin antagonists, serotonin agonists, substance P antagonists, and beta₃ adrenoreceptor agonists. An example of classification of such drugs by mechanism, together with representative members of the mechanistic classes, is given in Table 5.

TABLE 5
Classes of Drugs for Treatment of depression

| Class | Mechanism of Action | Examples |
|---|---|---|
| Selective Serotonin | Block presynaptic reuptake of serotonin. Exert little effect on | Escitalopram, Sertraline, |
| Reuptake Inhibitor | norepinephrine or dopamine reuptake. Level of serotonin in | Citalopram, Paroxetine, |
| (SSRI) | the synaptic cleft is increased. | Paroxetin, controlled |
| | | release, Fluoxetine, |
| | | Fluoxetine weekly, |
| | | Fluvoxamine, |
| | | olanzapine/fluoxetine |
| | | combination |
| Serotonergic/noradrenergic | Inhibit both serotonin reuptake and norepinephrine reuptake. | Venlafaxine; Reboxetine, |
| agents | Different drugs in this class can inhibit each receptor to | Milnacipran, Mirtazapine, |
| | different degrees. Do not affect histamine, acetylcholine, and | Nefazodone, Duloxetine |
| | adrenergic receptors. | |
| Serotonergic/noradrenergic/ | Several different mechanisms. Block norepinephrine, | Bupropion, Maprotiline, |
| dopaminergic agents | serotonin, and/or dopamine reuptake. Some have addictive | Mianserin, Trazodone, |
| | potential due to dopamine reuptake inhibition. | Dexmethylphenidate, |
| | | Methyphenidate, |
| | | Amineptine |
| Tricyclic Antidepressants | Block synaptic reuptake of serotonin and norepinephrine. | Amitriptyline, Amoxapine, |
| | Have little effect on dopamine. Strong blockers of | Clomipramine, |
| | muscarinic, histaminergic H1, and alpha-1-adrenergic | Desipramine, Doxepin, |
| | receptors. | Imipramine, Nortriptyline, |
| | | Protriptyline, Trimipramine |
| Irreversible Monoamine | Monoamine oxidase (MAO) metabolizes monoamines such as | Isocarboxazid, Phenelzine, |
| Oxidase Inhibitors | serotonin and norepinephrine. MAO inhibitors inhibit MAO, | Tranylcypromine, |
| | thus increasing levels of serotonin and norepinephrine. | Transdermal Selegiline |
| Reversible Monoamine | See above. Short acting, reversible inhibitor, inhibits | Moclobemide |
| Oxidase Inhibitors | deamination of serotonin, norepinephrine, and dopamine. | |
| Serotonergic/noradrenergic/ | Act to block all of serotonin, norepinephrine, and dopamine | DOV-216303, DOV-21947 |
| dopaminergic reuptake | reuptake. May have addictive potential due to dopamine | |
| inhibitors | reuptake inhibition. | |
| Noradrenergic/dopaminergic | Block reuptake of norepinephrine and dopamine | GW-353162 |
| agents | | |
| Serotonin Antagonists | Selective antagonist of one serotonin receptor (the 5-HT₁ | Agomelatine |
| | receptor) | |
| Serotonin Agonists | Partial agonist of the 5-HT_{1A} receptor. | Eptapirone, Vilazodone, |
| | | OPC-14523, MKC-242, |
| | | Gepirone ER |
| Substance P Antagonists | Modify levels of substance P, which is released during acute | Aprepitant, TAK-637, CP- |
| | stress. | 122721, E6006, R-763OPC- |
| | | GW-597599 |
| Beta₃ Adrenoreceptor | Indirectly inhibit norepinephrine reuptake. Also being | SR-58611 |
| Agonists | investigated for treatment of obesity and diabetes because | |
| | they stimulate lipolysis and thermogenesis. | |

In other embodiments, mechanistic classes of drugs used in the treatment of multiple sclerosis are used in conjunction with a method or composition of the invention. These drugs can be classed as, e.g., recombinant interferons, altered peptide ligands, chemotherapeutic agents, immunosuppressants, corticosteroids, monoclonal antibodies, chemokine receptor antagonists, AMPA receptor antagonists, recombinant human glial growth factors, T-cell receptor vaccines, and oral immunomodulators. An example of classification of such drugs by mechanism, together with representative members of the mechanistic classes, is given in Table 6.

TABLE 6
Classes of Drugs for Treatment of Multiple Sclerosis

| Class | Mechanism of Action | Examples |
|---|---|---|
| Recombinant | IFN-beta has numerous effects on the immune system. Exact | Interferon-beta-1b, |
| interferons | mechanism of action in MS not known | Interferon-beta-1a |
| Altered peptide | Ligands either templated on sequence of myelin basic protein, or | Glatiramer acetate, MBP- |
| ligands | containing randomly arranged amino acids (e.g., ala, lys, glu, tyr) | 8298, Tiplimotide, AG-284 |
| | whose structure resembles myelin basic protein, which is thought | |
| | to be an antigen that plays a role in MS. Bind to the T-cell | |
| | receptor but do not activate the T-cell because are not presented | |
| | by an antigen-presenting cell. | |
| Chemotherapeutic | Immunosuppressive. MS is thought to be an autoimmune | Mitoxantrone, |
| agents | disease, so chemotherapeutics that suppress immunity improve | Methotrexate, |
| | MS | Cyclophosphamide |
| Immunosuppressants | Act via a variety of mechanisms to dampen immune response. | Azathioprine, |
| | | Teriflunomide, Oral |
| | | Cladribine |
| Corticosteroids | Induce T-cell death and may up-regulate expression of adhesion | Methylprednisolone |
| | molecules in endothelial cells lining the walls of cerebral vessels, | |
| | as well as decreasing CNS inflammation. | |
| Monoclonal | Bind to specific targets in the autoimmune cascade that produces | Natalizumab, Daclizumab, |
| Antibodies | MS, e.g., bind to activated T-cells | Altemtuzumab, BMS- |
| | | 188667, E-6040, Rituximab, |
| | | M1 MAbs, ABT 874, T- |
| | | 0047 |
| Chemokine Receptor | Prevent chemokines from binding to specific chemokine | BX-471, MLN-3897, MLN- |
| Antagonists | receptors involved in the attraction of immune cells into the CNS | 1202 |
| | of multiple sclerosis patients, and inhibiting immune cell | |
| | migration into the CNS | |
| AMPA Receptor | AMPA receptors bind glutamate, an excitatory neurotransmitter, | E-2007 |
| Antagonists | which is released in excessive quantities in MS. AMPA | |
| | antagonists suppresses the damage caused by the glutamate | |
| Recombinant Human | GGF is associated with the promotion and survival of | Recombinant Human GGF2 |
| Glial Growth Factor | oligodendrocytes, which myelinate neurons of the CNS. rhGGF | |
| (GGF) | may help myelinate oligodendrocytes and protect the myelin | |
| | sheath. | |
| T-cell Receptor | Mimic the part of the receptor in T cells that attack myelin | NeuroVax |
| Vaccine | sheath, which activates regulatory T cells to decrease pathogenic | |
| | T-cells. | |
| Oral | Various effects on the immune response that can modulate the | Simvastatin, FTY-720, Oral |
| Immunomodulators | process of MS | Glatiramer Acetate, FTY- |
| | | 720, Pirfenidone, |
| | | Laquinimod |

In other embodiments, mechanistic classes of drugs used in the treatment of Parkinson's disease are used in conjunction with a method or composition of the invention. These classes include dopamine precursors, dopamine agonists, COMT inhibitors, MAO-B inhibitors, antiglutametergic agents, anticholinergic agents, mixed dopaminergic agents, adenosine A2a antagonists, alpha-2 adrenergic antagonists, antiapoptotic agents, growth factor stimulators, and cell replacements. An example of classification of such drugs by mechanism, together with representative members of the mechanistic classes, is given in Table 7.

TABLE 7
Classes of Drugs for Treatment of Parkinson's Disease

| Class | Mechanism of Action | Examples |
|---|---|---|
| Dopamine Precursors | Act as precursors in the synthesis of dopamine, the | Levodopa, Levodopa- |
| | neurotransmitter that is depleted in Parkinson's Disease. Usually | carbidopa, Levodopa- |
| | administered in combination with an inhibitor of the carboxylase | benserazide, Etilevodopa, |
| | enzyme that metabolizes levodopa. Some (e.g., Duodopa) are | Duodopa |
| | given by infusion, e.g., intraduodenal infusion | |
| Dopamine Agonists | Mimic natural dopamine by directly stimulating striatal dopamine | Bromocriptine, Cabergoline, |
| | receptors. May be subclassed by which of the five known | Lisuride, Pergolide, |
| | dopamine receptor subtypes the drug activates; generally most | Pramipexole, Ropinirole, |
| | effective are those that activate receptors the in the D2 receptor | Talipexole, Apomorphine, |
| | family (specifically D2 and D3 receptors). Some are formulated | Dihydroergocryptine, |
| | for more controlled release or transdermal delivery. | Lisuride, Piribedil, |
| | | Talipexole, Rotigotin CDS, |
| | | Sumanirole, SLV-308 |
| COMT Inhibitors | Inhibits COMT, the second major enzyme that metabolized | Entacapone, Tolcapone, |
| | levodopa. | Entacapone-Levodopa- |
| | | Carbidopa fixed |
| | | combination, |
| MAO-B Inhibitors | MAO-B metabolizes dopamine, and inhibitors of MAO-B thus | Selegiline, Rasagiline, |
| | prolong dopamine's half-life | Safinamide |
| Antiglutamatergic | Block glutamate release. Reduce levodopa-induced dyskinesia | Amantadine, Budipine, |
| Agents | | Talampanel, Zonisamide |
| Anticholinergic | Thought to inhibit excessive cholinergic activity that | Trihexyphenidyl, |
| Agents | accompanies dopamine deficiency | Benztropine, Biperiden |
| Mixed Dopaminergic | Act on several neurotransmitter systems, both dopaminergic and | NS-2330, Sarizotan |
| Agents | nondopaminergic. | |
| Adenosine A2a | Adenosine A2 antagonize dopamine receptors and are found in | Istradefylline |
| antagonists | conjunction with dopamine receptors. Antagonists of these | |
| | receptors may enhance the activity of dopamine receptors. | |
| Alpha-2 Adrenergic | Not known. | Yohimbine, Idazoxan, |
| Antagonists | | Fipamezole |
| Antiapoptotic Agents | Can slow the death of cells associated with the neurodegenerative | CEP-1347, TCH-346 |
| | process of Parkinson's disease. | |
| Growth Factor | Promote the survival and growth of dopaminergic cells. | GPI-1485, Glial-cell-line- |
| Stimulators | | derived Neurotrophic |
| | | Factor, SR-57667, PYM- |
| | | 50028 |
| Cell Replacement | Replace damaged neurons with health neurons. | Spheramine |
| Therapy | | |

The above classifications are exemplary only. It will be appreciated that a drug class need not be restricted to drugs used in the treatment of a single disease, but that a given mechanistic class may have members useful in the treatment of a number of diseases. For a example, MAO-B inhibitors are useful in the treatment of both Parkinson's disease and depression; as another example, statins are useful in the treatment of dyslipidemias but are also being found to have more general use in diseases where inflammation plays a major role, e.g., multiple sclerosis and other diseases.

Further classifications of drugs by mechanism are known in the art; often these classifications may be further classified by structure. Non-exclusive examples of drug classes useful in the methods and compositions of the invention, and representative members of these classes, include:

Sedative-Hypnotic Drugs, which include drugs that bind to the GABA_{A} receptor such as the benzodiazepines (including alprazolam, chlordiazepoxide, clorazepate, clonazepam, diazepam, estazolam, flurazepam, halazepam, lorazepam, midazolam, oxazepam, quazepam, temazepam, triazolam), the barbiturates (such as amobarbital, pentobarbital, phenobarbital, secobarbita), and non-benzodiazepines (such as zolpidem and zaleplon), as well as the benzodiazepine antagonists (such as flumazenil). Other sedative-hypnotic drugs appear to work through non-GABA-ergic mechanisms such as through interaction with serotonin and dopaminergic receptors, and include buspirone, isapirone, geprirone, and tandospirone. Older drugs work through mechanisms that are not clearly elucidated, and include chloral hydrate, ethchlorvynol, meprobamate, and paraldehyde.

In some embodiments, sedative-hypnotic drugs that interact with the GABA receptor, such as benzodiazepines and non-benzodiazepines, are further classified as to which subunit or subunits of the GABA_{A} receptor that they interact with, e.g., the α (which is further classified into six subtypes, including α-1,2,3, and 5), β (further classified as four different types), γ (three different types), δ, ε, π, ρ, etc. Such a classification can allow further refinement of associations between genetic variation and responsiveness to a given sedative-hypnotic that interacts with a particular subclass, and predictions for a new sedative-hypnotic that interacts with the same subclass of receptors.

Opioid analgesics and antagonists act on the opioid receptor. The majority of currently available opioid analgesics act primarily at the μ opioid receptor. However, interactions also occur with the δ and κ receptors. Similar to the sedative-hypnotics, in some embodiments opioid analgesics are further classed as to subtypes of receptors at which they primarily interact, thus allowing further refinement of the association between drug response and genetic variation, and higher predictability for a new drug, based on which receptor(s) it interacts with. Opioid analgesics include alfentanil, buprenorphine, butorphanol, codeine, dezocine, fentanyl, hydromorphone, levomethadyl acetate, levorphanol, meperidine, methadone, morphine sulfate, nalbuphine, oxycodone, oxymorphone, pentazocine, propoxyphene, remifentanil, sufentanil, tramadol; analgesic combinations such as codeine/acetaminophen, codeine/aspirin, hydrocodone/acetaminophen, hydrocodone/ibuprofen, oxycodone/acetaminophen, oxycodone/aspirin, propoxyphene/aspirin or acetaminophen. Opioid antagonists include nalmefene, naloxone, naltrexone. Antitussives include codeine, dextromethorphan.

Nonsteroidal anti-inflammatory drugs act primarily through inhibition of the synthesis of prostaglandins, e.g., through inhibition of COX-1, COX-2, or both. Older NSAIDS (e.g. salicylates) tend to be non-selective as to the type of COX inhibited, whereas newer drugs are quite selective (e.g., the COX-2 inhibitors). Non-selective COX inhibitors include aspirin, acetylsalicylic acid, choline salicylate, diclofenac, etodolac, fenoprofen, flurbiprofen, ibuprofen, indomethacin, ketoprofen, ketorolac, magnesium salicylate, meclofenamate, mefenamic acid, nabumetone, naproxen, oxaprozin, phenylbutazone, piroxicam, salsalate, salicylsalicylic acid, sodium salicylate, sodium thiosalicylate, sulindac, tenoxicam, tiaproven, azapropazone, carprofen, and tolmetin. Selective COX-2 inhibitors include celecoxib, etroricoxib, meloxicam, rofecoxib, and valdecoxib.

Histamine agonists and antagonists are classified according to receptor subtype. H₁ agonists or partial agonists include 2-(m-fluorophenyl)-histamine and antagonists include chlorpheniramine, scopolamine, mepyramine, terfenadine, astemizole, and triprolidine; further antagonists (which may be further classified by their chemical structures) include the ethanolamines carbinoxamine, dimenhydrinate, diphenhydramine, and doxylamine; the ethylaminediamines pyrilamine and tripelennamine; the piperazine derivatives dydroxyzine, cyclizine, fexofenadine and meclizine; the alkylamines brompheniramine and chlorpheniramine; and miscellaneous antagonists cyproheptadine, loratadine, cetrizine. H₂ agonists include dimaprit, impromidine, and amthamine; and antagonists (useful in the treatment of gastric acid secretion) include cimetidine, ranitidine, nizatidine, and famotidine; H₃ agonists include R-alpha-methylhistamine, imetit, and immepip and antagonists include thioperamide, iodophenpropit, and clobenpropit; and H₄ agonists include clobenpropit, imetit, and clozapine and antagonists include thioperamide. Available preparations include the H1 blockers azelastine, brompheniramine, buclizine, carbinoxamine, cetrizine, chlorpheniramine, clemastine, cyclizine, cyproheptadine, desloratidine, dimenhydrinate, diphenhydramine, emedastine, fexofenadine, hydroxyzine, ketotifen, levocabastine, loratadine, meclizine, olopatadine, phenindamine, and promoathazine.

Drugs used in asthma include sympatheticomimetics (used as “relievers,” or bronchodilators) such as albuterol, albuterol/lpratropium, bitolterol, ephedrine, epinephrine, formoterol, isoetharine, isoproterenol, levalbuterol, metaproterenol, pirbuterol, salmeterol, salmeterol/fluticasone, terbutaline; aerosol corticosteroids (used as “controllers,” or antiinflammatory agents) such as beclomethasone, budesonide, flunisolide, fluticasone, fluticasone/salmeterol, triamcinolone; leukotriene inhibitors such as montelukast, zafirlukast, zileuton; cormolyn sodium and nedocromil sodium; methylxanthines such as aminophylline, theophyllinem dyphylline, oxtriphylline, pentoxifylline; antimuscarinic drugs such as ipratropium; and antibodies such as omalizumab.

Erectile dysfunction drugs include cGMP enhancers such as sildenafil (Viagra), tadalafil, vardenafil, and alprostadil, and dopamine releasers such as apomorphine

Drugs used in the treatment of gastrointestinal disease act by a number of mechanisms. Drugs that counteract acidity (antacids) include aluminum hydroxide gel, calcium carbonate, combination aluminum hydroxide and magnesium hydroxide preparation. Drugs that act as proton pump inhibitors include esomeprazole, lansoprazole, pantoprazole, and rabeprazole. H2 histamine blockers include cimetidine, famotidine, nizatidine, ranitidine. Anticholinergic drugs include atropine, belladonna alkaloids tincture, dicyclomine, glycopyrrolate, I-hyoscyamine, methscopolamine, propantheline, scopolamine, tridihexethyl. Mucosal protective agents include misoprostol, sucralfate. Digestive enzymes include pancrelipase. Drugs for motility disorders and antiemetics include alosetron, cisapride, dolasetron, dronabinol, granisetron, metoclopramide, ondansetron, prochlorperazine, tegaserod. Antiinflammatory drugs used in gastrointestinal disease include balsalazide, budesonide, hydrocortisone, mesalamine, methylprednisone, olsalazine, sulfasalazine, infliximab. Antidiarrheal drugs include bismuth subsalicylate, difenoxin, diphenoxylate, kaolin/pectin, loperamide. Laxative drugs include bisacodyl, cascara sagrada, castor oil, docusate, glycerin liquid, lactulose, magnesium hydroxide [milk of magnesia, Epson Salt], methylcellulose, mineral oil, polycarbophpil, polyethylene glycol electrolyte solution, psyllium, sienna. Drugs that dissolve gallstones include monoctanoin, ursodiol.

Cholinoceptor-activating drugs, which act by activating muscarinic and/or nicotinic receptors include esters of choline (e.g., acetylcholine, metacholine, carbamic acid, carbachol, and bethanechol) and alkaloids (e.g., muscarine, pilocarpine, lobeline, and nicotine); cholinesterase-inhibiting drugs which typically act on the active site of cholinesterase include alcohols bearing a quaternary ammonium group (e.g., edrophonium), carbamates and related agents (e.g., neostigmine, physostigmine, pyridostigmine, ambenonium, and demercarium), and organic derivatives of phosphoric acid (e.g., echothiophate, soman, parthion, malathion); cholinoceptor-blocking drugs typically act as antagonists to nicotinic receptors (further classified as ganglion-blockers, such as hexamethonium, mecmylamine, teteraethylammonium, and trimethaphan; and neuromuscular junction blockers, see skeletal muscle relaxants) or antagonists to muscarinic receptors (e.g. atropine, propantheline, glycopyrrolate, pirenzepine, dicyclomine, tropicamide, ipatropium, banztropine, gallamine, methooctramine, AF-DX 116, telenzipine, trihexyphenidyl, darifenacin, scopolamine, homatropine, cyclopentolate, anisotropine, clidinium, isopropamide, mepenzolate, methscopolamine, oxyphenonium, propantheline, oxybutynin, oxyphencyclimine, propiverine, tolterodine, tridihexethyl), which can be further subclassed as to which muscarinic receptor is the primary site of the effect, e.g., M₁, M₂, M₃, M₄, or M₅, allowing greater predictability for an association between a genetic variation and a response for a new drug based on its primary site of effect. Available preparations of antimuscarinic drugs include but are not limited to atropine; beladonna alkaloids, extract, or tincture; clidinium; cyclopentolate; dicyclomine; flavoxate; glycopyrrolate; homatropine; 1-hysocyamine; ipratropium; mepenzolate; methantheline; methscopolamine; oxybtynin; prpantehline; scopolamine; tolterodine; tridihexethyl; tropicamide. Available preparations of ganglion blockers include mecamylamine and trimethaphan. Available cholinesterase regenerators include pralidoxime.

Adrenoceptor-activating drugs and other sympathomimetic drugs may be classified according to the receptor or receptors that they activate, e.g., alpha-one type (including subtypes A, B, D), alpha-two type (including subtypes A, B, and C), beta type (including subtypes 1, 2, and 3), and dopamine type (including subtypes 1, 2, 3, 4, and 5. Exemplary drugs include epinephrine, norepinephrine, phenylephrine, methoxamine, milodrine, ephedrine, xylometazoline, amphetamine, methamphetamine, phenmetrazine, methylphenidate, phenylpropanolamine, methylnorepinephrine, dobutamine, clonidine, BHT920, oxymetazoline, isoproterenol, procaterol, terbutaline, metaproterenol, albuterol, ritodrine, BRL37344, dopamine, fenoldopam, bromocriptine, quinpirol, dexmedetomidine, tyramine, cocaine (dopamine reuptake inhibitor), apraclonidine, brimonidine, ritodrine, terbutaline, and modafinil. Available preparations include amphetamine, apraclonidine, brimonidine, dexmedetomidine, dexmthylphenidate, dextroamphetamine, dipivefrin, dobutamine, dopamine, ephedrine, epinephrine, fenoldopam, hydroxyamphetamine, isoproterenol, mephentermine, metaraminol, methamphetamine, methoxamine, methylphenidate, midodrine, modafinil, naphazoline, norepinephrine, oxymetzoline, pemoine, phendimetrazine, phenylephrine, pseudoephedrine, tetrahydrozoline, and xylometaoline.

Adrenoceptor antagonist drugs may be classified by receptor Type In the same manner as adrenoceptor agonists, and include tolazoline, dibenamine, prazosin, terazosin, doxazosin, phenoxybenzamine, phentolamine, rauwoscine, yohimbine, labetalol, carvedilol, metoprololol, acebutolol, alprenolol, atenolol, betaxolol, celiprolol, esmolol, propanolol, carteolol, penbutolol, pindolol, timolol, butoxamine, ergotamine, dihydroergotamine, tamulosin, alfuzosin, indoramin, urapidil, bisoprolol, nadolol, sotalol, oxpenolol, bopindolol, medroxalol, and bucindolol. Available preparations include: alpha blockers doxazosin, phenoxybenzamine, phentolamine, prazosin, tamsulosin, terazosin, and tolazoline; and beta blockers acebutolol, atenolol, betaxolol, bisoprolol, carteolol, carvedilol, esmolol, labetolol, levobunolol, metiproanolol, nadolol, penbutolol, pinolol, propanolol, sotalol, timolol; and synthesis inhibitor metyrosine.

Antihypertensive agents include drugs that work by a variety of mechanisms and thus overlap with other classifications. Agents can include diuretics such as thiazide diuretics, and potassium sparing diuretics; drugs that act on the central nervous system such as methyldopa and clonidine; ganglion-blocking drugs, suprea; adrenergic neuron-blocking agents such as gunethidine, gunadrel, bethanidine, debrisoquin, and reserpine; adrenoceptor antagonists such as propanolol, metoprolol, nadolol, carteolol, atenolol, betaxolol, bisoprolol, pindolol, acebutolol, and penbutolol, labetalol, carvedilol, esmolol, pazosin, phentolamine and phenoxybenzamine; vasodilators such as hydralzaine, minoxidil, sodium nitroprusside, diazoxide, fenoldopam, and calcium channel blockers (e.g., verapamil, diltiazem, amlopidine, felopidine, isradipine, nicardipine, nifedipine, and nisoldipine); ACE-inhibitors such as captropril, enalapril, lisinopril, benazepril, fosinopril, moexipril, perindopril, quinapril, ramipril, and trandolapril; angiotensin receptor blocking agents such as losartan, valsartan, candesartan, eprosartan, irbesartan, and telmisartan. Preparations available include: beta adrenoceptor blockers acebutolol, atenolol, betaxolol, bisoprolol, carteolol, carvedilol, exmolol, labetalol, metoprolol, nadolol, penbutolol, pindolol, propanolol, timolol; centrally acting sympathoplegic drugs clonidine, gunabenz, guanfacine, methyldopa; postganglionic sympatheic nerve terminal blockers gunadrel, guanethidine, and reserpine; alpha one selective adrenoceptor blockers doxazosin, prazosin, terazosin; ganglion-blocking agent mecamylamine; vasodilators diazoxide, fenoldopam, hydralazine, minoxidil, nitroprusside; calcium channel blockers amlodipine, diltiazem, felodipine, isradipine, nicardipine, nisoldipine, nifedipine, verapamil; ACE inhibitors benazepril, captopril, enalapril, fosinopril, lisinopril, moexipril, perindopril, quinapril, ramipril, and trandolapril; and angiotensin receptor blockers candesartan, eprosartan, irbeartan, losartan, olmisartan, telmisartan, and valsartan.

Vasodilators used in angina pectoris include nitric oxide releasing drugs such as nitric and nitrous acid esters of polyalcohols such as nitroglycerin, isorbide dinitrate, amyl nitrite, and isosorbide mononitrate; calcium channel blockers such as amlodipine, felodipine, isradipine, nicardipine, nifedipine, nimodipine, nisoldipine, nitrendipine, bepridil, diltiazem, and verapamil; and beta-adrenoceptor-blocking drugs (see above). Available preparations include: nitrates and nitrites amyl nitrite, isosorbide dinitrate, isosorbide mononitrate, nitroglycerin; calcium channel blockers amlodipine bepridil, diltiazem, felodipine, isradipine, nicardipine, nifedipine, nimodipine, nisoldipine, and verapamil; and beta blockers acebutolol, atenolol, betaxolol, bisoprolol, carteolol, carvedilol, esmolol, labetolol, levobunolol, metiproanolol, nadolol, penbutolol, pinolol, propanolol, sotalol, timolol.

Drugs used in heart failure include cardiac glycosides such as digoxin; phosphodiesterase inhibitors such as inmrinone and milrinone; beta adrenoceptor stimulant such as those described; diuretics as discussed below; ACE inhibitors such as those discussed above; drugs that inhibit both ACE and neutral endopeptidase such as omaprtrilat; vasodilators such as synthetic brain natriuretic peptide (nesiritide) and bosentan; beta adrenoceptor blockers such as those described above. Available preparations include: digitalis digoxin; digitalis antibody digoxin immune Fab; sympathomimetics dobutamine and dopamine; ACE inhibitors captopril, enalapril, fosinopril, lisinopril, quinapril, ramipril, and trandolapril; angiotensin receptor blockers candesartan, wprosartan, irbesartan, losartan, olmesartan, telmisartan, and valsartan; beta blockers bisoprolol, carvedilol, and metoprolol.

Cardiac arrhythmia drugs include drugs that act by blocking sodium channels such as quinidine, amiodaron, disoprymide, flecamide, lidocaine, mexiletine, morcizine, procainamide, propafeneone, and tocamide; beta-adrenoceptor-blocking drugs such as propanolol, esmolol, and sotalol; drugs that prolong the effective refractory period by prolonging the action potential such as amiodarone, bretylium, sotalol, dofetilide, and ibutilide; calcium channel blockers such as verapamil, diltizem, and bepridil; and miscellaneous agents such as adenosine, digitalis, magnesium, and potassium. Available preparations include: the sodium channel blockers disopryamide, flecamide, lidocaine, miexiletine, moricizine, procainamide, propafenone, quinidine sulfate, quinidine gluconate, and quinidine polygalacturonate; the beta blockers acebutolol, esmolol, and propranolol; the action potential-prolonging agents amiodarone, bretylium, dofetilide, ibutilide, and sotalol; the calcium channel blockers bepridil, diltiazem, and verapamil; and adenosine and magnesium sulfate.

Diuretic agents include drugs that act as carbonic anhydrase inhibitors such as acetazoloamide, dichlorphenamide, methazolamide; loop diuretics such as furosemide, bumetanide, torsemide, ethacrynic acid, and mercurial diuretics; drugs that inhibit NaCl transport in the distal convoluted tubule and, in some cases, also act as carbonic anhydrase inhibitors, such as bendroflumethiazide, benzthiazide, chlorothiazide, chlorthalidone, hydrochlorothiazide, hydroflumethiazide, indapamide, methyclothiazide, metolazone, polythiazide, quinethazone, and trichlormethazide; potassium-sparing diuretics such as spironolactone, triamterene, eplerenone, and amiloride; osmotic diuretics such as mannitol; antidiuretic hormone agonists such as vasopressin and desmopressin; antidiuretic hormone antagonists such aslithium and demeclocycline. Available preparations include actetazolamide, amiloride, bendroflumethiazide, benzthiazide, brinzolamide, bumetanide, chlorothiazide, chlorthalidone, demeclocycline, dichlorphenamide, dorzolamide, eplerenone, ethacrynic acid, furosemide, hydrochlorothiazide, hydroflumethiazide, indapamide, mannitol, methazolamide, methyclothiazide, metolazone, polythiazide, quinethazone, apironolactone, torsemide, triamterene, and trichlormethiazide.

Serotonin and drugs that affect serotonin include serotonin agonists such as fenfluramine and dexfenfluramine, buspirone, sumatriptan, cisapride, tegaserod; seratonin antagonists p-chlorophenylalanine and p-chloroamphetamine, and reserpine; and the serotonin receptor antagonists phenoxybenzamine, cyproheptadine, ketanserin, ritanserin, and ondansetron; serotonin reuptake inhibitors are described elsewhere herein. Serotonin receptor agonists include almotriptan, eletriptan, frovatriptan, naratriptan, rizatriptan, sumatriptan, and zolmitriptan.

Ergot alkaloids are useful in the treatment of, e.g., migraine headache, and act on a variety of targets, including alpha adrenoceptors, serotonin receptors, and dopamine receptors. They include bromocriptine, cabergoline, pergolide, ergonovine, ergotamine, lysergic acid diethylamide, and methysergide. Available preparations include dihydroergotamine, ergonovine, ergotamine, ergotamine tartrate, and methylergonovine.

Vasoactive Peptides include aprepitant, bosentan.

Eicosanoids include prostaglandins, thomboxanes, and leukotrienes. Eicosanoid modulator drugs include alprostadil, bimatoprost, carboprost tromethamine, dinoprostone, epoprostenol, latanoprost, misoprostol, monteleukast, travaprost, treprostinil, unoprostone, zafirleukast, zileuton. Further eicosanoid modulators are discussed elsewhere herein as nonsteroidal antiinflammatory drugs (NSAIDs)

Drugs for the treatment of acute alcohol withdrawal include diazepam, lorazepam, oxazepam, thiamine; drugs for prevention of alcohol abuse include disulfuram, naltrexone; and drugs for the treatment of acute methanol or ethylene glycol poisoning include ethanol, fomepizole.

Antiseizure drugs include carbamazepine, clonazepam, clorazepate dipotassium, diazepam, ethosuximide, ethotoin, felbamate, fosphenytoin, gabapentin, lamotrigine, levetiracetam, lorazepam, mephenyloin, mephobarbital, oxycarbazepine, pentobarbital sodium, phenobarbital, phenyloin, primidone, tiagabine, topiramate, trimethadione, valproic acid.

General anesthetics include desflurane, dexmedetomidine, diazepam, droperidol, enflurane, etomidate, halothane, isoflurane, ketamine, lorazepam, methohexital, methoxyflurane, midazolam, nitrous oxide, propofol, sevoflurane, thiopental.

Local anesthetics include articaine, benzocaine, bupivacaine, butamben picrate, chloroprocaine, cocaine, dibucaine, dyclonine, levobupivacaine, lidocaine, lidocaine and etidocaine eutectic mixture, mepivacaine, pramoxine, prilocalne, procaine, proparacaine, ropivacaine, tetracaine.

Skeletal muscle relaxants include neuromuscular blocking drugs such as atracurium, cisatracurium, doxacurium, metocurine, mivacurium, pancuronium, pipecuronium, rocuronium, succinylcholine, tubocurarine, vecuronium; muscle relaxants (spasmolytics) such as baclofen, botulinum toxin type A, botulinum toxin type B, carisoprodol, chorphenesin, chlorzoxazone, cyclobenzaprine, dantrolene, diazepam, gabapentin, metaxalone, methocarbamol, orphenadrine, riluzole, and tizanidine.

Antipsychotic agents include aripiprazole, chlorpromazine, clozapine, fluphenazine, fluphenazine esters, haloperidol, haloperidol ester, loxapine, mesoridazine, molindone, olanzapine, perphenazine, pimozide, prochlorperazine, promazine, quetiapine, risperidone, thioridazine, thiothixene, trifluoperazine, triflupromazine, ziprasidone; mood stabilizers include carbamazepine, divalproex, lithium carbonate, and valproic acid.

Agents used in anemias include hematopoietic growth factors such as darbopoetin alfa, deferoxamine, epoetin alfa (erythropoetin, epo), filgrastim (G-CSF), folic acid, iron, oprelvekin (interleukin-11), pegfilgrastim, sargramostim (GM-CSF), vitamin B₁₂.

Disease-modifying antirheumatic drugs include anakinra, adalimumab, auranofin, aurothioglucose, etanercept, gold sodium thiomalate, hydroxychloroquine, infliximab, leflunomide, methotrexate, penicillamine, sulfasalazine. Drugs used in gout include allopurinol, colchicine, probenecid, sulfinpyrazone.

Drugs used in disorders of coagulation include abciximab, alteplase recombinant, aminocaproic acid, anisindione, antihemophilic factor [factor VIII, AHF], anti-inhibitor coagulant complex, antithrombin III, aprotinin, argatroban, bivalirudin, cilostazol, clopidogrel, coagulation factor VIIa recombinant, dalteparin, danaparoid, dipyridamole, enoxaparin, eptifibatide, Factor VIIa, Factor VIII, Factor IX, fondaparinux, heparin sodium, lepirudin, phytonadione [K₁], protamine, reteplase, streptokinase, tenecteplase, ticlopidine, tinzaparin, tirofiban, tranexamic acid, urokinase, warfarin.

Hypothalamic and pituitary hormones include bromocriptine, cabergoline, cetrorelix, chorionic gonadotropin [hCG], corticorelin ovine, corticotropin, cosyntropin, desmopressin, follitropin alfa, follitropen beta [FSH], ganirelix, gonadorelin acetate [GnRH], gonadorelin hydrochloride [GnRH], goserelin acetate, histrelin, leuprolide, menotropins [hMG], nafarelin, octreotide, oxytocin, pergolide, protirelin, sermorelin, somatrem, somatropin, thyrotropin alpha, triptorelin, urofollitropin, vasopressin.

Thyroid and antithyroid drugs include the thyroid agents: levothyroxine [T₄], liothyronine [T₃], liotrix [a 4:1 ratio of T₄:T₃], thyroid desiccated [USP]; and the antithyroid agents: diatrizoate sodium, iodide, iopanoic acid, ipodate sodium, methimazole, potassium iodide, propylthiouracil [PTU], thyrotropin; recombinant human TSH.

Adrenocorticosteroids and adrenocortical antagonists include the glucocorticoids for oral and parenteral use: betamethasone, betamethasone sodium phosphate, cortisone, dexamethasone, dexamethasone acetate, dexamethasone sodium phosphate, hydrocortisone [cortisol], hydrocortisone acetate, hydrocortisone cypionate, hydrocortisone sodium phosphate, hydrocortisone sodium succinate, methylprednisolone, methylprednisolone acetate, methylprednisolone sodium succinate, prednisolone, prednisolone acetate, prednisolone sodium phosphate, prednisolone tebutate, prednisone, triamcinolone, triamcinolone acetonide, triamcinolone diacetate, triamcinolone hexacetonide. Another class of adrenocorticoids are the mineralocorticoids, e.g., fludrocortisone acetate. The adrenal steroid antagonists include aminoglutethimide, ketoconazole, mitotane.

Gonadal hormones and inhibitors include the estrogens: conjugated estrogens, dienestrol, diethylstilbestrol diphosphate, esterified estrogens, estradiol cypionate in oil, estradiol, estradiol transdermal, estradiol valerate in oil, estrone aqueous suspension, estropipate, ethinyl estradiol; the progestins: hydroxyprogesterone caproate, levonorgestrel, medroxyprogesterone acetate, megestrol acetate, norethindrone acetate, norgestrel, progesterone; the androgens and the anabolic steroids: methyltestosterone, nandrolone decanoate, oxandrolone, oxymetholone, stanozolol, testolactone, testosterone aqueous, testosterone cypionate in oil, testosterone enanthate in oil, testosterone propionate in oil, testosterone transdermal system, testosterone pellets. Drugs may further be classed as antagonists and inhibitors of gonadal hormones: anastrozole, bicalutamide, clomiphene, danazol, dutasteride, exemestane, finasteride, flutamide, fulvestrant, letrozole mifepristone, nilutamide, raloxifene, tamoxifen, and toremifene.

Agents that affect bone mineral homeostasis include Vitamin E, its metabolites and analogs: calcifediol, calcitriol, cholecalciferol [D₃], dihydrotachysterol [DHT], doxercalciferol, ergocalciferol [D₂], and paricalcitol; calcium: calcium acetate [25% calcium], calcium carbonate [40% calcium], calcium chloride [27% calcium], calcium citrate [21% calcium], calcium glubionate [6.5% calcium]; calcium gluceptate [8% calcium], calcium gluconate [9% calcium], calcium lactate [13% calcium], and tricalcium phosphate [39% calcium]; phosphate and phosphate binders such as phosphate and sevelamer; and other drugs such as alendronate, calcitonin-salmon, etidronate, gallium nitrate, pamidronate, plicamycin, risedronate, sodium fluoride, teriparatide, tiludronate, zoledronic acid.

Beta-lactam antibiotics and other inhibitors of cell wall synthesis include the penicillins, such as amoxicillin, amoxicillin/potassium clavulanate, ampicillin, ampicillin/sulbactam sodium, carbenicillin, dicloxacillin, mezlocillin, nafcillin, oxacillin, penicillin G benzathine, penicillin G procaine, penicillin V, piperacillin, pipercillin and tazobactam sodium, ticarcillin, and ticarcillin/clavulanate potassium; the cephalosporins and other beta-lactam drugs, such as the narrow spectrum (first generation) cephalosporins, e.g., cefadroxil, cefazolin, cephalexin, cephalothin, cephapirin, and cephradine; the second generation (intermediate spectrum) cephalosporins, e.g., cefaclor, cefamandole, cefmetazole, cefonicid, cefotetan, cefoxitin, cefprozil, cefuroxime, and loracarbef; the broad spectrum (third- and fourth-generation cephalosporins, e.g., cefdinir, cefditoren, cefepime, cefixime, cefoperazone, cefotaxime, cefpodoxime proxetil, ceftazidime, ceftibuten, ceftizoxime, and ceftriaxone. Further classes include the carbapenem and monobactam, e.g., aztreonam, ertapenem, imipenen/cilastatin, and meropenem; and other drugs such as cycloserine (seromycin pulvules), fosfomycin, vancomycin.

Other antibiotics include chloramphenicol, the tetracyclines, e.g., demeclocycline, doxycycline, methacycline, minocycline, oxtetracycline, and tetracycline; the macrolides, e.g., azithromycin, clarithromycin, erythromycin; the ketolides, e.g., telithromycin; the lincomycins, e.g., clindamycin; the streptogramins, e.g., quinupristin and dalfopristin; and the oxazolidones, e.g., linezolid.

Aminoglycosides and spectinomycin antibiotics include amikacin, gentamicin, kanamycin, neomycin, netilmicin, paromomycin, spectinomycin, streptomycin, and tobramycin.

Sulfonamides, trimethoprim, and quinolone antibiotics include the general-purpose sulfonamides, e.g., sulfadiazine, sulfamethizole, sulfamethoxazole, sulfanilamide, and sulfisoxazole; the sulfonamides for special applications, e.g., mafenide, silver sulfadiazine, sulfacetamide sodium. Trimethoprims include trimethoprim, trimethoprim-sulfamethoxazole [co-trimoxazole, TMP-SMZ]; the quinolones and fluoroquinolones include cinoxacin, ciprofloxacin, enoxacin, gatifloxacin, levofloxacin, lomefloxacin, moxifloxacin, nalidixic acid, norfloxacin, ofloxacin, sparfloxacin, and trovafloxacin.

Antimycobacterial drugs include drugs used in tuberculosis, e.g., aminosalicylate sodium, capreomycin, cycloserine, ethambutol, ethionamide, isoniazid, pyrazinamide, rifabutin, rifampin, rifapentine, and streptomycin; and drugs used in leprosy, e.g., clofazimine, dapsone.

Antifungal agents include amphotericin B, butaconazole, butenafine, caspofungin, clotrimazole, econazole, fluconazole, flucytosine, griseofulvin, itraconazole, ketoconazole, miconazole, naftifine, natamycin, nystatin, oxiconazole, sulconazole, terbinafine, terconazole, tioconazole, tolnaftate, and voriconazole.

Antiviral agents include abacavir, acyclovir, adefovir, amantadine, amprenavir, cidofovir, delavirdine, didanosine, efavirenz, enfuvirtide, famciclovir, fomivirsen, foscarnet, ganciclovir, idoxuridine, imiquimod, indinavir, interferon alfa-2a, interferon alpha-2b, interferon-2b, interferon alfa-n3, interferon alfacon-1, lamivudine, lopinavir/ritonavir, nelfinavir, nevirapine, oseltamivir, palivizumab, peginterferon alfa-2a, peginterferon alfa-2b, penciclovir, ribavirin, rimantadine, ritonavir, saquinavir, stavudine, tenofovir, trifluridine, valacyclovir, valgancyclovir, zalcitabine, zanamivir, and zidovudine.

Further antimicrobial agents, disinfectants, antiseptics, and sterilants include the miscellaneous antimicrobial agents, e.g., methenamine hippurate, methenamine mandelate, metronidazole, mupirocin, nitrofurantoin, polymyxin B; and the disinfectants, antiseptics, and sterilants, e.g., benzalkonium, benzoyl peroxide, chlorhexidine gluconate, glutaraldehyde, hexachlorophene, iodine aqueous, iodine tincture, nitrofurazone, oxychlorosene sodium, providone-iodine, sliver nitrate, and thimerosal.

Antiprotozoal drugs include albendazole, atovaquone, atovaquone-proguanil, chloroquine, clindamycin, doxycycline, dehydroemetine, eflornithine, halofantrine, iodoquinol, mefloquine, melarsoprol, metronidazole, nifurtimox, nitazoxanide, paromomycin, pentamidine, primaquine, pyrimethamine, quinidine gluconate, quinine, sodium stibogluconate, sulfadoxine and pyrimethamine, and suramin.

Anthelmintic drugs include albendazole, bithionol, diethylcarbamazine, ivermectin, levamisole, mebendazole, metrifonate, niclosamide, oxamniquine, oxantel pamoate, piperazine, praziquantel, pyrantel pamoate, suramin, thiabendazole.

Immunopharmacological agents include abciximab, adalimumab, alefacept, alemtuzumab, anti-thymocyte globulin, azathioprine, basiliximab, BCG, cyclophosphamide, cyclosporine, daclizumab, etanercept, gemtuzumab, glatiramer, ibritumomab tiuxetan, immune globulin intravenous, infliximab, interferon alfa-2a, interferon alfa 2b, interferon beta-1a, interferon beta-1b, interferon gamma-1b, interleukin-2, IL-2, aldesleukin, leflunomide, levamisole, lymphocyte immune globulin, methylprednisolone sodium succinate, muromonab-CD3 [OKT3], mycophenolate mofetil, pegademase bovine, peginterferon alfa-2a, peginterferon alfa-2b, prednisone, RHₒ(D) immune globulin micro-dose, rituximab, sirolimus, tacrolimus [FK506], thalidomide, and trastuzumab.

Heavy metal chelators include deferoxamine, dimercaprol, edetate calcium [calcium EDTA], penicillamine, succimer, and unithiol.

b. Structural Classes of Drugs

In another example of drug classification embodiments, a drug may be classified according to its structural class or family; certain drugs may fall into more than one structural class or family. Thus, in some embodiments, drugs are classified according to structure. Drugs that have a common action may have different structures, and often one of the best predictors of a drugs likely action is its structure. By way of example only, certain classes of drugs may be further organized by chemical structure classes presented herein. One non-limiting example is antibiotics. Table 8, below, presents non-limiting examples of antibiotics further classified by illustrative chemical structure classes.

TABLE 8
Structural Classes of Antibiotic Drugs

| | |
|---|---|
| Structure Class | Examples of Antibiotics within Structure Class |
| Amino Acid Derivatives | Azaserine, Bestatin, Cycloserine, |
| | 6-diazo-5-oxo-L-norleucine |
| Aminoglycosides | Armastatin, Amikacin, Gentamicin, |
| | Hygromicin, Kanamycin, Streptomycin |
| Benzochinoides | Herbimycin |
| Carbapenems | Imipenem, Meropenem |
| Coumarin-glycosides | Novobiocin |
| Fatty Acid Derivatives | Cerulenin |
| Glucosamines | 1-deoxynojirimycin |
| Glycopeptides | Bleomycin, Vancomycin |
| Imidazoles | Metroidazole |
| Penicillins | Benzylpenicillin, Benzathine penicillin, |
| | Amoxycillin, Piperacillin |
| Macrolides | Amphotericin B, Azithromycin, Erythromycin |
| Nucleosides | Cordycepin, Formycin A, Tubercidin |
| Peptides | Cyclosporin A, Echinomycin, Gramicidin |
| Peptidyl Nucleosides | Blasticidine, Nikkomycin |
| Phenicoles | Chloramphenicol, Thiamphenicol |
| Polyethers | Lasalocid A, Salinomycin |
| Quinolones | 8-quinolinol, Cinoxacin, Ofloxacin |
| Steroids | Fusidic Acid |
| Sulphonamides | Sulfamethazine, Sulfadiazine, Trimethoprim |
| Tetracyclins | Oxytetracyclin, Minocycline, Duramycin |

In some embodiments, drugs are classed as optical isomers, where a class is two or more optical isomers, or racemate, of a compound of the same chemical formula. Thus, the invention includes methods and compositions for screening individuals for a genetic variation and/or phenotypic variation that predicts responsiveness to a first drug, and using this association to determine whether or not to modulate the treatment of an individual with a second drug, where the first and second drugs are optical isomers. In some embodiments, the first drug is a racemate and the second drug is a stereoisomer that is a component of the racemate. In some embodiments the first drug is a stereoisomer and the second drug is a racemate that includes the stereoisomer. In some embodiments the first drug is a first stereoisomer and the second drug is a second stereoisomer of a compound.

In some embodiments, drugs are classed as different crystal structures of the same formula. Thus, the invention includes methods and compositions for screening individuals for a genetic variation and/or phenotypic variation that predicts responsiveness to a first drug, and using this association to determine whether or not to modulate the treatment of an individual with a second drug, where the first and second drugs are members of a class of drugs of the same chemical formula but different crystal structures.

In some embodiments, drugs are classed by structural components common to the members of the class. Thus, the invention includes methods and compositions for screening individuals for a genetic variation and/or phenotypic variation that predicts responsiveness to a first drug, and using this association to determine whether or not to modulate the treatment of an individual with a second drug, where the first and second drugs are members of a class of drugs that contain the same structural component. By way of example only, a drug may be structurally classified as an acyclic ureide; acylureide; aldehyde; amino acid analog; aminoalkyl ether (clemastine, doxylamine); aminoglycoside; anthracycline; azalide; azole; barbituate; benzodiazapene; carbamate (e.g., felbamate, meprobamate, emylcamate, phenprobamate); carbapenam; carbohydrate; carboxamide (e.g., carbamazepine, oxcarbazepine); carotenoid (e.g., lutein, zeaxanthin); cephalosporin; cryptophycin; cyclodextrin; diphenylpropylamine; expanded porphyrin (e.g. rubyrins, sapphyrins); fatty acid; glycopeptide; higher alcohol; hydantoins (e.g., phenyloin); hydroxylated anthroquinone; lincosamide; lipid; lipid related compound; macrolide; mustard; nitrofuran; nitroimidazole; non-natural nucleotide; non-natural nucleoside; oligonucleotide; organometallic compound; oxazolidinedione; penicillin; phenothiazine derivative (alimemazine, promethazine); phenylpiperidine; phthalocyanine; piperazine derivative (e.g., cetrizine, meclozine); platinum complex (e.g., cis-platin); polyene; polyketide; polypeptide; porphyrin; prostaglandin (e.g., misoprostol, enprostil); purine; pyrazolone; pyrimidine; pyrrolidine (levetiracetam); quinolone; quinone; retinoid (e.g., isotretinoin, tretinoin); salicylate; sphingolipid; steroid (e.g., prednisone, triamcinolone, hydrocortisone); substituted alkylamine (e.g., talastine, chlorphenamine); substituted ethylene diamine(mepyramine, thonzylamine); succinimide (ethosuximide, phensuximide, mesuximide); sulfa; sulfonamide(sulfathiazole, mafenide); sulfone; taxane; tetracycline (e.g., chlortetracycline, oxytetracline); texaphyrin (e.g., Xcytrin, Antrin); thiazide; thiazolidinedione; tocopherol, tocotrienol, triazine (e.g., lamotrigine); urea; xanthine (theobromine, aminophylline); and zwitterion.

c. Implementation

The present inventors have recognized that one or more genetic variations and/or phenotypes associated with a response to a first drug are useful in predicting probable responses to a second drug in the same class, and, if necessary, in guiding possible modulations of the administration of the second drug. In some embodiments, the first and second drugs are the same drug; in some embodiments, the first and second drugs are different drugs. Such predictions can be used in, e.g., clinical trials or in therapeutic practice to increase the probability that the second drug is administered to an appropriate patient population and/or is administered in such a way as to maximize the probability of a positive (e.g., therapeutic) response and minimize the probability of a negative (e.g., adverse) response.

Implementing the methods and compositions of the invention is a matter of routine use of information and techniques that are well-known and well-established in the art. A number of databases of genetic variations, such as SNPs, are well-established, as described herein; indeed, a large number of human SNPs have been mapped and described. Methods of testing large numbers of genetic variations with rapidity and accuracy are known and in routine use in the art. Response to drugs are currently noted and cataloged for individuals enrolled in research studies, such as clinical trials, and include therapeutic responses (as well as the degree of response) and non-therapeutic responses, e.g., adverse effects. Mechanistic and structural classes of drugs are known, so that drugs in each class can be related to one another; numerous exemplary classifications are presented herein. Sophisticated and powerful software for associating one factor with another (e.g. genetic variation with drug response) are available, as are the statistical methods necessary for such software. Thus, the methods and compositions of the invention require no more than routine experimentation in their implementation.

### V. Screening and Treatment Methods and Compositions

Methods of the invention include methods of screening and treatment of an individual suffering from a disorder include screening an individual in need of treatment for a disorder for a genetic variation indicating a predisposition to a response to first drug and administering or not administering a second drug to the individual based on the results of the screening. In some embodiments the first drug and the second drug are the same; in other embodiments they are different, e.g., different members of a class of drugs. Such screening can be used, for example, to identify individuals who may benefit (or not benefit) from treatment with a drug, individuals who may be enrolled (or excluded) from a clinical trial, and/or individuals who may suffer (or not suffer) an adverse reaction from a drug. In some embodiments one or more phenotypes may also be included in the screening step.

In some embodiments, methods of the invention include methods of screening and treatment of an individual suffering from a disorder of blood glucose regulation, e.g., an insulin resistance disorder, that include screening an individual in need of treatment for a disorder of blood glucose regulation, e.g., an insulin resistance disorder, for a genetic variation indicating a predisposition to a response to a first insulin sensitizer; and administering or not administering a second insulin sensitizer to the individual based on the results of the screening. In some embodiments, the first and second insulin sensitizers are the same. In some embodiments, the first and second insulin sensitizers are different, e.g., different members of a single class of drugs. Such screening can be used, for example, to identify individuals who may benefit (or not benefit) from treatment with an insulin sensitizer, individuals who may be enrolled (or excluded) from a clinical trial, and/or individuals who may suffer (or not suffer) an adverse reaction from an insulin sensitizer. In some embodiments one or more phenotypes may also be included in the screening step.

Genetic variations, e.g., SNPs, used in the screening steps of the methods herein may be any genetic variations that have been found to be associated with a type of responsiveness to drug, e.g., an insulin sensitizer that is of interest, e.g., susceptibility to adverse effects of drug, e.g., an insulin sensitizer. In some embodiments, one or more genetic variations are SNPs found in the associations studies described herein. In some embodiments, one or more genetic variations are SNPs available on databases, and these SNPs are genotyped.

Once a SNP or other genetic variation has been identified, various compositions useful in screening, diagnosis, prognosis, and the like, may be made. These include nucleic acids, polypeptides, antibodies, and the like. Such compositions and methods are described in detail in U.S. application Ser. No. 10/447,685, filed May 28, 2003, entitled “Liver Related Disease Compositions and Methods;” U.S. Provisional Patent Application No. 60/648,957, filed Jan. 31, 2005, entitled “Compositions and Methods for Treating, Preventing, and Diagnosing Alzheimer's Disease;” U.S. Provisional Patent Application No. 60/653,672, filed Feb. 16, 2005, entitled “Parkinson's Disease-Related Disease Composition and Methods;” U.S. application Ser. No. 11/344,975, filed Jan. 31, 2006, entitled “Genetic Basis of Alzheimer's Disease and Diagnosis and Treatment Thereof;” U.S. application Ser. No. 11/299,298, filed Dec. 9, 2005, entitled “Markers for Metabolic Syndrome Obesity and Insulin Resistance;” U.S. Provisional Patent Application No. 60/781,483, filed Mar. 10, 2006, entitled “Markers for Breast Cancer;” U.S. Provisional Patent Application No. 60/760,198, filed Jan. 19, 2006, entitled “Markers for Myocardial Infarction;” and U.S. Provisional Patent Application No. 60/811,318, filed Jun. 6, 2006, entitled “Markers for Addiction,” all of which are incorporated herein by reference.

### A. Nucleic Acids

The term “drug response nucleic acid,” “drug response associated genomic region,” or “associated genomic region” means a nucleic acid, or fragment, derivative, variant or complement thereof, associated with a response to a drug (wherein, as used herein, “response” or “responsiveness” includes the lack of an effect in the individual from the drug) including, for example, coding and non-coding regions of an associated gene, and/or genomic regions spanning regions extending upstream and about downstream of the nucleic acid of an associated gene, and variants thereof. The term “associated gene” as used herein refers to a gene that is associated with a response to a drug. In some embodiments the associated genomic region extends from about 10 kb upstream to about 10 kb downstream of the associated gene. In some embodiments the region extends from about 5 kb upstream to about 5 kb downstream of the associated gene. In some embodiments the region extends from about 2 kb upstream to about 2 kb downstream of the associated gene. In some embodiments the region extends from about 1 kb upstream to about 1 kb downstream of the associated gene. In some embodiments, the associated genomic region includes regulatory regions that modulate expression of an associated gene. The invention also contemplates nucleic acids that are products of an associated gene, e.g., RNA transcripts and splicing variants, modifications or derivatives thereof, etc. The invention also contemplates nucleic acids that are not within a gene that are nonetheless associated with a response to a drug, and these nucleic acids are also encompassed by the term “drug response nucleic acid.” The nucleic acids of the invention may contain one or more associated polymorphisms (e.g., SNPs). For example, the sequence of an associated gene in an individual may contain one or more alleles associated with a drug response, one or more alleles associates with a lack of response, or a combination thereof. The term also includes nucleic acids similarly related to genes in an associated gene pathway. The term “associated gene pathway” generally refers to genes and gene products comprising a drug response pathway, and may include one or more genes that act upstream or downstream of an associated gene in a drug response pathway; or any gene whose gene product interacts with, binds to, competes with, induces, enhances or inhibits, directly or indirectly, the expression or activity of an associated gene; or any gene whose expression or activity is induced, enhanced or inhibited, directly or indirectly, by an associated gene; or any gene whose gene product is induced, enhanced or inhibited, directly or indirectly, by an associated gene. An associated gene pathway may refer to one or more genes.

The term “insulin sensitizer response nucleic acid” or “insulin sensitizer response associated genomic region” means a nucleic acid, or fragment, derivative, variant or complement thereof, associated with a response to an insulin sensitizer (wherein, as used herein, “response” or “responsiveness” includes the lack of an effect in the individual from the insulin sensitizer) including, for example, coding and non-coding regions of an associated gene, and/or genomic regions spanning regions extending upstream and about downstream of the nucleic acid of an associated gene, and variants thereof. In some embodiments the region extends from about 10 kb upstream to about 10 kb downstream of the nucleic acid. In some embodiments the region extends from about 5 kb upstream to about 5 kb downstream of the nucleic acid. In some embodiments the region extends from about 2 kb upstream to about 2 kb downstream of the nucleic acid. In some embodiments the region extends from about 1 kb upstream to about 1 kb downstream of the nucleic acid. The invention also contemplates nucleic acids that are not within a gene that are nonetheless associated with a response to an insulin sensitizer, and these nucleic acids are also encompassed by the term “insulin sensitizer response nucleic acid.” The term also includes nucleic acids similarly related to genes in an associated gene pathway.

A drug response nucleic acid, e.g., an insulin sensitizer response nucleic acid, can include coding sequence and/or non-coding sequence. It can comprise, consist essentially of, or consist of the exon or intron encompassing such position. It can be of variable length. In some embodiments such a nucleic acid can be less than 500,000, 100,000, 50,000, 10,000, 5,000, 1,000, 500, 100, 10 or 5 nucleotides in length. In some embodiments such a nucleic acid can be greater than 5, 10, 50, 100, 300, 600, 900, 1,000, 3,000, 6,000, 9,000, 10,000, 30,000, 60,000, 90,000, 100,000, 300,000, 600,000, or 900,000 nucleotides in length.

In one embodiment, a drug response nucleic acid, e.g., an insulin sensitizer response nucleic acid, is one that can specifically hybridize to an associated genomic region encompassing a nucleic acid position known to be a genetic variation associated with response to the drug, e.g., insulin sensitizer, e.g., a SNP, or an associated genomic region comprising a nucleic acid in a haplotype block with the position. Methods for identifying variants in a common haplotype block are provided in U.S. Pat. No. 6,969,589, assigned to the same assignee as the present application.

The term “drug response polypeptide” refers to any peptide, polypeptide, or fragment, derivative or variant thereof, associated with responsiveness to drug (wherein, as used herein, “response” or “responsiveness” includes the lack of an effect in the individual from the drug), including a peptide or polypeptide regulated or encoded, in whole or in part, by an associated gene or genomic regions immediately upstream or downstream of an associated gene, or fragment, variants, derivative, or modifications thereof. The term also includes such polypeptides up- or down-stream in an associated gene pathway.

The term “insulin sensitizer response polypeptide” refers to any peptide, polypeptide, or fragment, derivative or variant thereof, associated with responsiveness to an insulin sensitizer (wherein, as used herein, “response” or “responsiveness” includes the lack of an effect in the individual from the insulin sensitizer), including a peptide or polypeptide regulated or encoded, in whole or in part, by an associated gene or genomic regions immediately upstream or downstream of an associated gene, or fragment, variants, derivative, or modifications thereof. The term also includes such polypeptides up- or down-stream in an associated gene pathway.

The term “stringent conditions” refers to conditions for hybridization of complementary nucleic acid wherein the presence of a nucleic acid may be detected. Different stringency conditions may be utilized under different circumstances. Stringent conditions depend on, for example, length of the nucleic acids, temperature and buffers. Generally, stringent conditions are selected to be about 5° C. lower than the thermal melting point (Tm) of a specific sequence at a defined ionic strength and pH. The Tm is the temperature (under defined ionic strength, pH and nucleic acid concentration) at which 50% of the complementary nucleic acids hybridize to a target nucleic acid at equilibrium. As target nucleic acids are generally present in excess, at Tm, 50% of the complementary nucleic acids are occupied at equilibrium. Typically, stringent conditions include a salt concentration of at least about 0.01 to 1.0 M Na ion concentration (or other salts) at pH 7.0 to 8.3 and the temperature is at least about 30° C. for short probes (e.g., 10 to 50 nucleotides). Stringent conditions can also be achieved with the addition of destabilizing agents such as formamide. For example, conditions of 5×SSPE (750 mM NaCl, 50 mM NaPhosphate, 5 mM EDTA, pH 7.4) and a temperature of 25-30° C. are suitable for allele-specific nucleic acid hybridizations. In certain embodiments, sample nucleic acid comprises target nucleic acid and complementary nucleic acids are immobilized on a substrate.

The terms “isolated” and “purified” refer to a material that is substantially or essentially removed from or concentrated in its natural environment. For example, an isolated nucleic acid may be one that is separated from the nucleic acids that normally flank it or other nucleic acids or components (proteins, lipids, etc.) in a sample. In another example, a polypeptide is purified if it is substantially removed from or concentrated in its natural environment. Methods for purification and isolation of nucleic acids are well known in the art.

The term “nucleic acid,” refers to a deoxyribonucleotide, ribonucleotide and/or a mimetic thereof, whether singular or in polymers, naturally occurring or non-naturally occurring, double-stranded or single-stranded, translated (e.g., gene) or untranslated (e.g. regulatory region), or any fragments, derivatives or complements thereof. A nucleic acid includes analogs (e.g., phosphorothioates, phosphoramidates, methyl phosphonate, chiral-methyl phosphonates, 2-O-methyl ribonucleotides) or modified nucleic acids (e.g., modified backbone residues or linkages) or nucleic acids that are combined with carbohydrate, lipids, protein or other materials or peptide nucleic acids (PNAs). A nucleic acid can include one or more polymorphisms, variations or mutations. Examples of nucleic acids include oligonucleotides, nucleotides, polynucleotides, nucleic acid sequences, genomic sequences, antisense nucleic acids, probes, primers, genes, regulatory regions, introns, exons, open-reading frames, binding agents, target nucleic acids and allele specific nucleic acids.

The terms “polypeptide,” “peptide,” “oligopeptide” and “protein” are used interchangeably to refer to a polymer of amino acids, PNAs or mimetics, of no specific length and to all fragments, isoforms, variants, derivatives and modifications thereof. A polypeptide may be naturally and non-naturally occurring. The term isoform refers to different gene products resulting from the same gene, e.g., due to alternative splicing. The term variant when used to describe a polypeptide refers to variations in amino acid sequences, whether or not such variations result in conservative or non-conservative substitutions. The term modification include tags, labels, post-translational modifications or other chemical or biological modifications. In one embodiment a polypeptide is purified.

“Response to a drug,” e.g., “Response to an insulin sensitizer” or “drug response,” e.g., “insulin sensitizer response” is as described herein, and includes therapeutic and non-therapeutic responses (e.g., adverse effects). A nucleic acid associated with response to drug, e.g., an insulin sensitizer is one that is expressed differently in individuals having a phenotype of response to the drug as compared to individuals not having the same phenotype of response to the drug, or a nucleic acid having one or more variants associated with response to the drug.

Tables 9-11 identify SNPs from the analysis of individual genotypes that have significant association with drug response (nominal p-value <0.001), e.g., edema. Table 9 provides a collection of 345 variant sites having forms associated with susceptibility or resistance to drug response, e.g., edema. Further information about the variant sites provided in Table 9 is shown in Tables 10 and 11. The variant sites occur in or proximal to the genes provided in Table 10. Further information about the variant sites, their alleles, and the statistical analysis identifying them as associated with drug response, e.g. edema, is provided in Table 11.

Table 9, column 1, entitled “rsID,” lists a SNP identification number from dbSNP (NCBI) for each variant. The NCBI dbSNP database is publicly accessible (ncbi.nlm.nih.gov/projects/SNP/).

Table 9, column 2, entitled “ssID,” contains a submission identifier for Applicants' submission to dbSNP.

Table 9, column 3, entitled “Chr” identifies the chromosome on which the variant is mapped.

Table 9, column 4, entitled “Accession”, identifies the accession number for the contig containing each variant according to NCBI Build 35 of the human genome.

Table 9, column 5, entitled “Position”, identifies the position of each variant in the contig identified in column 4.

Table 9, column 6, entitled “Assayed Sequence”, is a nucleotide sequence encompassing the variant that may be used to identify the variant in a sample, e.g., by hybridization. For example, the assayed sequence may be tiled on an array for hybridization to and thereby identification of the variant.

Additional variants (and their associated gene regions) that can be used to determine an appropriate drug treatment regimen for an individual include, but are not limited to, those in haplotype blocks with the variants identified in Table 9. Such variants can be identified according to U.S. Pat. No. 6,969,589, assigned to the same assignee as the present application. A variant in a haplotype block with a variant of Table 9 that is associated with a drug response (e.g., edema) is also associated with the drug response. More specifically, a variant allele in a haplotype pattern with a variant allele of Table 1 that is associated with a drug response (e.g., edema or lack thereof) is also associated with a drug response.

Table 10 lists the genetic variants of Table 9 by rsID and ssID, the name of the gene within 10 kb of the variant, as well as the location of the variant with respect to the gene, as follows: “up” indicates that the variant is located upstream of the coding region of the gene; “down” indicates that the variant is located downstream of the coding region of the gene; “intron” indicates that the variant is located within an intron of the gene; “nonsyn” indicates that the variant is located in the coding region of the gene and is a non-synonymous polymorphism; and “syn” indicates that the variant is located in the coding region of the gene and is a synonymous polymorphism. The genes in Table 10 correspond to an old annotation of the genome, so the SNP-gene mappings may change slightly as the annotations are updated in this region. For example, additional genes may be mapped to this region. However, the ssIDs, rsIDs and accession number-based positions are typically stable in terms of defining the SNP positions. As such, even though the nucleotide positions may change, the SNP positions provided here will still be identifiable to one of ordinary skill in the art by, e.g., their rsID and/or ssID numbers.

Table 11 lists the genetic variants of Table 9 by rsID and ssID, their alleles (Allele 1 and Allele 2), the relative allele frequency of Allele 1 in the cases and controls, and the odds ratios and p values computed using logistic regression. These statistics indicate that the variant is associated with drug response (e.g., edema). Specifically, the heterozygous odds ratio is defined as the odds of edema in persons with one copy of the predisposing allele (“associated allele”) divided by the odds of edema in persons with no copies of the predisposing allele. For rare traits, the heterozygous odds ratio is closely related to the heterozygous relative risk, which is the ratio of the risk of presenting the trait in persons with one copy of the predisposing allele to the risk in persons with no copies of the predisposing allele. Logistic regression is a tool for association analysis from which odds ratios were estimated, under a multiplicative model of genetic risk; an analysis of deviance of the edema trait, adjusting for principal components that represent population structure and experimental variability, was used to estimate the significance of the association. The p-value is the likelihood that the deviance attributable to SNP genotypes would be as extreme as the observed deviance in the absence of a true association between the genotype and edema. If the relative allele frequency of Allele 1 is greater in the cases than the controls, then Allele 1 is associated with a given drug response (e.g., edema) and Allele 2 is associated with the lack of that drug response. If the relative allele frequency of Allele 1 is greater in the controls than in the cases, then Allele 1 is associated with the lack of a given drug response (e.g., edema) and Allele 2 is associated with the presence or susceptibility to the drug response.

Table 12 provides a set of six SNPs that form a haplotype block found to be associated with drug response (e.g., edema). This haplotype block is found on chromosome 14 (genes: SERPINA10 and SERPINA6); three of the 6 SNPs in the associated haplotype are nonsynonymous SNPs in SERPINA10. Table 12 lists the six SNPs, their rsIDs, and their locations in the genome with respect to the SERPINA10 and SERPINA 6 genes.

TABLE 12
Associated Haplotype

| SNP Index | rsID | Location |
|---|---|---|
| 1 | 2232700 | SERPINA10 non-synonymous |
| 2 | 941591 | SERPINA10 non-synonymous |
| 3 | 941590 | SERPINA10 non-synonymous |
| 4 | 3827896 | SERPINA6 5′, SERPINA10 3′ |
| 5 | 8015929 | SERPINA6 5′, SERPINA10 3′ |
| 6 | 941601 | SERPINA6 intron |

The variants, polymorphisms, alleles and associated genomic regions identified herein can be used to identify, isolate and amplify nucleic acids associated with drug response (e.g., edema). Such nucleic acids can be used for prognostics, diagnostics, theranostics and further study of the drug response.

In some embodiments, a set of nucleic acids is provided that can specifically hybridize to at least 2 variants, or at least 3 variants, at least 4 variants, at least 5 variants, at least 6 variants, at least 7 variants, at least 8 variants, at least 9 variants, at least 10 variants, at least 15 variants, at least 20 variants, at least 25 variants, at least 30 variants, at least 35 variants, at least 40 variants, at least 45 variants, at least 50 variants, at least 60 variants, at least 70 variants, at least 80 variants, at least 90 variants, or at least 100 variants associated with a response to drug, e.g., an insulin sensitizer or variants in common haplotype blocks thereof.

A nucleic acid can be single-stranded or double-stranded. It can also be coding (e.g., exon) or non-coding sequence (e.g., introns, exon outside coding region, and 3′ or 5′ untranslated regions) or a combination of coding and non-coding nucleic acids. In one embodiment, a coding drug nucleic acid, e.g., insulin sensitizer response nucleic acid, is one that can specifically hybridize to the complete coding region of an associated genomic region, or to one or more exons of an associated genomic region, or to one or more open reading frames of an associated genomic region, or the complementary sequence thereof.

A nucleic acid provided herein can be fused to another molecule, such as a tag sequence, a reporter gene or a fusion protein. A sequence tag encodes a polypeptide that can assist in isolation or purification of the protein product (e.g., glutathione-S-transferase (GST) fusion protein or a hemagglutinin A (HA) polypeptide). A reporter gene encodes an easily assayed protein and is often used to replace other coding regions whose protein products are difficult to assay. A fusion protein is formed by the expression of a hybrid nucleic acid made by combining two coding nucleic acid sequences.

Conditions for nucleic acid hybridization vary depending on the buffers used, length of nucleic acids, ionic strength, temperature, etc. The term “stringency conditions” for hybridization refers to the incubation and wash conditions (e.g., conditions of temperature and buffer concentration) that permit hybridization of a first nucleic acid to a second nucleic acid. The first nucleic acid may be perfectly (e.g. 100%) complementary to the second or may share some degree of complementarity, which is less than perfect (e.g., more than 70%, 75%, 85%, or 95%). For example, certain high stringency conditions can be used which distinguish perfectly complementary nucleic acids from those less complementary. High stringency, moderate stringency and low stringency conditions for nucleic acid hybridization are known in the art. Ausubel, F. M. et al., “Current Protocols in Molecular Biology” (John Wiley & Sons 1998), pages 2.10.1-2.10.16; 6.3.1-6.3.6. The exact conditions which determine the stringency of hybridization depend not only on ionic strength (e.g., 0.2×SSC, 0.1×SSC), temperature (e.g., room temperature, 42° C., 68° C.) and the concentration of destabilizing agents such as formamide or denaturing agents such as SDS, but also on factors such as the length of the nucleic acid sequence, base composition, percent mismatch between hybridizing sequences and the frequency of occurrence of subsets of that sequence within other non-identical sequences. Thus, equivalent conditions can be determined by varying one or more of these parameters while maintaining a similar degree of identity or similarity between the two nucleic acid molecules. Typically, conditions are used such that sequences at least about 60%, at least about 70%, at least about 80%, at least about 90% or at least about 95% or more identical to each other remain hybridized to one another. By varying hybridization conditions from a level of stringency at which no hybridization occurs to a level at which hybridization is first observed, conditions which will allow a given sequence to hybridize (e.g., selectively) with the most similar sequences in the sample can be determined. Exemplary conditions are described in Krause, et al., Methods in Enzymology, (1991) 200:546-556 and in Ausubel, et al., “Current Protocols in Molecular Biology”, (John Wiley & Sons 1998), which describes the determination of washing conditions for moderate or low stringency conditions. Washing is the step in which conditions are usually set so as to determine a minimum level of complementarity of the hybrids. Generally, starting from the lowest temperature at which only homologous hybridization occurs, each ° C. by which the final wash temperature is reduced (holding SSC concentration constant) allows an increase by 1% in the maximum extent of mismatching among the sequences that hybridize. Generally, doubling the concentration of SSC results in an increase in TM of ˜17° C. Using these guidelines, the washing temperature can be determined empirically for high, moderate or low stringency, depending on the level of mismatch sought. For example, a low stringency wash can comprise washing in a solution containing 0.2×SSC/0.1% SDS for 10 min at room temperature; a moderate stringency wash can comprise washing in a prewarmed solution (42° C.) solution containing 0.2×SSC/0.1% SDS for 15 min at 42° C.; and a high stringency wash can comprise washing in prewarmed (68° C.) solution containing 0.1×SSC/0.1% SDS for 15 min at 68° C. Furthermore, washes can be performed repeatedly or sequentially to obtain a desired result as known in the art. Equivalent conditions can be determined by varying one or more of the parameters given as an example, as known in the art, while maintaining a similar degree of identity or similarity between the target nucleic acid and the primer or probe used. Specific examples of hybridization conditions and procedures provided in U.S. Provisional Patent Application No. 60/648,957, filed Jan. 31, 2005, entitled “Compositions and Methods for Treating, Preventing, and Diagnosing Alzheimer's Disease;” U.S. Provisional Patent Application No. 60/653,672, filed Feb. 16, 2005, entitled “Parkinson's Disease-Related Disease Composition and Methods;” U.S. Provisional Patent Application No. 60/643,006, filed Jan. 11, 2005, entitled “Markers for Metabolic Syndrome Obesity and Insulin Resistance;” U.S. application Ser. No. 10/284,444, filed Oct. 31, 2005, entitled “Human Genomic Polymorphism;” U.S. application Ser. No. 10/447,685, filed May 28, 2003, entitled “Liver Related Disease Compositions and Methods;” and U.S. Application Ser. No. [unknown], docket no. 300/1081-10, filed Sep. 27, 2006, entitled “Genetic Basis of Rheumatoid Arthritis and Diagnosis and Treatment Thereof.”

In some embodiments, the nucleic acids herein are perfectly complementary to identified genomic regions. In some embodiments, the nucleic acids herein comprise the genomic regions identified herein. Furthermore, a nucleic acid can be isolated and/or purified, as described above. Nucleic acids can be isolated and amplified using polymerase chain reaction (PCR) techniques known in the art. See Erlich, H. A., “PCR Technology: Principles and Applications for DNA Amplification” (ed. Freeman Press, NY, N.Y., 1992); Innis M. A., et al., “PCR Protocols: A Guide to Methods and Applications” (Eds. Academic Press, San Diego, Calif., 1990); U.S. application Ser. No. 10/174,101, filed on Jun. 17, 2002, entitled “Methods for Storage of Reaction Cocktails”; U.S. Pat. No. 6,898,531, issued on May 24, 2005, entitled “Algorithms for Selection of Primers Pairs”; U.S. Pat. No. 6,740,510, filed on Jan. 2, 2002, entitled “Methods for Amplification of Nucleic Acids”, U.S. Application Serial No. 10/236,480, filed on Sep. 5, 2002, entitled “Methods for Amplification of Nucleic Acids”; and U.S. application Ser. No. 10/341,832, filed on Jan. 14, 2003, entitled “Apparatus and Methods for Selecting PCR Primers Pairs (Short Range PCR Primer Picking),” the disclosures of which are incorporated herein in their entirety.

In some embodiments, the nucleic acids used in the invention are purified. There are various degrees of purity. While a nucleic acid can be purified to homogeneity, preparations in which a nucleic acid is not purified to homogeneity are also useful where the nucleic acid retains a desired function even in the presence of considerable amount of other components. In some embodiments, nucleic acids are substantially free of cellular material which includes preparations of a nucleic acid having less than about 30% (dry weight) other nucleic acids (e.g., contaminating nucleic acids), less than about 20% other nucleic acids, less than about 10% other nucleic acids, or less than about 5% other nucleic acids.

Nucleic acids that are substantially free of chemical precursors or other chemicals generally include those that are separated from chemicals that are involved in its synthesis. In one embodiment, the nucleic acids are substantially free of chemical precursors or other chemicals such that a preparation of the nucleic acid has less than about 30% (dry weight) chemical precursors or other chemicals, or less than about 20% chemical precursors or other chemicals, or less than about 10% chemical precursors or other chemicals or than about 5% chemical precursors or other chemicals.

### Probes and Primers

The nucleic acids herein can be used as probes and primers in various assays. The terms “probe(s)” and “primer(s)” refer to nucleic acids that hybridize, in whole or in part, in a base specific manner to a complementary strand. Probes and primers include peptide nucleic acids, such as those described in Nielsen et al. (1991) Science 254:1497-1500.

Typically, the term “primer” refers to a single-stranded nucleic acid that can act as a point of initiation of template directed DNA synthesis, such as PCR. In addition to PCR, other suitable isolation, and amplification methods include, for example, the ligase chain reaction (LCR) (see Wu and Wallace, Genomics, 4:560 (1989), Landegren et al., Science, 241:1077 (1988), transcription amplification (Kwoh et al., Proc. Natl. Acad. Sci. USA, 86:1173 (1989)), self-sustained sequence replication (Guatelli et al., Proc. Natl. Acad. Sci. USA, 87:1874 (1990)) and nucleic acid based sequence amplification (NASBA). The latter two amplification methods involve isothermal reactions based on isothermal transcription that produces both single stranded RNA (ssRNA) and double stranded DNA (dsDNA) as the amplified products in a ratio of approximately 30-100 fold more ssRNA than dsDNA. See, e.g., U.S. patent application Ser. No. 11/058,432, filed Feb. 14, 2005, entitled “Selection Probe Amplification,” the disclosure of which is incorporated herein in its entirety.

PCR reactions can be designed based on the human genome sequence and the associated genomic regions or variants. For example, where a variant is located in an exon, such exon can be isolated and amplified using primers that are complementary to the nucleotide sequences at both ends of the exon. Similarly, where a variant is located in an intron, the entire intron can be isolated and amplified using primers that are complementary to the nucleotide sequences at both ends of the intron. See, e.g., U.S. Pat. No. 6,898,531, issued on May 24, 2005, entitled “Algorithms for Selection of Primers Pairs;”U.S. application Ser. No. 10/341,832, filed on Jan. 14, 2003, entitled “Apparatus and Methods for Selecting PCR Primers Pairs;” and U.S. application Ser. No. 10/236,480, filed Sep. 5, 2002, entitled “Methods for Amplification of Nucleic Acids,” the disclosures of which are incorporated herein in their entirety.

In some embodiments, a probe or a primer contains a region of at least about 10 contiguous nucleotides, or about 15 contiguous nucleotides, or about 20 contiguous nucleotides, or about 30 contiguous nucleotides, or about 50 contiguous nucleotides, or between about 10 and about 50, or between about 10 and about 40, or between about 10 and about 30 contiguous nucleotides that can specifically hybridize to a complementary nucleic acid sequence (e.g., a drug response nucleic acid such as an insulin sensitizer response nucleic acid). In addition, in some embodiments a primer is between about 10 and about 100, or between about 10 and about 50, or between about 15 and about 35, or between about 16 and about 24, or between about 18 and about 22, or between about 26 and about 34, or between about 28 and about 32, or about 18, 19, 20, 21, or 22 nucleotides in length, or about 28, 29, 30, 31, or 32 nucleotides in length. In some embodiments, a probe is between about 10 and about 60, or between about 10 and about 50, or between about 15 and about 35, or between about 20 and about 30, or about 22, 23, 24, 25, 26, 27, or 28 nucleotides in length.

In order to isolate, amplify, and/or detect the presence of a nucleic acid associated with a response to drug, e.g., an insulin sensitizer, a probe or primer or set of such probes or primers may include at least 1 variant, or at least 2 variants, or at least 3 variants, or at least 4 variants associated with a response to an insulin sensitizer or variants in common haplotype blocks with such variants.

In one embodiment, a probe or primer is at least about 70% identical, or at least about 80% identical, or at least about 90% identical, or at least about 95% identical, or about 100% identical to a contiguous drug response nucleic acid, e.g., insulin sensitizer response nucleic acid comprising at least one drug response, e.g., insulin sensitizer response, associated variant. In other embodiments a probe or a primer is complementary to a nucleotide sequence that is at least 70% identical, or at least about 80% identical, or at least about 90% identical, or at least about 95% identical, or about 100% identical to a contiguous drug, e.g., insulin sensitizer, response nucleic acid comprising at least one insulin sensitizer response-associated variant.

In any embodiment, a probe or primer may be labeled (e.g., radioisotope, fluorescent compound, enzyme, or enzyme co-factor). The probes and primers herein can be optionally labeled with, for example, a radioactive, fluorescent, biotinylated or chemiluminescent label. Labeled nucleic acids are useful for detection of a hybridization complex and can be used as probes for diagnostic and screening assays.

Labeled probes can be used in cloning of full-length cDNA or genomic DNA by screening cDNA or genomic libraries. Classical methods of constructing cDNA libraries are taught in Sambrook et al., supra. These methods provide for the production of cDNA from mRNA and the insertion of the cDNA into viral or other expression vectors. Typically, libraries of mRNA comprising poly(A) tails can be produced with poly(T) primers. Similarly, cDNA libraries can be produced using the nucleic acid herein as primers. Libraries of cDNA can be made either from selected tissues (e.g., normal or diseased tissue), or from tissues of a mammal treated with, for example, a pharmaceutical agent. Alternatively, many cDNA libraries are available commercially. In one embodiment, members of the cDNA library are larger than a nucleic acid hybridization probe, and can contain the whole cDNA native sequence.

Genomic DNA can be isolated in a manner similar to the isolation of full-length cDNA. Briefly, the nucleic acids herein, or fragments, derivatives or complement thereof, can be used to probe a library of genomic DNA. Such libraries can be in vectors suitable for carrying large segments of a genome, such as P1 or YAC, as described in detail in Sambrook et al., 9.4-9.30. In addition, genomic sequences can be isolated from human BAC libraries, which are commercially available from Research Genetics, Inc., Huntsville, Ala., USA, for example. As an alternative, full-length cDNA, genomic DNA, or any nucleic acid, fragment, derivative or complement thereof, can be obtained by synthesis.

### B. Polypeptides

The invention further provides polypeptides useful in screening, diagnostics, prognostics, prevention, treatment, or study of responses to the drugs described herein, e.g., to an insulin sensitizer. Polypeptides of the invention include those encoded by or regulated by associated genomic regions comprising the variants of nucleic acids of the invention. The polypeptides, e.g., insulin sensitizer response polypeptides herein may be naturally occurring or recombinantly produced using methods known in the art.

A polypeptide of the invention, e.g., a polypeptide associated with a response to a drug such as an insulin sensitizer may be one that is expressed differently in individuals having a phenotype of a response, e.g., a response to a drug such as an insulin sensitizer, as compared to individuals not having the same phenotype of response, or one that is regulated or encoded in whole or in part by a nucleic acid associated with a drug response, e.g., a response to an insulin sensitizer. In one example, a polypeptide associated with a drug response, e.g., a response to an insulin sensitizer can be recombinantly produced using an expression vector having a non-coding regulatory region associated with a drug response, e.g., a response to an insulin sensitizer, operably linked to an associated genomic region coding sequence, e.g., a drug response gene such as an insulin sensitizer response gene, in an expression vector. The expression vector is introduced into a host cell under conditions appropriate for expression. The polypeptide can then be isolated from the host cell using standard protein purification techniques.

In one embodiment, a polypeptide associated with a drug response, e.g., a response to an insulin sensitizer can be produced by inserting a vector comprising a coding nucleic acid associated with a drug response, e.g., a response to an insulin sensitizer and then purifying the polypeptide expressed by the host cell.

In some embodiments, the polypeptides are purified. There are various degrees of purity. While a polypeptide can be purified to homogeneity, preparations in which a polypeptide is not purified to homogeneity are also useful where the polypeptide retains a desired function even in the presence of considerable amount of other components. In some embodiments, polypeptides are substantially free of cellular material which includes preparations of a polypeptide having less than about 30% (dry weight) other polypeptides (e.g., contaminating polypeptides), less than about 20% other polypeptides, less than about 10% other polypeptides, or less than about 5% other polypeptides.

When a polypeptide is recombinantly produced, it can also be substantially free of culture medium. In some embodiments, culture medium represents less than about 20% of the volume of the polypeptide preparation, or less than about 10% of the volume of the polypeptide preparation or less than about 5% of the volume of the polypeptide preparation. Polypeptides that are substantially free of chemical precursors or other chemicals generally include those that are separated from chemicals that are involved in its synthesis. In one embodiment, the polypeptides are substantially free of chemical precursors or other chemicals such that a preparation of the polypeptides has less than about 30% (dry weight) chemical precursors or other chemicals, or less than about 20% chemical precursors or other chemicals, or less than about 10% chemical precursors or other chemicals or than about 5% chemical precursors or other chemicals.

As used herein, two polypeptides are substantially homologous when their amino acid sequences are at least about 45% homologous, or at least about 75% homologous, or at least about 85% homologous, or greater than about 95% homologous. To determine the percent homology of two polypeptides, the amino acid sequences are aligned for optimal comparison purposes. The amino acid residues at corresponding positions are compared. The percent homology between two amino acid sequences is a function of the number of identical positions shared by the sequences (e.g. percent homology equals the number of identical positions/total number of positions times 100).

Some polypeptides (e.g., conservative variants) may have a lower degree of sequence homology but are still able to perform one or more of the same functions. Conservative substitutions that can maintain the same function include replacements among aliphatic amino acids methionine, valine, leucine and isoleucine; interchange of the hydroxyl residues serine and threonine; exchange of acidic residues aspartic and glutamic acids; substitution between amide residues asparagine and glutamine, exchange between basic residues lysine and arginine, and replacements among aromatic residues phenylalanine, tyrosine and tryptophan. Alanine and glycine may also result in conservative substitutions.

Other polypeptides that may not be able to perform one or more of the same functions may be variants containing one or more non-conservative amino acid substitutions or deletions, insertions, inversions or substitution of one or more amino acid residues. Amino acids that are essential for function of a polypeptide can be identified by various methods known in the art, such as site-directed mutagenesis or alanine-scanning mutagenesis. See Cunningham et al., (1989) Science, 244:1081-1085. The latter procedure can introduce a single alanine mutation at every residue in the molecule. The resulting variants are then tested for biological activity in vitro or in vivo. Residues that are critical for polypeptide activity or inactivity are identified by comparing the two variants (with and without the alanine mutation). Polypeptide activity can also be determined by structural analysis such as crystallization, nuclear magnetic resonance or photoaffinity labeling. See Smith et al, (1992) J. Mol. Biol., 224:899-904; and de Vos et al. (1992) Science, 255:306-312.

### Fusion Proteins

Any polypeptides herein can be made part of a fusion protein. The term “fusion protein” or “fusion polypeptide” as used herein refers to a protein that has all or a substantial portion of a first polypeptide linked at the N- or C-terminus to all or a portion of a second polypeptide. For example, fusion proteins of the invention include a drug response polypeptide, e.g., an insulin sensitizer response polypeptide (a polypeptide associated with a response to drug, e.g., an insulin sensitizer) operatively linked to a non-drug-response polypeptide, e.g., non-insulin sensitizer response polypeptide or a heterologous polypeptide having an amino acid sequence not substantially homologous to a drug response amino acid sequence, e.g., an insulin sensitizer response amino acid sequence. A further example is a first drug response polypeptide, e.g., a first insulin sensitizer response polypeptide (a polypeptide associated with a response to an insulin sensitizer) operatively linked to a second drug response polypeptide, e.g., a second insulin sensitizer response polypeptide. “Operatively linked” indicates that the polypeptide and the heterologous protein are fused, for example, the non-insulin sensitizer response polypeptide can be fused to the N-terminus or C-terminus of the insulin sensitizer response polypeptide. In one embodiment, the fusion polypeptide does not affect the function of drug response polypeptide, e.g., the insulin sensitizer response polypeptide. Examples of fusion polypeptide that do not affect the function of a polypeptide include a GST-fusion polypeptides in which the drug response polypeptide, e.g., insulin sensitizer response polypeptide sequences are fused to the C-terminus of the GST sequences. Other types of fusion polypeptides include enzymatic fusion polypeptides, for example β-galactosidase fusions, yeast two-hybrid GAL fusions, poly-His fusions and Ig fusions. Fusion polypeptides, especially poly-His fusions, can facilitate the purification of recombinant polypeptide. In some host cells, such as mammalian cells, expression and secretion of a drug response polypeptide, e.g., an insulin sensitizer response polypeptide can be increased using a heterologous signal sequence. Therefore, in one embodiment, a drug response polypeptide, e.g., an insulin sensitizer response polypeptide may be fused to a heterologous signal sequence at its N-terminus. In another embodiment, a fusion protein may comprise of a drug response polypeptide, e.g., an insulin sensitizer response polypeptide and various portions of immunoglobulin constant regions such as the Fc portion. Fc portions are useful in therapy and diagnosis and may result in improved pharmacokinetic properties. Fc portions can also be used in high-throughput screening assays to identify binding molecules, agonists and antagonists. See, e.g., Bennett et al.; J. of Molec. Recog., (1995) 8:52-58 and Johanson et al., (1995) J. of Biol. Chem., 270, 16:9459-9471. In one embodiment, soluble fusion proteins comprise of a drug response polypeptide, e.g., an insulin sensitizer response polypeptide and one or more of the constant regions of heavy or light chains of immunoglobulins (e.g. IgG, IgM, IgA, IgD, IgE).

A fusion protein can be produced by standard recombinant DNA techniques as described herein. For example, DNA fragments coding for the different polypeptide sequences are ligated together in accordance with conventional techniques. The fusion gene can be synthesized by conventional techniques such as automated DNA synthesizers. Alternatively, PCR amplification of nucleic acid fragments can be carried out using anchor primers which give rise to complementary overhangs between two consecutive nucleic acid fragments that can subsequently be annealed and reamplified to generate a chimeric nucleic acid sequence. Moreover, many expression vectors are commercially available that already encode a fusion moiety (e.g., a GST protein). A nucleic acid encoding a polypeptide herein can be cloned into such an expression vector such that the fusion moiety is linked in-frame to the polypeptide.

### C. Antibodies

Any of the polypeptides herein, or fragments, derivatives, or complements thereof, can be used as an immunogen (e.g. epitope) to generate polypeptide-specific antibodies. Antibodies can be used to detect, isolate and inhibit the activity of one or more polypeptides of the invention, e.g., drug response polypeptides such as insulin sensitizer response polypeptides.

To generate antibodies, a polypeptide or a fragment thereof is used as an epitope. In some embodiments, an epitope is at least 6 amino acids, at least 9 amino acids, at least 20 amino acids, at least 40 amino acids, or at least 80 amino acids in length. The epitope or polypeptide fragment can comprise a domain, segment or motif that can be identified by analysis using well-known methods, for example, signal polypeptides, extracellular domains, transmembrane segments or loops, ligand binding regions, zinc finger domains, DNA binding domains, acylation sites, glycosylation sites or phosphorylation sites.

Examples of antibodies contemplated by the present invention include polyclonal, monoclonal, humanized, chimeric, single chain antibodies, antibody fragments such as Fab fragments, F(ab′)2 fragments, fragments produced by FAb expression library, anti-idiotypic (anti-Id) antibodies and epitope-binding fragments of any of the above.

Polyclonal antibodies are prepared by immunizing a suitable subject (e.g., goats, rabbits, rats, mice or humans) with a desired antigen. The antibody titer in the immunized subject can be monitored over time using methods known in the art, such as by using an enzyme linked immunosorbent assay (ELISA). The antibodies can then be isolated from the subject (e.g., from blood) and further purified using techniques, such as protein A chromatography, to obtain the IgG fraction.

At an appropriate time after immunization, such as when the antibody titers are highest, antibody-producing cells can be obtained from the subject and used for the preparation of monoclonal antibodies. Monoclonal antibodies are populations of antibodies that contain only one species of an antigen-binding site and are capable of immunoreacting with only one particular epitope of insulin sensitizer response polypeptides. A monoclonal antibody composition, therefore, typically displays a single binding affinity for a particular polypeptide with which it immunoreacts.

There are numerous methods known in the art for producing monoclonal antibodies. In one example, monoclonal antibodies can be obtained by fusing individual lymphocytes (typically splenocytes) from an immunized animal (typically a mouse or a rat) with cells derived from an immortal B lymphocyte tumor (typically a myeloma) to produce a hybridoma. The culture supernatants of the resulting hybridoma cells are screened to identify a hybridoma producing a monoclonal antibody that specifically binds to a polypeptide of interest. Other techniques for producing hybridoma include the human B cell hybridoma technique described in Kozbor et al. (1983) Immunol. Today, 4:72; the EBV-hybridoma technique and the trioma techniques.

Alternatively, monoclonal antibodies can be identified and isolated by screening a combinatorial immunoglobulin library, such as an antibody phage display library. The library can be screened with one or more of the polypeptides herein. Identified members are then isolated using techniques known in the art. Kits for generating and screening phage display libraries are commercially available. See for example, the Pharmacia Recombinant Phage Antibody System, Catalog No. 27-9400-01, and the Stratagene SurjZAPTM Phage Display Kit, Catalog No. 240612. Other methods and reagents for generating and screening antibody display libraries are disclosed in PCT Publication No. WO 92/01047; PCT Publication No. WO 90/02809; Fuchs et al. (1991) Bio/Technology, 9:1370-1372; Hay et al. (1992) Hum. Antibod. Hybridomas, 3:81-85; Huse et al. (1989) Science 246:1275-1281; Griffith et al. (1993) EMBO J. 12:725-734.

The monoclonal antibodies are chimeric and humanized. Humanized monoclonal antibodies can be obtained using standard recombinant DNA techniques in which the variable region genes (e.g., of a rodent antibody), are cloned into a mammalian expression vector containing the appropriate human light change and heavy chain region genes. In this example, the resulting chimeric monoclonal antibodies has the antigen-binding capacity from the variable region of the rodent but is significantly less immunogenic because of the humanized light and heavy chain regions. See, e.g., Surender K. Vaswani, Ann. (1998) Allergy Asthma. Immunol. 81:105-119.

Any of the antibodies can further be coupled to a substance (label) for detection of a polypeptide-antibody binding complex. Examples of labels include, enzymes, prosthetic groups, fluorescent materials, luminescent materials, bioluminescent materials, or radioactive materials. Examples of suitable enzymes include, for example, horseradish peroxidase, alkaline phosphatase, β-galactosidase, or acetylcholinesterase. Examples of suitable prosthetic group complexes include, for example, streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin. An example of a luminescent material is luminol. Examples of bioluminescent materials include luciferase, luciferin and aequorin. Examples of suitable radioactive material include 125I, 131I, 35S or 3H.

The antibodies can be used to isolate one or more polypeptides of the invention, e.g., a drug response polypeptide such as an insulin sensitizer response polypeptides using standard techniques such as affinity chromatography or immunoprecipitation. The antibodies can also be used to detect the presence or absence of a particular polypeptide (e.g., a polypeptide associated with a response to a drug such as an insulin sensitizer) in a cell, cell lysate, cell supernatant, tissue sample or elsewhere. In some embodiments, the antibodies can further be used to inhibit or suppress the activity of such polypeptides by specifically binding to the polypeptides.

### D. Screening Assays

The nucleic acids, polypeptides, antibodies and other compositions herein may be utilized as reagents (e.g., in pre-packaged kits) in the methods of the invention, e.g., for screening prior to determining whether or not to administer a drug, e.g., an insulin sensitizer to an individual. These may be used alone or in combination, either with each other or with phenotypic information related to the drug response. The phenotypic information related to the drug response may be determined in an association study as described above, or may be phenotypic information previously known to be associated with the drug response. Examples of such phenotypic information are detailed above and may include, e.g., medical history of an individual and/or relatives thereof (e.g., number of years with type 2 diabetes, prior treatment regimens (e.g., dosage of prior treatment with a glitazone)), laboratory test results, and/or and simple measurements (e.g., weight, height, girth, gender, etc.) Such screening can be used, for example, to identify individuals who may benefit (or not benefit) from treatment with the drug, e.g., insulin sensitizer, individuals who may be enrolled (or excluded) from a clinical trial, and/or individuals who may suffer (or not suffer) an adverse reaction from the drug, e.g., insulin sensitizer.

A variety of methods may be used to screen for response to a drug, e.g., an insulin sensitizer. The following methods are provided as examples and not as limitations of means to screen for response to a drug, e.g., an insulin sensitizer.

### E. Detection of Drug Response Nucleic Acids

Screening steps of the methods of the invention can include detection of presence, increased level or decreased level of one or more nucleic acids, or fragments, derivatives, variants or complements thereof, associated with a response to a drug, e.g., an insulin sensitizer.

Detection of nucleic acids and genetic variations in an individual may be made using any method known in the art. Examples of such methods include, for instance, Southern or northern analyses, in situ hybridizations analyses, single stranded conformational polymorphism analyses, polymerase chain reaction analyses and nucleic acid microarray analyses. Such analyses may reveal both quantitative and qualitative aspects of the expression pattern of drug response polypeptides, e.g., insulin sensitizer response polypeptides. In particular, such analyses may reveal expression patterns or polypeptides associated with a response to a drug, e.g., an insulin sensitizer.

In one example, a diagnosis or prognosis is made using a test sample containing genomic DNA or RNA obtained from the individual to be tested. The individual can be an adult, child or fetus. The individual can be a human. The test sample can be from any source which contains genomic DNA or RNA including, e.g., blood, amniotic fluid, cerebrospinal fluid, skin, muscle, buccal or conjunctival mucosa, placenta, gastrointestinal tract or other organs. A test sample of DNA from fetal cells or tissue can be obtained by appropriate methods such as by amniocentesis or chorionic villus sampling, or from the mother's blood. The test sample is subjected to one or more tests to identify the presence or absence of a nucleic acid of interest or a genetic variant of interest.

In one embodiment, Southern blot, northern blot or similar analyses methods are used to identify the presence or absence of a nucleic acid of interest or a genetic variant of interest using complementary nucleic acid probes associated with a response to a drug, e.g., an insulin sensitizer. The nucleic acid probes can be labeled before being contacted with the sample.

In hybridization analysis, the sample is maintained under conditions sufficient to allow for specific hybridization of the nucleic acid probe to the target nucleic acid. In one embodiment, the labeled nucleic acid probe and target nucleic acid specifically hybridize with no mismatches. Specific hybridization can be performed under stringent conditions disclosed herein and can be detected using standard methods. Hybridization is indicative of the presence or absence of a target nucleic acid. Specific hybridization to a nucleic acid or variant associated with a response to an insulin sensitizer is an indication that an individual will have the response if administered an insulin sensitizer, which can be either the same insulin sensitizer for which the individual is screened, or a different insulin sensitizer. More than one probe can be used concurrently.

In one embodiment, a nucleic acid probe is an allele-specific probe. See Saild, R. et al., (1986) Nature 324:163-166. Allele-specific probes can used to identify the presence or absence of one or more variants in a test sample of DNA obtained from an individual. A target nucleic acid is amplified using any method herein or known in the art. Flanking sequences may also be amplified. In the case of Southern analysis, the amplified target nucleic acid is dot-blotted, using standard methods and the blot is then contacted with an allele specific nucleic acid probe. See Ausubel, F. et al., “Current Protocols in Molecular Biology” (eds. John Wiley & Sons). Detection of specific hybridization of an allele-specific probe to a target nucleic acid associated with a response to a drug, e.g., an insulin sensitizer is an indication that an individual will have the response if administered a drug, e.g. insulin sensitizer. In some embodiments, the administered drug, e.g., insulin sensitizer, is the same as the drug for which specific hybridization is detected. In other embodiments, the administered drug, e.g., insulin sensitizer, is a different drug but in the same drug class as the drug for which specific hybridization is detected. Methods for preparing allele specific probes are known in the art.

Allele-specific probes are nucleic acids, mimetics, or a combination thereof, of approximately 10-50 base pairs or approximately 15-30 base pairs that specifically hybridize to one or more target nucleic acids. Target nucleic acids are any of the nucleic acids herein.

In one example, a target nucleic acid is a nucleic acid associated with a drug response, e.g., a response to an insulin sensitizer. Sets of nucleic acid probes that may be useful in identifying such target nucleic acids can be complementary to 1 or more, 2 or more, 3 or more, 4 or more, or 5 or more variants associated with a response to a drug, e.g., an insulin sensitizer. Such nucleic acid probes may be part of a set or in a kit (e.g., for use in Southern analysis or other techniques). Such nucleic acid probes can be allele-specific probes.

Another method for detecting nucleic acids associated with a response to a drug, e.g., a response to an insulin sensitizer, is northern analysis. Northern analysis can be used to identify gene expression patterns (e.g., mRNA) of drug response polypeptides, e.g., insulin sensitizer response polypeptides. See Ausubel, F. et al., “Current Protocols in Molecular Biology” (eds. John Wiley & Sons 1999). For northern analysis, a test sample of RNA is obtained from an individual by appropriate means. Specific hybridization of a nucleic acid probe that is complementary to the RNA sequence encoding a polypeptide associated with a response to a drug, e.g., an insulin sensitizer is an indication that an individual will have the response if administered the drug, e.g., insulin sensitizer, or another drug in the same class of drugs. A nucleic acid probe can be labeled. A nucleic acid probe can be an allele-specific probe, or may include kits or collections of probes with more than one of such probes.

Alternative diagnostic and prognostic methods employ amplification of target nucleic acids associated with a response to a drug, e.g., an insulin sensitizer, e.g., by PCR. This is especially useful for the target nucleic acids present in very low quantities. In one embodiment, amplification of target nucleic acid probes associated with a response to a drug, e.g., an insulin sensitizer indicates their presence and is an indication that an individual will have the response if administered the drug, e.g., insulin sensitizer, or if administered another drug in the same class of drugs. In another embodiment, allele specific primers are use to amplify genomic DNA associated with a response to a drug, e.g., an insulin sensitizer as an indication that an individual will have the response if administered the drug, e.g., insulin sensitizer, or another drug in the same class.

In another embodiment, cDNA is obtained from target RNA nucleic acids by reverse transcription. Nucleic acid sequences within the cDNA are then used as templates for amplification reactions. Nucleic acids used as primers in the reverse transcription and amplification reaction steps can be chosen from any of the nucleic acids herein. For detection of amplified products, the nucleic acid amplification may be performed using labeled nucleic acids. Alternatively, enough amplified product may be made such that the product may be visualized by standard ethidium bromide staining or by utilizing other suitable nucleic acid staining method.

Microarrays can also be utilized for screening for responsiveness to a drug, e.g., an insulin sensitizer. Microarrays comprise probes that are complementary to target nucleic acid sequences from an individual. A microarray probe can be allele specific. In one embodiment, the microarray comprises a plurality of different probes, each coupled to a surface of a substrate in different known locations and each, capable of binding complementary strands. See, e.g., U.S. Pat. No. 5,143,854 and PCT Publication Nos. WO 90/15070 and WO 92/10092. These microarrays can generally be produced using mechanical synthesis methods or light directed synthesis methods that incorporate a combination of photolithographic methods and solid phase oligonucleotide synthesis methods. See Fodor et al., (1991) Science 251:767-777; and U.S. Pat. No. 5,424,186. Techniques for the mechanical synthesis of microarrays are described in, for example, U.S. Pat. No. 5,384,261.

Once a microarray is prepared, a target or sample nucleic acid (e.g., DNA or RNA) is hybridized to the microarray before the microarray is scanned. Typical hybridization and scanning procedures are described in PCT Publication Nos. WO 92/10092 and WO 95/11995, and U.S. Pat. No. 5,424,186. Briefly, target nucleic acid sequences that include one or more previously identified variants or polymorphisms are amplified (optional) and labeled by well-known amplification techniques, such as PCR. Primers that are complementary to both strands of the target sequence (upstream and downstream from a variant or polymorphism) may be used to amplify the target region. Asymmetric PCR techniques may be used. After labeling with a detectable tag, the target nucleic acid is hybridized with the microarray under appropriate conditions. Upon completion of hybridization and washing and/or staining of the microarray, the microarray is scanned to determine the position on the microarray to which the target sequence hybridizes. The hybridization data obtained from the scan is typically in the form of fluorescence intensities as a function of location on the microarray.

Although primarily described in terms of a single detection block, such as for the detection of a single polymorphism, microarrays can include multiple detection blocks, and thus be capable of analyzing multiple specific polymorphisms. In an alternative arrangement, detection blocks may be grouped within a single microarray or in multiple separate microarrays so that varying optimal conditions may be used during the hybridization of the target to the microarray. For example, it may be desirable to provide for the detection of polymorphisms that fall within G-C rich stretches of a genomic sequence separately from those that fall in A-T rich segments for optimization of hybridization conditions. Additional description of use of nucleic acid microarrays for detection of polymorphisms can be found, for example, in U.S. Pat. Nos. 5,858,659 and 5,837,832, the entire teachings of which are incorporated by reference herein.

Other methods to detect variant nucleic acids include, for example, direct manual sequencing (Church and Gilbert, (1988) Proc. Natl. Acad. Sci. USA 81:1991-1995; Sanger, F. et al. (1977) Proc. Natl. Acad. Sci. USA 74:5463-5467; and U.S. Pat. No. 5,288,644); automated fluorescent sequencing; single-stranded conformation polymorphism assays; clamped denaturing gel electrophoresis; denaturing gradient gel electrophoresis (Sheffield, V. C. et al. (1981) Proc. Natl. Acad. Sci. USA 86:232-236), mobility shift analysis (Orita, M. et al. (1989) Proc. Natl. Acad. Sci. USA 86:2766-2770), restriction enzyme analysis (Flavell et al. (1978) Cell 15:25; Geever, et al. (1981) Proc. Natl. Acad. Sci. USA 78:5081); heteroduplex analysis; chemical mismatch cleavage (Cotton et al. (1985) Proc. Natl. Acad. Sci. USA 85:4397-4401); RNase protection assays (Myers, R. M. et al. (1985) Science 230:1242); and use of polypeptides which recognize nucleotide mismatches, such as E. coli mutS protein.

### F. Detection of Drug Response Polypeptides

Detecting the presence, level of expression, activity and location of a drug response polypeptide, e.g., insulin sensitizer response polypeptides, may also be used as a screening tool to determine whether or not an individual will respond to a drug, e.g., an insulin sensitizer. Briefly, detection of the presence, level of expression or enhanced activity of polypeptides associated with a response to a drug, e.g., an insulin sensitizer is an indication that an individual will have the response if administered the drug, e.g., insulin sensitizer, or another drug in the same class. Proteins may be analyzed from any tissue or cell type. Analyses can be made in vivo or in vitro.

Methods to detect and isolate polypeptides are known to those of skill in the art and include, for example, enzymes linked immunosorbent assays (ELISAs), immunoprecipitations, immunofluorescence, immunoblotting, western blotting, spectroscopy, colorimetry, electrophoresis and isoelectric focusing. See U.S. Pat. No. 4,376,110; see also Ausubel, F. et al., “Current Protocols in Molecular Biology” (Eds. John Wiley & Sons, chapter 10). Protein detection and isolation methods employed may also be those described in Harlow and Lane (Harlow, E. and Lane, D., “Antibodies: A Laboratory Manual,” Cold Spring Harbor Laboratory Press, Cold Spring Harbor, N.Y., 1998).

In one embodiment, the presence, amount and location of polypeptides associated with a response to an insulin sensitizer can be determined using a probe or an antibody that specifically binds one or more polypeptides associated with the response to the insulin sensitizer.

In one embodiment, a probe or antibody is labeled directly or indirectly. Direct labeling involves coupling (physically linking) a detectable substance to an antibody or a probe. Indirect labeling involves the reactivity of the probe with another reagent that is directly labeled. An example of indirect labeling includes, for example, detection of a primary antibody using a fluorescently labeled secondary antibody and end labeling of a DNA probe with biotin such that it can be detected with fluorescently labeled streptavidin.

A solid support may be utilized to immobilize either the antibody or probe or the sample. In one example, a sample may be immobilized onto a solid support such as a flat surface, columns, beads, optical fibers etc., which is capable of immobilizing cells, cell particles, or soluble proteins. The support may then be washed with suitable buffers followed by treatment with a detectably labeled antibody. The amount of bound labeled antibody on the solid support may then be detected by conventional means. Well known supports include glass, polystyrene, polypropylene, polyethylene, dextran, nylon, amylases, natural and modified celluloses, polyacrylamides, gabbros, nitrocellulose and magnetite.

The antibodies herein can be linked to an enzyme and used in enzyme immunoassay. See Voller, “The Enzyme Linked Immunosorbent Assay (ELISA)”, Diagnostic Horizons 2:1-7 (Microbiological Associates Quarterly Publication, Walkersville, Md. 1978); Maggio, “Enzyme Immunoassay” (CRC Press, Boca Raton, Fla. 1980); Ishikawa, et al., “Enzyme Immunoassay” (Kgaku Shoin, Tokyo, 1981). The enzyme which is bound to the antibody will react with an appropriate substrate, e.g., a chromogenic substrate, in such a manner as to produce a chemical moiety which can be detected, for example, by spectrophotometric, fluorimetric or by visual means. Enzymes that can be used to label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. Detection can be accomplished by calorimetric methods which employ a chromogenic substrate for the enzyme. Detection can also be accomplished by visual comparison of the extent of enzymatic reaction of a substrate in comparison with similarly prepared standards.

Detection may also be accomplished using any of a variety of other immunoassays. For example, by radioactively labeling the antibodies or antibody fragments, it is possible to detect fingerprint gene wild type or mutant peptides through the use of a radioimmunoassay. See Weintraub, B., “Principles of Radioimmunoassays, Seventh Training Course on Radioligand Assay Techniques” (The Endocrine Society, March, 1986). The radioactive isotope can be detected by such means as the use of a gamma counter or a scintillation counter or by autoradiography.

It is also possible to label the antibody with a fluorescent compound. When the fluorescently labeled antibody is exposed to light of the proper wave length, its presence can be detected due to fluorescence. Among the most commonly used fluorescent labeling compounds are fluorescein isothiocyanate, rhodamine, phycoerythrin, phycocyanin, allophycocyanin, o-phthaldehyde and fluorescamine. The fluorescently labeled antibody can be coupled with light microscopic, flow cytometric or fluorimetric detection. In one example, antibodies, or fragments thereof, may be employed histologically, as in immunofluorescence or immunoelectron microscopy, for in situ detection of a polypeptide associated with a response to drug, e.g., an insulin sensitizer. In situ detection may be accomplished by removing a histological specimen from a patient, such as by biopsy. The specimen is then applied with a labeled antibody described herein. The antibody or fragment can be applied by overlaying the labeled antibody or fragment onto the sample. This procedure allows for the determination of the presence, absence, amount and location of a polypeptide of interest.

The antibody can also be detectably labeled using fluorescence emitting metals such as 152Eu, or others of the lanthanide series. These metals can be attached to the antibody using such metal chelating groups as diethylenetriaminepentacetic acid (DTPA) or ethylenediaminetetraacetic acid (EDTA).

The antibody also can be delectably labeled by coupling it to a chemiluminescent compound. The presence of the chemiluminescent-tagged antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

Likewise, a bioluminescent compound may be used to label the antibodies herein. Bioluminescence is a type of chemiluminescence found in biological systems in which a catalytic protein increases the efficiency of the chemiluminescent reaction. The presence of a bioluminescent protein is determined by detecting the presence of luminescence. In some embodiments, the bioluminescent compounds for purposes of labeling antibodies are luciferin, luciferase and aequorin.

In one embodiment, the presence (or absence) of a polypeptide associated with a response to a drug, e.g., an insulin sensitizer in a sample (e.g., a cell, cell lysate, tissue, whether in vivo or in vitro) can be established by contacting the sample with an antibody and then detecting a binding complex. The presence of a polypeptide associated with a response to a drug, e.g., an insulin sensitizer in a sample is an indication that an individual will have the response if administered the drug, or another drug in the same class, e.g., another insulin sensitizer.

In another embodiment, the level of expression or composition of a polypeptide associated with a response to a drug, e.g., an insulin sensitizer in a test sample is compared with the level of expression of the same polypeptide in a control sample. A control sample can be a known level of expression of the polypeptide, or a level of expression in a sample from an individual with a known response to the drug, e.g., insulin sensitizer.

Alterations in the level of expression or composition of a drug response polypeptide, e.g., an insulin sensitizer polypeptide may be indicative that an individual will have the response if administered the drug, e.g., insulin sensitizer, or another drug in the same class. In one example, a test sample from an individual is assessed for a change in expression (e.g., level of transcription) and/or composition (e.g., splicing variants) of a polypeptide associated with a response to a drug, e.g., an insulin sensitizer. Detection of an increased level of expression of a polypeptide associated with a response to a drug, e.g., an insulin sensitizer may be an indication of an increased probability that the individual will respond to the drug, e.g., insulin sensitizer, or to another drug in the same class. On the contrary, detection of a reduced level of a polypeptide associated with a response to a drug, e.g., an insulin sensitizer may be indicative of, for example, a reduced probability that the individual will respond to the drug, e.g., insulin sensitizer.

### VI. Detection of Drug Response Phenotypes

Association studies using genetic variations as well as phenotypic variations are described in U.S. patent application Ser. No. 11/043,689, filed Jan. 24, 2005, entitled “Associations Using Genotypes and Phenotypes” which is incorporated herein by reference.

Data on a one or more phenotypes can be measured or received (e.g., from a database or medical records). Data on the group of phenotypes can be measured or received prior to, after, and/or concurrent with the generation of the genetic association data. In some embodiments, data on the phenotypes is generated by a practitioner of the present invention by, for example, observation (e.g., gross phenotypic trait such as height, weight, BMI, gender, malformation or other physical abnormality, etc.), biochemical testing (e.g., blood or urine analysis), or other diagnostic test (e.g., X-ray, MRI, CAT scan, CT scan, Doppler shift, etc.) Additional examples of phenotype data that may be received/collected about individuals can include phenotype data about previous medical conditions or medical history (e.g., whether an individual has had surgery, experienced a particular illness, given vaginal or nonvaginal childbirth, been diagnosed with mental illness, has allergies, etc.). In some embodiments, phenotype data may also be received/collected on the individuals' family history. For example, data can be collected on relatives exhibiting a given phenotype (e.g., a particular drug response or disease, etc.)

### VII. Treatments

Methods of the invention can include treating, or not treating, an individual based on the results of a screening step to determine if the individual is responsive to a drug, e.g., an insulin sensitizer. Treatment includes administering or not administering a drug, e.g., an insulin sensitizer. In some embodiments, the drug, e.g., insulin sensitizer, for which the individual is screened, is the same as the drug that is administered or not administered. In some embodiments, the drug, e.g., insulin sensitizer, for which the individual is screened, is a different drug (e.g., in the same class as the drug that is administered or not administered). Thus, in some embodiments a drug, e.g., an insulin sensitizer is administered to the individual based on the results of the screening step. In other embodiments, a drug, e.g., an insulin sensitizer is not administered to the individual based on the results of the screening step. The administration or lack of administration may be in conjunction with other treatments, such as the use of another drug, e.g., another insulin sensitizer, or other drugs. The administration or lack of administration may be in a clinical setting as part of the course of treatment of the condition (e.g., insulin resistance disorder) and/or as part of a study, e.g., a clinical trial.

Drugs, such as insulin sensitizers and other therapeutic agents used for treatment can be formulated to various preparations suitable for various administration routes, using conventional carriers. For example, for oral administration, they are formulated in the form of tablet, capsule, granule, powder, liquid preparation and the like. Conventional excipients, binders, lubricants, coloring matters, disintegrators and the like can be used upon preparing solid preparations for oral administration.

Excipients include, for example, lactose, starch, talc, magnesium stearate, microcrystalline cellulose, methyl cellulose, carboxymethyl cellulose, glycerol, sodium alginate and arabic gum. Binders used include polyvinyl alcohol, polyvinylether, ethyl cellulose, arabic gum, shellac and sucrose, and lubricants used include magnesium stearate, and talc. Further, coloring materials and disintegrators known in the art can be used. Tablets may be coated by well known methods.

Liquid preparations may be aqueous or oily suspension, solution, syrup, elixir and the like, and they can be prepared by conventional methods. When injectable preparations are formulated, to the compounds of the present invention may be added pH regulating agent, buffering agent, stabilizing agent, isotonicity, local anesthetic and the like and then preparations for subcutaneous, intramuscular or intravenous injections can be made by conventional methods. When a suppository is made, oily bases such as cacao butter, polyethylene glycols, Witepsol.RTM. (Dynamite Nobel Company) and the like may be used as base.

Preparations for other types of administration, such as by inhalation, transdermally, intranasally, intrabuccally, and the like, are known in the art and may also be used with a drug, such as an insulin sensitizer, that is administered according to the methods of the invention. See, e.g., Remingtons Pharmaceutical Sciences, 20th Ed., Lippincott Williams & Wilkins., 2000.

The dosage of such preparations is varied depending upon the condition, body weight, age, etc. of the patient and is not the same for all the patients. In some embodiments, it is set such that the dosage of the compounds of the present invention is in the range of about 0.01 to 2000 mg/day per adult patient, or about 0.1 to 1000 mg/day per adult patient, or about 0.1 to 500 mg/day per adult patient, or about 0.5 to 300 mg/day per adult patient, or about 1 to 250 mg/day per adult patient, or about 1 to 150 mg/day per adult patient, or about 5 to 150 mg/day per adult patient, or about 10 to 120 mg/day per adult patient, or about 5 to about 250 mg/day per adult patient, or about 20 to about 240 mg/day per adult patient, or about 40 to about 220 mg/day per adult patient, or about 60 to about 180 mg/day per adult patient, or about 80 to about 160 mg/day per adult patient. The preparation can be divided and administered from one to four times per day. In some embodiments, the preparation is administered about once per week, or about twice per week, or about three times per week, or about four times per week, or about five times per week, or about six times per week. In some embodiments, the preparation is administered about once per day. In some embodiments, the preparation is administered about twice per day.

In some embodiments, the preparation contains an insulin sensitizer. In some embodiments, the insulin sensitizer is a TZD PPAR modulator. In some embodiment, the insulin sensitizer is netoglitazone. In some embodiments, the insulin sensitizer is given in a dose of 1 to 200 mg/day per adult patient. In some embodiments, the TZD-PPAR modulator is given in a dose of about 1 to 200 mg/day per adult patient. In some embodiments, the netoglitazone is given in a dose of about 5 to about 250 mg/day per adult patient, or about 20 to about 240 mg/day per adult patient, or about 40 to about 220 mg/day per adult patient, or about 60 to about 180 mg/day per adult patient, or about 80 to about 160 mg/day per adult patient. In such embodiments, the insulin sensitizer, e.g., a TZD-PPAR modulator such as netoglitazone, can be given in a formulation that includes mannitol, a fluidizing agent, e.g., talc, a disintegrant, e.g., crosprovidone, a lubricant, e.g., magnesium stearate, and additional ingredients such as hydroxypropylcellulose, propylene glycol, and titanium dioxide.

In one embodiment, the drug administered is netoglitazone, given in a once-daily oral dose of 80-160 mg/day, and prepared in a formulation that is about 68%-78%, d-mannitol, about 0.1 to about 2% talc, about 3-7% crosprovidone, about 2-4% hydroxypropylcellulose, about 1-3% magnesium stearate, coated with a film that is about 1-3% (of total weight) hydroxypropylcellulose, about 0.1-1% propylene glycol, about 0.2-2% titanium dioxide, and about 0.1-0.5% talc, with the remainder active ingredient, i.e., netoglitazone (all percentages are w/w; percentages are given for tablet containing 20 mg netoglitazone and would be adjusted as appropriate for other sizes, e.g., 5 mg or 10 mg). In one embodiment the drug administered in netoglitazone, given in a once-daily oral dose of 80-160 mg/day, and prepared in a 20 mg formulation that is about 73%, d-mannitol, about 1% talc, about 5.2% crosprovidone, about 3.2% hydroxypropylcellulose, about 2.1% magnesium stearate, coated with a film that is about 2.4% (of total weight) hydroxypropylcellulose, about 0.5% propylene glycol, about 0.7% titanium dioxide, and about 0.3% talc, with the remainder active ingredient, i.e., netoglitazone (percentages for 20 mg tablet).

In some embodiments, the administration of the drug, e.g., insulin sensitizer is modulated based on the results of the screening step. Such modulations are as described herein.

### VIII. Kits

The present invention also contemplates kits for predicting if an individual responds or does not respond to a drug, e.g., an insulin sensitizer. Such kits can be used, for example, to identify individuals who may benefit (or not benefit) from treatment with a drug, e.g., insulin sensitizer, individuals who may be enrolled (or excluded) from a clinical trial, and/or individuals who may suffer (or not suffer) an adverse reaction from a drug, e.g. insulin sensitizer. In some embodiments the drug, e.g., insulin sensitizer, for which the kit is used is the same as the drug for which a prediction is made as to the individual's response. In some embodiments the drug, e.g., insulin sensitizer, for which the kit is used is a different drug in the same class as the drug for which a prediction is made as to the individual's response.

The kits herein can include at least one diagnostic tool in suitable packaging. In some embodiments the kit further contains a set of written instructions. Kits useful in screening, diagnosis and prognosis include reagents comprising, for example, nucleic acid probes or primers (for amplification, reverse transcriptase and detection), restriction enzymes (e.g., for RFLP analysis), allele-specific probes, and antisense nucleic acids, antibodies and other protein binding probes, any of which may be labeled. Kits may also comprise instructions and apparati for performing phenotypic analyses, the results of which may be used in combination with nucleic acid or protein analyses.

In some embodiments, the diagnostic tool provides means for identifying one or more genetic variations in an individual. Examples of diagnostic tools that can be used to identify genetic variations include, but are not limited to, a primer, a probe, an immunoassay, a chip based DNA assay, a PCR assay, a Taqman™ assay, a sequencing based assay, and the like. In some embodiments, such tools can provide means for detecting 1 or more genetic variations, or 3 or more genetic variations, or 30 or more genetic variations, or 300 or more genetic variations, or 3,000 or more genetic variations, or 30,000 or more genetic variations, or 300,000 or more genetic variations, or 3,000,000 or more genetic variations. In some embodiments, such genetic variations are SNPs.

In some embodiments, a diagnostic tool that identifies genetic variations scans at least about 10,000 bases, at least about t 20,000 bases, at least about 50,000 bases, at least about 100,000 bases, at least about 200,000 bases, at least about 500,000 bases, at least about 1,000,000 bases, or at least about 2,000,000 bases, at least about 5,000,000 bases, at least about 10,000,000 bases, at least about 20,000,000 bases, at least about 50,000,000 bases, at least about 100,000,000 bases, at least about 200,000,000 bases, at least about 500,000,000 bases, at least about 1,000,000,000 bases, at least about 2,000,000,000 bases, or at least about 3,000,000,000 bases of genetic material from an individual. In some embodiments, all, or substantially all, of an individual's genome is scanned, e.g., sequenced. In certain embodiments, not all associated SNPs need to be scanned to determine if an individual is or is not responsive to a drug, e.g., an insulin sensitizer.

In some embodiments a diagnostic tool that identifies genetic variations scans less than about 100,000,000 bases, less than 50,000,000 bases, less than 10,000,000 bases, less than 5,000,000 bases, less than 2,000,000 bases, less than 1,000,000 bases, less than 500,000 bases, less than 200,000 bases, less than 100,000 bases, less than 50,000 bases, less than 20,000 bases, less than 10,000 bases, less than 5,000 bases, less than 2,000 bases, less than 1,000 bases, less than 500 bases, less than 200 bases, less than 100 bases, less than 50 bases, less than 20 bases or less than 10 bases.

In some embodiments, SNPs scanned and genotyped from part or all of the genome using a kit of the invention are used in an association study. In other embodiments, only a subset of those SNPs scanned are used in an association study.

In some embodiments, a diagnostic tool includes in a kit of the invention provides means for detecting and/or quantifying one or more phenotypes (e.g., protein expression, clinical test results, medical history, simple measurements, etc.) in an individual. Examples of such diagnostic tools include, but are not limited to blood tests (e.g., PSA, blood glucose levels, etc.); other biochemical tests (e.g., pregnancy tests, allergy tests, etc.), self-diagnosis tests (e.g., breast exam, skin exam, IQ exam, etc.); review of medical history (e.g., number of years with type 2 diabetes, prior treatment regimens, etc.) and simple measurements (e.g., weight, height, girth, gender, etc.)

In some embodiments, a kit comprises at least two diagnostic tools: one to detect and/or quantify genetic variation(s) in an individual and one to detect and/or quantify phenotypic trait(s) of the individual. In some embodiments, the written instructions provide guidelines for using the results from the diagnostic tools to predict whether an individual has or does not have a phenotype-of-interest.

The results of the association studies and/or kits herein can be used, directly or indirectly, in drug discovery, clinical trials and other discovery efforts with partners. In some embodiments, the present invention contemplates computer readable databases comprising data on genetic variations and, in some embodiments, a group of phenotypes of individuals. The databases can be accessible on-line or by other medium. The databases can be used to perform virtual association studies to correlate phenotypes and/or genotypes with a phenotype-of-interest. For example, in some embodiments, databases herein can be used to perform virtual association studies by using one of the phenotypes as a phenotype-of-interest in a new study.

For example, the association studies and/or kits herein can be used to predict if an individual will or will not have a response to a drug, e.g., an insulin sensitizer based on their genotypes at a set of SNPs or subset thereof and/or a set or subset of phenotypes.

In some embodiments, such a response to a drug, e.g., an insulin sensitizer may be to a drug or product that has been pulled off the market due to unpredictable adverse effects in a small group of individuals or to one that did not obtain regulatory approval due to a large number of individuals experiencing unanticipated effects in clinical trials. In some embodiments, such a response to a drug, e.g., an insulin sensitizer may be to a drug or product that is different from, but in the same class as, a drug that has been pulled off the market due to unpredictable adverse effects in a small group of individuals or to one that did not obtain regulatory approval due to a large number of individuals experiencing unanticipated effects in clinical trials.

In some embodiments, the response may be an adverse response, and the studies and/or kits may be used to exclude individuals predicted to have an adverse response from treatment or from a research study, e.g., a clinical trial, such as a clinical trial to study a new drug or a clinical trial to study a drug that has been pulled of the market or that did not obtain regulatory approval. Alternatively, such individuals may be treated or included in the study, but appropriate adjustments may be made in their treatment based on the predicted adverse effect. In some embodiments, the response is an adverse response and a decision to treat or not treat the individual with the drug is based on a combination of factors that may include the likelihood of the adverse response, the type of adverse response, the severity of the disease or condition for which the individual is being treated, and other clinical criteria.

In some embodiments, the response may be a therapeutic response, and the individual is selected for treatment or for inclusion in a research study based on the predicted therapeutic effect. A prognosis based on the predicted response may also be made. In some embodiments, other treatments may be used or not used in conjunction with the drug, based on the predicted therapeutic response.

In some embodiments, the association studies and/or kits herein can be used to assist in determining a course of treatment for an individual, based on their genotypes at a set of SNPs or subset thereof and/or a set or subset of phenotypes. In some embodiments, the association studies and/or kits herein can be used to assess whether a brand name drug should be used, or if a cheaper generic may be substituted instead, based on their genotypes at a set of SNPs or subset thereof and/or a set or subset of phenotypes. For example, an association study can be performed to identify genetic loci associated with a positive clinical response to the generic alternative.

For example, there are a multitude of drugs on the market for treating depression including SSRIs (selective serotonin reuptake inhibitors), TCAs (tricyclic antidepressants), MAOIs (monoamine oxidase inhibitors), and triazolopyridines. Association studies may be performed to identify polymorphic loci associated with the efficacy of each of these types of drugs, and those loci could then be used to screen patient populations to determine which class of drugs would be most efficacious for a given individual. For each drug, a case group comprises individuals with depression that had an efficacious response to the drug, and a control group comprises individuals that did not have an efficacious response to the drug. Associated SNPs are identified as those that have a significantly different allele frequency in the cases than in the controls. For each class of drug, thresholds are determined that will identify individuals with a high (e.g. >80%, or >90% or >95%, or >98%) chance of having an efficacious response. An individual in need of antidepressant therapy is screened for the SNPs that are associated with each of the drug types, and a clinician determines an appropriate therapy choice for the individual based on the individual's genotype information and the thresholds determined for each class of drug. As will be apparent, the same sort of studies and screening may be done for insulin sensitizer drugs.

### IX. Business Methods

The invention also provides business methods.

Information concerning the characteristics (effectiveness, safety, and efficiency) of a given drug, e.g., insulin sensitizer is extremely valuable to the pharmaceutical industry and can save a company substantial money in lost revenue due to failures in clinical trials. The information may be used in decisions regarding the drug itself, or in decisions regarding another drug in the same class of drugs, or both.

Thus, in some embodiments, a collaborator or partner (e.g., a drug company) can use the association studies or kits herein to correlate between genomic and/or phenotype differences, and insulin sensitizer response (or lack thereof) or insulin sensitizer tolerance. Furthermore, the ability to predict an insulin sensitizer response, can subsequently be used to stratify patients into various groups. The groups may be, for example, those that respond to an insulin sensitizer versus those that do not respond, or those that respond to an insulin sensitizer without toxic effects versus those that are observed to have toxic effects. This may be useful for such company to overcome negative clinical trial results, obtain regulatory approval faster, and recoup losses. This can also save millions of dollars in unsuccessful clinical trials and fruitless research and development efforts.

Thus, in one embodiment, a therapeutic may be marketed with a kit as disclosed herein that is capable of segregating individuals that will respond in an acceptable manner to a drug from those that will not (e.g., individuals who will experience adverse side effects, minimal beneficial effects or no beneficial effects). Additional methods of using an association study for pharmacogenomics are disclosed in e.g., U.S. Provisional No. 60/566,302, filed Apr. 28, 2004, and entitled “Methods of Genetic Analysis”; U.S. Provisional No. 60/590,534, filed Jul. 22, 2004, and entitled “Methods of Genetic Analysis”; and U.S. Provisional No. 10/956,224, filed Sep. 30, 2004, and entitled “Methods of Genetic Analysis”, which are incorporated herein in their entirety by reference for all purposes.

### X. Example

Reference now will be made in detail to various embodiments and particular applications of the invention. While the invention will be described in conjunction with the various embodiments and applications, it will be understood that such embodiments and applications are not intended to limit the invention. On the contrary, the invention is intended to cover alternatives, modifications and equivalents that may be included within the spirit and scope of the invention.

An association study was performed to identify SNPs associated with edema in response to treatment with glitazones, in particular pioglitazone (Actos) and rosiglitazone (Avandia). Findings from this study provide an opportunity for increased understanding of human biology, disease and drug effects. Of particular interest is the use of the genetic loci identified for the development of pharmacogenomic-based tests to guide drug selection and to target therapy for selected patient subsets.

Saliva samples were collected from 823 subjects who experienced edema while on thiazolidinedione therapy and 2177 subjects who did not (for use as controls). The DNA from these subjects was used in a whole genome association study for discovery of SNPs associated with edema secondary to thiazolidinedione therapy.

Two board-certified physicians adjudicated the edema cases used in the while genome analysis. The following criteria were used for selecting the edema cases: severity of edema (trace or mild cases were excluded), clinical assessment vs subject complaint (physician assessment required), and medical intervention was involved to address the edema (specifically, the glitazone dose was decreased, the glitazone dose was discontinued, or a diuretic was initiated). Of the edema cases collected, 666 were chosen for genotyping based on these adjudication criteria. Of the controls collected, 1726 were matched (on a population basis) to the edema cases by sex, drug and dose, ethnicity, and insulin useand were genotyped. Approximately 1000 randomly selected SNPs were used to evaluate population structure in the samples, and to adjust for ancestry in association analyses (Price, A. L., Patterson, N.J., Plenge, R. M., et al (2006) Principal components analysis corrects for stratification in genome-wide association studies. Nature Genetics 38, 904-909). Each subject's DNA sample was individually genotyped to interrogate 287,161 SNPs across the entire genome. These SNPs were selected to tag bins of common SNPs in linkage disequilibrium for both Caucasian and Asian populations; it has been shown that such SNP subsets capture much of the common variation across the genome (Hinds, D. A., Stuve, L. L., Nilsen, G. B., et al (2005). Whole-genome patterns of common DNA variation in three human populations. Science 307, 1072-1079).

A total of 666 cases and 1726 controls were genotyped, and the resulting genotypes were analyzed to identify SNP alleles and/or haplotypes associated with incidence of edema. All of the samples were amplified. Approximately ⅔ of the samples were amplified using “selection probe amplification” (see U.S. patent application Ser. No. 11/058,432, filed Feb. 14, 2005, entitled “Selection Probe Amplification”). In brief, selection probe amplification is a technique for isolating or selecting multiple sequences from a nucleic acid sample by employing multiple unique selection probes in a single medium. Each selection probe has a sequence that is complementary to a unique target sequence that may be present in the sample under consideration. Single-stranded (e.g., denatured, double-stranded) selection probes anneal or hybridize with sample sequences having the unique target sequences specified by (e.g., complementary to) the selection probe sequences. Sequences from the sample that do not anneal or hybridize with the selection probes are separated from the bound sequences by an appropriate technique. The bound sequences can then be freed to provide a mixture of isolated target sequences, which can be used as needed for the application at hand. Samples subjected to selection probe amplification were fragmented prior to labeling using DNaseI in 1× One-Phor-All Buffer Plus (GE Healthcare). The reaction was incubated at 37° C. for 6 minutes, followed by a 95° C. incubation for 5 minutes. Following fragmentation, the samples were labeled. To each fragmented DNA sample, 4 μl of 0.5 mM biotin mix (ddUTP/dUTP; Roche) and 2 μl of rTdT (400 U/ml; terminal deoxynucleotidyl transferase; Roche) were added. The plate was sealed with a clear, plastic seal, vortexed briefly, and spun down in a SORVALL centrifuge for 15 seconds at 1000 r.p.m. The plate was placed into a thermocycler and incubated at 37° C. for 90 minutes, followed by a 95° C. incubation for 5 minutes. After the 95° C. incubation, the plate was held at 4° C.

The remaining ˜⅓ of the samples were amplified using multiplex (˜232-plex) short-range PCR (SR-PCR). Multi-well (384-well) primer plates, each containing ˜464 primers per well, were allowed to thaw at room temperature for 15 minutes and were spun down for one minute at 1000 r.p.m. in the SORVALL centrifuge. Tubes containing target DNA were spun down for 20 seconds at 14,000 r.p.m. in the EPPENDORF centrifuge. The short-range PCR (SR-PCR) reactions contained 0.061 M Trizma, 0.017 M (NH₄)₂SO₄, 3.7 mM MgCl₂, 0.03 M Tricine, 0.56× Enhancer (Epicenter Technologies), 4% DMSO, 0.05 M KCl, 0.542 mM each dNTP, 2.08× TITANIUM™ Taq DNA polymerase (Clontech), 3.3 μM of each PCR primer (a total of ˜464 primers, or ˜232 primer pairs), 15 ng target DNA, and enough MILLIPORE water to make the final volume 6 μl. SR-PCR was performed in the 384-well primer plates (now referred to as “PCR plates”), which were sealed (using the PlateLoc at 173° C. for 2.5 seconds (Velocity 11, Menlo Park, Calif.)), placed on ice, centrifuged at 1000 r.p.m. for 15 seconds in a table-top SORVALL centrifuge, vortexed for six seconds, and spun down again at 1000 r.p.m. for 15 seconds. The sealed PCR plates were stored on ice prior to amplification. Thermocyclers were preheated to 90° C. prior to placing the PCR plates into the machines. The short-range PCR program is provided in Table 12.

Once the PCR was complete, the PCR plates were removed from the machines and were either subjected to pooling immediately or were stored at 4° C. (if the PCR products were to be stored for longer than one week, they were stored at −20° C.

TABLE 13
Short-range PCR program

| Temperature | Time | | # cycles |
|---|---|---|---|
| 96° C. | 5 | min. | 1 |
| 96° C. | 2 | sec. | 55 |
| 53° C. | 2 | min. | |
| 50° C. | 15 | min. | 1 |

| | | |
|---|---|---|
| 4° C. | hold | 1 |

PCR products in a single PCR plate were pooled together. The seals of the PCR plates were pierced with a plate piercer and one pooling boat was placed on top of each PCR plate. Each PCR plate-pooling boat assembly was inverted and placed into the bucket of a table-top centrifuge. The PCR plates were spun at 1000 r.p.m. for one minute to transfer the PCR product from the PCR plates to the pooling boats. The PCR plate-pooling boat assemblies were removed from the centrifuge and the assembly was swirled to mix the contents of the pooling boats (i.e., the pooled PCR products from the PCR plate). The pooled PCR products were decanted from the pooling boat into a PCR pool tube, which was capped and set aside. The pooled PCR products were used immediately, or were stored at 4° C. (if the pooled PCR products were to be stored for longer than one week, they were stored at −20° C.

After pooling, 125 μl of each pooled PCR product was transferred to a 96-well PCR plate (now referred to as the “SAP plate”), which was subsequently sealed and spun down. The following was added to each aliquot of PCR product: 15 μl 10× One-Phor-All buffer (GE Healthcare) and 10 μl of 1 U/μl SAP (Promega Corporation), and the contents were mixed by pipetting up and down several times. The SAP plates were sealed, placed into a thermocycler, and subjected to incubation at 37° C. for 30 minutes followed by an 80° C. incubation for 20 minutes. After the 80° C. incubation, the SAP plates were held at 4° C.

The SAP-treated PCR products were purified using a vacuum filter apparatus. First, the entire contents of each well (150 μl) in the SAP plate was transferred to a separate well of a PALL ACROPREP 3K vacuum filter plate (“purification plate”). The empty wells of the purification plate were sealed with a plastic seal. The purification plate was placed on top of a vacuum manifold and the vacuum manifold was switched on. It was ensured that the vacuum pressure read >20 mm Hg. The vacuum was continued until all samples were dried. Once all samples were dried, the vacuum manifold was switched off, and 40 μl of molecular biology-grade water was added to each well. The purification plate was sealed incubated at room temperature for a minimum of 30 minutes on a flat surface, during which the plate was subjected to low-speed vortexing for one minute every five minutes. Optionally, the plate was incubated overnight at 4° C. to increase recovery of the DNA. The sample in a well was used to wash the membrane in that well three times before the sample was transferred to a “purified DNA plate.” The purified DNA plate was sealed with a plastic seal. The purified PCR products were immediately quantified or stored at 4° C. (if the purified PCR products were to be stored for longer than one week, they were stored at −20° C.

The purified PCR products in the purified DNA plates were quantified using optical density (OD) readings. First, 196 μl of MILLIPORE water was transferred to each well of a flat-bottomed clear GREINER 96-well OD plate (“quantification plate”). The purified DNA plate was vortexed and spun down at 1500 r.p.m. using the SORVALL LEGEND centrifuge for 15 seconds. Four microliters of PCR product from each well of the purified DNA plates were transferred to the corresponding location of the quantification plate. Both plates were sealed with CYCLE plate seals. The quantification plate was vortexed for 10-15 seconds using the multi-tube vortexer and spun down for 15 seconds at 1500 r.p.m. using the SORVALL LEGEND centrifuge. The DNA concentration was determined by measuring the OD of the diluted samples in the quantification plate using a TECAN microplate reader according to the manufacturer's directions.

The SAP-treated PCR products were labeled to allow detection after hybridization. Each well of a DNA label plate contained the following: 45 μM biotin mix (ddUTP/dUTP) (Roche), 1× One-Phor-All buffer, 24 U/μl TdT (terminal deoxynucleotidyl transferase; Roche), 32 μg SAP-treated PCR product, and MILLIPORE water to a final volume of 50 μl. The reaction cocktail was mixed by pipetting up and down several times, and the DNA label plate was sealed with a plastic seal. The DNA label plate was placed into a thermocycler and was held at 37° C. for 90 minutes, heated to 99° C. for 10 minutes, and then cooled and held at 4° C. After labeling, the labeled PCR products (i.e., “labeled target DNA”) was immediately hybridized to oligonucleotide microarrays or was stored at −20° C.

All samples were hybridized to oligonucleotide arrays (Affymetrix, Inc., Santa Clara, Calif.). For the samples subjected to SPA, the final concentrations of the components of the hybridization reaction cocktail was 3.0 M TMACl, 10 mM Tris (pH 7.8 or 8.0), 0.01% Triton X-100, 0.05 nM b-948 control oligo, 3.7% formamide, 1663 μg/ml herring sperm DNA, 92.59 μg/ml antisense oligo, and 185.19 μg/ml labeled target in a total volume of 270 μl. For the PCR-amplified samples, the final concentrations of the components of the hybridization reaction cocktail was 2.8 M TMACl, 9.2 mM Tris(pH 7.8 or 8.0), 0.01% Triton X-100, 0.05 nM b-948 control oligo, 5.1% formamide, 365.04 μg/ml herring sperm DNA, and 117.99 μg/ml labeled target in a total volume of 271.2 μl. The contents were mixed by pipetting the solution up and down several times.

The samples were hybridized to oligonucleotide microarrays referred to as “wafers” (Affymetrix, Inc., Santa Clara, Calif.). Each wafer contains 49 arrays of oligonucleotide probes, each of which is equivalent to a single DNA chip (Affymetrix, Inc., Santa Clara, Calif.). (In fact, chips may be manufactured by cutting arrays out of wafers and individually packaging them.) One hybridization reaction cocktail was transferred into a first array on a wafer. This process was repeated for all other hybridization reaction cocktails. (That is, each array on a wafer received only one hybridization reaction cocktail.) The wafers were incubated at 48° C. in a rotisserie incubator (“hybridization oven”) where they were rotated at ˜20 r.p.m. overnight (˜22-24 hours).

The wafers were retrieved from the rotisserie incubator, assembled into flow cells, and placed on a fluidics station for staining. The wafers were rinsed with a solution of 1×MES and 0.01% Triton X-100 at room temperature prior to staining. Three stain solutions were used, and after each stain step the flow cells were drained and the wafers were rinsed with 1×MES and 0.01% Triton X-100; the stain steps and rinses took place at room temperature. The first stain solution applied to the wafers was 1×MES, 0.01% Triton X-100, 2.5 mg/ml BSA, and 5 μg/ml streptavidin. The second stain solution applied to the wafers was 1×MES, 0.01% Triton X-100, 2.5 mg/ml BSA, and 1.25 μg/ml biotinylated anti-streptavidin antibody. The third stain solution applied to the wafers was 1×MES, 0.01% Triton X-100, 2.5 mg/ml BSA, and 1 μg/ml streptavidin-Cy-chrome. After staining and a final rinse with 1×MES and 0.01% Triton X-100, the wafers were subjected to stringency washes. The wafers were washed twice with 6×SSPE and 0.01% Triton X-100 at ˜37° C., followed by a wash with 0.2×SSPE and 0.01% Triton X-100 at ˜37° C. with no intervening rinse with 1×MES and 0.01% Triton X-100. The flow cells were then filled with 0.2×SSPE and placed in a ˜37° C. convection oven for one hour. Finally, the 0.2×SSPE was removed and the wafers were rinsed with 1×MES and 0.01% Triton X-100. After draining the flow cells they were refilled with fresh 1×MES and 0.01% Triton X-100. After the wafers were stained and washed, they were removed from the fluidics station, a back plate was added to the flow cell assembly, and taken to the scanner room for scanning.

The laser was allowed to warm up for 15 minutes before scanning was initiated. The wafers were scanned with the arc scanner, and if the images appeared to be out of focus or misaligned the scan the scanner was adjusted and the scan restarted. Once all the wafers were scanned, any wafers that were not successfully scanned were rescanned. If the scan was successful, the DAT files were submitted to the database. The wafers were removed from the scanner platform, and stored in a refrigerator at 4° C. until no longer needed. The DAT files were analyzed to determine the genotypes of each individual at each SNP location, and the allele frequencies in the case group (those who exhibited edema) were compared to those of the control group to determine SNPs and haplotype patterns associated with an increased incidence of edema in response to thiazolidinedione therapy (see U.S. patent application Ser. No. 10/970,761, filed Oct. 20, 2004, entitled “Analysis Methods and Apparatus for Individual Genotyping,” and U.S. patent application Ser. No. 11/173,809, filed Jul. 1, 2005, entitled “Algorithm for Estimating Accuracy of Genotype Assignment.”

Tests for association of SNP genotypes with edema were based on logistic regressions of edema status on genotype under a multiplicative risk model, with the inclusion of principal components that represent population structure (Price, A. L., Patterson, N.J., Plenge, R. M., et al (2006) Principal components analysis corrects for stratification in genome-wide association studies. Nature Genetics 38, 904-909) as covariates. A total of 345 SNPs were identified that yielded a p-value <0.001 in the association analysis of edema against SNP genotype, and these SNPs are provided in Tables 9-11, as described above. For each SNP, identifiers are provided (rsID and ssID in the NCBI dbSNP database (ncbi.nlm.nih.gov/projects/SNP)), as are locations on the human genome (NCBI Build 35). Also provided are allele frequencies within cases and controls, estimated odds ratios, mappings of SNPs to genes and p-values for association tests.

The heterozygous odds ratio is defined as the odds of edema in persons with one copy of the predisposing allele (“associated allele”) divided by the odds of edema in persons with no copies of the predisposing allele. For rare traits, the heterozygous odds ratio is closely related to the heterozygous relative risk, which is the ratio of the risk of presenting the trait in persons with one copy of the predisposing allele to the risk in persons with no copies of the predisposing allele. Logistic regression is a tool for association analysis from which odds ratios were estimated, under a multiplicative model of genetic risk; an analysis of deviance of the edema trait, adjusting for principal components that represent population structure and experimental variability, was used to estimate the significance of the association. The p-value is the likelihood that the deviance attributable to SNP genotypes would be as extreme as the observed deviance in the absence of a true association between the genotype and edema.

A haplotype analysis was also completed to discover haplotype patterns potentially associated with edema. The fastPHASE program (Scheet, P., Stephens, M. (2006) A fast and flexible statistical model for large-scale population genotype data: Applications to inferring missing genotypes and haplotypic phase. Am J Hum Genet 78, 629-644) was used to phase the genotype data (i.e., to determine which allele of each SNP is on which chromosome). Haplotype allele frequencies obtained from haplotype samplings were used for association tests to avoid problems related to using haplotypes resulting from maximum likelihood estimates. The haplotype trend regression test (Zaykin, D. V., Westfall, P. H., Young, S. S., Karnoub, M. C., Wagner, M. J., Ehm, M. G. (2002). Testing association of statistically inferred haplotypes with discrete and continuous traits in samples of unrelated individuals. Human Heredity 53, 79-91) was performed for all haplotype sliding windows of sizes 1 through 9, while ignoring haplotype alleles with very low frequencies (<5%). Permutations of the phenotypes were carried out to adjust estimates of significance for the number of tests performed.

Haplotype analyses were first done on an intermediate data set of 364 edema cases and 751 controls. One significantly associated (p−value=1.1×10⁻⁸) six-SNP haplotype was identified on chromosome 14 (genes: SERPINA10 and SERPINA6) with a haplotype frequency of 7.2%. This same haplotype when tested for association on the remaining samples (278 edema cases and 925 controls) yielded a p-value of 0.029 and relative risk of ˜1.4. The haplotype frequency was 6.8% in the second sample set. Three of the 6 SNPs in the associated haplotype are nonsynonymous SNPs in SERPINA10 (Table 12.)

SERPINA10 (serpin peptidase inhibitor, clade A, member 10) encodes a protein that inhibits the activated coagulation factors X and XI. Nonsense mutation in this gene are associated with venous thrombosis. SERPINA6 (serpin peptidase inhibitor, clade A, member 6) encodes a protein with corticosteroid-binding properties and may be an indicator for both insulin resistance and low grade inflammation.

The six-SNP haplotype may be used along with the other significantly-associated SNPs (Tables 9-11), for example, to develop diagnostics (e.g., in vitro diagnostic multivariate index assay, or “IVDMIA”) for identification of individuals who are predisposed to edema in response to thiazolidinedione therapy, or to further study the biological basis for drug response.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

TABLE 9

SNP information (Chromosome, Accession and Position
in Human Genome, NCBI Build 35; rsID and ssID in
NCBI dbSNP database)

| | | | | | |
|---|---|---|---|---|---|
| rsID | ssID | Chr | Accession | Position | Assayed Sequence |

| | | | | | |
|---|---|---|---|---|---|
| 234 | 24431570 | 7 | NC_000007.11 | 105155086 | GGCAGAGACTGAATNAAGGGTTGACCCAG |
| 4007 | 23498869 | 11 | NC_000011.8 | 33844118 | CAGACCTTCTTCCANCTGTAAAATTCCCA |
| 32401 | 23446086 | 5 | NC_000005.8 | 167279187 | AAGAGGTAACCCCTNGACCTAAGAGGAAA |
| 163299 | 23975386 | 5 | NC_000005.8 | 78224171 | ATTTCTTGAGAAGTNCAACAACACAGTGA |
| 179660 | 24083405 | 14 | NC_000014.7 | 30398905 | CAAAATTTGCTTAGNTAACTTCCCCGGGG |
| 197894 | 24045715 | 6 | NC_000006.9 | 95631109 | AGATTTGAGAAAAGNTTCAAAATGCAAAT |
| 247052 | 24537584 | 16 | NC_000016.8 | 56524719 | TAGTGTGTGCTACTNCCTATTTGGATAAC |
| 261712 | 23831465 | X | NC_000023.8 | 117771508 | GCTAGTAGATTTCANGTCTATTAACAGTC |
| 399516 | 24684478 | 6 | NC_000006.9 | 145852542 | CTTGCTCTCTAATCNTCAGCATCTTCGTG |
| 478239 | 24225486 | 19 | NC_000019.8 | 6005842 | TAGAATGCACAAATNAGCATAAAAGAAAA |
| 489977 | 24512768 | 18 | NC_000018.8 | 36845848 | AAATATGAATAATTNTTTGACCAGTACTT |
| 510970 | 24520470 | 9 | NC_000009.9 | 14099456 | ACCATAAAAAGACANTTCTCAGCAGAGAC |
| 518497 | 24002359 | 12 | NC_000012.9 | 112565502 | GGCAATGAGGCTTANGTATTTGTGTTTCT |
| 528374 | 24431617 | 9 | NC_000009.9 | 12821669 | ATGCTCAACAGCCANTAAGATCTTTCAGA |
| 579596 | 24674812 | 18 | NC_000018.8 | 32037028 | AAATGCACATGGATNGTTTTGACCACAGC |
| 622946 | 24167210 | 12 | NC_000012.9 | 31304592 | CCGTTTGACAAGGTNTCTGGCTGATATAA |
| 658812 | 23513964 | 18 | NC_000018.8 | 65030515 | TAAGAAGCATTGATNCAGGAAATATCTTA |
| 703406 | 23608899 | 10 | NC_000010.8 | 119429569 | CCAGGCAATTCATCNTGTCTCAAGGCAGA |
| 713286 | 23356479 | 8 | NC_000008.9 | 9209145 | AATGAGAGCTTGTANATAAAAGTCCTTGC |
| 747532 | 24476902 | 10 | NC_000010.8 | 49426368 | TTCTGCAACCTGCTNCAACCAGGACTCTT |
| 855965 | 23608911 | 10 | NC_000010.8 | 119433749 | CTGGCTCCCCGCTCNATGGTTTTCAGCTG |
| 871962 | 24296057 | 3 | NC_000003.9 | 150061313 | TGAGGGTGAAATTTNGAGAGCTGAGGAAA |
| 882685 | 24305633 | 2 | NC_000002.9 | 211405508 | TTAAGGCAGTGCCCNAACTTTTATCTATT |
| 886126 | 23389735 | 12 | NC_000012.9 | 110141934 | AGCCCAATAGTCTGNAGTTTGTCCAGAAG |
| 888784 | 23247790 | 5 | NC_000005.8 | 168632880 | GCCAAGTTTCTACCNGAGGCTTGGCTTCC |
| 891978 | 23753076 | 5 | NC_000005.8 | 170942076 | TAGACTTTGGAGCCNATTTTCTTCTTTCA |
| 902271 | 24189394 | 15 | NC_000015.8 | 27023816 | CATCCTCTATTTTGNACGCTCAGTCACCC |
| 903346 | 24642423 | 3 | NC_000003.9 | 27350521 | AACACCTGTACACANAACTGCAGGGCCCT |
| 962521 | 23367125 | 7 | NC_000007.11 | 141746776 | GTAAGAATGTCTTCNTTACAGAATAATGC |
| 967702 | 24458545 | 15 | NC_000015.8 | 35746009 | TCTGCTATTACCAGNACTCTTCAGTATTA |
| 1012968 | 23577636 | 13 | NC_000013.9 | 107988754 | TTCACTTTAGAAATNAATGGATTATTAGT |
| 1017672 | 24666309 | 15 | NC_000015.8 | 90782428 | TCTGGCCATCCCTGNCTGGAACCTGTTCT |
| 1025623 | 24650193 | 12 | NC_000012.9 | 31298139 | TTAGCATACGTTTTNTAATAATTATGTTA |
| 1026071 | 24092882 | 11 | NC_000011.8 | 13321288 | TAAATGTGGGACCTNAGATTTGAACCCTA |
| 1076184 | 24579881 | 17 | NC_000017.9 | 32016863 | AGAATGAGTTCTTTNTGCAGTTCCATTGG |
| 1078504 | 23552724 | 17 | NC_000017.9 | 72878596 | AGATGATTTAGGCTNAGAAAGAGGAGGCC |
| 1106449 | 23961108 | 5 | NC_000005.8 | 173550629 | GATCGATATCTGTANTTTGACTTTCTTAT |
| 1106722 | 23792505 | 14 | NC_000014.7 | 62397299 | CTGTCTTAGGCACTNAGCCACAATCTCAA |
| 1130866 | 24652879 | 2 | NC_000002.9 | 85805399 | TTGCAGCCCTCACANTCTGGTTCTGGAAG |
| 1137895 | 23420207 | 19 | NC_000019.8 | 55623776 | GTGAGGCCTGGGACNTTTTTAAGATCGCT |
| 1144963 | 24137593 | 12 | NC_000012.9 | 67621892 | ACCCCCAGCAAAACNGGATTTGTTTGTTT |
| 1156822 | 23868266 | 2 | NC_000002.9 | 41736479 | CTAAAATCTACTTTNATATTTCTCAGCCT |
| 1161098 | 24707116 | 12 | NC_000012.9 | 66133727 | ATTTTGACTGGCGCNGCAGAATGAGGAGG |
| 1194586 | 24284512 | 1 | NC_000001.8 | 151130491 | TCTCCTGGAAAGACNGGCTCTCTCAGGTT |
| 1261166 | 23938179 | 12 | NC_000012.9 | 32134651 | CGTGACTTATAATTNGTAGACACTCAGTG |
| 1341513 | 24503921 | 13 | NC_000013.9 | 69785536 | TTTATTTTCACCCANAAATTTACATAGAA |
| 1348994 | 23688892 | 3 | NC_000003.9 | 110037946 | TGTTTTCAATGTTTNTGCTGATTTTTTTC |
| 1354444 | 23240905 | 3 | NC_000003.9 | 155040058 | CTTCTGGAATATCANTGAATCTTGAATGT |
| 1361933 | 24695804 | 20 | NC_000020.9 | 45902724 | TACCTTATCCCCATNGAGACGTCGCTGGA |
| 1369905 | 24345666 | 3 | NC_000003.9 | 106154941 | CTATAAGGCCGTACNTTGTCTCCTTTCAT |
| 1370116 | 24159111 | 3 | NC_000003.9 | 21187137 | TAGTTATGACTTCANACCAAAATATTACT |
| 1389173 | 23354097 | 4 | NC_000004.9 | 28503381 | TATGTCATACCATTNATCGTGATGCTCAA |
| 1437937 | 23914146 | 2 | NC_000002.9 | 150576382 | AATATGAGACATCANAATACAGAGAATGA |
| 1450631 | 23435575 | 5 | NC_000005.8 | 165860873 | TGCTCTAATTTCCCNATGTCGGTTACTAT |
| 1452807 | 24545243 | 8 | NC_000008.9 | 78251054 | CTTACAAGGCCAGCNATATAGGCAGCCAT |
| 1472424 | 23183508 | 4 | NC_000004.9 | 83092653 | GTACAGTACTGTTTNGAAGGTTTTATGCT |
| 1482674 | 23411034 | 5 | NC_000005.8 | 44404007 | AATTCAATCAGAGTNCCAGTCACTAATAA |
| 1485208 | 24290135 | 3 | NC_000003.9 | 3938035 | ATTTCTAAGCTCTANGTAGGTGAAATCCC |
| 1500065 | 23730828 | 12 | NC_000012.9 | 33082138 | CAGGTTTATTATTANAAAAGTCAGTTGTT |
| 1507599 | 23552695 | 7 | NC_000007.11 | 85866910 | TTATGACCATGTGANTAACAAAAATTCTG |
| 1524408 | 24673906 | 7 | NC_000007.11 | 54000446 | TAAAACTATAACACNCAGTTTCCAGAGCA |
| 1543976 | 24627613 | 2 | NC_000002.9 | 33904858 | TTAGGGTTAGTTGTNACAGATTGCTCAGT |
| 1584769 | 24074833 | 10 | NC_000010.8 | 130845591 | TCAAATTGAAGTTTNAATTGGTAAATGGG |
| 1663588 | 24137580 | 12 | NC_000012.9 | 67563432 | GCGGCATGGGGAGGNCAGAATAGATAAGG |
| 1671413 | 24410446 | 8 | NC_000008.9 | 13270959 | GATTGCCTCAGCCTNTGATGATGTTTAGA |
| 1745836 | 24431150 | 13 | NC_000013.9 | 46235847 | AATATTTGCAGGATNGTCTTAGAATTCAT |
| 1748952 | 24603865 | 14 | NC_000014.7 | 95221437 | CAGGGAGTTCTTCANAAGTGGTTATCCTA |
| 1798255 | 23328953 | 12 | NC_000012.9 | 32178526 | GGGAGGCAAGTGTGNTAGTCCAAGTAAAA |
| 1831369 | 24171766 | 9 | NC_000009.9 | 122395096 | CAGACTTCCTCCTTNTAGGACTCTCTGAG |
| 1862505 | 24217412 | 16 | NC_000016.8 | 26568403 | AGTGTGATCTGAATNTTTGGGTATCCTTC |
| 1876533 | 23907570 | 4 | NC_000004.9 | 77574991 | TAGCAGATAATGTTNAAATTAAGGAATGA |
| 1880692 | 24336556 | 11 | NC_000011.8 | 80015717 | AAAGGCCATTTGTTNTTGGGTGTAACAAT |
| 1905325 | 24422327 | 11 | NC_000011.8 | 83393793 | GGAGGCATCATACANCCTGACTTAGAAAT |
| 1935153 | 24568806 | 10 | NC_000010.8 | 72580474 | TGAAGTCAGAGTCANGAGGGCCAGCTGCA |
| 1989309 | 23887548 | 4 | NC_000004.9 | 22237615 | AATTTTCAACTCATNTGAAAACATGATGA |
| 2008058 | 24206129 | 20 | NC_000020.9 | 45910042 | CAATTACTATTGGCNGTATCTTTCTCTTT |
| 2010711 | 24300319 | 4 | NC_000004.9 | 22209776 | AGACAATACATGAANGAATGCAACTGTGT |
| 2025122 | 24417281 | 6 | NC_000006.9 | 917898 | CTGTCCTCCCCACCNCACTGTAAGTTCTT |
| 2028088 | 24307687 | 3 | NC_000003.9 | 87069473 | AAAAAATAACCACANTGCCACCAAAATAT |
| 2034796 | 24345729 | 4 | NC_000004.9 | 14789147 | TGGCTTCCGGGCTANTGAATTTAATTTGA |
| 2037293 | 24730486 | X | NC_000023.8 | 33369292 | TACCCCTTAGCTTANAAGCAAAAAAGAAA |
| 2061717 | 24328026 | 5 | NC_000005.8 | 101336908 | TTGAGGAATGTACANATTTTATTCTGTTG |
| 2073145 | 24451923 | 20 | NC_000020.9 | 55624040 | TTGCTGGGGCCTCTNGGCTGCAGAAAGAA |
| 2078131 | 23545887 | 18 | NC_000018.8 | 44615005 | ATATGCCCAATCTANTCTGACCTTCACCC |
| 2119099 | 24564146 | 10 | NC_000010.8 | 13010830 | CATCTCTCCAAAGANGCTCAGGTCTCCGT |
| 2148694 | 24072811 | 20 | NC_000020.9 | 53214464 | CTTTGATAATTTGGNGTCTTAGTTTGTTT |
| 2217368 | 24629913 | 2 | NC_000002.9 | 83626483 | AAAATCAGTGGGCCNGATACTAGAGATAG |
| 2240142 | 23475360 | 16 | NC_000016.8 | 2752648 | TCCCGCAGTAGAAGNTTAGTTAGACGTGG |
| 2270927 | 24189133 | 5 | NC_000005.8 | 75627466 | ATATGCAAATTTCANTATTAACTTTACAA |
| 2277594 | 24126822 | 15 | NC_000015.8 | 99680410 | GGCAAGCAGGCCCCNGGCATTTCAAAGCG |
| 2277937 | 24022954 | 5 | NC_000005.8 | 153779358 | TCCTCTGAGCACTCNGGCATTTGTCATTG |
| 2278381 | 23935144 | 5 | NC_000005.8 | 146808542 | GTTCAGGGCTCTTTNTATACCTGAGGCCT |
| 2281628 | 24099502 | 14 | NC_000014.7 | 33059625 | TTTCAGGAATAAAANCAACACAATTATTT |
| 2285646 | 23549839 | 7 | NC_000007.11 | 87119089 | CCCTGACCAAAAAANAAAAGATTTTTCAT |
| 2351325 | 23458376 | 8 | NC_000008.9 | 111912018 | TGTTATATACTAGANTTTGATAGCTATTA |
| 2375592 | 24684301 | 19 | NC_000019.8 | 17494933 | GCCTTGAAAGCACCNGAGAGTCTAGTCTC |
| 2388062 | 23321060 | 12 | NC_000012.9 | 37589369 | TTATATATCTTTCCNTTTATTTAGGTCTT |
| 2412711 | 23960627 | 15 | NC_000015.8 | 40484523 | CTCAATTGACATTTNGCTCCAGTGTCGAG |
| 2418978 | 24524819 | 10 | NC_000010.8 | 109511663 | TTATCTAAATTCTGNTCTTCACTCAATAT |
| 2426778 | 23452529 | 20 | NC_000020.9 | 56726884 | GTTCCGGGAAGTCCNTCTCCAGCAGGAAC |
| 2437095 | 23374778 | 6 | NC_000006.9 | 129756982 | GCAGCACCCTGTATNTGATATTAATATTT |
| 2477202 | 23173633 | 1 | NC_000001.8 | 178978146 | TTCTTTCAGCCCTGNTCTCTAAAGGATGG |
| 2550620 | 24195805 | 16 | NC_000016.8 | 77230099 | CACTTCAGATTCCANTCGAATACATTAGA |
| 2550621 | 24195806 | 16 | NC_000016.8 | 77233032 | GCAGATGCCCGGCTNGTGGTTAACCCAAG |
| 2579875 | 23534378 | 8 | NC_000008.9 | 130490615 | GCCGCATCTCTGATNTCAGAGCATTTACA |
| 2664538 | 23574543 | 20 | NC_000020.9 | 44073632 | AGGACTCTACACCCNGGACGGCAATGCTG |
| 2682968 | 24278304 | 3 | NC_000003.9 | 60561564 | AGCTCCTACATGTCNTTTTGAGACCTTAT |
| 2704789 | 24166412 | 2 | NC_000002.9 | 146347039 | ACGTGATTACAAATNGGGAAAAGAGGGGC |
| 2714678 | 23531563 | 7 | NC_000007.11 | 78938528 | TATACACACAGAAANAAATACTGGTGAGA |
| 2738591 | 24195821 | 16 | NC_000016.8 | 77239356 | AAATTTCATATGAANGGTGGGCTTTTCTT |
| 2764951 | 23199194 | 1 | NC_000001.8 | 212495364 | AACTGAAGATTCCCNTTTATTTTTCTCCA |
| 2825163 | 24205434 | 21 | NC_000021.7 | 19111893 | CATTATCACAAAACNTAATACCTGAAGAT |
| 2851391 | 24551945 | 21 | NC_000021.7 | 43360473 | AACCTGACCCTCGGNGTGTCTGTCTGTAA |
| 2910104 | 24107752 | 12 | NC_000012.9 | 67388296 | TTCAGTTAGTCTAANTTATGAGGATATAT |
| 2991345 | 23837190 | 1 | NC_000001.8 | 41638420 | ATGGCTCTTCGTCCNATGATTCTAAAGCC |
| 3117888 | 23334891 | 9 | NC_000009.9 | 108107382 | AAAAGAATTTTTAGNTCCTATGTCATAGT |
| 3732768 | 23673768 | 3 | NC_000003.9 | 152595266 | CATGCAATCACATCNCAGCAGCAGTTGAT |
| 3741601 | 24441058 | 12 | NC_000012.9 | 67461289 | GGCAAAGTATTTGTNATTAGGAATATCTG |
| 3746229 | 23631096 | 19 | NC_000019.8 | 62465914 | GCAGGCTTAGTCACNTTGATGGATCTGTC |
| 3775561 | 24662115 | 4 | NC_000004.9 | 185728296 | GGCTTTTAAAGTTCNCACAGACAGGCATC |
| 3781575 | 23498800 | 11 | NC_000011.8 | 33841895 | CCTGTCTTACTTGGNGTTGTCGAGTTCCT |
| 3795294 | 23668004 | 1 | NC_000001.8 | 23489462 | ACTATATAGACAAANGCATGAGAGCACAA |
| 3822196 | 24372212 | 4 | NC_000004.9 | 68443565 | CATTATTTATCATCNTCAACATTATTGCT |
| 3827896 | 23654688 | 14 | NC_000014.7 | 93831174 | TAAAGGAAGGCAGCNGAGTATATTGGGAA |
| 3844055 | 23188349 | 1 | NC_000001.8 | 64231244 | GCATGAGTACAAGCNGAGGTTCACATACC |
| 3850225 | 24654763 | 6 | NC_000006.9 | 143077620 | ACACAATTCTTAATNTTTGGATCAGGCAT |
| 4076941 | 23810202 | 11 | NC_000011.8 | 11218143 | CCATTATCACCTTANTTACAGCAATCTCT |
| 4140768 | 24704028 | 12 | NC_000012.9 | 15002771 | TGCCCAACGTGTGANGTCATGCCACCAAG |
| 4142436 | 23375459 | 9 | NC_000009.9 | 1444200 | ATATCTTTCCTTTGNATCAAACAGAACAG |
| 4305582 | 23714130 | 4 | NC_000004.9 | 57869929 | TTGCTCAAATAAACNGTTAATTTGTCTAA |
| 4345821 | 23853978 | 1 | NC_000001.8 | 84619991 | TCAGCTGCTGAAGCNACTGAATTACAATG |
| 4396895 | 24332928 | 3 | NC_000003.9 | 86646876 | AGATTGTGTCTTCANTGTTAAATTAGATA |
| 4397143 | 23258338 | 5 | NC_000005.8 | 176709628 | GACTGGAGATGCACNAGGGGCCAGATTGT |
| 4398609 | 24356811 | 5 | NC_000005.8 | 101316787 | TGTTGGGGTGTCTANCAATAGGGCCTGAT |
| 4439342 | 23873163 | 1 | NC_000001.8 | 192734757 | CAACATGCTGATTANTCAAGTTAACACCT |
| 4461009 | 24224897 | 15 | NC_000015.8 | 57589900 | ACATTCCAGTAGAANAAGTAAAAAGCCAA |
| 4465511 | 23399579 | 13 | NC_000013.9 | 40852457 | AGATAAAGCAGACANCAACAAAGCAGAGA |
| 4565762 | 23655425 | 1 | NC_000001.8 | 49050520 | AGCCATAAAAAGAGNGTACGCCAACCCCC |
| 4690001 | 23891796 | 4 | NC_000004.9 | 2912146 | CTTAATCTTGATTANTGGGCTCAGAATGT |
| 4699197 | 23237336 | 4 | NC_000004.9 | 106999527 | CTATTACCAAGTTCNGTCATTAGAGCAGT |
| 4706308 | 24418999 | 6 | NC_000006.9 | 88882061 | AAAACAGCATGGGCNCAGGCCAGTTCAAA |
| 4709984 | 24146213 | 6 | NC_000006.9 | 166166746 | AATCAGATTTCAAANCTCCAGTTCCATTT |
| 4718891 | 24440338 | 7 | NC_000007.11 | 68351912 | ACTCAGCTTCCTATNTGTTTTTGTAAGCC |
| 4746136 | 24119040 | 10 | NC_000010.8 | 74971000 | AAGCACGGGTATCTNTACACAAATAAGTT |
| 4791962 | 24592048 | 17 | NC_000017.9 | 10109505 | ATGAACTCATCTATNTTTTTATACTCTAC |
| 4845963 | 23843186 | 1 | NC_000001.8 | 10905354 | CAGAGCACAGGGAGNACCTGCGGCTTTTA |
| 4859897 | 23160853 | 4 | NC_000004.9 | 79277659 | TCCCAATAGAAATGNTTGAAAATATGAAA |
| 4876347 | 24199406 | 8 | NC_000008.9 | 112062315 | GTGACAACCCTTTANGCTGTGGTAACAAA |
| 4943826 | 23302101 | 11 | NC_000011.8 | 80730697 | TGTAACTGGCTGAANTGAAATTGACTACA |
| 4973848 | 23274490 | 3 | NC_000003.9 | 27043430 | CTCATGTTGGATCTNCCTCAAGGCATTCC |
| 4976401 | 23739713 | 5 | NC_000005.8 | 136439072 | AAGGGCCAAGTGATNCAGGTTTTCCAGAA |
| 4976685 | 23258293 | 5 | NC_000005.8 | 176698783 | TCTGCTCTGCCTTCNGTACTTCCCGCGGC |
| 4982207 | 24578093 | 14 | NC_000014.7 | 20102464 | TTCTCGTTATTGGGNCACAAGAAAAAGCA |
| 4982689 | 24720936 | 14 | NC_000014.7 | 22391199 | TACCAGTGGTAGTTNTGATTACATAAGTA |
| 5910439 | 24729468 | X | NC_000023.8 | 117820679 | ATGTAGCCAATTTGNTGTTAAAAAAATAG |
| 5957080 | 24723435 | X | NC_000023.8 | 117853530 | GGGACTGTTTTTCCNCAAAGGTTTATCTT |
| 5980169 | 23816457 | X | NC_000023.8 | 7591549 | GTCCTTTCTGACTGNCAGGTGTTATACAA |
| 5992689 | 23787011 | 22 | NC_000022.8 | 16274395 | TTTAATGTTTATCCNTATGCATTTATCAC |
| 6070619 | 24598466 | 20 | NC_000020.9 | 56816552 | AGCCGTGTCATTAGNATGCGTCTTAGAAT |
| 6444052 | 24641801 | 3 | NC_000003.9 | 186545142 | CCAACACAGAGTACNGCCTTAATCGTATT |
| 6469330 | 24017213 | 8 | NC_000008.9 | 111980132 | AATTGTTGCATGTTNTCCATCAGTAGTAA |
| 6481643 | 24624089 | 10 | NC_000010.8 | 30178417 | GCTCAAATTGGAGANAGACTATCCTATAG |
| 6563127 | 24453495 | 13 | NC_000013.9 | 79114148 | AGAATAGGCAAACANTGAATGCAATATTG |
| 6574271 | 24134154 | 14 | NC_000014.7 | 75650408 | CCAGTTGTTTGGGCNCTCATCTGGGAAGC |
| 6596200 | 24356774 | 5 | NC_000005.8 | 101301550 | TTGCCAAGGCCGATNTTGAGAGGATATTT |
| 6681627 | 24276577 | 1 | NC_000001.8 | 170858399 | ATCACCTTAATTGTNTTTTATCTAGGTTC |
| 6698091 | 24257347 | 1 | NC_000001.8 | 38405967 | TAATTCGAGGCTGTNGGGCTGAGGACCCT |
| 6707185 | 23182125 | 2 | NC_000002.9 | 127063258 | ATCTGACTTTAAAANTTAAAAAGACAATT |
| 6725580 | 24634209 | 2 | NC_000002.9 | 83623589 | TTATTTGATACCGANATAGGCAATTTTAA |
| 6766574 | 23289463 | 3 | NC_000003.9 | 188173316 | TCCCCTTAGGAGCANGTGGGAAGAAGAGG |
| 6775742 | 24282737 | 3 | NC_000003.9 | 69856688 | AGGGTCTCTGTAGCNGGAACTCTCAGGTC |
| 6813595 | 24630779 | 4 | NC_000004.9 | 154334392 | ATAAGTTCTTCCAGNCCAGGATGGCTTTC |
| 6860010 | 23324332 | 5 | NC_000005.8 | 26679348 | AATATTGTGAAACANTCTGAGCGCAAAAT |
| 6866940 | 23758509 | 5 | NC_000005.8 | 129125537 | CAGTGTTCTAACTCNTAAGTGGGAGCTGA |
| 6897128 | 24188971 | 5 | NC_000005.8 | 75489276 | ACTCTAGTGGTAACNAATTCACATAAACA |
| 6921677 | 24373670 | 6 | NC_000006.9 | 16206923 | ATTTGGCAGTCTTGNGAGTCAAAAGCATA |
| 6941698 | 24684483 | 6 | NC_000006.9 | 145856838 | CATAGAATAAGTTANGGAGCAGTCCCTCT |
| 6962459 | 24675203 | 7 | NC_000007.11 | 55080681 | ATCTAGAAGGAAATNGGACTTTTTAATAT |
| 6968649 | 23433229 | 7 | NC_000007.11 | 16369925 | AGAAATATTGTCAGNGCAAAAGGGCTAGG |
| 6969869 | 24382469 | 7 | NC_000007.11 | 16899639 | ATTATAAGATTGCTNGGATAAAACAAAGT |
| 7002174 | 24084138 | 8 | NC_000008.9 | 78188119 | AATTGCGTTTCTTTNATGATAAATCAATT |
| 7121669 | 24040026 | 11 | NC_000011.8 | 33836123 | CGTCGGGACCCTCCNGGGCTAGCGCGCTT |
| 7177340 | 23544375 | 15 | NC_000015.8 | 78349914 | CCTAGGGTCTATGGNTTTTGTTGCCATTG |
| 7257225 | 24682133 | 19 | NC_000019.8 | 37267178 | TGCAGAGAGATACANACGAATGCCCAGCT |
| 7328107 | 23983332 | 13 | NC_000013.9 | 44379639 | GGAGCAGTGGAAGCNTAGGTATTCTCTTC |
| 7429119 | 23755325 | 3 | NC_000003.9 | 186484013 | ACCTGTTTCTGGCTNCCCGGCAGAACCTC |
| 7429509 | 23673764 | 3 | NC_000003.9 | 152583865 | TGGTATTATGATAGNAGGCTTAGATTCAA |
| 7449280 | 24017409 | 5 | NC_000005.8 | 34513495 | TTATGTCACAGGGGNTTAGAGGACACAGG |
| 7525479 | 24257106 | 1 | NC_000001.8 | 68031561 | CCTGAGTTATCCATNGACAAGAGAATGAG |
| 7570033 | 23255656 | 2 | NC_000002.9 | 153717890 | TCAAAGGGCAACTTNCTTAAAGCATTTTT |
| 7587023 | 23301107 | 2 | NC_000002.9 | 15060921 | CAATTTCATATGCANCCCATGTGAGCTTT |
| 7631594 | 24134893 | 3 | NC_000003.9 | 2026065 | GAATTCATGATCCANGTAATAGTACTAAA |
| 7632294 | 24342534 | 3 | NC_000003.9 | 157833085 | TAAAATTCAGATTCNGGGCCTGCCGTGGT |
| 7635836 | 23275312 | 3 | NC_000003.9 | 27116122 | AGGTGCCTTGCACANTGCGTACCACATAG |
| 7657964 | 24645587 | 4 | NC_000004.9 | 5584399 | GTGAATGAACGAATNATGATGGCCAACAG |
| 7666299 | 24236069 | 4 | NC_000004.9 | 84012513 | AAGCCGAAGGATGCNGAAATGTGGCACTG |
| 7668673 | 24398923 | 4 | NC_000004.9 | 7037746 | CGGTATGTTGCAAGNGGAAGTACTTTTTC |
| 7670013 | 23742709 | 4 | NC_000004.9 | 169952720 | CACATTAATTCCTGNGTAAGAAAATTATA |
| 7757529 | 24497301 | 6 | NC_000006.9 | 80007796 | TGCTGTGTTTTACCNATGCAAATGCTGGA |
| 7802597 | 23534456 | 7 | NC_000007.11 | 11568272 | CCAAGAACCCCATTNTGAAGTTGTCCTAG |
| 7807431 | 23351577 | 7 | NC_000007.11 | 70439026 | TTTGGCCCGATGGGNGTATGGATAAATTC |
| 7832077 | 24094060 | 8 | NC_000008.9 | 80958240 | CCCCTTTAGCCAAANTGCACTTAGGATAA |
| 7845273 | 23589693 | 8 | NC_000008.9 | 80945459 | TTAAGAATAGTTGANATGGCAATTATGAA |
| 7855874 | 24305257 | 9 | NC_000009.9 | 28562086 | TGGATATAAACTCTNTTCTTGGCATGTAA |
| 7909516 | 23385028 | 10 | NC_000010.8 | 77555343 | CAGAGTAAAATTGCNTACCATCTGTCAAG |
| 7942997 | 23623726 | 11 | NC_000011.8 | 94935943 | GTTAGTCTGTATTANGAAAGGGGACTGAA |
| 7964255 | 23797169 | 12 | NC_000012.9 | 92288002 | ATCATCATCTCTATNCACACTGGGAATTA |
| 7969224 | 24428656 | 12 | NC_000012.9 | 84592343 | GGTAGAACCATAGANTGTAAGTATCAGTT |
| 8011140 | 24572712 | 14 | NC_000014.7 | 99280123 | CCAGTTGCTGAGGTNGGTAAAAGGTCGCC |
| 8024294 | 23743009 | 15 | NC_000015.8 | 91824136 | TTATCAGTACACAANCAGGTCACCTGACT |
| 8032896 | 24523605 | 15 | NC_000015.8 | 78358161 | AGGCCCAGGGAATANGCTGCCCAAAGTGA |
| 8080460 | 24705581 | 17 | NC_000017.9 | 66980486 | TATGAACTCCCTTANAGTAGGTGGGTGCA |
| 8091352 | 23479883 | 18 | NC_000018.8 | 13506802 | AGGAGAGTGGCCTTNTATCAGCCTGTGTT |
| 8103560 | 24225485 | 19 | NC_000019.8 | 5991869 | GTTCTGTATCTTGANGGTGGTATTGTTAC |
| 8108576 | 23631018 | 19 | NC_000019.8 | 62450419 | AGTCTTTAAATATTNTAATGGTTCGTGAA |
| 9300160 | 24001822 | 12 | NC_000012.9 | 23672817 | TGTTCACTTTCTTCNTTCAAGGAGCAGTT |
| 9306955 | 24651330 | 4 | NC_000004.9 | 37793416 | AAGACATATCATTCNCACTATAATTCCAA |
| 9314751 | 23431158 | 9 | NC_000009.9 | 86129423 | CAGCACTTTACAAGNTTCAGAAAACTCCA |
| 9367359 | 23768890 | 6 | NC_000006.9 | 49602200 | ACCTGAGTGTTGCCNATGCGGATCTACTC |
| 9395504 | 24687509 | 6 | NC_000006.9 | 49602892 | AGGAGAAATTACTGNATGAGAACAAATGA |
| 9543383 | 24520751 | 13 | NC_000013.9 | 72953806 | TCCATCTACGCACANTAAAAAGGCATTAT |
| 9574385 | 23971198 | 13 | NC_000013.9 | 78730524 | AACTAGTATGTTTANTCTATTTTTCTTTA |
| 9725695 | 23186933 | 1 | NC_000001.8 | 221647620 | TACAAGGAAGTTAANATCTAGAGCGATCA |
| 9809173 | 23274970 | 3 | NC_000003.9 | 27086229 | CTCCCTCTGTGTAANTTCCTTGGAATACA |
| 9826662 | 24296064 | 3 | NC_000003.9 | 150061426 | GTCTAAGAACAATGNAAATCCATTGAAGA |
| 9831663 | 23224477 | 3 | NC_000003.9 | 3935462 | ATTTGTAGTTTTGCNGATAAAGAACACTT |
| 9893651 | 24121997 | 17 | NC_000017.9 | 10126697 | CCAATAAGTTCATCNGTGTTCTAAACTAT |
| 9996218 | 23160392 | 4 | NC_000004.9 | 79107679 | GTATCTTTGTTCACNTTGTTCATGGCTTC |
| 1001186 | 423914250 | 4 | NC_000004.9 | 31933142 | CTTTTTAAAAATCANCCTTAAGATTGCCA |
| 10057630 | 23410686 | 5 | NC_000005.8 | 44363621 | AGCACAAATTCTTTNCTGTATGTGGAGAC |
| 10113434 | 24088899 | 8 | NC_000008.9 | 112004972 | GAATCTATAAATTCNGCTTCATGTCATGA |
| 10173398 | 23696624 | 2 | NC_000002.9 | 153180502 | CTTTATATTATCTGNGTTGTCAGTTTTTA |
| 10185471 | 23246611 | 2 | NC_000002.9 | 180535112 | TCATAACTCTCTCGNGAGTGATAACATCT |
| 10234682 | 24392434 | 7 | NC_000007.11 | 21881299 | GGGAACCTTGAGGGNAGGATAAGTTGAAT |
| 10234702 | 24392436 | 7 | NC_000007.11 | 21881361 | GAGGTGGGAGAGGANCTTTCCATTTTAGA |
| 10269805 | 24429491 | 7 | NC_000007.11 | 16837773 | TTACATTCTAACTANCCTTCAAGATCCAA |
| 10269951 | 24388660 | 7 | NC_000007.11 | 20879159 | AAACATCCGGAAGANGCAATGGCAGCTAT |
| 10276660 | 23442018 | 7 | NC_000007.11 | 96007051 | ACAGCATACAAAGGNGTAATATGAAGTAA |
| 10483450 | 24099451 | 14 | NC_000014.7 | 33054924 | AGAAAATTGAATAANAATGGTAGCTAAGC |
| 10484415 | 24383938 | 6 | NC_000006.9 | 67185250 | ACTCCTCTTCCATTNACATTATTGTTGAA |
| 10504647 | 24084733 | 8 | NC_000008.9 | 78235557 | ATAGTCAAGTTGAANGAATATACATTTTT |
| 10514501 | 24501683 | 16 | NC_000016.8 | 79070531 | TGTGGGAACAACTANTTGTGCATGGAATC |
| 10779375 | 24268680 | 1 | NC_000001.8 | 216245073 | TTATTCATCTATTANAGAAAGTAGCAAAA |
| 10787862 | 24575921 | 10 | NC_000010.8 | 120551865 | CTGTGCACTGGGCANTGCGCTGATAGGCA |
| 10793571 | 24552848 | 10 | NC_000010.8 | 44768383 | CAGGTCAAGTCTAGNTAGCTGTGGGGCAG |
| 10818896 | 23499368 | 9 | NC_000009.9 | 98253743 | TCAGTTCAGAATCCNCAGAAAAGTTAGTG |
| 10819626 | 24554249 | 9 | NC_000009.9 | 130105310 | CCCTGGCTGGTTACNTAGGGCTACCTGTC |
| 10842192 | 24001678 | 12 | NC_000012.9 | 23661565 | GGAAAAGCTTGGATNCAAAAGTAAATAAT |
| 10842616 | 23428232 | 12 | NC_000012.9 | 25843638 | CATCTTGGAAAATANGCATTTATATGTTT |
| 10844149 | 24364474 | 12 | NC_000012.9 | 32218425 | AGTCAGATGTATCTNTTTTTTCCTTTTTA |
| 10866652 | 23753035 | 5 | NC_000005.8 | 170933070 | TAACTACTATGCAANAGGAACACTGACCT |
| 10873421 | 23654501 | 14 | NC_000014.7 | 91888484 | AATATGGTATAGCCNTACAGGTGAATTTA |
| 10933481 | 24333006 | 2 | NC_000002.9 | 150566491 | TTCTCCTCAGATCANTATTATTTCAGAGG |
| 10984781 | 24088381 | 9 | NC_000009.9 | 119806337 | TGCACACTTTAGAGNTTAGAAAATCCAGG |
| 11023815 | 23589025 | 11 | NC_000011.8 | 15963815 | ACACATTGTTACTGNTCCCACCACAGAAT |
| 11044839 | 23990157 | 12 | NC_000012.9 | 19801039 | GCTCCACAACCCTCNGGCAATACCTAAAT |
| 11077567 | 24213923 | 17 | NC_000017.9 | 66988039 | AAACAGTAGTTGCCNGCTTCCCACGTGCA |
| 11078544 | 23792983 | 17 | NC_000017.9 | 4909235 | CTCTGGGTTCAGCCNGTCTCCTGTCATTC |
| 11126919 | 23691626 | 2 | NC_000002.9 | 83647732 | TGAATTTGGGCTTTNCTCGATTCATGATT |
| 11144705 | 23490584 | 9 | NC_000009.9 | 75819842 | ATTGCTTATGAGCTNTGGAATTAAGTGGT |
| 11151508 | 24505283 | 18 | NC_000018.8 | 65285990 | GTTTGTGTAAATACNCAGTTTCCTGTATC |
| 11171159 | 24359037 | 12 | NC_000012.9 | 37887390 | AATGGATACACTGANGGTTAGTGGCTCCT |
| 11176454 | 24214546 | 12 | NC_000012.9 | 65538533 | TTTTTTGACTTTTTNTTGCAGATTCTAGC |
| 11177315 | 24611408 | 12 | NC_000012.9 | 67308563 | CAGGAAACCTTGTCNAATGCGTGATTTTA |
| 11209403 | 23848737 | 1 | NC_000001.8 | 69392083 | CTCTCTTTTAACAANGCAGTGCTCAAGAT |
| 11209405 | 24251883 | 1 | NC_000001.8 | 69396620 | TACTTTGTCCACAGNGCACTGAGCTCTGG |
| 11582478 | 24276808 | 1 | NC_000001.8 | 213058598 | AAAAGCTACAAATTNATGAAGTATCTAGG |
| 11642164 | 24189783 | 16 | NC_000016.8 | 9664807 | TGTGTGCATTATGCNAAGCAAGGAATACT |
| 11651604 | 24705544 | 17 | NC_000017.9 | 66948104 | CGGGCCATAAAACCNAGACCGCCAGAAAC |
| 11715174 | 24287551 | 3 | NC_000003.9 | 72145688 | ACAAGCTTGTTAGCNGATGAGCTGGGACA |
| 11733801 | 24236071 | 4 | NC_000004.9 | 84016807 | TTTATTTTTTTGCCNATAAGAAAGATCCC |
| 11820556 | 23629748 | 11 | NC_000011.8 | 122579926 | TTGATACGACTTGANTACCCAAGGCTGAG |
| 11833134 | 23952248 | 12 | NC_000012.9 | 22157469 | CACTTGTAACCTTCNGTGATTAGATCCAG |
| 11892547 | 23720603 | 2 | NC_000002.9 | 195208025 | GAAATGCTAAATGGNGAAAGCAATCTGAG |
| 11914104 | 23646668 | 22 | NC_000022.8 | 35577118 | CTGGCGGTCTGTTCNCGTCAACATTTAGA |
| 11994590 | 23457881 | 8 | NC_000008.9 | 111886247 | TTGGAATCCAAAGCNTGTCTCTTTTGAGA |
| 12018552 | 23514195 | 13 | NC_000013.9 | 69728319 | TTCAACACTGTCCCNTATCTTTCTATACT |
| 12054491 | 23907649 | 4 | NC_000004.9 | 111168295 | ACTCAAAGCCAAGTNTTAGACTAGCAGAA |
| 12149483 | 24046430 | 16 | NC_000016.8 | 78950963 | ATAACATAAAAAGTNTTCATTCACTCGCT |
| 12162084 | 24217409 | 16 | NC_000016.8 | 26556972 | TACAAATGGTCACANAACTTACCCTACAC |
| 12199003 | 23773687 | 6 | NC_000006.9 | 55304546 | GAGAGCAATGCTTANGTGATGCAAATGGA |
| 12204525 | 24622323 | 6 | NC_000006.9 | 45899891 | CTGGAATCAAGGTCNCCTTCTTGGTCTTT |
| 12590632 | 24109096 | 14 | NC_000014.7 | 51099071 | ATTCCCTCACTCCANCCCAAGGGCAATTT |
| 12724393 | 24251682 | 1 | NC_000001.8 | 218284920 | CCAGAGATTACAGCNTGAAGGGTTTTGAG |
| 13026628 | 24654234 | 2 | NC_000002.9 | 195339111 | TAAAATCCTACATANCTCCCTTGGGCACT |
| 13037781 | 23558482 | 20 | NC_000020.9 | 30968953 | CTCTAGGAGCCCCTNGCCCTTGCAGCCCA |
| 13067759 | 23274631 | 3 | NC_000003.9 | 27052735 | AAGATGAGGCCCCANGGTTTTGGAATGCT |
| 13157045 | 23923070 | 5 | NC_000005.8 | 106126646 | CTACACAAAAATTANTCACTTGGGCAGGG |
| 13235422 | 24429406 | 7 | NC_000007.11 | 16827280 | AAGAATACTATCTTNTTTTCTCACCACAG |
| 13289879 | 24579023 | 9 | NC_000009.9 | 10034824 | GCAACTTCATTGGANTAGACAAGACATGA |
| 13409142 | 23193345 | 2 | NC_000002.9 | 32577788 | CAGCGGAATTAGACNCAGGACTTTGGTTT |
| 16839587 | 23925797 | 2 | NC_000002.9 | 203863556 | AAGAAGAAGATTTTNTAGTTCTGTTTATG |
| 16872491 | 23409841 | 5 | NC_000005.8 | 35200652 | AGTCCAGTTCAGAGNTGATGCCAGGATTA |
| 16873956 | 23410589 | 5 | NC_000005.8 | 44348246 | AATAATGGATGTTANCACTTAAGCCTCTG |
| 16881360 | 24088842 | 8 | NC_000008.9 | 111994964 | GTCCAACTTTCCCANTCTACCCCAACTCA |
| 16892924 | 23313927 | 5 | NC_000005.8 | 23886969 | CTAACATTTTGTTGNTTCTACCACCTTTA |
| 16894082 | 23380583 | 6 | NC_000006.9 | 83094644 | GGATAACAGTGGGANGGTGAGGCAAAAGC |
| 16899163 | 24072251 | 8 | NC_000008.9 | 125058541 | CAAAATATGAGCTCNTGGTCTAACTACAT |
| 16902330 | 23459589 | 5 | NC_000005.8 | 34525106 | TGCCCATGTTCTGANTTTATCAGGCCAGC |
| 16929452 | 23997965 | 12 | NC_000012.9 | 25863384 | ATCTTGTTTTGGCANCTTGATGACTACAT |
| 16944026 | 23955249 | 12 | NC_000012.9 | 113226862 | GCACAGGGGACTCCNGACAGATGTGATAT |
| 16969422 | 24003810 | 15 | NC_000015.8 | 76113254 | ATATCATTTTTCCTNTTTACTTGTACTTT |
| 16976054 | 23799463 | 16 | NC_000016.8 | 26561984 | TCATTGACACAGTTNACATGCCAGGGTCA |
| 17001863 | 24079440 | 22 | NC_000022.8 | 39083808 | ATCTAGCAGCATGANTCATCAGCTCTGGT |
| 17012108 | 23403601 | 4 | NC_000004.9 | 128401930 | TCCGATTTGCAGTTNTAGTTCGACTAAAT |
| 17014088 | 23237521 | 4 | NC_000004.9 | 89637609 | TTTCTATAAAAGTTNGTGATACAATGATG |
| 17029364 | 24152380 | 4 | NC_000004.9 | 154250677 | TTAGACACCCTCTCNGTGGGGCAAAATTG |
| 17035504 | 23896631 | 12 | NC_000012.9 | 103284080 | ACTACTTGGACAGTNACCTGAACATCTCA |
| 17077144 | 23368681 | 5 | NC_000005.8 | 173620078 | GTGGTTGAACCTTGNAGAAATGTGTTAGA |
| 17081286 | 24004586 | 13 | NC_000013.9 | 24336550 | AGGGACACATTCAGNACCCAATAACTGTA |
| 17101420 | 24099431 | 14 | NC_000014.7 | 33052891 | AGGAGAAGACTTGCNTGCCCAGGCTTGCT |
| 17131701 | 23853902 | 1 | NC_000001.8 | 84613522 | AATATAAAACTAAANGAGATGAACATTGG |
| 17137494 | 24074774 | 10 | NC_000010.8 | 15651878 | TCAAGAAAAGATGANGTTTGCATTCTCTA |
| 17143871 | 23361050 | 7 | NC_000007.11 | 20921548 | GAACGTAGTACTCCNTTTGACTTTGAGAA |
| 17159146 | 24159741 | 7 | NC_000007.11 | 8892097 | TTGGTGCATTTAGTNCAAACAGCTCCCAA |
| 17170923 | 23739703 | 5 | NC_000005.8 | 136430319 | GAGATTATTTGTAANCACAGTGTTTCATG |
| 17198844 | 24417517 | 6 | NC_000006.9 | 1107048 | ATGAATAGAAGCATNTTTGTGTCTACAAC |
| 17249385 | 24426511 | 6 | NC_000006.9 | 136312771 | CTTATATTTAAGACNGCTTAGATTTTTTA |
| 17254424 | 24416402 | 8 | NC_000008.9 | 59188993 | AAACTTCATAAAGCNAGGAAAGAAGATAA |
| 17263496 | 24419002 | 5 | NC_000005.8 | 13790444 | TCACCTCCTCGGCTNTCCTTTTTGTGTTA |
| 17283421 | 24730426 | X | NC_000023.8 | 32620908 | CTTTATGCAGGATTNAGTTTTACAGGATA |
| 17287745 | 24622099 | 5 | NC_000005.8 | 142635208 | TCAGTACTTTTAAGNCAATGCAACTTTAA |
| 17289925 | 24221325 | 3 | NC_000003.9 | 186917362 | CTTACCTGGTGGCTNGTTCGTGGAATTTA |
| 17315298 | 24634262 | 1 | NC_000001.8 | 174003454 | TCCTGGACTGGCTTNACTGTACTCTCCCA |
| 17315903 | 24524731 | 16 | NC_000016.8 | 19277945 | TTAAAAAAAATCTTNTGTGGTTGGCTATC |
| 17348962 | 24542111 | 8 | NC_000008.9 | 68852348 | GACGACAGATGTCANAAAACATAAAAGTA |
| 17378751 | 24545185 | 8 | NC_000008.9 | 78585280 | AGTTCTGAAGATTTNCTTTGAGTTTTTAA |
| 17399569 | 24242512 | 1 | NC_000001.8 | 3302116 | GGGCTCACAACGGGNGGTCATGGTTGCGG |
| 17412366 | 24649394 | 2 | NC_000002.9 | 146509545 | CTAGATAGGGGAACNGAGCAGCTAAATGA |
| 17426593 | 24651660 | 6 | NC_000006.9 | 32716055 | AGACCATGCCTGATNGGTGTTTTACACAT |
| 17428526 | 24540628 | 11 | NC_000011.8 | 70229376 | AGCCAGGGAAGCCANCCATCCAAGAGGGA |
| 17429548 | 23711465 | 4 | NC_000004.9 | 77667273 | GCAAAAACCATAGCNTTATTGGGCTTGGG |
| 17495754 | 24562333 | 8 | NC_000008.9 | 82315105 | AATACGATGGTGACNTTTCAAAAATCTGG |
| 17529372 | 24655122 | 4 | NC_000004.9 | 41607138 | CTTTTTCAGGCTTGNAAATGCTCATGCTA |
| 17541270 | 24456879 | 18 | NC_000018.8 | 10361035 | ATTTTAATCTGGTTNCACATTTGTCGTCA |
| 17572655 | 24573436 | 12 | NC_000012.9 | 67371477 | AAAAGAGAAAATTGNAAAAAGTAGGTGAG |
| 17573852 | 24474156 | 18 | NC_000018.8 | 34590858 | TACATTCTTTGGGTNTGAACATAGTTTTA |
| 17622991 | 24681239 | 5 | NC_000005.8 | 131960652 | TAACTCTGATAGGTNATGAGGAGCCAACC |
| 17652287 | 24284076 | 2 | NC_000002.9 | 20257865 | TCTGATCGTAAAACNGTGGACGCTGAGCA |
| 17659437 | 24276737 | 1 | NC_000001.8 | 174038044 | TTAAAATATATCAANGTATCTGCAGTCCG |
| 17719112 | 24194884 | 7 | NC_000007.11 | 41446700 | GATTGAACAGGACTNTTTGTTAATTCTAC |
| 17763421 | 24392771 | 4 | NC_000004.9 | 30136015 | AACATCATTTTTACNGTTATTCTTAAGAT |
| 17784735 | 24406001 | 8 | NC_000008.9 | 12902034 | ACATTTCATTGCAGNGATAAGGGATAGGG |
| 17793991 | 24509420 | 8 | NC_000008.9 | 62729142 | CAGCAATGAGGCAANTAAAATGCACTTGA |
| 17796970 | 24308693 | 4 | NC_000004.9 | 181860565 | TCAAGGTCGATATANTGATTTCTGAACAA |

TABLE 10

| | | Gene transcripts within 10 kb of SNP | | |
|---|---|---|---|---|
| | | (from NCBI Gene) | | |
| SNP information | | NCBI | | |
| (NCBI dbSNP) | | Gene ID, | Gene | Location wrt |
| rsID | ssID | Build 35.1 | Name | gene* |
| 4007 | 23498869 | 4005 | LMO2 | intron |
| 32401 | 23446086 | 57451 | ODZ2 | intron |
| 163299 | 23975386 | 411 | ARSB | intron |
| 247052 | 24537584 | 1258 | CNGB1 | intron |
| 261712 | 23831465 | 401616 | LOC40161 | intron |
| 478239 | 24225486 | 5990 | RFX2 | intron |
| 510970 | 24520470 | 4781 | NFIB | intron |
| 528374 | 24431617 | 286343 | C9orf150 | down |
| 579596 | 24674812 | 55034 | MOCOS | intron |
| 622946 | 24167210 | 441632 | LOC44163 | up |
| 713286 | 23356479 | 441336 | LOC44133 | intron |
| 747532 | 24476902 | 58504 | ARHGAP2; | intron |
| 871962 | 24296057 | 1359 | CPA3 | up |
| 871962 | 24296057 | 1360 | CPB1 | down |
| 886126 | 23389735 | 23316 | CUTL2 | intron |
| 888784 | 23247790 | 6586 | SLIT3 | intron |
| 902271 | 24189394 | 321 | APBA2 | intron |
| 1017672 | 24666309 | 8128 | SIAT8B | intron |
| 1025623 | 24650193 | 441632 | LOC44163 | up |
| 1026071 | 24092882 | 406 | ARNTL | intron |
| 1076184 | 24579881 | 79154 | MGC4172 | up |
| 1076184 | 24579881 | 79893 | ZNF403 | intron |
| 1078504 | 23552724 | 10801 | MSF | intron |
| 1106722 | 23792505 | 27133 | KCNH5 | intron |
| 1130866 | 24652879 | 6439 | SFTPB | nonsynonymous |
| 1137895 | 23420207 | 4606 | MYBPC2 | up |
| 1137895 | 23420207 | 6689 | SPIB | 3′UTR |
| 1144963 | 24137593 | 1368 | CPM | up |
| 1194586 | 24284512 | 57198 | ATP8B2 | intron |
| 1348994 | 23688892 | 50852 | TRIM | intron |
| 1482674 | 23411034 | 2255 | FGF10 | intron |
| 1663588 | 24137580 | 1368 | CPM | intron |
| 1671413 | 24410446 | 10395 | DLC1 | intron |
| 1745836 | 24431150 | 2098 | ESD | down |
| 1748952 | 24603865 | 9623 | TCL1B | up |
| 1748952 | 24603865 | 27004 | TCL6 | down |
| 1798255 | 23328953 | 636 | BICD1 | intron |
| 1831369 | 24171766 | 138882 | OR1N2 | synonymous |
| 1876533 | 23907570 | 8987 | GENX-341 | up |
| 1905325 | 24422327 | 1740 | DLG2 | intron |
| 2025122 | 24417281 | 285768 | LOC28576 | intron |
| 2028088 | 24307687 | 389136 | FLJ38507 | down |
| 2073145 | 24451923 | 81030 | ZBP1 | nonsynonymous |
| 2078131 | 23545887 | 9811 | KIAA0427 | intron |
| 2119099 | 24564146 | 83643 | CCDC3 | intron |
| 2240142 | 23475360 | 6923 | TCEB2 | down |
| 2240142 | 23475360 | 23524 | SRRM2 | synonymous |
| 2270927 | 24189133 | 22987 | SV2C | nonsynonymous |
| 2277594 | 24126822 | 5046 | PCSK6 | intron |
| 2277937 | 24022954 | 55568 | GALNT10 | 3′UTR |
| 2278381 | 23935144 | 1809 | DPYSL3 | intron |
| 2281628 | 24099502 | 64067 | NPAS3 | intron |
| 2285646 | 23549839 | 55972 | MCFP | intron |
| 2375592 | 24684301 | 25796 | PGLS | down |
| 2375592 | 24684301 | 199786 | BCNP1 | up |
| 2388062 | 23321060 | 144402 | CPNE8 | up |
| 2412711 | 23960627 | 825 | CAPN3 | intron |
| 2412711 | 23960627 | 64397 | SH3BP3 | down |
| 2426778 | 23452529 | 79716 | NPEPL1 | down |
| 2426778 | 23452529 | 391258 | LOC39125 | down |
| 2437095 | 23374778 | 3908 | LAMA2 | intron |
| 2550620 | 24195805 | 51741 | WWOX | intron |
| 2550621 | 24195806 | 51741 | WWOX | intron |
| 2579875 | 23534378 | 137196 | MGC2743 [symbol] | intron |
| 2664538 | 23574543 | 4318 | MMP9 | nonsynonymous |
| 2682968 | 24278304 | 2272 | FHIT | intron |
| 2738591 | 24195821 | 51741 | WWOX | intron |
| 2764951 | 23199194 | 7399 | USH2A | intron |
| 2851391 | 24551945 | 875 | CBS | intron |
| 2910104 | 24107752 | 57122 | NUP107 | intron |
| 2991345 | 23837190 | 59269 | HIVEP3 | down |
| 3732768 | 23673768 | 116931 | TRALPUSI | synonymous |
| 3746229 | 23631096 | 390980 | LOC39098 | down |
| 3775561 | 24662115 | 3660 | IRF2 | intron |
| 3781575 | 23498800 | 4005 | LMO2 | intron |
| 3795294 | 23668004 | 6920 | TCEA3 | intron |
| 3822196 | 24372212 | 2798 | GNRHR | intron |
| 3827896 | 23654688 | 866 | SERPINA6 | down |
| 3827896 | 23654688 | 51156 | SERPINA1 | up |
| 3844055 | 23188349 | 4919 | ROR1 | intron |
| 4140768 | 24704028 | 397 | ARHGDIB | intron |
| 4397143 | 23258338 | 10636 | RGS14 | up |
| 4397143 | 23258338 | 10960 | LMAN2 | intron |
| 4461009 | 24224897 | 145773 | MGC2669 [symbol] | intron |
| 4465511 | 23399579 | 79612 | FLJ22054 | down |
| 4465511 | 23399579 | 387922 | LOC38792 | down |
| 4565762 | 23655425 | 84871 | FLJ14442 | intron |
| 4690001 | 23891796 | 118 | ADD1 | intron |
| 4699197 | 23237336 | 79807 | FLJ13273 | intron |
| 4746136 | 24119040 | 159195 | USP54 | intron |
| 4976401 | 23739713 | 6695 | SPOCK | intron |
| 4976685 | 23258293 | 10960 | LMAN2 | intron |
| 4982207 | 24578093 | 390443 | h461 | up |
| 4982689 | 24720936 | 4323 | MMP14 | down |
| 5957080 | 24723435 | 392529 | LOC39252 | up |
| 6681627 | 24276577 | 9910 | HHL | up |
| 6766574 | 23289463 | 6480 | SIAT1 | intron |
| 6775742 | 24282737 | 440962 | LOC44096 | intron |
| 6897128 | 24188971 | 22987 | SV2C | intron |
| 7121669 | 24040026 | 4005 | LMO2 | down |
| 7177340 | 23544375 | 400411 | LOC40041 | intron |
| 7429509 | 23673764 | 64805 | P2RY12 | intron |
| 7429509 | 23673764 | 116931 | TRALPUS [symbol] | intron |
| 7525479 | 24257106 | 391047 | LOC39104 | intro |
| 7657964 | 24645587 | 55351 | STK32B | intron |
| 7666299 | 24236069 | 79966 | SCD4 | intron |
| 7668673 | 24398923 | 57533 | TBC1D14 | intron |
| 7670013 | 23742709 | 23022 | KIAA0992 | intron |
| 7757529 | 24497301 | 9324 | HMGN3 | up |
| 7802597 | 23534456 | 23249 | KIAA0960 | intron |
| 7807431 | 23351577 | 64409 | WBSCR17 | intron |
| 7855874 | 24305257 | 158038 | FLJ31810 | intron |
| 7909516 | 23385028 | 83938 | C10orf11 | intron |
| 7964255 | 23797169 | 11163 | NUDT4 | intron |
| 8024294 | 23743009 | 390641 | LOC39064 | up |
| 8032896 | 24523605 | 400411 | LOC40041 | up |
| 8091352 | 23479883 | 753 | C18orf1 | intron |
| 8103560 | 24225485 | 5990 | RFX2 | intron |
| 8108576 | 23631018 | 390980 | LOC39098 | intron |
| 9300160 | 24001822 | 6660 | SOX5 | intron |
| 9306955 | 24651330 | 10744 | PTTG2 | down |
| 9314751 | 23431158 | 79670 | ZCCHC6 | down |
| 9314751 | 23431158 | 81689 | HBLD2 | up |
| 9367359 | 23768890 | 389396 | LOC38939 | nonsynonymous |
| 9395504 | 24687509 | 389396 | LOC38939 | down |
| 9826662 | 24296064 | 1359 | CPA3 | up |
| 9826662 | 24296064 | 1360 | CPB1 | down |
| 9996218 | 23160392 | 246175 | CNOT6L | up |
| 10057630 | 23410686 | 2255 | FGF10 | intron |
| 10173398 | 23696624 | 114793 | FMNL2 | intron |
| 10483450 | 24099451 | 64067 | NPAS3 | intron |
| 10779375 | 24268680 | 440715 | LOC44071 | intron |
| 10793571 | 24552848 | 83937 | RASSF4 | up |
| 10818896 | 23499368 | 9568 | GPR51 | intron |
| 10819626 | 24554249 | 392395 | LOC39239 | intron |
| 10842192 | 24001678 | 6660 | SOX5 | intron |
| 10844149 | 24364474 | 636 | BICD1 | intron |
| 10873421 | 23654501 | 123041 | SLC24A4 | intron |
| 11023815 | 23589025 | 55553 | SOX6 | intron |
| 11144705 | 23490584 | 5125 | PCSK5 | intron |
| 11151508 | 24505283 | 220164 | DOK5L | intron |
| 11177315 | 24611408 | 5908 | RAP1B | intron |
| 11582478 | 24276808 | 2104 | ESRRG | down |
| 11733801 | 24236071 | 79966 | SCD4 | intron |
| 11820556 | 23629748 | 79827 | ASAM | up |
| 11914104 | 23646668 | 4689 | NCF4 | up |
| 11914104 | 23646668 | 400926 | FLJ90680 | up |
| 12199003 | 23773687 | 389400 | UNQ9356 | nonsynonymous |
| 12724393 | 24251682 | 11221 | DUSP10 | intron |
| 13037781 | 23558482 | 388795 | LOC38879 | nonsynonymous |
| 13409142 | 23193345 | 57448 | BIRC6 | intron |
| 16839587 | 23925797 | 65065 | ALS2CR17 | intron |
| 16872491 | 23409841 | 5618 | PRLR | intron |
| 16873956 | 23410589 | 2255 | FGF10 | intron |
| 16899163 | 24072251 | 439941 | LOC43994 | down |
| 16969422 | 24003810 | 23102 | KIAA1055 | intron |
| 17001863 | 24079440 | 158 | ADSL | intron |
| 17001863 | 24079440 | 27352 | RUTBC3 | up |
| 17014088 | 23237521 | 55008 | HERC6 | intron |
| 17029364 | 24152380 | 85462 | KIAA1727 | intron |
| 17081286 | 24004586 | 54513 | TDRD4 | intron |
| 17101420 | 24099431 | 64067 | NPAS3 | intron |
| 17137494 | 24074774 | 8516 | ITGA8 | intron |
| 17170923 | 23739703 | 6695 | SPOCK | intron |
| 17249385 | 24426511 | 27115 | PDE7B | intron |
| 17254424 | 24416402 | 90362 | MGC3932 [symbol] | intron |
| 17263496 | 24419002 | 1767 | DNAH5 | nonsynonymous |
| 17283421 | 24730426 | 1756 | DMD | intron |
| 17287745 | 24622099 | 2908 | NR3C1 | down |
| 17289925 | 24221325 | 10644 | IMP-2 | intron |
| 17399569 | 24242512 | 63976 | PRDM16 | intron |
| 17426593 | 24651660 | 3117 | HLA-DQA1 | intron |
| 17428526 | 24540628 | 399921 | LOC39992 | intron |
| 17429548 | 23711465 | 339965 | FLJ25770 | intron |
| 17495754 | 24562333 | 392238 | LOC39223 | up |
| 17572655 | 24573436 | 57122 | NUP107 | intron |
| 17622991 | 24681239 | 10111 | RAD50 | intron |
| 17784735 | 24406001 | 286032 | FLJ36980 | intron |
| 17793991 | 24509420 | 444 | ASPH | intron |

*up and down refer to smaller and larger positions in chromosomal coordinates (NCBI Build 35) than gene transcript boundaries [symbol]
indicates data missing or illegible when filed

TABLE 11

| | | | | | | Association | |
|---|---|---|---|---|---|---|---|
| | | | | | | Analysis | |
| | | | | | | Results (Logistic | |
| SNP information | | | | Frequency of | | Regression) | |
| (NCBI dbSNP) | | | | Allele 1 | | Odds | |
| rsID | ssID | Allele 1 | Allele 2 | Cases | Controls | Ratio | p value |
| 13235422 | 24429406 | G | A | 0.654688 | 0.736152 | 1.415128 | 9.88E−07 |
| 2285646 | 23549839 | C | A | 0.801056 | 0.854027 | 1.590412 | 1.18E−06 |
| 7177340 | 23544375 | G | C | 0.708916 | 0.62823 | 1.445205 | 1.43E−06 |
| 11994590 | 23457881 | G | A | 0.737288 | 0.668252 | 1.381468 | 5.42E−06 |
| 10269805 | 24429491 | T | A | 0.609327 | 0.684009 | 1.35486 | 7.13E−06 |
| 2550620 | 24195805 | A | C | 0.433225 | 0.51036 | 1.368931 | 1E−05 |
| 8108576 | 23631018 | T | C | 0.77769 | 0.839975 | 1.453104 | 1.11E−05 |
| 4876347 | 24199406 | C | T | 0.334356 | 0.400355 | 1.391855 | 1.35E−05 |
| 518497 | 24002359 | C | A | 0.786482 | 0.837774 | 1.424053 | 2.02E−05 |
| 6707185 | 23182125 | G | A | 0.821782 | 0.760359 | 1.459809 | 2.17E−05 |
| 11209405 | 24251883 | G | A | 0.592284 | 0.656122 | 1.327429 | 2.59E−05 |
| 9306955 | 24651330 | A | C | 0.360414 | 0.433524 | 1.319575 | 2.67E−05 |
| 12590632 | 24109096 | A | G | 0.940164 | 0.963822 | 1.929385 | 2.68E−05 |
| 10113434 | 24088899 | T | C | 0.341743 | 0.403892 | 1.377698 | 2.7E−05 |
| 2412711 | 23960627 | C | G | 0.230047 | 0.290662 | 1.417059 | 3.2E−05 |
| 3746229 | 23631096 | T | A | 0.763274 | 0.823799 | 1.406854 | 3.4E−05 |
| 10269951 | 24388660 | A | G | 0.554745 | 0.61996 | 1.340718 | 3.76E−05 |
| 7855874 | 24305257 | G | A | 0.273292 | 0.329988 | 1.345261 | 4.43E−05 |
| 6444052 | 24641801 | C | T | 0.427907 | 0.505316 | 1.304741 | 4.76E−05 |
| 7802597 | 23534456 | C | A | 0.896072 | 0.852174 | 1.539967 | 4.94E−05 |
| 622946 | 24167210 | A | G | 0.236476 | 0.290018 | 1.363515 | 5.23E−05 |
| 6969869 | 24382469 | G | A | 0.266412 | 0.199942 | 1.372327 | 5.73E−05 |
| 11126919 | 23691626 | C | T | 0.584992 | 0.518801 | 1.302683 | 6.26E−05 |
| 8080460 | 24705581 | T | C | 0.818336 | 0.877873 | 1.461257 | 6.91E−05 |
| 1437937 | 23914146 | A | G | 0.301391 | 0.241736 | 1.34001 | 7.2E−05 |
| 179660 | 24083405 | A | G | 0.425627 | 0.510688 | 1.332518 | 7.52E−05 |
| 12199003 | 23773687 | C | T | 0.567208 | 0.644654 | 1.304318 | 8.05E−05 |
| 6574271 | 24134154 | T | C | 0.684654 | 0.625486 | 1.333548 | 8.61E−05 |
| 6681627 | 24276577 | C | A | 0.918712 | 0.881416 | 1.560786 | 9.18E−05 |
| 10234702 | 24392436 | A | G | 0.428019 | 0.360182 | 1.316777 | 9.31E−05 |
| 7807431 | 23351577 | C | T | 0.667712 | 0.720694 | 1.338059 | 0.000101 |
| 2991345 | 23837190 | T | C | 0.482998 | 0.412478 | 1.293151 | 0.000109 |
| 17014088 | 23237521 | C | T | 0.609063 | 0.542233 | 1.297739 | 0.000111 |
| 7632294 | 24342534 | C | T | 0.892012 | 0.848771 | 1.470113 | 0.000117 |
| 1130866 | 24652879 | G | A | 0.421947 | 0.479894 | 1.297846 | 0.000117 |
| 8032896 | 24523605 | C | T | 0.380805 | 0.321133 | 1.314186 | 0.000117 |
| 8011140 | 24572712 | G | A | 0.375963 | 0.44013 | 1.306818 | 0.000117 |
| 2375592 | 24684301 | A | G | 0.532558 | 0.479689 | 1.29328 | 0.000124 |
| 11733801 | 24236071 | T | G | 0.5693 | 0.512375 | 1.306874 | 0.000125 |
| 7449280 | 24017409 | T | C | 0.01473 | 0.034591 | 2.51844 | 0.000128 |
| 11715174 | 24287551 | A | C | 0.534404 | 0.603885 | 1.297987 | 0.000134 |
| 4718891 | 24440338 | G | C | 0.266181 | 0.213358 | 1.371068 | 0.000138 |
| 17159146 | 24159741 | A | T | 0.909021 | 0.870816 | 1.488326 | 0.000143 |
| 1017672 | 24666309 | G | C | 0.380989 | 0.432898 | 1.305965 | 0.000146 |
| 7121669 | 24040026 | T | G | 0.880401 | 0.831945 | 1.429423 | 0.000148 |
| 10011864 | 23914250 | A | G | 0.54288 | 0.49047 | 1.298856 | 0.00015 |
| 9809173 | 23274970 | C | G | 0.761206 | 0.805375 | 1.35248 | 0.000158 |
| 4565762 | 23655425 | C | T | 0.035842 | 0.067358 | 1.90759 | 0.000158 |
| 10866652 | 23753035 | C | G | 0.285933 | 0.35282 | 1.312362 | 0.000162 |
| 1876533 | 23907570 | T | C | 0.511799 | 0.454754 | 1.284549 | 0.000163 |
| 1144963 | 24137593 | C | T | 0.326357 | 0.392535 | 1.303615 | 0.000167 |
| 871962 | 24296057 | G | A | 0.62069 | 0.672872 | 1.301852 | 0.000169 |
| 17426593 | 24651660 | T | C | 0.857473 | 0.814099 | 1.400692 | 0.000176 |
| 1482674 | 23411034 | T | G | 0.063191 | 0.100362 | 1.673217 | 0.000179 |
| 1862505 | 24217412 | T | G | 0.862916 | 0.896509 | 1.467975 | 0.00018 |
| 2910104 | 24107752 | T | C | 0.639731 | 0.568012 | 1.302104 | 0.00018 |
| 10057630 | 23410686 | T | C | 0.933282 | 0.897102 | 1.644441 | 0.000181 |
| 13067759 | 23274631 | C | T | 0.316514 | 0.27246 | 1.321572 | 0.000185 |
| 3822196 | 24372212 | A | G | 0.804572 | 0.753864 | 1.34332 | 0.00019 |
| 9574385 | 23971198 | A | G | 0.911168 | 0.941567 | 1.618824 | 0.000196 |
| 9300160 | 24001822 | A | G | 0.384202 | 0.442714 | 1.283703 | 0.000198 |
| 10842192 | 24001678 | A | T | 0.544481 | 0.605039 | 1.290955 | 0.000198 |
| 1194586 | 24284512 | C | T | 0.46 | 0.396034 | 1.291657 | 0.000201 |
| 17170923 | 23739703 | T | C | 0.450077 | 0.40291 | 1.287917 | 0.000201 |
| 4709984 | 24146213 | A | G | 0.122671 | 0.173066 | 1.421726 | 0.000203 |
| 1905325 | 24422327 | A | G | 0.568779 | 0.526159 | 1.2875 | 0.000205 |
| 5957080 | 24723435 | A | G | 0.344595 | 0.404272 | 1.236836 | 0.000206 |
| 10514501 | 24501683 | A | C | 0.883308 | 0.920649 | 1.484039 | 0.000214 |
| 10819626 | 24554249 | A | C | 0.597393 | 0.650235 | 1.284246 | 0.000214 |
| 1354444 | 23240905 | A | G | 0.457692 | 0.528436 | 1.275162 | 0.000214 |
| 4982689 | 24720936 | G | C | 0.711747 | 0.659892 | 1.3182 | 0.000216 |
| 9996218 | 23160392 | A | G | 0.176651 | 0.223762 | 1.37544 | 0.000219 |
| 4973848 | 23274490 | C | T | 0.341743 | 0.297242 | 1.307235 | 0.00022 |
| 2477202 | 23173633 | G | T | 0.666957 | 0.61166 | 1.314848 | 0.000221 |
| 10234682 | 24392434 | A | G | 0.418197 | 0.354089 | 1.305785 | 0.000221 |
| 17035504 | 23896631 | G | C | 0.876534 | 0.910408 | 1.48859 | 0.000227 |
| 9367359 | 23768890 | A | G | 0.471893 | 0.415711 | 1.284083 | 0.000228 |
| 2008058 | 24206129 | T | C | 0.922427 | 0.949495 | 1.632944 | 0.000234 |
| 7657964 | 24645587 | G | C | 0.67907 | 0.62263 | 1.292624 | 0.000235 |
| 1798255 | 23328953 | T | C | 0.348926 | 0.419317 | 1.285695 | 0.000235 |
| 11078544 | 23792983 | C | T | 0.778024 | 0.72397 | 1.331351 | 0.000238 |
| 4976401 | 23739713 | C | T | 0.217969 | 0.177553 | 1.368727 | 0.000241 |
| 9395504 | 24687509 | C | T | 0.553599 | 0.612257 | 1.278969 | 0.000247 |
| 7964255 | 23797169 | G | C | 0.706738 | 0.756789 | 1.304193 | 0.000247 |
| 882685 | 24305633 | C | G | 0.645482 | 0.712647 | 1.293705 | 0.000251 |
| 16969422 | 24003810 | T | C | 0.870968 | 0.830048 | 1.409852 | 0.000252 |
| 12149483 | 24046430 | A | T | 0.766287 | 0.817673 | 1.358999 | 0.000258 |
| 10173398 | 23696624 | G | A | 0.656105 | 0.71194 | 1.291899 | 0.000258 |
| 1137895 | 23420207 | G | C | 0.678135 | 0.734742 | 1.308005 | 0.000261 |
| 962521 | 23367125 | A | G | 0.271493 | 0.231729 | 1.319674 | 0.000262 |
| 2119099 | 24564146 | C | T | 0.836078 | 0.882075 | 1.430457 | 0.000263 |
| 2388062 | 23321060 | A | G | 0.198805 | 0.151525 | 1.400201 | 0.000271 |
| 4859897 | 23160853 | G | A | 0.850394 | 0.807065 | 1.38955 | 0.000273 |
| 16976054 | 23799463 | T | C | 0.839724 | 0.872189 | 1.409362 | 0.000274 |
| 1831369 | 24171766 | C | T | 0.682753 | 0.732196 | 1.311235 | 0.000277 |
| 7631594 | 24134893 | C | T | 0.275039 | 0.328597 | 1.299835 | 0.000278 |
| 2851391 | 24551945 | T | C | 0.395678 | 0.447722 | 1.281858 | 0.000281 |
| 17573852 | 24474156 | C | G | 0.14826 | 0.190504 | 1.389059 | 0.000283 |
| 4076941 | 23810202 | T | C | 0.578221 | 0.637574 | 1.277915 | 0.000287 |
| 6813595 | 24630779 | C | G | 0.542879 | 0.600942 | 1.273396 | 0.000291 |
| 7429509 | 23673764 | C | T | 0.728951 | 0.772156 | 1.309933 | 0.000293 |
| 3117888 | 23334891 | T | C | 0.438609 | 0.50287 | 1.269127 | 0.000296 |
| 6469330 | 24017213 | A | C | 0.647692 | 0.590692 | 1.306599 | 0.000296 |
| 1880692 | 24336556 | A | G | 0.482699 | 0.538208 | 1.290352 | 0.0003 |
| 2217368 | 24629913 | T | C | 0.587597 | 0.528402 | 1.271634 | 0.0003 |
| 4142436 | 23375459 | T | C | 0.133531 | 0.100173 | 1.453683 | 0.000301 |
| 6860010 | 23324332 | T | C | 0.624043 | 0.564847 | 1.279465 | 0.000305 |
| 7845273 | 23589693 | A | T | 0.861538 | 0.896719 | 1.509636 | 0.000308 |
| 6866940 | 23758509 | A | G | 0.18097 | 0.138247 | 1.413401 | 0.00031 |
| 2351325 | 23458376 | A | G | 0.806854 | 0.753404 | 1.33198 | 0.000311 |
| 399516 | 24684478 | C | A | 0.553042 | 0.605092 | 1.273161 | 0.000313 |
| 2764951 | 23199194 | T | C | 0.18219 | 0.133292 | 1.39376 | 0.000314 |
| 16944026 | 23955249 | T | C | 0.901074 | 0.868857 | 1.461342 | 0.000316 |
| 17029364 | 24152380 | A | G | 0.789086 | 0.835338 | 1.35082 | 0.000317 |
| 1025623 | 24650193 | A | G | 0.273616 | 0.317051 | 1.308793 | 0.000324 |
| 2682968 | 24278304 | A | C | 0.61743 | 0.558958 | 1.271493 | 0.000328 |
| 6563127 | 24453495 | A | G | 0.887172 | 0.917994 | 1.487471 | 0.000331 |
| 17796970 | 24308693 | A | G | 0.733945 | 0.78169 | 1.321682 | 0.000334 |
| 7909516 | 23385028 | C | T | 0.663859 | 0.714412 | 1.299678 | 0.000334 |
| 261712 | 23831465 | C | T | 0.563163 | 0.49427 | 1.217579 | 0.000334 |
| 2277937 | 24022954 | T | C | 0.766462 | 0.72 | 1.305882 | 0.000335 |
| 579596 | 24674812 | T | C | 0.472222 | 0.534524 | 1.274053 | 0.000336 |
| 2825163 | 24205434 | G | A | 0.707729 | 0.750612 | 1.323629 | 0.000345 |
| 6897128 | 24188971 | A | G | 0.575221 | 0.519462 | 1.271926 | 0.000346 |
| 2025122 | 24417281 | G | A | 0.844316 | 0.888518 | 1.423684 | 0.000346 |
| 17652287 | 24284076 | C | T | 0.664606 | 0.718252 | 1.288917 | 0.000349 |
| 11151508 | 24505283 | G | C | 0.838782 | 0.791404 | 1.355642 | 0.00035 |
| 7666299 | 24236069 | A | G | 0.56 | 0.624067 | 1.267862 | 0.000352 |
| 197894 | 24045715 | G | C | 0.956559 | 0.918223 | 1.767853 | 0.000353 |
| 7429119 | 23755325 | T | C | 0.158706 | 0.203209 | 1.357379 | 0.000354 |
| 2240142 | 23475360 | C | T | 0.806785 | 0.851576 | 1.37177 | 0.000356 |
| 2281628 | 24099502 | A | G | 0.391339 | 0.447559 | 1.276633 | 0.00036 |
| 10844149 | 24364474 | G | A | 0.702703 | 0.760803 | 1.313403 | 0.000362 |
| 1452807 | 24545243 | C | G | 0.281442 | 0.221234 | 1.320504 | 0.000364 |
| 10873421 | 23654501 | G | A | 0.275373 | 0.230455 | 1.316218 | 0.000365 |
| 9543383 | 24520751 | T | C | 0.902093 | 0.864559 | 1.45851 | 0.000365 |
| 2579875 | 23534378 | T | C | 0.808901 | 0.750498 | 1.364507 | 0.000365 |
| 16839587 | 23925797 | A | G | 0.944853 | 0.968227 | 1.87846 | 0.000372 |
| 903346 | 24642423 | G | C | 0.381988 | 0.339148 | 1.284418 | 0.000377 |
| 4439342 | 23873163 | C | T | 0.733114 | 0.673507 | 1.296199 | 0.000378 |
| 4007 | 23498869 | T | A | 0.830261 | 0.7763 | 1.345992 | 0.000381 |
| 2061717 | 24328026 | T | C | 0.509745 | 0.448394 | 1.278286 | 0.000381 |
| 2714678 | 23531563 | G | T | 0.536732 | 0.585841 | 1.278436 | 0.000389 |
| 11176454 | 24214546 | T | C | 0.801902 | 0.75753 | 1.344444 | 0.000391 |
| 17793991 | 24509420 | C | A | 0.54 | 0.5984 | 1.27987 | 0.000393 |
| 1584769 | 24074833 | A | T | 0.533026 | 0.484117 | 1.267858 | 0.000399 |
| 7969224 | 24428656 | G | A | 0.86276 | 0.822209 | 1.382785 | 0.000399 |
| 3844055 | 23188349 | A | G | 0.292188 | 0.337597 | 1.286745 | 0.000405 |
| 7670013 | 23742709 | T | C | 0.714509 | 0.765403 | 1.306064 | 0.000407 |
| 4305582 | 23714130 | T | G | 0.743339 | 0.793171 | 1.34819 | 0.00041 |
| 658812 | 23513964 | A | C | 0.691285 | 0.733238 | 1.315089 | 0.000411 |
| 11171159 | 24359037 | C | T | 0.86 | 0.813504 | 1.37192 | 0.000412 |
| 4690001 | 23891796 | C | T | 0.749587 | 0.793734 | 1.325199 | 0.000414 |
| 17254424 | 24416402 | A | G | 0.877916 | 0.915976 | 1.474699 | 0.000417 |
| 1361933 | 24695804 | C | T | 0.588235 | 0.636873 | 1.269773 | 0.00042 |
| 6725580 | 24634209 | G | A | 0.539474 | 0.47491 | 1.254704 | 0.000422 |
| 17263496 | 24419002 | G | A | 0.865583 | 0.905498 | 1.446981 | 0.000424 |
| 2550621 | 24195806 | C | T | 0.46467 | 0.527499 | 1.278431 | 0.000427 |
| 713286 | 23356479 | T | C | 0.883704 | 0.8476 | 1.431892 | 0.000428 |
| 1156822 | 23868266 | A | T | 0.447863 | 0.385906 | 1.287558 | 0.000436 |
| 17429548 | 23711465 | A | G | 0.413077 | 0.360154 | 1.276956 | 0.000437 |
| 2278381 | 23935144 | C | A | 0.973926 | 0.951804 | 1.906288 | 0.000437 |
| 17249385 | 24426511 | G | A | 0.825348 | 0.780363 | 1.325454 | 0.000441 |
| 17719112 | 24194884 | G | C | 0.79784 | 0.844491 | 1.346263 | 0.000441 |
| 11833134 | 23952248 | T | C | 0.646789 | 0.701356 | 1.279856 | 0.000447 |
| 17287745 | 24622099 | A | G | 0.685385 | 0.635719 | 1.29031 | 0.000454 |
| 3732768 | 23673768 | G | A | 0.726154 | 0.766627 | 1.295762 | 0.000456 |
| 16902330 | 23459589 | A | G | 0.029231 | 0.056213 | 1.855187 | 0.000469 |
| 17763421 | 24392771 | C | T | 0.410982 | 0.465196 | 1.273553 | 0.000479 |
| 10483450 | 24099451 | C | T | 0.648773 | 0.69494 | 1.282745 | 0.000481 |
| 13026628 | 24654234 | C | T | 0.875385 | 0.839466 | 1.395316 | 0.000485 |
| 16894082 | 23380583 | T | C | 0.924847 | 0.948817 | 1.610727 | 0.000487 |
| 17428526 | 24540628 | A | G | 0.885366 | 0.85122 | 1.423318 | 0.000488 |
| 17572655 | 24573436 | G | A | 0.789474 | 0.73768 | 1.322727 | 0.000489 |
| 17784735 | 24406001 | T | A | 0.873476 | 0.906671 | 1.439511 | 0.00049 |
| 12162084 | 24217409 | G | A | 0.81762 | 0.853896 | 1.357766 | 0.000491 |
| 10933481 | 24333006 | T | C | 0.52709 | 0.472485 | 1.260083 | 0.000495 |
| 6941698 | 24684483 | G | C | 0.516304 | 0.566787 | 1.261901 | 0.000496 |
| 13037781 | 23558482 | C | T | 0.389561 | 0.348235 | 1.281774 | 0.000498 |
| 11077567 | 24213923 | A | T | 0.246154 | 0.198512 | 1.345999 | 0.0005 |
| 4465511 | 23399579 | A | G | 0.205607 | 0.250906 | 1.341333 | 0.000505 |
| 4791962 | 24592048 | A | G | 0.534884 | 0.473093 | 1.265944 | 0.000509 |
| 6481643 | 24624089 | T | C | 0.219685 | 0.262981 | 1.308886 | 0.000511 |
| 4140768 | 24704028 | A | C | 0.525502 | 0.576401 | 1.263598 | 0.000513 |
| 11023815 | 23589025 | C | G | 0.655956 | 0.592692 | 1.265337 | 0.000516 |
| 3775561 | 24662115 | T | A | 0.532209 | 0.590188 | 1.261012 | 0.000518 |
| 7570033 | 23255656 | C | T | 0.719675 | 0.657985 | 1.283173 | 0.000528 |
| 13289879 | 24579023 | A | G | 0.95421 | 0.930778 | 1.648648 | 0.000528 |
| 5992689 | 23787011 | G | A | 0.861196 | 0.815836 | 1.373199 | 0.000539 |
| 8024294 | 23743009 | G | A | 0.895678 | 0.854895 | 1.423223 | 0.000539 |
| 17622991 | 24681239 | G | A | 0.824387 | 0.78149 | 1.328991 | 0.000542 |
| 3781575 | 23498800 | T | C | 0.841577 | 0.789661 | 1.355825 | 0.000543 |
| 9314751 | 23431158 | A | G | 0.752294 | 0.695716 | 1.299981 | 0.000544 |
| 4398609 | 24356811 | A | C | 0.491499 | 0.54873 | 1.268666 | 0.000547 |
| 1261166 | 23938179 | T | C | 0.125 | 0.171182 | 1.376217 | 0.00055 |
| 1543976 | 24627613 | A | G | 0.39954 | 0.346541 | 1.261321 | 0.000556 |
| 10185471 | 23246611 | T | C | 0.749235 | 0.696124 | 1.286977 | 0.000559 |
| 10842616 | 23428232 | C | G | 0.788401 | 0.735723 | 1.336419 | 0.00056 |
| 17399569 | 24242512 | C | T | 0.916535 | 0.942308 | 1.55996 | 0.000561 |
| 4396895 | 24332928 | G | A | 0.410745 | 0.478934 | 1.289893 | 0.000566 |
| 11177315 | 24611408 | C | G | 0.770124 | 0.717109 | 1.307456 | 0.00057 |
| 10787862 | 24575921 | C | A | 0.46089 | 0.508279 | 1.260612 | 0.000571 |
| 3795294 | 23668004 | T | C | 0.872699 | 0.906213 | 1.422753 | 0.000577 |
| 528374 | 24431617 | A | G | 0.363166 | 0.414121 | 1.266763 | 0.000578 |
| 1663588 | 24137580 | A | G | 0.11435 | 0.148862 | 1.418555 | 0.000578 |
| 11642164 | 24189783 | C | G | 0.643731 | 0.583676 | 1.264523 | 0.000578 |
| 247052 | 24537584 | T | C | 0.227132 | 0.272271 | 1.301166 | 0.00058 |
| 10779375 | 24268680 | T | C | 0.52093 | 0.464713 | 1.262524 | 0.000581 |
| 10484415 | 24383938 | C | G | 0.808824 | 0.759582 | 1.352208 | 0.000582 |
| 1161098 | 24707116 | G | A | 0.194239 | 0.151602 | 1.347249 | 0.000583 |
| 7635836 | 23275312 | G | C | 0.790644 | 0.832643 | 1.332298 | 0.000594 |
| 2704789 | 24166412 | G | A | 0.659699 | 0.613118 | 1.290693 | 0.000595 |
| 1370116 | 24159111 | A | G | 0.502304 | 0.452367 | 1.265414 | 0.000609 |
| 4345821 | 23853978 | T | C | 0.645482 | 0.698767 | 1.277135 | 0.000611 |
| 2418978 | 24524819 | T | C | 0.579066 | 0.529273 | 1.260302 | 0.000613 |
| 17101420 | 24099431 | A | G | 0.596121 | 0.645397 | 1.280214 | 0.000616 |
| 6070619 | 24598466 | C | T | 0.929467 | 0.903264 | 1.514456 | 0.000617 |
| 3741601 | 24441058 | A | G | 0.727907 | 0.678699 | 1.287782 | 0.00063 |
| 1348994 | 23688892 | T | C | 0.278716 | 0.237547 | 1.33006 | 0.00063 |
| 4397143 | 23258338 | A | C | 0.667178 | 0.616833 | 1.279571 | 0.000631 |
| 6766574 | 23289463 | T | C | 0.735679 | 0.6811 | 1.291665 | 0.000632 |
| 4746136 | 24119040 | G | A | 0.876147 | 0.832355 | 1.409562 | 0.000633 |
| 2270927 | 24189133 | G | C | 0.175697 | 0.132757 | 1.364668 | 0.000634 |
| 888784 | 23247790 | T | C | 0.311828 | 0.360154 | 1.270641 | 0.000635 |
| 1500065 | 23730828 | C | T | 0.751863 | 0.708743 | 1.302269 | 0.000642 |
| 4845963 | 23843186 | G | A | 0.682722 | 0.632413 | 1.270224 | 0.000643 |
| 11820556 | 23629748 | A | G | 0.891036 | 0.856257 | 1.421626 | 0.000644 |
| 17077144 | 23368681 | C | A | 0.844037 | 0.798122 | 1.344544 | 0.000645 |
| 2277594 | 24126822 | C | T | 0.963918 | 0.982554 | 2.159487 | 0.000652 |
| 32401 | 23446086 | C | T | 0.226334 | 0.290559 | 1.317075 | 0.000655 |
| 2078131 | 23545887 | G | A | 0.773256 | 0.729003 | 1.308603 | 0.000657 |
| 7942997 | 23623726 | T | C | 0.959283 | 0.931761 | 1.689491 | 0.000663 |
| 8103560 | 24225485 | T | C | 0.598462 | 0.644557 | 1.274597 | 0.000663 |
| 902271 | 24189394 | T | C | 0.835637 | 0.867891 | 1.382999 | 0.000664 |
| 2148694 | 24072811 | C | T | 0.136923 | 0.101962 | 1.409981 | 0.00067 |
| 855965 | 23608911 | A | G | 0.355049 | 0.421942 | 1.267427 | 0.000671 |
| 17495754 | 24562333 | G | A | 0.947492 | 0.919976 | 1.573269 | 0.000674 |
| 2037293 | 24730486 | C | A | 0.28192 | 0.339218 | 1.236556 | 0.000677 |
| 2034796 | 24345729 | G | A | 0.4825 | 0.543987 | 1.260992 | 0.000678 |
| 17529372 | 24655122 | C | G | 0.804517 | 0.845606 | 1.346702 | 0.000679 |
| 17081286 | 24004586 | C | T | 0.557443 | 0.616447 | 1.250761 | 0.00068 |
| 17283421 | 24730426 | G | A | 0.893762 | 0.929149 | 1.37411 | 0.000688 |
| 2010711 | 24300319 | A | T | 0.632006 | 0.584048 | 1.26335 | 0.000688 |
| 10818896 | 23499368 | T | C | 0.819099 | 0.851051 | 1.37078 | 0.000689 |
| 6968649 | 23433229 | C | G | 0.523774 | 0.591701 | 1.253881 | 0.000691 |
| 11144705 | 23490584 | A | G | 0.886964 | 0.924261 | 1.472116 | 0.000691 |
| 17137494 | 24074774 | C | T | 0.876667 | 0.902469 | 1.476201 | 0.000694 |
| 6921677 | 24373670 | C | T | 0.174923 | 0.215634 | 1.333697 | 0.000694 |
| 17012108 | 23403601 | T | C | 0.533639 | 0.585832 | 1.249222 | 0.000696 |
| 9831663 | 23224477 | A | C | 0.420245 | 0.473606 | 1.258044 | 0.000699 |
| 1671413 | 24410446 | T | C | 0.247415 | 0.292984 | 1.289258 | 0.000708 |
| 8091352 | 23479883 | A | T | 0.622324 | 0.687023 | 1.263477 | 0.000711 |
| 478239 | 24225486 | C | A | 0.599384 | 0.64651 | 1.271304 | 0.000719 |
| 7257225 | 24682133 | T | C | 0.328125 | 0.379811 | 1.272448 | 0.000728 |
| 2437095 | 23374778 | G | T | 0.222136 | 0.177981 | 1.318554 | 0.00073 |
| 7587023 | 23301107 | C | T | 0.552995 | 0.607122 | 1.256572 | 0.000732 |
| 11209403 | 23848737 | T | C | 0.739844 | 0.791236 | 1.299439 | 0.000732 |
| 17131701 | 23853902 | A | G | 0.646248 | 0.698998 | 1.272377 | 0.000737 |
| 11582478 | 24276808 | T | G | 0.716039 | 0.765734 | 1.309977 | 0.000738 |
| 1106722 | 23792505 | A | G | 0.897081 | 0.920569 | 1.550633 | 0.000739 |
| 17378751 | 24545185 | C | T | 0.848101 | 0.890041 | 1.390019 | 0.00074 |
| 1989309 | 23887548 | A | C | 0.640138 | 0.692332 | 1.282932 | 0.000742 |
| 7328107 | 23983332 | A | C | 0.306785 | 0.358615 | 1.27559 | 0.000744 |
| 747532 | 24476902 | C | T | 0.483746 | 0.425719 | 1.25606 | 0.000748 |
| 4976685 | 23258293 | G | A | 0.36476 | 0.415472 | 1.276726 | 0.00075 |
| 5910439 | 24729468 | G | A | 0.65251 | 0.599464 | 1.213568 | 0.000755 |
| 16873956 | 23410589 | A | G | 0.930982 | 0.897398 | 1.55791 | 0.000756 |
| 1507599 | 23552695 | C | A | 0.61226 | 0.669336 | 1.265702 | 0.000759 |
| 7757529 | 24497301 | G | C | 0.618056 | 0.564678 | 1.25554 | 0.000762 |
| 17348962 | 24542111 | C | T | 0.704918 | 0.753598 | 1.276459 | 0.000763 |
| 489977 | 24512768 | G | T | 0.722042 | 0.681219 | 1.292307 | 0.000768 |
| 9725695 | 23186933 | C | T | 0.765568 | 0.703378 | 1.315053 | 0.000775 |
| 17659437 | 24276737 | T | C | 0.816923 | 0.860568 | 1.346371 | 0.00078 |
| 2426778 | 23452529 | A | G | 0.439417 | 0.493227 | 1.246918 | 0.000783 |
| 2073145 | 24451923 | C | T | 0.396789 | 0.34909 | 1.263053 | 0.000784 |
| 7002174 | 24084138 | A | G | 0.491852 | 0.427826 | 1.256521 | 0.000785 |
| 13157045 | 23923070 | C | T | 0.70339 | 0.659442 | 1.271513 | 0.000787 |
| 163299 | 23975386 | G | T | 0.825617 | 0.777778 | 1.327375 | 0.000795 |
| 16929452 | 23997965 | A | G | 0.856887 | 0.812945 | 1.381363 | 0.000797 |
| 6962459 | 24675203 | A | T | 0.091581 | 0.124785 | 1.427884 | 0.000798 |
| 1745836 | 24431150 | C | T | 0.213405 | 0.166074 | 1.318863 | 0.000798 |
| 1748952 | 24603865 | A | G | 0.546923 | 0.497027 | 1.242941 | 0.000798 |
| 7668673 | 24398923 | C | T | 0.782031 | 0.735068 | 1.295449 | 0.000801 |
| 2028088 | 24307687 | C | T | 0.693344 | 0.748645 | 1.288433 | 0.000803 |
| 1935153 | 24568806 | A | G | 0.365727 | 0.414278 | 1.257629 | 0.000811 |
| 12204525 | 24622323 | T | C | 0.377112 | 0.435541 | 1.253952 | 0.000814 |
| 5980169 | 23816457 | T | C | 0.888345 | 0.846213 | 1.349219 | 0.000815 |
| 16881360 | 24088842 | T | C | 0.862126 | 0.816056 | 1.386017 | 0.000824 |
| 11914104 | 23646668 | C | A | 0.775194 | 0.816568 | 1.324138 | 0.000827 |
| 2738591 | 24195821 | C | T | 0.480769 | 0.542037 | 1.261319 | 0.00083 |
| 1472424 | 23183508 | A | G | 0.412711 | 0.374777 | 1.263837 | 0.000833 |
| 13409142 | 23193345 | C | T | 0.384555 | 0.328597 | 1.263285 | 0.000833 |
| 12724393 | 24251682 | C | G | 0.611111 | 0.671732 | 1.262925 | 0.000836 |
| 1524408 | 24673906 | T | A | 0.412809 | 0.371122 | 1.257511 | 0.00084 |
| 703406 | 23608899 | A | G | 0.409021 | 0.46886 | 1.250577 | 0.00084 |
| 17143871 | 23361050 | T | C | 0.690476 | 0.730337 | 1.278536 | 0.000851 |
| 886126 | 23389735 | C | T | 0.735835 | 0.678215 | 1.289951 | 0.000858 |
| 891978 | 23753076 | C | T | 0.335123 | 0.405565 | 1.258161 | 0.000858 |
| 6775742 | 24282737 | A | G | 0.700155 | 0.638725 | 1.266871 | 0.00087 |
| 17412366 | 24649394 | C | A | 0.888393 | 0.922087 | 1.49931 | 0.000872 |
| 4943826 | 23302101 | T | C | 0.815467 | 0.848592 | 1.341664 | 0.000873 |
| 17315298 | 24634262 | C | T | 0.816514 | 0.859953 | 1.343578 | 0.000881 |
| 17289925 | 24221325 | T | C | 0.999227 | 0.991374 | 10.4357 | 0.000882 |
| 17001863 | 24079440 | A | G | 0.855932 | 0.890065 | 1.391412 | 0.000884 |
| 3850225 | 24654763 | A | G | 0.691617 | 0.635723 | 1.265174 | 0.000885 |
| 6596200 | 24356774 | A | G | 0.55138 | 0.609994 | 1.24929 | 0.000885 |
| 17198844 | 24417517 | G | C | 0.904908 | 0.870414 | 1.422168 | 0.000885 |
| 7525479 | 24257106 | G | C | 0.744977 | 0.696439 | 1.27675 | 0.000887 |
| 1012968 | 23577636 | T | G | 0.935535 | 0.90247 | 1.526401 | 0.000893 |
| 510970 | 24520470 | T | C | 0.805215 | 0.847543 | 1.327329 | 0.000897 |
| 17541270 | 24456879 | A | G | 0.906347 | 0.934141 | 1.487849 | 0.000897 |
| 4699197 | 23237336 | C | T | 0.904762 | 0.871082 | 1.4186 | 0.000906 |
| 1106449 | 23961108 | C | A | 0.314732 | 0.369578 | 1.257652 | 0.00091 |
| 10504647 | 24084733 | C | G | 0.495327 | 0.433215 | 1.252818 | 0.000917 |
| 967702 | 24458545 | T | C | 0.688095 | 0.635766 | 1.275847 | 0.00092 |
| 12018552 | 23514195 | G | A | 0.707055 | 0.651533 | 1.262172 | 0.000921 |
| 16872491 | 23409841 | T | C | 0.855505 | 0.814815 | 1.341256 | 0.000922 |
| 3827896 | 23654688 | G | A | 0.545669 | 0.603762 | 1.263401 | 0.000924 |
| 1450631 | 23435575 | T | C | 0.082181 | 0.113838 | 1.474431 | 0.000926 |
| 11044839 | 23990157 | C | T | 0.868078 | 0.828449 | 1.36165 | 0.000929 |
| 1076184 | 24579881 | T | A | 0.128981 | 0.09194 | 1.418842 | 0.000931 |
| 1026071 | 24092882 | A | G | 0.745313 | 0.696535 | 1.283282 | 0.000933 |
| 16899163 | 24072251 | A | G | 0.792645 | 0.83363 | 1.318356 | 0.000939 |
| 234 | 24431570 | A | G | 0.517002 | 0.460721 | 1.245281 | 0.000941 |
| 4461009 | 24224897 | T | A | 0.805172 | 0.841192 | 1.342714 | 0.000944 |
| 12054491 | 23907649 | G | A | 0.864969 | 0.898099 | 1.398778 | 0.000945 |
| 10276660 | 23442018 | G | T | 0.867994 | 0.902299 | 1.41142 | 0.000946 |
| 16892924 | 23313927 | T | C | 0.768576 | 0.804632 | 1.3362 | 0.000946 |
| 4982207 | 24578093 | C | T | 0.597372 | 0.649558 | 1.256642 | 0.000948 |
| 11651604 | 24705544 | G | C | 0.813898 | 0.863882 | 1.352075 | 0.000954 |
| 10984781 | 24088381 | A | G | 0.728814 | 0.77023 | 1.288624 | 0.000956 |
| 10793571 | 24552848 | C | T | 0.68 | 0.626506 | 1.25732 | 0.000956 |
| 1341513 | 24503921 | C | A | 0.664596 | 0.720562 | 1.26587 | 0.000957 |
| 1485208 | 24290135 | C | G | 0.470138 | 0.525867 | 1.249001 | 0.00096 |
| 1078504 | 23552724 | G | A | 0.853354 | 0.894737 | 1.392206 | 0.000963 |
| 11892547 | 23720603 | T | G | 0.794017 | 0.745104 | 1.312482 | 0.000963 |
| 17315903 | 24524731 | T | C | 0.790404 | 0.827149 | 1.33754 | 0.000964 |
| 7832077 | 24094060 | T | C | 0.897218 | 0.925369 | 1.468758 | 0.000964 |
| 1389173 | 23354097 | G | C | 0.684579 | 0.634661 | 1.261412 | 0.000973 |
| 6698091 | 24257347 | T | C | 0.464341 | 0.511256 | 1.24378 | 0.000981 |
| 2664538 | 23574543 | A | G | 0.600746 | 0.659437 | 1.246888 | 0.000986 |
| 9826662 | 24296064 | A | C | 0.614486 | 0.561862 | 1.250575 | 0.000986 |
| 4706308 | 24418999 | G | T | 0.393568 | 0.338624 | 1.25873 | 0.00099 |
| 9893651 | 24121997 | C | T | 0.934911 | 0.894495 | 1.480726 | 0.00099 |
| 1369905 | 24345666 | G | A | 0.06378 | 0.089004 | 1.522872 | 0.000997 |

1. A method of screening of an individual suffering from a disorder of blood glucose regulation comprising screening the individual for one or more genetic variations indicating a predisposition to a response to an insulin sensitizer.

2. The method of claim 1 wherein the disorder of blood glucose regulation is an insulin resistance disorder.

3. The method of claim 1 further comprising administering or not administering an insulin sensitizer to the individual based on the results of said screening of said individual.

4-5. (canceled)

6. The method of claim 3 wherein if an insulin sensitizer is administered to the individual, the method further comprises modulating the administration of the insulin sensitizer based on the results of said screening of said individual.

7. The method of claim 6 wherein said modulating comprises administering another therapeutic agent in addition to the insulin sensitizer.

8. The method of claim 6 wherein said modulating comprises adjusting the dosage of the insulin sensitizer, route of administration of the insulin sensitizer, frequency of administration of the insulin sensitizer, type of carrier of the insulin sensitizer, duration of treatment with the insulin sensitizer, enantiomeric form of the insulin sensitizer, crystal form of the insulin sensitizer, or a combination thereof, compared to what it would have been if said screening had not been performed.

9. (canceled)

10. The method of claim 3 wherein said administering or not administering of an insulin sensitizer is performed as part of a drug trial of the insulin sensitizer.

11. The method of claim 1 wherein the disorder of blood glucose regulation is selected from the group consisting of diabetes, obesity, and Syndrome X.

12. The method of claim 1 wherein the disorder of blood glucose regulation is diabetes.

13-14. (canceled)

15. The method of claim 1, wherein the one or more genetic variations are single nucleotide polymorphisms (SNPs) selected from those provided in Table 9.

16. The method of claim 15 wherein the one or more genetic variations comprise a SNP selected from those corresponding to the RefSNP ID numbers 2232700, 941591, 941590, 3827896, 8015929, and 941601.

17. The method of claim 1 wherein the response to the insulin sensitizer for which the individual is screened is a therapeutic response.

18. The method of claim 1 wherein the response to the insulin sensitizer for which the individual is screened is an adverse effect.

19. The method of claim 18 wherein the adverse effect is selected from the group consisting of peripheral edema, dependent edema, generalized edema, pitting edema, weight increase, anemia, hypoglycemia, headache, increase in micturation frequency, diarrhea, increased appetite, transient ischemic attack, elevated liver enzymes, and combinations thereof.

20. The method of claim 1 wherein the insulin sensitizer for which the individual is screened and that is administered or not administered to the individual is a thiazolidinedione PPAR modulator.

21. The method of claim 20 wherein the thiazolidinedione PPAR modulator for which the individual is screened is selected from the group consisting of rosiglitazone, pioglitazone, troglitazone, netoglitazone, and 5-BTZD; and the insulin sensitizer that is administered or not administered to the individual is selected from the group consisting of rosiglitazone, pioglitazone, troglitazone, netoglitazone, and 5-BTZD.

22. The method of claim 21 wherein the insulin sensitizer for which the individual is screened and that is administered or not administered to the individual is netoglitazone.

23. (canceled)

24. The method of claim 20 wherein the insulin sensitizer for which the individual is screened is a thiazolidinedione PPAR modulator and the insulin sensitizer that is administered or not administered to the individual is netoglitazone.

25. The method of claim 20 wherein the insulin sensitizer for which the individual is screened is a thiazolidinedione PPAR modulator selected from the group consisting of rosiglitazone, pioglitazone, troglitazone, netoglitazone and 5-BTZD and the insulin sensitizer that is administered or not administered to the individual is netoglitazone.

26. The method of claim 1 wherein the disorder of blood glucose regulation is Type II diabetes, the genetic variation is a SNP or a plurality of SNPs, the insulin sensitizer is netoglitazone, and the response is a therapeutic response.

27. The method of claim 1 wherein the disorder of blood glucose regulation is Type II diabetes, the genetic variation is a SNP or a plurality of SNPs, the insulin sensitizer is netoglitazone, and the response is an adverse effect.

28. The method of claim 1 wherein the screening comprises genotyping at least 1 genetic variation of the individual.

29-31. (canceled)

32. The method of claim 1 wherein the screening comprises genotyping at least 10,000 genetic variations of the individual.

33. The method of claim 1 wherein the screening comprises genotyping at least about 100,000 genetic variations.

34. The method of claim 1 wherein said screening further comprises identifying one or more phenotypes of the individual indicating said predisposition.

35. The method of claim 34, wherein said one or more phenotypes are selected from the group comprising gender, number of years with type 2 diabetes, and dose of thiozolidineodione therapy.

36. The method of claim 1 further comprising converting the results of said screening into data that is capable of transmission.

37. The method of claim 36 further comprising transmitting said data to a location different from the location at which the data was created.

38. An isolated nucleic acid that specifically hybridizes to a genomic sequence from 10 kb upstream to 10 kb downstream of an insulin sensitizer response nucleic acid, for use in diagnostics, prognostics, prevention, treatment, or study of a disorder of blood glucose regulation.

39-43. (canceled)

44. A method for predicting a presence or absence of a predisposition toward response to an insulin sensitizer in an individual comprising contacting a sample obtained from the individual with a nucleic acid of claim 38; and detecting the presence or absence of a hybridization complex, wherein the presence or absence of a hybridization complex is predictive of the presence or absence of said predisposition toward response to said insulin sensitizer in said individual.

45. The method of claim 44 further comprising administering or not administering an effective amount of an insulin sensitizer to said patient, based on the presence or absence of the hybridization complex.

46-47. (canceled)

48. An isolated polypeptide encoded by a nucleic acid of claim 38 for use in screening, diagnostics, prognostics, prevention, treatment, or study of response to an insulin sensitizer.

49-52. (canceled)


SEQUENCE LISTING (345 sequences)

SEQ ID NO: 1 - DNA, 29 bp, Homo sapiens
  misc_feature 15: n = A,T,C or G
ggcagagactgaatnaagggttgacccag

SEQ ID NO: 2 - DNA, 29 bp, Homo sapiens
  misc_feature 15: n = A,T,C or G
cagaccttcttccanctgtaaaattccca

SEQ ID NO: 3 - DNA, 29 bp, Homo sapiens
  misc_feature 15: n = A,T,C or G
aagaggtaacccctngacctaagaggaaa

SEQ ID NO: 4 - DNA, 29 bp, Homo sapiens
  misc_feature 15: n = A,T,C or G
atttcttgagaagtncaacaacacagtga

SEQ ID NO: 5 - DNA, 29 bp, Homo sapiens
  misc_feature 15: n = A,T,C or G
caaaatttgcttagntaacttccccgggg

SEQ ID NO: 6 - DNA, 29 bp, Homo sapiens
  misc_feature 15: n = A,T,C or G
agatttgagaaaagnttcaaaatgcaaat

SEQ ID NO: 7 - DNA, 29 bp, Homo sapiens
  misc_feature 15: n = A,T,C or G
tagtgtgtgctactncctatttggataac

SEQ ID NO: 8 - DNA, 29 bp, Homo sapiens
  misc_feature 15: n = A,T,C or G
gctagtagatttcangtctattaacagtc

SEQ ID NO: 9 - DNA, 29 bp, Homo sapiens
  misc_feature 15: n = A,T,C or G
cttgctctctaatcntcagcatcttcgtg

SEQ ID NO: 10 - DNA, 29 bp, Homo sapiens
  misc_feature 15: n = A,T,C or G
tagaatgcacaaatnagcataaaagaaaa

SEQ ID NO: 11 - DNA, 29 bp, Homo sapiens
  misc_feature 15: n = A,T,C or G
aaatatgaataattntttgaccagtactt

SEQ ID NO: 12 - DNA, 29 bp, Homo sapiens
  misc_feature 15: n = A,T,C or G
accataaaaagacanttctcagcagagac

SEQ ID NO: 13 - DNA, 29 bp, Homo sapiens
  misc_feature 15: n = A,T,C or G
ggcaatgaggcttangtatttgtgtttct

SEQ ID NO: 14 - DNA, 29 bp, Homo sapiens
  misc_feature 15: n = A,T,C or G
atgctcaacagccantaagatctttcaga

SEQ ID NO: 15 - DNA, 29 bp, Homo sapiens
  misc_feature 15: n = A,T,C or G
aaatgcacatggatngttttgaccacagc

SEQ ID NO: 16 - DNA, 29 bp, Homo sapiens
  misc_feature 15: n = A,T,C or G
ccgtttgacaaggtntctggctgatataa

SEQ ID NO: 17 - DNA, 29 bp, Homo sapiens
  misc_feature 15: n = A,T,C or G
taagaagcattgatncaggaaatatctta

SEQ ID NO: 18 - DNA, 29 bp, Homo sapiens
  misc_feature 15: n = A,T,C or G
ccaggcaattcatcntgtctcaaggcaga

SEQ ID NO: 19 - DNA, 29 bp, Homo sapiens
  misc_feature 15: n = A,T,C or G
aatgagagcttgtanataaaagtccttgc

SEQ ID NO: 20 - DNA, 29 bp, Homo sapiens
  misc_feature 15: n = A,T,C or G
ttctgcaacctgctncaaccaggactctt

SEQ ID NO: 21 - DNA, 29 bp, Homo sapiens
  misc_feature 15: n = A,T,C or G
ctggctccccgctcnatggttttcagctg

SEQ ID NO: 22 - DNA, 29 bp, Homo sapiens
  misc_feature 15: n = A,T,C or G
tgagggtgaaatttngagagctgaggaaa

SEQ ID NO: 23 - DNA, 29 bp, Homo sapiens
  misc_feature 15: n = A,T,C or G
ttaaggcagtgcccnaacttttatctatt

SEQ ID NO: 24 - DNA, 29 bp, Homo sapiens
  misc_feature 15: n = A,T,C or G
agcccaatagtctgnagtttgtccagaag

SEQ ID NO: 25 - DNA, 29 bp, Homo sapiens
  misc_feature 15: n = A,T,C or G
gccaagtttctaccngaggcttggcttcc

SEQ ID NO: 26 - DNA, 29 bp, Homo sapiens
  misc_feature 15: n = A,T,C or G
tagactttggagccnattttcttctttca

SEQ ID NO: 27 - DNA, 29 bp, Homo sapiens
  misc_feature 15: n = A,T,C or G
catcctctattttgnacgctcagtcaccc

SEQ ID NO: 28 - DNA, 29 bp, Homo sapiens
  misc_feature 15: n = A,T,C or G
aacacctgtacacanaactgcagggccct

SEQ ID NO: 29 - DNA, 29 bp, Homo sapiens
  misc_feature 15: n = A,T,C or G
gtaagaatgtcttcnttacagaataatgc

SEQ ID NO: 30 - DNA, 29 bp, Homo sapiens
  misc_feature 15: n = A,T,C or G
tctgctattaccagnactcttcagtatta

SEQ ID NO: 31 - DNA, 29 bp, Homo sapiens
  misc_feature 15: n = A,T,C or G
ttcactttagaaatnaatggattattagt

SEQ ID NO: 32 - DNA, 29 bp, Homo sapiens
  misc_feature 15: n = A,T,C or G
tctggccatccctgnctggaacctgttct

SEQ ID NO: 33 - DNA, 29 bp, Homo sapiens
  misc_feature 15: n = A,T,C or G
ttagcatacgttttntaataattatgtta

SEQ ID NO: 34 - DNA, 29 bp, Homo sapiens
  misc_feature 15: n = A,T,C or G
taaatgtgggacctnagatttgaacccta

SEQ ID NO: 35 - DNA, 29 bp, Homo sapiens
  misc_feature 15: n = A,T,C or G
agaatgagttctttntgcagttccattgg

SEQ ID NO: 36 - DNA, 29 bp, Homo sapiens
  misc_feature 15: n = A,T,C or G
agatgatttaggctnagaaagaggaggcc

SEQ ID NO: 37 - DNA, 29 bp, Homo sapiens
  misc_feature 15: n = A,T,C or G
gatcgatatctgtantttgactttcttat

SEQ ID NO: 38 - DNA, 29 bp, Homo sapiens
  misc_feature 15: n = A,T,C or G
ctgtcttaggcactnagccacaatctcaa

SEQ ID NO: 39 - DNA, 29 bp, Homo sapiens
  misc_feature 15: n = A,T,C or G
ttgcagccctcacantctggttctggaag

SEQ ID NO: 40 - DNA, 29 bp, Homo sapiens
  misc_feature 15: n = A,T,C or G
gtgaggcctgggacntttttaagatcgct

SEQ ID NO: 41 - DNA, 29 bp, Homo sapiens
  misc_feature 15: n = A,T,C or G
acccccagcaaaacnggatttgtttgttt

SEQ ID NO: 42 - DNA, 29 bp, Homo sapiens
  misc_feature 15: n = A,T,C or G
ctaaaatctactttnatatttctcagcct

SEQ ID NO: 43 - DNA, 29 bp, Homo sapiens
  misc_feature 15: n = A,T,C or G
attttgactggcgcngcagaatgaggagg

SEQ ID NO: 44 - DNA, 29 bp, Homo sapiens
  misc_feature 15: n = A,T,C or G
tctcctggaaagacnggctctctcaggtt

SEQ ID NO: 45 - DNA, 29 bp, Homo sapiens
  misc_feature 15: n = A,T,C or G
cgtgacttataattngtagacactcagtg

SEQ ID NO: 46 - DNA, 29 bp, Homo sapiens
  misc_feature 15: n = A,T,C or G
tttattttcacccanaaatttacatagaa

SEQ ID NO: 47 - DNA, 29 bp, Homo sapiens
  misc_feature 15: n = A,T,C or G
tgttttcaatgtttntgctgatttttttc

SEQ ID NO: 48 - DNA, 29 bp, Homo sapiens
  misc_feature 15: n = A,T,C or G
cttctggaatatcantgaatcttgaatgt

SEQ ID NO: 49 - DNA, 29 bp, Homo sapiens
  misc_feature 15: n = A,T,C or G
taccttatccccatngagacgtcgctgga

SEQ ID NO: 50 - DNA, 29 bp, Homo sapiens
  misc_feature 15: n = A,T,C or G
ctataaggccgtacnttgtctcctttcat

SEQ ID NO: 51 - DNA, 29 bp, Homo sapiens
  misc_feature 15: n = A,T,C or G
tagttatgacttcanaccaaaatattact

SEQ ID NO: 52 - DNA, 29 bp, Homo sapiens
  misc_feature 15: n = A,T,C or G
tatgtcataccattnatcgtgatgctcaa

SEQ ID NO: 53 - DNA, 29 bp, Homo sapiens
  misc_feature 15: n = A,T,C or G
aatatgagacatcanaatacagagaatga

SEQ ID NO: 54 - DNA, 29 bp, Homo sapiens
  misc_feature 15: n = A,T,C or G
tgctctaatttcccnatgtcggttactat

SEQ ID NO: 55 - DNA, 29 bp, Homo sapiens
  misc_feature 15: n = A,T,C or G
cttacaaggccagcnatataggcagccat

SEQ ID NO: 56 - DNA, 29 bp, Homo sapiens
  misc_feature 15: n = A,T,C or G
gtacagtactgtttngaaggttttatgct

SEQ ID NO: 57 - DNA, 29 bp, Homo sapiens
  misc_feature 15: n = A,T,C or G
aattcaatcagagtnccagtcactaataa

SEQ ID NO: 58 - DNA, 29 bp, Homo sapiens
  misc_feature 15: n = A,T,C or G
atttctaagctctangtaggtgaaatccc

SEQ ID NO: 59 - DNA, 29 bp, Homo sapiens
  misc_feature 15: n = A,T,C or G
caggtttattattanaaaagtcagttgtt

SEQ ID NO: 60 - DNA, 29 bp, Homo sapiens
  misc_feature 15: n = A,T,C or G
ttatgaccatgtgantaacaaaaattctg

SEQ ID NO: 61 - DNA, 29 bp, Homo sapiens
  misc_feature 15: n = A,T,C or G
taaaactataacacncagtttccagagca

SEQ ID NO: 62 - DNA, 29 bp, Homo sapiens
  misc_feature 15: n = A,T,C or G
ttagggttagttgtnacagattgctcagt

SEQ ID NO: 63 - DNA, 29 bp, Homo sapiens
  misc_feature 15: n = A,T,C or G
tcaaattgaagtttnaattggtaaatggg

SEQ ID NO: 64 - DNA, 29 bp, Homo sapiens
  misc_feature 15: n = A,T,C or G
gcggcatggggaggncagaatagataagg

SEQ ID NO: 65 - DNA, 29 bp, Homo sapiens
  misc_feature 15: n = A,T,C or G
gattgcctcagcctntgatgatgtttaga

SEQ ID NO: 66 - DNA, 29 bp, Homo sapiens
  misc_feature 15: n = A,T,C or G
aatatttgcaggatngtcttagaattcat

SEQ ID NO: 67 - DNA, 29 bp, Homo sapiens
  misc_feature 15: n = A,T,C or G
cagggagttcttcanaagtggttatccta

SEQ ID NO: 68 - DNA, 29 bp, Homo sapiens
  misc_feature 15: n = A,T,C or G
gggaggcaagtgtgntagtccaagtaaaa

SEQ ID NO: 69 - DNA, 29 bp, Homo sapiens
  misc_feature 15: n = A,T,C or G
cagacttcctccttntaggactctctgag

SEQ ID NO: 70 - DNA, 29 bp, Homo sapiens
  misc_feature 15: n = A,T,C or G
agtgtgatctgaatntttgggtatccttc

SEQ ID NO: 71 - DNA, 29 bp, Homo sapiens
  misc_feature 15: n = A,T,C or G
tagcagataatgttnaaattaaggaatga

SEQ ID NO: 72 - DNA, 29 bp, Homo sapiens
  misc_feature 15: n = A,T,C or G
aaaggccatttgttnttgggtgtaacaat

SEQ ID NO: 73 - DNA, 29 bp, Homo sapiens
  misc_feature 15: n = A,T,C or G
ggaggcatcatacancctgacttagaaat

SEQ ID NO: 74 - DNA, 29 bp, Homo sapiens
  misc_feature 15: n = A,T,C or G
tgaagtcagagtcangagggccagctgca

SEQ ID NO: 75 - DNA, 29 bp, Homo sapiens
  misc_feature 15: n = A,T,C or G
aattttcaactcatntgaaaacatgatga

SEQ ID NO: 76 - DNA, 29 bp, Homo sapiens
  misc_feature 15: n = A,T,C or G
caattactattggcngtatctttctcttt

SEQ ID NO: 77 - DNA, 29 bp, Homo sapiens
  misc_feature 15: n = A,T,C or G
agacaatacatgaangaatgcaactgtgt

SEQ ID NO: 78 - DNA, 29 bp, Homo sapiens
  misc_feature 15: n = A,T,C or G
ctgtcctccccaccncactgtaagttctt

SEQ ID NO: 79 - DNA, 29 bp, Homo sapiens
  misc_feature 15: n = A,T,C or G
aaaaaataaccacantgccaccaaaatat

SEQ ID NO: 80 - DNA, 29 bp, Homo sapiens
  misc_feature 15: n = A,T,C or G
tggcttccgggctantgaatttaatttga

SEQ ID NO: 81 - DNA, 29 bp, Homo sapiens
  misc_feature 15: n = A,T,C or G
taccccttagcttanaagcaaaaaagaaa

SEQ ID NO: 82 - DNA, 29 bp, Homo sapiens
  misc_feature 15: n = A,T,C or G
ttgaggaatgtacanattttattctgttg

SEQ ID NO: 83 - DNA, 29 bp, Homo sapiens
  misc_feature 15: n = A,T,C or G
ttgctggggcctctnggctgcagaaagaa

SEQ ID NO: 84 - DNA, 29 bp, Homo sapiens
  misc_feature 15: n = A,T,C or G
atatgcccaatctantctgaccttcaccc

SEQ ID NO: 85 - DNA, 29 bp, Homo sapiens
  misc_feature 15: n = A,T,C or G
catctctccaaagangctcaggtctccgt

SEQ ID NO: 86 - DNA, 29 bp, Homo sapiens
  misc_feature 15: n = A,T,C or G
ctttgataatttggngtcttagtttgttt

SEQ ID NO: 87 - DNA, 29 bp, Homo sapiens
  misc_feature 15: n = A,T,C or G
aaaatcagtgggccngatactagagatag

SEQ ID NO: 88 - DNA, 29 bp, Homo sapiens
  misc_feature 15: n = A,T,C or G
tcccgcagtagaagnttagttagacgtgg

SEQ ID NO: 89 - DNA, 29 bp, Homo sapiens
  misc_feature 15: n = A,T,C or G
atatgcaaatttcantattaactttacaa

SEQ ID NO: 90 - DNA, 29 bp, Homo sapiens
  misc_feature 15: n = A,T,C or G
ggcaagcaggccccnggcatttcaaagcg

SEQ ID NO: 91 - DNA, 29 bp, Homo sapiens
  misc_feature 15: n = A,T,C or G
tcctctgagcactcnggcatttgtcattg

SEQ ID NO: 92 - DNA, 29 bp, Homo sapiens
  misc_feature 15: n = A,T,C or G
gttcagggctctttntatacctgaggcct

SEQ ID NO: 93 - DNA, 29 bp, Homo sapiens
  misc_feature 15: n = A,T,C or G
tttcaggaataaaancaacacaattattt

SEQ ID NO: 94 - DNA, 29 bp, Homo sapiens
  misc_feature 15: n = A,T,C or G
ccctgaccaaaaaanaaaagatttttcat

SEQ ID NO: 95 - DNA, 29 bp, Homo sapiens
  misc_feature 15: n = A,T,C or G
tgttatatactagantttgatagctatta

SEQ ID NO: 96 - DNA, 29 bp, Homo sapiens
  misc_feature 15: n = A,T,C or G
gccttgaaagcaccngagagtctagtctc

SEQ ID NO: 97 - DNA, 29 bp, Homo sapiens
  misc_feature 15: n = A,T,C or G
ttatatatctttccntttatttaggtctt

SEQ ID NO: 98 - DNA, 29 bp, Homo sapiens
  misc_feature 15: n = A,T,C or G
ctcaattgacatttngctccagtgtcgag

SEQ ID NO: 99 - DNA, 29 bp, Homo sapiens
  misc_feature 15: n = A,T,C or G
ttatctaaattctgntcttcactcaatat

SEQ ID NO: 100 - DNA, 29 bp, Homo sapiens
  misc_feature 15: n = A,T,C or G
gttccgggaagtccntctccagcaggaac

SEQ ID NO: 101 - DNA, 29 bp, Homo sapiens
  misc_feature 15: n = A,T,C or G
gcagcaccctgtatntgatattaatattt

SEQ ID NO: 102 - DNA, 29 bp, Homo sapiens
  misc_feature 15: n = A,T,C or G
ttctttcagccctgntctctaaaggatgg

SEQ ID NO: 103 - DNA, 29 bp, Homo sapiens
  misc_feature 15: n = A,T,C or G
cacttcagattccantcgaatacattaga

SEQ ID NO: 104 - DNA, 29 bp, Homo sapiens
  misc_feature 15: n = A,T,C or G
gcagatgcccggctngtggttaacccaag

SEQ ID NO: 105 - DNA, 29 bp, Homo sapiens
  misc_feature 15: n = A,T,C or G
gccgcatctctgatntcagagcatttaca

SEQ ID NO: 106 - DNA, 29 bp, Homo sapiens
  misc_feature 15: n = A,T,C or G
aggactctacacccnggacggcaatgctg

SEQ ID NO: 107 - DNA, 29 bp, Homo sapiens
  misc_feature 15: n = A,T,C or G
agctcctacatgtcnttttgagaccttat

SEQ ID NO: 108 - DNA, 29 bp, Homo sapiens
  misc_feature 15: n = A,T,C or G
acgtgattacaaatngggaaaagaggggc

SEQ ID NO: 109 - DNA, 29 bp, Homo sapiens
  misc_feature 15: n = A,T,C or G
tatacacacagaaanaaatactggtgaga

SEQ ID NO: 110 - DNA, 29 bp, Homo sapiens
  misc_feature 15: n = A,T,C or G
aaatttcatatgaanggtgggcttttctt

SEQ ID NO: 111 - DNA, 29 bp, Homo sapiens
  misc_feature 15: n = A,T,C or G
aactgaagattcccntttatttttctcca

SEQ ID NO: 112 - DNA, 29 bp, Homo sapiens
  misc_feature 15: n = A,T,C or G
cattatcacaaaacntaatacctgaagat

SEQ ID NO: 113 - DNA, 29 bp, Homo sapiens
  misc_feature 15: n = A,T,C or G
aacctgaccctcggngtgtctgtctgtaa

SEQ ID NO: 114 - DNA, 29 bp, Homo sapiens
  misc_feature 15: n = A,T,C or G
ttcagttagtctaanttatgaggatatat

SEQ ID NO: 115 - DNA, 29 bp, Homo sapiens
  misc_feature 15: n = A,T,C or G
atggctcttcgtccnatgattctaaagcc

SEQ ID NO: 116 - DNA, 29 bp, Homo sapiens
  misc_feature 15: n = A,T,C or G
aaaagaatttttagntcctatgtcatagt

SEQ ID NO: 117 - DNA, 29 bp, Homo sapiens
  misc_feature 15: n = A,T,C or G
catgcaatcacatcncagcagcagttgat

SEQ ID NO: 118 - DNA, 29 bp, Homo sapiens
  misc_feature 15: n = A,T,C or G
ggcaaagtatttgtnattaggaatatctg

SEQ ID NO: 119 - DNA, 29 bp, Homo sapiens
  misc_feature 15: n = A,T,C or G
gcaggcttagtcacnttgatggatctgtc

SEQ ID NO: 120 - DNA, 29 bp, Homo sapiens
  misc_feature 15: n = A,T,C or G
ggcttttaaagttcncacagacaggcatc

SEQ ID NO: 121 - DNA, 29 bp, Homo sapiens
  misc_feature 15: n = A,T,C or G
cctgtcttacttggngttgtcgagttcct

SEQ ID NO: 122 - DNA, 29 bp, Homo sapiens
  misc_feature 15: n = A,T,C or G
actatatagacaaangcatgagagcacaa

SEQ ID NO: 123 - DNA, 29 bp, Homo sapiens
  misc_feature 15: n = A,T,C or G
cattatttatcatcntcaacattattgct

SEQ ID NO: 124 - DNA, 29 bp, Homo sapiens
  misc_feature 15: n = A,T,C or G
taaaggaaggcagcngagtatattgggaa

SEQ ID NO: 125 - DNA, 29 bp, Homo sapiens
  misc_feature 15: n = A,T,C or G
gcatgagtacaagcngaggttcacatacc

SEQ ID NO: 126 - DNA, 29 bp, Homo sapiens
  misc_feature 15: n = A,T,C or G
acacaattcttaatntttggatcaggcat

SEQ ID NO: 127 - DNA, 29 bp, Homo sapiens
  misc_feature 15: n = A,T,C or G
ccattatcaccttanttacagcaatctct

SEQ ID NO: 128 - DNA, 29 bp, Homo sapiens
  misc_feature 15: n = A,T,C or G
tgcccaacgtgtgangtcatgccaccaag

SEQ ID NO: 129 - DNA, 29 bp, Homo sapiens
  misc_feature 15: n = A,T,C or G
atatctttcctttgnatcaaacagaacag

SEQ ID NO: 130 - DNA, 29 bp, Homo sapiens
  misc_feature 15: n = A,T,C or G
ttgctcaaataaacngttaatttgtctaa

SEQ ID NO: 131 - DNA, 29 bp, Homo sapiens
  misc_feature 15: n = A,T,C or G
tcagctgctgaagcnactgaattacaatg

SEQ ID NO: 132 - DNA, 29 bp, Homo sapiens
  misc_feature 15: n = A,T,C or G
agattgtgtcttcantgttaaattagata

SEQ ID NO: 133 - DNA, 29 bp, Homo sapiens
  misc_feature 15: n = A,T,C or G
gactggagatgcacnaggggccagattgt

SEQ ID NO: 134 - DNA, 29 bp, Homo sapiens
  misc_feature 15: n = A,T,C or G
tgttggggtgtctancaatagggcctgat

SEQ ID NO: 135 - DNA, 29 bp, Homo sapiens
  misc_feature 15: n = A,T,C or G
caacatgctgattantcaagttaacacct

SEQ ID NO: 136 - DNA, 29 bp, Homo sapiens
  misc_feature 15: n = A,T,C or G
acattccagtagaanaagtaaaaagccaa

SEQ ID NO: 137 - DNA, 29 bp, Homo sapiens
  misc_feature 15: n = A,T,C or G
agataaagcagacancaacaaagcagaga

SEQ ID NO: 138 - DNA, 29 bp, Homo sapiens
  misc_feature 15: n = A,T,C or G
agccataaaaagagngtacgccaaccccc

SEQ ID NO: 139 - DNA, 29 bp, Homo sapiens
  misc_feature 15: n = A,T,C or G
cttaatcttgattantgggctcagaatgt

SEQ ID NO: 140 - DNA, 29 bp, Homo sapiens
  misc_feature 15: n = A,T,C or G
ctattaccaagttcngtcattagagcagt

SEQ ID NO: 141 - DNA, 29 bp, Homo sapiens
  misc_feature 15: n = A,T,C or G
aaaacagcatgggcncaggccagttcaaa

SEQ ID NO: 142 - DNA, 29 bp, Homo sapiens
  misc_feature 15: n = A,T,C or G
aatcagatttcaaanctccagttccattt

SEQ ID NO: 143 - DNA, 29 bp, Homo sapiens
  misc_feature 15: n = A,T,C or G
actcagcttcctatntgtttttgtaagcc

SEQ ID NO: 144 - DNA, 29 bp, Homo sapiens
  misc_feature 15: n = A,T,C or G
aagcacgggtatctntacacaaataagtt

SEQ ID NO: 145 - DNA, 29 bp, Homo sapiens
  misc_feature 15: n = A,T,C or G
atgaactcatctatntttttatactctac

SEQ ID NO: 146 - DNA, 29 bp, Homo sapiens
  misc_feature 15: n = A,T,C or G
cagagcacagggagnacctgcggctttta

SEQ ID NO: 147 - DNA, 29 bp, Homo sapiens
  misc_feature 15: n = A,T,C or G
tcccaatagaaatgnttgaaaatatgaaa

SEQ ID NO: 148 - DNA, 29 bp, Homo sapiens
  misc_feature 15: n = A,T,C or G
gtgacaaccctttangctgtggtaacaaa

SEQ ID NO: 149 - DNA, 29 bp, Homo sapiens
  misc_feature 15: n = A,T,C or G
tgtaactggctgaantgaaattgactaca

SEQ ID NO: 150 - DNA, 29 bp, Homo sapiens
  misc_feature 15: n = A,T,C or G
ctcatgttggatctncctcaaggcattcc

SEQ ID NO: 151 - DNA, 29 bp, Homo sapiens
  misc_feature 15: n = A,T,C or G
aagggccaagtgatncaggttttccagaa

SEQ ID NO: 152 - DNA, 29 bp, Homo sapiens
  misc_feature 15: n = A,T,C or G
tctgctctgccttcngtacttcccgcggc

SEQ ID NO: 153 - DNA, 29 bp, Homo sapiens
  misc_feature 15: n = A,T,C or G
ttctcgttattgggncacaagaaaaagca

SEQ ID NO: 154 - DNA, 29 bp, Homo sapiens
  misc_feature 15: n = A,T,C or G
taccagtggtagttntgattacataagta

SEQ ID NO: 155 - DNA, 29 bp, Homo sapiens
  misc_feature 15: n = A,T,C or G
atgtagccaatttgntgttaaaaaaatag

SEQ ID NO: 156 - DNA, 29 bp, Homo sapiens
  misc_feature 15: n = A,T,C or G
gggactgtttttccncaaaggtttatctt

SEQ ID NO: 157 - DNA, 29 bp, Homo sapiens
  misc_feature 15: n = A,T,C or G
gtcctttctgactgncaggtgttatacaa

SEQ ID NO: 158 - DNA, 29 bp, Homo sapiens
  misc_feature 15: n = A,T,C or G
tttaatgtttatccntatgcatttatcac

SEQ ID NO: 159 - DNA, 29 bp, Homo sapiens
  misc_feature 15: n = A,T,C or G
agccgtgtcattagnatgcgtcttagaat

SEQ ID NO: 160 - DNA, 29 bp, Homo sapiens
  misc_feature 15: n = A,T,C or G
ccaacacagagtacngccttaatcgtatt

SEQ ID NO: 161 - DNA, 29 bp, Homo sapiens
  misc_feature 15: n = A,T,C or G
aattgttgcatgttntccatcagtagtaa

SEQ ID NO: 162 - DNA, 29 bp, Homo sapiens
  misc_feature 15: n = A,T,C or G
gctcaaattggaganagactatcctatag

SEQ ID NO: 163 - DNA, 29 bp, Homo sapiens
  misc_feature 15: n = A,T,C or G
agaataggcaaacantgaatgcaatattg

SEQ ID NO: 164 - DNA, 29 bp, Homo sapiens
  misc_feature 15: n = A,T,C or G
ccagttgtttgggcnctcatctgggaagc

SEQ ID NO: 165 - DNA, 29 bp, Homo sapiens
  misc_feature 15: n = A,T,C or G
ttgccaaggccgatnttgagaggatattt

SEQ ID NO: 166 - DNA, 29 bp, Homo sapiens
  misc_feature 15: n = A,T,C or G
atcaccttaattgtnttttatctaggttc

SEQ ID NO: 167 - DNA, 29 bp, Homo sapiens
  misc_feature 15: n = A,T,C or G
taattcgaggctgtngggctgaggaccct

SEQ ID NO: 168 - DNA, 29 bp, Homo sapiens
  misc_feature 15: n = A,T,C or G
atctgactttaaaanttaaaaagacaatt

SEQ ID NO: 169 - DNA, 29 bp, Homo sapiens
  misc_feature 15: n = A,T,C or G
ttatttgataccganataggcaattttaa

SEQ ID NO: 170 - DNA, 29 bp, Homo sapiens
  misc_feature 15: n = A,T,C or G
tccccttaggagcangtgggaagaagagg

SEQ ID NO: 171 - DNA, 29 bp, Homo sapiens
  misc_feature 15: n = A,T,C or G
agggtctctgtagcnggaactctcaggtc

SEQ ID NO: 172 - DNA, 29 bp, Homo sapiens
  misc_feature 15: n = A,T,C or G
ataagttcttccagnccaggatggctttc

SEQ ID NO: 173 - DNA, 29 bp, Homo sapiens
  misc_feature 15: n = A,T,C or G
aatattgtgaaacantctgagcgcaaaat

SEQ ID NO: 174 - DNA, 29 bp, Homo sapiens
  misc_feature 15: n = A,T,C or G
cagtgttctaactcntaagtgggagctga

SEQ ID NO: 175 - DNA, 29 bp, Homo sapiens
  misc_feature 15: n = A,T,C or G
actctagtggtaacnaattcacataaaca

SEQ ID NO: 176 - DNA, 29 bp, Homo sapiens
  misc_feature 15: n = A,T,C or G
atttggcagtcttgngagtcaaaagcata

SEQ ID NO: 177 - DNA, 29 bp, Homo sapiens
  misc_feature 15: n = A,T,C or G
catagaataagttanggagcagtccctct

SEQ ID NO: 178 - DNA, 29 bp, Homo sapiens
  misc_feature 15: n = A,T,C or G
atctagaaggaaatnggactttttaatat

SEQ ID NO: 179 - DNA, 29 bp, Homo sapiens
  misc_feature 15: n = A,T,C or G
agaaatattgtcagngcaaaagggctagg

SEQ ID NO: 180 - DNA, 29 bp, Homo sapiens
  misc_feature 15: n = A,T,C or G
attataagattgctnggataaaacaaagt

SEQ ID NO: 181 - DNA, 29 bp, Homo sapiens
  misc_feature 15: n = A,T,C or G
aattgcgtttctttnatgataaatcaatt

SEQ ID NO: 182 - DNA, 29 bp, Homo sapiens
  misc_feature 15: n = A,T,C or G
cgtcgggaccctccngggctagcgcgctt

SEQ ID NO: 183 - DNA, 29 bp, Homo sapiens
  misc_feature 15: n = A,T,C or G
cctagggtctatggnttttgttgccattg

SEQ ID NO: 184 - DNA, 29 bp, Homo sapiens
  misc_feature 15: n = A,T,C or G
tgcagagagatacanacgaatgcccagct

SEQ ID NO: 185 - DNA, 29 bp, Homo sapiens
  misc_feature 15: n = A,T,C or G
ggagcagtggaagcntaggtattctcttc

SEQ ID NO: 186 - DNA, 29 bp, Homo sapiens
  misc_feature 15: n = A,T,C or G
acctgtttctggctncccggcagaacctc

SEQ ID NO: 187 - DNA, 29 bp, Homo sapiens
  misc_feature 15: n = A,T,C or G
tggtattatgatagnaggcttagattcaa

SEQ ID NO: 188 - DNA, 29 bp, Homo sapiens
  misc_feature 15: n = A,T,C or G
ttatgtcacaggggnttagaggacacagg

SEQ ID NO: 189 - DNA, 29 bp, Homo sapiens
  misc_feature 15: n = A,T,C or G
cctgagttatccatngacaagagaatgag

SEQ ID NO: 190 - DNA, 29 bp, Homo sapiens
  misc_feature 15: n = A,T,C or G
tcaaagggcaacttncttaaagcattttt

SEQ ID NO: 191 - DNA, 29 bp, Homo sapiens
  misc_feature 15: n = A,T,C or G
caatttcatatgcancccatgtgagcttt

SEQ ID NO: 192 - DNA, 29 bp, Homo sapiens
  misc_feature 15: n = A,T,C or G
gaattcatgatccangtaatagtactaaa

SEQ ID NO: 193 - DNA, 29 bp, Homo sapiens
  misc_feature 15: n = A,T,C or G
taaaattcagattcngggcctgccgtggt

SEQ ID NO: 194 - DNA, 29 bp, Homo sapiens
  misc_feature 15: n = A,T,C or G
aggtgccttgcacantgcgtaccacatag

SEQ ID NO: 195 - DNA, 29 bp, Homo sapiens
  misc_feature 15: n = A,T,C or G
gtgaatgaacgaatnatgatggccaacag

SEQ ID NO: 196 - DNA, 29 bp, Homo sapiens
  misc_feature 15: n = A,T,C or G
aagccgaaggatgcngaaatgtggcactg

SEQ ID NO: 197 - DNA, 29 bp, Homo sapiens
  misc_feature 15: n = A,T,C or G
cggtatgttgcaagnggaagtactttttc

SEQ ID NO: 198 - DNA, 29 bp, Homo sapiens
  misc_feature 15: n = A,T,C or G
cacattaattcctgngtaagaaaattata

SEQ ID NO: 199 - DNA, 29 bp, Homo sapiens
  misc_feature 15: n = A,T,C or G
tgctgtgttttaccnatgcaaatgctgga

SEQ ID NO: 200 - DNA, 29 bp, Homo sapiens
  misc_feature 15: n = A,T,C or G
ccaagaaccccattntgaagttgtcctag

SEQ ID NO: 201 - DNA, 29 bp, Homo sapiens
  misc_feature 15: n = A,T,C or G
tttggcccgatgggngtatggataaattc

SEQ ID NO: 202 - DNA, 29 bp, Homo sapiens
  misc_feature 15: n = A,T,C or G
cccctttagccaaantgcacttaggataa

SEQ ID NO: 203 - DNA, 29 bp, Homo sapiens
  misc_feature 15: n = A,T,C or G
ttaagaatagttganatggcaattatgaa

SEQ ID NO: 204 - DNA, 29 bp, Homo sapiens
  misc_feature 15: n = A,T,C or G
tggatataaactctnttcttggcatgtaa

SEQ ID NO: 205 - DNA, 29 bp, Homo sapiens
  misc_feature 15: n = A,T,C or G
cagagtaaaattgcntaccatctgtcaag

SEQ ID NO: 206 - DNA, 29 bp, Homo sapiens
  misc_feature 15: n = A,T,C or G
gttagtctgtattangaaaggggactgaa

SEQ ID NO: 207 - DNA, 29 bp, Homo sapiens
  misc_feature 15: n = A,T,C or G
atcatcatctctatncacactgggaatta

SEQ ID NO: 208 - DNA, 29 bp, Homo sapiens
  misc_feature 15: n = A,T,C or G
ggtagaaccatagantgtaagtatcagtt

SEQ ID NO: 209 - DNA, 29 bp, Homo sapiens
  misc_feature 15: n = A,T,C or G
ccagttgctgaggtnggtaaaaggtcgcc

SEQ ID NO: 210 - DNA, 29 bp, Homo sapiens
  misc_feature 15: n = A,T,C or G
ttatcagtacacaancaggtcacctgact

SEQ ID NO: 211 - DNA, 29 bp, Homo sapiens
  misc_feature 15: n = A,T,C or G
aggcccagggaatangctgcccaaagtga

SEQ ID NO: 212 - DNA, 29 bp, Homo sapiens
  misc_feature 15: n = A,T,C or G
tatgaactcccttanagtaggtgggtgca

SEQ ID NO: 213 - DNA, 29 bp, Homo sapiens
  misc_feature 15: n = A,T,C or G
aggagagtggccttntatcagcctgtgtt

SEQ ID NO: 214 - DNA, 29 bp, Homo sapiens
  misc_feature 15: n = A,T,C or G
gttctgtatcttganggtggtattgttac

SEQ ID NO: 215 - DNA, 29 bp, Homo sapiens
  misc_feature 15: n = A,T,C or G
agtctttaaatattntaatggttcgtgaa

SEQ ID NO: 216 - DNA, 29 bp, Homo sapiens
  misc_feature 15: n = A,T,C or G
tgttcactttcttcnttcaaggagcagtt

SEQ ID NO: 217 - DNA, 29 bp, Homo sapiens
  misc_feature 15: n = A,T,C or G
aagacatatcattcncactataattccaa

SEQ ID NO: 218 - DNA, 29 bp, Homo sapiens
  misc_feature 15: n = A,T,C or G
cagcactttacaagnttcagaaaactcca

SEQ ID NO: 219 - DNA, 29 bp, Homo sapiens
  misc_feature 15: n = A,T,C or G
acctgagtgttgccnatgcggatctactc

SEQ ID NO: 220 - DNA, 29 bp, Homo sapiens
  misc_feature 15: n = A,T,C or G
aggagaaattactgnatgagaacaaatga

SEQ ID NO: 221 - DNA, 29 bp, Homo sapiens
  misc_feature 15: n = A,T,C or G
tccatctacgcacantaaaaaggcattat

SEQ ID NO: 222 - DNA, 29 bp, Homo sapiens
  misc_feature 15: n = A,T,C or G
aactagtatgtttantctatttttcttta

SEQ ID NO: 223 - DNA, 29 bp, Homo sapiens
  misc_feature 15: n = A,T,C or G
tacaaggaagttaanatctagagcgatca

SEQ ID NO: 224 - DNA, 29 bp, Homo sapiens
  misc_feature 15: n = A,T,C or G
ctccctctgtgtaanttccttggaataca

SEQ ID NO: 225 - DNA, 29 bp, Homo sapiens
  misc_feature 15: n = A,T,C or G
gtctaagaacaatgnaaatccattgaaga

SEQ ID NO: 226 - DNA, 29 bp, Homo sapiens
  misc_feature 15: n = A,T,C or G
atttgtagttttgcngataaagaacactt

SEQ ID NO: 227 - DNA, 29 bp, Homo sapiens
  misc_feature 15: n = A,T,C or G
ccaataagttcatcngtgttctaaactat

SEQ ID NO: 228 - DNA, 29 bp, Homo sapiens
  misc_feature 15: n = A,T,C or G
gtatctttgttcacnttgttcatggcttc

SEQ ID NO: 229 - DNA, 29 bp, Homo sapiens
  misc_feature 15: n = A,T,C or G
ctttttaaaaatcanccttaagattgcca

SEQ ID NO: 230 - DNA, 29 bp, Homo sapiens
  misc_feature 15: n = A,T,C or G
agcacaaattctttnctgtatgtggagac

SEQ ID NO: 231 - DNA, 29 bp, Homo sapiens
  misc_feature 15: n = A,T,C or G
gaatctataaattcngcttcatgtcatga

SEQ ID NO: 232 - DNA, 29 bp, Homo sapiens
  misc_feature 15: n = A,T,C or G
ctttatattatctgngttgtcagttttta

SEQ ID NO: 233 - DNA, 29 bp, Homo sapiens
  misc_feature 15: n = A,T,C or G
tcataactctctcgngagtgataacatct

SEQ ID NO: 234 - DNA, 29 bp, Homo sapiens
  misc_feature 15: n = A,T,C or G
gggaaccttgagggnaggataagttgaat

SEQ ID NO: 235 - DNA, 29 bp, Homo sapiens
  misc_feature 15: n = A,T,C or G
gaggtgggagagganctttccattttaga

SEQ ID NO: 236 - DNA, 29 bp, Homo sapiens
  misc_feature 15: n = A,T,C or G
ttacattctaactanccttcaagatccaa

SEQ ID NO: 237 - DNA, 29 bp, Homo sapiens
  misc_feature 15: n = A,T,C or G
aaacatccggaagangcaatggcagctat

SEQ ID NO: 238 - DNA, 29 bp, Homo sapiens
  misc_feature 15: n = A,T,C or G
acagcatacaaaggngtaatatgaagtaa

SEQ ID NO: 239 - DNA, 29 bp, Homo sapiens
  misc_feature 15: n = A,T,C or G
agaaaattgaataanaatggtagctaagc

SEQ ID NO: 240 - DNA, 29 bp, Homo sapiens
  misc_feature 15: n = A,T,C or G
actcctcttccattnacattattgttgaa

SEQ ID NO: 241 - DNA, 29 bp, Homo sapiens
  misc_feature 15: n = A,T,C or G
atagtcaagttgaangaatatacattttt

SEQ ID NO: 242 - DNA, 29 bp, Homo sapiens
  misc_feature 15: n = A,T,C or G
tgtgggaacaactanttgtgcatggaatc

SEQ ID NO: 243 - DNA, 29 bp, Homo sapiens
  misc_feature 15: n = A,T,C or G
ttattcatctattanagaaagtagcaaaa

SEQ ID NO: 244 - DNA, 29 bp, Homo sapiens
  misc_feature 15: n = A,T,C or G
ctgtgcactgggcantgcgctgataggca

SEQ ID NO: 245 - DNA, 29 bp, Homo sapiens
  misc_feature 15: n = A,T,C or G
caggtcaagtctagntagctgtggggcag

SEQ ID NO: 246 - DNA, 29 bp, Homo sapiens
  misc_feature 15: n = A,T,C or G
tcagttcagaatccncagaaaagttagtg

SEQ ID NO: 247 - DNA, 29 bp, Homo sapiens
  misc_feature 15: n = A,T,C or G
ccctggctggttacntagggctacctgtc

SEQ ID NO: 248 - DNA, 29 bp, Homo sapiens
  misc_feature 15: n = A,T,C or G
ggaaaagcttggatncaaaagtaaataat

SEQ ID NO: 249 - DNA, 29 bp, Homo sapiens
  misc_feature 15: n = A,T,C or G
catcttggaaaatangcatttatatgttt

SEQ ID NO: 250 - DNA, 29 bp, Homo sapiens
  misc_feature 15: n = A,T,C or G
agtcagatgtatctnttttttccttttta

SEQ ID NO: 251 - DNA, 29 bp, Homo sapiens
  misc_feature 15: n = A,T,C or G
taactactatgcaanaggaacactgacct

SEQ ID NO: 252 - DNA, 29 bp, Homo sapiens
  misc_feature 15: n = A,T,C or G
aatatggtatagccntacaggtgaattta

SEQ ID NO: 253 - DNA, 29 bp, Homo sapiens
  misc_feature 15: n = A,T,C or G
ttctcctcagatcantattatttcagagg

SEQ ID NO: 254 - DNA, 29 bp, Homo sapiens
  misc_feature 15: n = A,T,C or G
tgcacactttagagnttagaaaatccagg

SEQ ID NO: 255 - DNA, 29 bp, Homo sapiens
  misc_feature 15: n = A,T,C or G
acacattgttactgntcccaccacagaat

SEQ ID NO: 256 - DNA, 29 bp, Homo sapiens
  misc_feature 15: n = A,T,C or G
gctccacaaccctcnggcaatacctaaat

SEQ ID NO: 257 - DNA, 29 bp, Homo sapiens
  misc_feature 15: n = A,T,C or G
aaacagtagttgccngcttcccacgtgca

SEQ ID NO: 258 - DNA, 29 bp, Homo sapiens
  misc_feature 15: n = A,T,C or G
ctctgggttcagccngtctcctgtcattc

SEQ ID NO: 259 - DNA, 29 bp, Homo sapiens
  misc_feature 15: n = A,T,C or G
tgaatttgggctttnctcgattcatgatt

SEQ ID NO: 260 - DNA, 29 bp, Homo sapiens
  misc_feature 15: n = A,T,C or G
attgcttatgagctntggaattaagtggt

SEQ ID NO: 261 - DNA, 29 bp, Homo sapiens
  misc_feature 15: n = A,T,C or G
gtttgtgtaaatacncagtttcctgtatc

SEQ ID NO: 262 - DNA, 29 bp, Homo sapiens
  misc_feature 15: n = A,T,C or G
aatggatacactganggttagtggctcct

SEQ ID NO: 263 - DNA, 29 bp, Homo sapiens
  misc_feature 15: n = A,T,C or G
ttttttgactttttnttgcagattctagc

SEQ ID NO: 264 - DNA, 29 bp, Homo sapiens
  misc_feature 15: n = A,T,C or G
caggaaaccttgtcnaatgcgtgatttta

SEQ ID NO: 265 - DNA, 29 bp, Homo sapiens
  misc_feature 15: n = A,T,C or G
ctctcttttaacaangcagtgctcaagat

SEQ ID NO: 266 - DNA, 29 bp, Homo sapiens
  misc_feature 15: n = A,T,C or G
tactttgtccacagngcactgagctctgg

SEQ ID NO: 267 - DNA, 29 bp, Homo sapiens
  misc_feature 15: n = A,T,C or G
aaaagctacaaattnatgaagtatctagg

SEQ ID NO: 268 - DNA, 29 bp, Homo sapiens
  misc_feature 15: n = A,T,C or G
tgtgtgcattatgcnaagcaaggaatact

SEQ ID NO: 269 - DNA, 29 bp, Homo sapiens
  misc_feature 15: n = A,T,C or G
cgggccataaaaccnagaccgccagaaac

SEQ ID NO: 270 - DNA, 29 bp, Homo sapiens
  misc_feature 15: n = A,T,C or G
acaagcttgttagcngatgagctgggaca

SEQ ID NO: 271 - DNA, 29 bp, Homo sapiens
  misc_feature 15: n = A,T,C or G
tttatttttttgccnataagaaagatccc

SEQ ID NO: 272 - DNA, 29 bp, Homo sapiens
  misc_feature 15: n = A,T,C or G
ttgatacgacttgantacccaaggctgag

SEQ ID NO: 273 - DNA, 29 bp, Homo sapiens
  misc_feature 15: n = A,T,C or G
cacttgtaaccttcngtgattagatccag

SEQ ID NO: 274 - DNA, 29 bp, Homo sapiens
  misc_feature 15: n = A,T,C or G
gaaatgctaaatggngaaagcaatctgag

SEQ ID NO: 275 - DNA, 29 bp, Homo sapiens
  misc_feature 15: n = A,T,C or G
ctggcggtctgttcncgtcaacatttaga

SEQ ID NO: 276 - DNA, 29 bp, Homo sapiens
  misc_feature 15: n = A,T,C or G
ttggaatccaaagcntgtctcttttgaga

SEQ ID NO: 277 - DNA, 29 bp, Homo sapiens
  misc_feature 15: n = A,T,C or G
ttcaacactgtcccntatctttctatact

SEQ ID NO: 278 - DNA, 29 bp, Homo sapiens
  misc_feature 15: n = A,T,C or G
actcaaagccaagtnttagactagcagaa

SEQ ID NO: 279 - DNA, 29 bp, Homo sapiens
  misc_feature 15: n = A,T,C or G
ataacataaaaagtnttcattcactcgct

SEQ ID NO: 280 - DNA, 29 bp, Homo sapiens
  misc_feature 15: n = A,T,C or G
tacaaatggtcacanaacttaccctacac

SEQ ID NO: 281 - DNA, 29 bp, Homo sapiens
  misc_feature 15: n = A,T,C or G
gagagcaatgcttangtgatgcaaatgga

SEQ ID NO: 282 - DNA, 29 bp, Homo sapiens
  misc_feature 15: n = A,T,C or G
ctggaatcaaggtcnccttcttggtcttt

SEQ ID NO: 283 - DNA, 29 bp, Homo sapiens
  misc_feature 15: n = A,T,C or G
attccctcactccancccaagggcaattt

SEQ ID NO: 284 - DNA, 29 bp, Homo sapiens
  misc_feature 15: n = A,T,C or G
ccagagattacagcntgaagggttttgag

SEQ ID NO: 285 - DNA, 29 bp, Homo sapiens
  misc_feature 15: n = A,T,C or G
taaaatcctacatanctcccttgggcact

SEQ ID NO: 286 - DNA, 29 bp, Homo sapiens
  misc_feature 15: n = A,T,C or G
ctctaggagcccctngcccttgcagccca

SEQ ID NO: 287 - DNA, 29 bp, Homo sapiens
  misc_feature 15: n = A,T,C or G
aagatgaggccccanggttttggaatgct

SEQ ID NO: 288 - DNA, 29 bp, Homo sapiens
  misc_feature 15: n = A,T,C or G
ctacacaaaaattantcacttgggcaggg

SEQ ID NO: 289 - DNA, 29 bp, Homo sapiens
  misc_feature 15: n = A,T,C or G
aagaatactatcttnttttctcaccacag

SEQ ID NO: 290 - DNA, 29 bp, Homo sapiens
  misc_feature 15: n = A,T,C or G
gcaacttcattggantagacaagacatga

SEQ ID NO: 291 - DNA, 29 bp, Homo sapiens
  misc_feature 15: n = A,T,C or G
cagcggaattagacncaggactttggttt

SEQ ID NO: 292 - DNA, 29 bp, Homo sapiens
  misc_feature 15: n = A,T,C or G
aagaagaagattttntagttctgtttatg

SEQ ID NO: 293 - DNA, 29 bp, Homo sapiens
  misc_feature 15: n = A,T,C or G
agtccagttcagagntgatgccaggatta

SEQ ID NO: 294 - DNA, 29 bp, Homo sapiens
  misc_feature 15: n = A,T,C or G
aataatggatgttancacttaagcctctg

SEQ ID NO: 295 - DNA, 29 bp, Homo sapiens
  misc_feature 15: n = A,T,C or G
gtccaactttcccantctaccccaactca

SEQ ID NO: 296 - DNA, 29 bp, Homo sapiens
  misc_feature 15: n = A,T,C or G
ctaacattttgttgnttctaccaccttta

SEQ ID NO: 297 - DNA, 29 bp, Homo sapiens
  misc_feature 15: n = A,T,C or G
ggataacagtggganggtgaggcaaaagc

SEQ ID NO: 298 - DNA, 29 bp, Homo sapiens
  misc_feature 15: n = A,T,C or G
caaaatatgagctcntggtctaactacat

SEQ ID NO: 299 - DNA, 29 bp, Homo sapiens
  misc_feature 15: n = A,T,C or G
tgcccatgttctgantttatcaggccagc

SEQ ID NO: 300 - DNA, 29 bp, Homo sapiens
  misc_feature 15: n = A,T,C or G
atcttgttttggcancttgatgactacat

SEQ ID NO: 301 - DNA, 29 bp, Homo sapiens
  misc_feature 15: n = A,T,C or G
gcacaggggactccngacagatgtgatat

SEQ ID NO: 302 - DNA, 29 bp, Homo sapiens
  misc_feature 15: n = A,T,C or G
atatcatttttcctntttacttgtacttt

SEQ ID NO: 303 - DNA, 29 bp, Homo sapiens
  misc_feature 15: n = A,T,C or G
tcattgacacagttnacatgccagggtca

SEQ ID NO: 304 - DNA, 29 bp, Homo sapiens
  misc_feature 15: n = A,T,C or G
atctagcagcatgantcatcagctctggt

SEQ ID NO: 305 - DNA, 29 bp, Homo sapiens
  misc_feature 15: n = A,T,C or G
tccgatttgcagttntagttcgactaaat

SEQ ID NO: 306 - DNA, 29 bp, Homo sapiens
  misc_feature 15: n = A,T,C or G
tttctataaaagttngtgatacaatgatg

SEQ ID NO: 307 - DNA, 29 bp, Homo sapiens
  misc_feature 15: n = A,T,C or G
ttagacaccctctcngtggggcaaaattg

SEQ ID NO: 308 - DNA, 29 bp, Homo sapiens
  misc_feature 15: n = A,T,C or G
actacttggacagtnacctgaacatctca

SEQ ID NO: 309 - DNA, 29 bp, Homo sapiens
  misc_feature 15: n = A,T,C or G
gtggttgaaccttgnagaaatgtgttaga

SEQ ID NO: 310 - DNA, 29 bp, Homo sapiens
  misc_feature 15: n = A,T,C or G
agggacacattcagnacccaataactgta

SEQ ID NO: 311 - DNA, 29 bp, Homo sapiens
  misc_feature 15: n = A,T,C or G
aggagaagacttgcntgcccaggcttgct

SEQ ID NO: 312 - DNA, 29 bp, Homo sapiens
  misc_feature 15: n = A,T,C or G
aatataaaactaaangagatgaacattgg

SEQ ID NO: 313 - DNA, 29 bp, Homo sapiens
  misc_feature 15: n = A,T,C or G
tcaagaaaagatgangtttgcattctcta

SEQ ID NO: 314 - DNA, 29 bp, Homo sapiens
  misc_feature 15: n = A,T,C or G
gaacgtagtactccntttgactttgagaa

SEQ ID NO: 315 - DNA, 29 bp, Homo sapiens
  misc_feature 15: n = A,T,C or G
ttggtgcatttagtncaaacagctcccaa

SEQ ID NO: 316 - DNA, 29 bp, Homo sapiens
  misc_feature 15: n = A,T,C or G
gagattatttgtaancacagtgtttcatg

SEQ ID NO: 317 - DNA, 29 bp, Homo sapiens
  misc_feature 15: n = A,T,C or G
atgaatagaagcatntttgtgtctacaac

SEQ ID NO: 318 - DNA, 29 bp, Homo sapiens
  misc_feature 15: n = A,T,C or G
cttatatttaagacngcttagatttttta

SEQ ID NO: 319 - DNA, 29 bp, Homo sapiens
  misc_feature 15: n = A,T,C or G
aaacttcataaagcnaggaaagaagataa

SEQ ID NO: 320 - DNA, 29 bp, Homo sapiens
  misc_feature 15: n = A,T,C or G
tcacctcctcggctntcctttttgtgtta

SEQ ID NO: 321 - DNA, 29 bp, Homo sapiens
  misc_feature 15: n = A,T,C or G
ctttatgcaggattnagttttacaggata

SEQ ID NO: 322 - DNA, 29 bp, Homo sapiens
  misc_feature 15: n = A,T,C or G
tcagtacttttaagncaatgcaactttaa

SEQ ID NO: 323 - DNA, 29 bp, Homo sapiens
  misc_feature 15: n = A,T,C or G
cttacctggtggctngttcgtggaattta

SEQ ID NO: 324 - DNA, 29 bp, Homo sapiens
  misc_feature 15: n = A,T,C or G
tcctggactggcttnactgtactctccca

SEQ ID NO: 325 - DNA, 29 bp, Homo sapiens
  misc_feature 15: n = A,T,C or G
ttaaaaaaaatcttntgtggttggctatc

SEQ ID NO: 326 - DNA, 29 bp, Homo sapiens
  misc_feature 15: n = A,T,C or G
gacgacagatgtcanaaaacataaaagta

SEQ ID NO: 327 - DNA, 29 bp, Homo sapiens
  misc_feature 15: n = A,T,C or G
agttctgaagatttnctttgagtttttaa

SEQ ID NO: 328 - DNA, 29 bp, Homo sapiens
  misc_feature 15: n = A,T,C or G
gggctcacaacgggnggtcatggttgcgg

SEQ ID NO: 329 - DNA, 29 bp, Homo sapiens
  misc_feature 15: n = A,T,C or G
ctagataggggaacngagcagctaaatga

SEQ ID NO: 330 - DNA, 29 bp, Homo sapiens
  misc_feature 15: n = A,T,C or G
agaccatgcctgatnggtgttttacacat

SEQ ID NO: 331 - DNA, 29 bp, Homo sapiens
  misc_feature 15: n = A,T,C or G
agccagggaagccanccatccaagaggga

SEQ ID NO: 332 - DNA, 29 bp, Homo sapiens
  misc_feature 15: n = A,T,C or G
gcaaaaaccatagcnttattgggcttggg

SEQ ID NO: 333 - DNA, 29 bp, Homo sapiens
  misc_feature 15: n = A,T,C or G
aatacgatggtgacntttcaaaaatctgg

SEQ ID NO: 334 - DNA, 29 bp, Homo sapiens
  misc_feature 15: n = A,T,C or G
ctttttcaggcttgnaaatgctcatgcta

SEQ ID NO: 335 - DNA, 29 bp, Homo sapiens
  misc_feature 15: n = A,T,C or G
attttaatctggttncacatttgtcgtca

SEQ ID NO: 336 - DNA, 29 bp, Homo sapiens
  misc_feature 15: n = A,T,C or G
aaaagagaaaattgnaaaaagtaggtgag

SEQ ID NO: 337 - DNA, 29 bp, Homo sapiens
  misc_feature 15: n = A,T,C or G
tacattctttgggtntgaacatagtttta

SEQ ID NO: 338 - DNA, 29 bp, Homo sapiens
  misc_feature 15: n = A,T,C or G
taactctgataggtnatgaggagccaacc

SEQ ID NO: 339 - DNA, 29 bp, Homo sapiens
  misc_feature 15: n = A,T,C or G
tctgatcgtaaaacngtggacgctgagca

SEQ ID NO: 340 - DNA, 29 bp, Homo sapiens
  misc_feature 15: n = A,T,C or G
ttaaaatatatcaangtatctgcagtccg

SEQ ID NO: 341 - DNA, 29 bp, Homo sapiens
  misc_feature 15: n = A,T,C or G
gattgaacaggactntttgttaattctac

SEQ ID NO: 342 - DNA, 29 bp, Homo sapiens
  misc_feature 15: n = A,T,C or G
aacatcatttttacngttattcttaagat

SEQ ID NO: 343 - DNA, 29 bp, Homo sapiens
  misc_feature 15: n = A,T,C or G
acatttcattgcagngataagggataggg

SEQ ID NO: 344 - DNA, 29 bp, Homo sapiens
  misc_feature 15: n = A,T,C or G
cagcaatgaggcaantaaaatgcacttga

SEQ ID NO: 345 - DNA, 29 bp, Homo sapiens
  misc_feature 15: n = A,T,C or G
tcaaggtcgatatantgatttctgaacaa